(19) United States
(12) Patent Application Publication
(10) Pub. No.: US 2022/0218752 A1
(43) Pub. Date: Jul. 14, 2022

(54) LOCKR-MEDIATED RECRUITMENT OF CAR T CELLS

(71) Applicants: University of Washington, Seattle, WA (US); FRED HUTCHINSON CANCER RESEARCH CENTER, Seattle, WA (US)

(72) Inventors: David BAKER, Seattle, WA (US); Scott BOYKEN, Seattle, WA (US); Marc Joseph LAJOIE, Seattle, WA (US); Robert A. LANGAN, Seattle, WA (US); Stanley R. RIDDELL, Seattle, WA (US); Alexander SALTER, Seattle, WA (US)

(21) Appl. No.: 17/610,083

(22) PCT Filed: May 18, 2020
(86) PCT No.: PCT/US2020/033463

§ 371 Date: Nov. 9, 2021

Related U.S. Application Data
(60) Provisional application No. 62/964,024, filed on Jan. 21, 2020.
(60) Provisional application No. 62/848,840, filed on May 16, 2019.

(51) Int. Cl. A61K 35/17; C12N 15/86; C12N 5/0783; C12N 15/62; A61K 38/17; C07K 14/47; C07K 14/725; C07K 14/705; C07K 14/005

(52) U.S. Cl. CPC A61K 35/17; C12N 15/86; C12N 5/0636; C12N 15/625; A61K 38/1774; C07K 14/4747; C07K 2319/30; C07K 14/70596; C07K 14/005; C12N 2740/15043; C12N 2800/107; C12N 2510/00; C12N 2501/2302; C07K 14/7051

(57) ABSTRACT

Disclosed are protein switches that can sequester bioactive peptides and/or binding domains, holding them in an inactive (“off”) state, until combined with a second designed polypeptide called the key, which induces a conformational change that activates (“on”) the bioactive peptide or binding domain only when the protein switch components are co/localized when bound to their targets, components of such protein switches, and their use.

### CROSS REFERENCE

This application claims priority to U.S. Provisional Patent Application Ser. Nos. 62/848,840 filed May 16, 2019 and 62/964,024 filed Jan. 21, 2020, each incorporated by reference herein in its entirety.

### FEDERAL FUNDING STATEMENT

This invention was made with government support under Grant No. CHE-1629214 awarded by the National Science Foundation, Grant No. HDTRA1-18-1-0001 awarded by the Defense Threat Reduction Agency, and Grant No. R01 CA114536 awarded by the National Institutes of Health. The government has certain rights in the invention.

### REFERENCE TO SEQUENCE LISTING SUBMITTED ELECTRONICALLY VIA EFS-WEB

This application contains a Sequence Listing submitted as an electronic text file named “19-852-PCT_Sequence-Listing_ST25.txt”, having a size in bytes of 32 MB, and created on May 14, 2020. The information contained in this electronic file is hereby incorporated by reference in its entirety pursuant to 37 CFR § 1.52(e)(5).

### BACKGROUND

Biology is adept at integrating multiple signals to control function; however, natural systems are highly evolved for specific functions that make them difficult to repurpose. Engineering systems that can integrate combinations of binding events and predictively respond remains an outstanding challenge. Such a system would be particularly useful for targeting cells based on recognition of a combination of surface markers: most mammalian cell types differ from other tissues only in the combinations of markers present on their surfaces.

### SUMMARY

In one aspect, the disclosure provides methods of increasing selectivity of a cell for a chimeric antigen receptor (CAR) T cell therapy comprising

(a) contacting cells with a first cage polypeptide fused to a first binding domain, wherein the first cage polypeptide comprises (i) a structural region and (ii) a latch region further comprising one or more bioactive peptides, wherein the structural region interacts with the latch region to prevent activity of the one or more bioactive peptides in the absence of colocalization with a key polypeptide and wherein the first binding domain is capable of binding to a first cell moiety present on or within a cell; and

(b) contacting the cell with a first key polypeptide fused to a second binding domain, wherein upon colocalization with the first cage polypeptide, the first key polypeptide is capable of binding to the cage structural region to activate the one or more bioactive peptides, wherein the second binding domain is capable of binding to a second cell moiety present on or within the cell,

wherein the first cell moiety and the second cell moiety are different or the same.

In another aspect, the disclosure provides methods of increasing selectivity of cells that are interacting with each other for a chimeric antigen receptor T cell therapy comprising:

(a) contacting two or more cells with a first cage polypeptide fused to a first binding domain, wherein the first cage polypeptide comprises (i) a structural region and (ii) a latch region further comprising one or more bioactive peptides, wherein the structural region interacts with the latch region to prevent activity of the one or more bioactive peptides in the absence of colocalization with a key polypeptide and wherein the first binding domain is capable of binding to a first cell moiety present on a synapse between the two or more cells; and

(b) contacting the two or more cells with a first key polypeptide fused to a second binding domain, wherein upon colocalization with the first cage polypeptide, the first key polypeptide is capable of binding to the cage structural region to activate the one or more bioactive peptides, wherein the second binding domain is capable of binding to a second cell moiety present on the synapse between the two or more cells,

wherein the first cell surface moiety and the second cell surface moiety are the same or different.

In a further aspect, the disclosure provides methods of targeting heterogeneous cells (more than two different cell types) for a chimeric antigen receptor T cell therapy, wherein a first cell moiety and a second cell moeity are present on the first cell and a first cell moiety and a third cell moiety are present on the second cell, comprising, comprising:

(a) contacting two or more cells with a first cage polypeptide fused to a first binding domain, wherein the first cage polypeptide comprises (i) a structural region and (ii) a latch region further comprising one or more bioactive peptides, and wherein the structural region interacts with the latch region to prevent activity of the one or more bioactive peptides in the absence of colocalization with a key polypeptide and wherein the first binding domain is capable of binding to a first cell moiety present on or within the two or more cells;

(b) contacting the two or more cells with a first key polypeptide fused to a second binding domain, wherein upon colocalization, the first key polypeptide is capable of binding to the cage structural region to activate the one or more bioactive peptides and wherein the second binding domain is capable of binding to a second cell moiety present on a cell that also comprises the first cell moiety, and

(c) contacting the two or more cells with a second key polypeptide fused to a third binding domain, wherein upon colocalization, the second key polypeptide is capable of binding to the cage structural region to activate the one or more bioactive peptides and wherein the third binding domain is capable of binding to a third cell moiety in a cell that comprises the first cell moiety,

wherein the first cell moiety, the second cell moiety, and the third cell moiety are different and the cell that comprises the second cell moiety and the cell that comprises the third cell moiety are different.

In one aspect, the disclosure provides methods of reducing off-target activity for a chimeric antigen receptor T cell therapy comprising

(a) contacting two or more cells with a first cage polypeptide fused to a first binding domain, wherein the first cage polypeptide comprises (i) a structural region and (ii) a latch region further comprising one or more bioactive peptides, and wherein the structural region interacts with the latch region to prevent activity of the one or more bioactive peptides in the absence of colocalization with a key polypeptide and wherein the first binding domain is capable of binding to a first cell moiety present on a cell;

(b) contacting the two or more cells with a first key polypeptide fused to a second binding domain, wherein upon colocalization, the first key polypeptide is capable of binding to the cage structural region to activate the one or more bioactive peptides and wherein the second binding domain is capable of binding to a second cell moiety present on a cell that also comprises the first cell moiety, and

(c) contacting the two or more cells with a decoy cage polypeptide fused to a third binding domain, wherein the decoy cage polypeptide comprises a decoy structural region, which upon colocalization with the key polypeptide and the first cage polypeptide, is capable of preferentially binding to the first key polypeptide and wherein the third binding domain is capable of binding to a third cell moiety in a cell that comprises the first cell moiety and the second cell moiety.

In another aspect, the disclosure provides protein complexes comprising (i) a first cage polypeptide fused to a first binding domain and (ii) a first key polypeptide fused to a second binding domain, wherein the first cage polypeptide comprises (i) a structural region and (ii) a latch region further comprising one or more bioactive peptides, wherein the first key polypeptide binds to the cage structural region, wherein the one or more bioactive peptides are activated, and wherein the first binding domain binds to a first cell moiety present on or within a cell or on a synapse of two interacting cells and the second binding domain binds to a second cell moiety present on or within the cell or on a synapse of the two interacting cells, wherein the first cell moiety and the second cell moiety are different or the same.

In a further aspect, the disclosure provides protein complexes comprising (i) a first key polypeptide fused to a first binding domain and (ii) a decoy cage polypeptide fused to a second binding domain, wherein the first key polypeptide binds to the decoy cage polypeptide, and wherein the first binding domain binds to a first cell moiety present on or within a cell or on a synapse of two interacting cells and the second binding domain binds to a second cell moiety present on or within the cell or on a synapse of the two interacting cells, wherein the first cell moiety and the second cell moiety are different or the same.

In one aspect, the disclosure provides compositions comprising

(a) a first cage polypeptide fused to a first binding domain or a polynucleotide encoding the same, wherein the first cage polypeptide comprises (i) a structural region and (ii) a latch region further comprising one or more bioactive peptides, wherein the structural region interacts with the latch region to prevent activity of the one or more bioactive peptides in the absence of colocalization with a key polypeptide and wherein the first binding domain is capable of binding to a first cell moiety present on or within a cell; and

(b) a first key polypeptide fused to a second binding domain or a polynucleotide encoding the same, wherein upon colocalization with the first cage polypeptide, the first key polypeptide is capable of binding to the cage structural region to activate the one or more bioactive peptides, wherein the second binding domain is capable of binding to a second cell moiety present on or within the cell,

wherein the first cell moiety and the second cell moiety are different or the same and wherein the cell is a target for a chimeric antigen receptor (CAR) T cell therapy.

In another aspect, the disclosure provides compositions comprising

(a) a first cage polypeptide comprising (i) a structural region, (ii) a latch region further comprising one or more bioactive peptides, and (iii) a first binding domain wherein the structural region interacts with the latch region to prevent activity of the one or more bioactive peptides;

(b) a first key polypeptide capable of binding to the cage structural region to activate the one or more bioactive peptides, wherein the key polypeptide comprises a second binding domain, wherein the first binding domain and the second binding domain bind to (i) different moieties on the surface of the same cell, (ii) the same moiety on the surface of the same cell, (iii) different moieties at the synapse between two cells that are in contact, or (iv) the same moiety at the synapse between two cells that are in contact; and

(c) cells comprising one or more chimeric antigen receptor(s) that bind to the one or more bioactive peptides when the one or more bioactive peptides are activated.

In a further aspect, the disclosure provides compositions comprising

(a) one or more expression vectors encoding and/or cells expressing:
(i) a first cage polypeptide comprising (i) a structural region, (ii) a latch region further comprising one or more bioactive peptides, and (iii) a first binding domain wherein the structural region interacts with the latch region to prevent activity of the one or more bioactive peptides; and
(ii) a first key polypeptide capable of binding to the cage structural region to activate the one or more bioactive peptides, wherein the key polypeptide comprises a second binding domain,

wherein the first binding domain and the second binding domain bind to (i) different moieties on the surface of the same cell, (ii) the same moiety on the surface of the same cell, (iii) different moieties at the synapse between two cells that are in contact, or (iv) the same moiety at the synapse between two cells that are in contact; and
(b) (i) cells comprising one or more chimeric antigen receptor(s) that bind to the one or more bioactive peptides when the one or more bioactive peptides are activated; and/or (ii) one or more fusion protein, nucleic acid, vector, and/or the cell of the disclosure.

In one aspect, the disclosure provides methods for cell targeting, comprising

(a) contacting a biological sample containing cells with
(i) a cage polypeptide comprising (i) a structural region, (ii) a latch region further comprising one or more bioactive peptides, and (iii) a first binding domain that targets a cell of interest, wherein the structural region interacts with the latch region to prevent activity of the one or more bioactive peptides; and
(ii) a key polypeptide comprising a second binding domain that targets the cell of interest, wherein the first binding domain and the second binding domain bind to (i) different moieties on the surface of the same cell, (ii) the same moiety on the surface of the same cell, (iii) different moieties at the synapse between two cells that are in contact, or (iv) the same moiety at the synapse between two cells that are in contact;

wherein the contacting occurs for a time and under conditions to promote binding of the cage polypeptide and the key polypeptide to the cell of interest, and to promote binding of the key polypeptide to the cage structural region to displace the latch region and activate the one or more bioactive peptides only when the cage polypeptide and the key polypeptide are co-localized to the cell of interest;

(b) contacting the biological sample with one or more effector molecule(s) under conditions to promote binding of the one or more effector molecules selected from the fusion proteins, nucleic acids, vectors, and/or cells of the disclosure under conditions to promote binding of the one or more effector molecules to the one or more activated bioactive peptides to produce an effector molecule-bioactive peptide complex; and

(c) optionally detecting the effector molecule-bioactive peptide complex, wherein the effector molecule-bioactive peptide complex provides a measure of the cell of interest in the biological sample.

In another aspect, the disclosure provides fusion proteins comprising:

(a) an extracellular binding domain;

(b) a transmembrane domain;

(c) an intracellular signaling component; and

(d) optionally, a selection marker.

### DESCRIPTION OF THE FIGURES

FIG. 1a-g. A de novo designed protein switch performs AND logic on the cell surface. a. The ability to compute logic operations on the surface of cells could increase targeting selectivity, provide flexibility for heterogeneous tissue, and avoid healthy tissue. b. Structure of new Cage design used to create Co-LOCKR; the x-ray crystal structure (white) matches the computational design model (green) with 1.1 Å RMSD across all backbone atoms. Cross-sections illustrate asymmetric packing of hydrophobic residues (red square) and an asymmetric hydrogen bond network (blue square). c. Schematic of colocalization-dependent protein switches tuned such that Cage and Key do not interact in solution but strongly interact when colocalized on a surface. Co-LOCKR subunits bind to a surface via a targeting domain. d. Flow cytometry discriminates Her2⁺/EGFR⁺ cells in a mixed population of K562 cells expressing Her2-eGFP, EGFR-iRFP, both, or neither. e. Schematic depicting ‘AND’ logic in which recruitment of an Effector protein occurs when Cage and Key are colocalized on the surface of the same cell. f. The mixed population of K562 cells from FIG. 1c was incubated with 111 nM Her2-targeted Cage, 111 nM EGFR-targeted Key, and 50 nM Bcl2-AF594. Bcl2 binding was only observed for the K562/Her2/EGFR cells. g. The mixed population of K562 cells from FIG. 1c was incubated with a dilution series of Her2-targeted Cage and EGFR-targeted Key. In addition, 50 nM Bcl2-AF594 was either co-incubated with Co-LOCKR (solid lines) or added after washing the cells (dashed lines). The gray shaded region of the plot represents colocalization-independent activation in which excess amounts of Cage and Key outcompete Cage-Key-Bcl2 complexes (formed in solution) from binding to the target cells. Bcl2 binding is reported relative to K562 cells incubated with 3000 nM Her2-targeted Cage, 3000 nM EGFR-targeted Key, and 50 nM Bcl2-AF594.

FIG. 2a-d. Tuning Co-LOCKR sensitivity. a. Design model of Co-LOCKR with the Bim functional peptide in yellow. Three buried hydrophobic amino acids were mutated to either Ala or Ser to weaken the Cage-Latch affinity, thereby favoring Cage-Key binding. b. Tuned Co-LOCKR variants exhibit greater colocalization-dependent activation than the unmutated parental variant. CL_C_{H}K_{E} variants recruiting Bcl2-AF594 were evaluated by flow cytometry using the mixed population of K562 cells from FIG. 1c. The data shown represent 12.3 nM CL_C_{H}K_{E}, and FIG. 9c shows the complete dilution series for each variant. c. Confocal microscopy of HEK293T cell lines shows that Co-LOCKR switches recruit Bcl2-AF680 Effector proteins only where Her2 and EGFR are colocalized. Each cell line was incubated with CL_C_{H}K_{E} (I269S Cage) and Bcl2-AF680 before imaging. NucBlue™ is a nuclear stain, eGFP indicates Her2 localization, mCherry™ indicates EGFR localization, AF680 indicates Bcl2 binding in response to Co-LOCKR activation, and white indicates the intersection of Her2-eGFP and EGFR-mCherry™ signal. Scale bars are 10 Uncropped versions of these images are included in FIG. 21a-c. d. Heat map showing the intensity of AF680 signal (Co-LOCKR activation) versus eGFP (Her2) and mCherry™ (EGFR) pixel intensity. Calculations were based on the uncropped 293T/Her2/EGFR image in FIG. 21a.

FIG. 3a-d. Co-LOCKR performs 2- and 3-input logic operations in mixed cell populations. a. Co-LOCKR was used to recruit Bcl2-AF594 for two populations of K562 cells expressing different combinations of Her2, EGFR, and EpCAM. Marker expression for each cell line and identity of the Cage and Key targeting domains are indicated below each bar plot. Red highlighting indicates the expected magnitude of Bcl2-AF594 signal based on relative antigen expression. b. Schematic of [Her2 AND either EGFR OR EpCAM] logic mechanism. c. [Ag₁ AND either Ag₂ OR Ag₃] logic combinations were used to recruit Bcl2-AF594. d. Schematic of [Her2 AND EpCAM NOT EGFR] logic mechanism. The Decoy acts as a sponge to sequester the Key, thereby preventing Cage activation. e. CL_C_{H}K_{Ep}D_{E} was used to recruit Bcl2-AF594. The parental Cage (left) was compared to the I287A Cage (right). The magnitude of signal for CL_C_{H}K_{Ep}D_{E} is reduced compared to the CL_C_{H}K_{Ep} likely because the Decoy competes for Key binding in solution; however, adequate signal remains to compute [Her2 AND EpCAM NOT EGFR] logic. For all panels, population 1 was [K562/EpCAM^{lo}, K562/EGFR/EpCAM^{lo}, K562/EpCAM^{lo}/Her2, and K562/EGFR/EpCAM^{lo}/Her2], and population 2 was [K562/EpCAM^{lo}, K562/EGFR/EpCAM^{lo}, K562/EpCAM^{hi}/Her2, and K562/EGFR/EpCAM^{hi}/Her2]. Error bars represent SEM of 6 independent replicates for K562 and K562/EGFR and 3 independent replicates for all others.

FIG. 4a-i. Co-LOCKR directs CAR T cell specificity using 2- and 3-input logic operations. a,d,g. Mean IFN-γ concentration in cell supernatants 24 hours after co-culture of Cage, Key, and K562 cells with CAR T cells. Marker expression for each cell line and identity of the Cage and Key targeting domains are indicated below each bar plot. Red highlighting indicates the expected magnitude of signal based on the target cell's relative antigen expression. Error bars represent SEM of n=4 (a) or 3 (d,g) healthy T cell donors. AND/NOT logic is demonstrated with EpCAM^{lo} target K562 cells because T cell effector function was leaky for EpCAM^{hi} target cells (see FIG. 515a). b,e,h. CAR T cell proliferation in response to [Her2 AND EpCAM] (c), [Her2 AND EGFR OR EpCAM] (e), or [Her2 AND EpCAM NOT EGER] (h) logic. Bar plots are the percent of T cells that have undergone at least one cell division by 72 hours after co-culture of CAR T cells, Cage, Key, and target K562 cells. Histograms show flow cytometric analysis of CFSE dye dilution gated on CD8⁺ lymphocytes. The data are representative of n=3 biological replicates with healthy T cell donors. c,f,i. CAR T cell cytotoxicity against mixed populations of target Raji cells expressing combinations of Her2, EpCAM, and EGFR. Line graphs show mean frequency of Raji target cells after 0 or 48 hours of co-culture with CAR T cells. n=4 (c,f) or 3 (1) healthy donors. Arrows indicate cell lines targeted by Co-LOCKR.

FIG. 5a-c. Computational design of Co-LOCKR. a. Overview of how LOCKRa was designed in Langan et al. (9). An existing homotrimer (10) was connected into a single polypeptide chain, and the Cage/Latch interface was tuned so that Key binding would induce activation. b. Computational design of Co-LOCKR. All side chains were removed from the LOCKRa backbone except for the residues involved in the existing hydrogen bond networks and the Cage-Latch interface. A new Rosetta design run searched for asymmetric hydrogen bond networks and then asymmetrically designed the core and surface residues. The resulting helical bundle was shortened so as to reduce aggregation, and the Cage-Latch and Cage-Key interfaces were tuned to achieve colocalization dependence. Decoys were created by redesigning the Co-LOCKR Cage to remove the Bim functional peptide and tuning their affinity for the Key. c. Cross-sections of LOCKRa and Co-LOCKR showing core redesign to replace C3 symmetric hydrophobic packing with a new hydrogen bond network (left) or asymmetric hydrophobic packing (right). d. LOCKRa and Co-LOCKR share 60.8% sequence identity (pairwise sequence identity performed using Geneious software, global alignment with free end gaps).

FIG. 6. Redesign of LOCKR Cage reduces aggregation. The Langan et al. (9) LOCKRa Cage and asymLOCKR (top) and three new variants of the Co-LOCKR Cage (bottom) with 0, 7, or 10 residues deleted from the C-terminus of their latch were evaluated by Size Exclusion Chromatography using a Superdex™ 75 Increase 10/300 GL column (GE).

FIG. 7a-c. The Co-LOCKR system is controlled by a thermodynamic mechanism based on reversible protein-protein interactions. Co-localizing Cage and Key on the same surface results in a large increase in local concentration, shifting the binding equilibrium. According to the thermodynamic mechanism, a complex can form in solution (a) or on a surface (b). Our flow cytometry data shows that any pre-complexed Co-LOCKR that occurs in solution does not lead to appreciable staining of single-antigen target cells. c. Colocalization shifts the response curve to the left so that activation can occur at lower concentrations of Co-LOCKR proteins.

FIG. 8a-b. The strengths of Cages and Decoys can be tuned by modulating the Cage-Latch, Cage-Key, Decoy-Latch, and Decoy-Key interfaces. Residues involved in the Cage-Latch and Cage-Key interface are colored orange. Bim is shown in magenta. We rationally reduced the affinity of these interfaces by replacing large hydrophobic amino acids with small hydrobophic amino acids or serine. a. Side view of the Cage in an ‘off’ conformation. b. Side view of the Key. c. Cross-section of the Cage in an ‘off’ conformation.

FIG. 9a-e. Mutations in the Cage-Latch interface can predictably tune the sensitivity of Co-LOCKR switches. a. Design model of Co-LOCKR with the Bim functional peptide in yellow. Three buried hydrophobic amino acids were mutated to either Ala or Ser to weaken the Cage-Latch affinity, thereby favoring Cage-Key binding. This panel is reproduced from FIG. 2a. b. Colocalization-independent activation was evaluated using biolayer interferometry (Octet). A dilution series of CL_C_{H}K_{E} was evaluated for binding to biotinylated Bcl2 immobilized on a streptavidin Octet tip. More disruptive mutations increased the sensitivity of the switch. c. Tuned Co-LOCKR variants exhibit greater colocalization-dependent activation sensitivity and responsiveness than the parental Co-LOCKR variant. Dilution series of CL_C_{H}K_{E} variants were evaluated by flow cytometry using the mixed population of K562 cells from FIG. 1c. Bcl2-AF594 was recruited to K562/Her2/EGFR cells (solid lines), with minimal binding to K562, K562/Her2, and K562/EGFR cells (dotted lines represent maximum off-target binding signal). More disruptive mutations increased the sensitivity of the switch, and the I269S variant exhibited the greatest switch activation. On-target binding peaked at ˜37 nM for the parental variant and ˜12 nM for the mutated variants. d. Switch activation of the I269S variant was enhanced for low CL_C_{H}K_{E} concentrations by incubating cells in larger volumes prior to flow cytometry. e. On-target but not off-target switch activation increased when 2 nM of the CL_C_{H}K_{E} I269S variant was incubated with target cells in larger incubation volumes.

FIG. 10a-c. Co-LOCKR variants were evaluated for colocalization-dependent activation in a mixed population of K562 cells expressing Her2-eGFP, EGFR-iRFP, both, or neither. Co-LOCKR Cage variants and Keys were mixed, serially diluted, and evaluated for on-target activation (a), off-target activation (b), and specificity (on-target/max off-target, c) as measured by Bcl2-AF594 binding. Variant 1269S had the highest on-target activation, the parental Cage had the lowest off-target activation, and variant I287A had the best fold targeting specificity. On-target binding peaked at ˜37 nM Cage and Key for the parental variant and ˜12 nM Cage and Key for the tuned variants. Each bar represents a single data point.

FIG. 11a-b. Expression levels of EGFR, EpCAM, and Her2 on K562 and Raji tumor cells. Flow cytometric analysis of EGFR (red), EpCAM (blue), and Her2 (green) expression on the indicated K562 (a) or Raji (b) cell lines. All antibodies were used in the PE channel to permit quantitation of the number of surface molecules using Quantibrite beads.

FIG. 12a-c. Co-LOCKR ‘AND’ logic distinguishes cancer cell lines based on their combinations of surface antigens. a. Targeting domains directly fused to Bim were used to measure relative expression of Her2, EGFR, and EpCAM based on Bcl2-AF594. b. Co-LOCKR distinguished A431 (Her2^{low}/EGFR^{high}/EpCAM^{low}) and SKBR3 (Her2^{high}/EGFR^{low}/EpCAM^{low}) based on their endogenous levels of antigen expression. K562/Her2/EGFR/EpCAM^{KO} cells were used as a specificity control. Co-LOCKR activation was measured by Bcl2-AF594 recruitment. c. Consistent with a stoichiometric mechanism of activation, Co-LOCKR signal is limited by amount of lesser-expressed surface antigen. Furthermore, activation signal is higher when one of the antigens is expressed at high levels compared to when both antigens are expressed at low levels. This suggests that Co-LOCKR can act as a thresholding gate to avoid cells with low antigen expression. Indeed, this may account for the preferential targeting of K562 cells expressing high levels of EpCAM in FIG. 3a. The vertical axis is Bcl2-AF594 recruitment by Co-LOCKR, and the horizontal axis is Bcl2-AF594 recruitment by Bim-DARPin targeted to the lesser-expressed antigen in the logical operation.

FIG. 13. Using scFvs for Co-LOCKR targeting in a mixed population of K562 cells expressing Her2-eGFP, EGFR-iRFP, both, or neither. Cage_I269S targeted against Her2 via a Anti-Her2 scFv was combined with Key targeted against EGFR via a Cetuximab scFv. This mixture was serially diluted and evaluated for the ability to specifically target K562 cells co-expressing Her2 and EGFR as measured by Bcl2-AF594 binding. The solid line was unwashed, and the dashed line was washed within 30 minutes of analysis.

FIG. 14a-b. Tuning Cage and Decoy variants to perform [Her2 AND EpCAM NOT EGFR] logic. a. Cages with strong Cage-Latch interfaces exhibit weak ‘AND’ activation and tight ‘NOT’ deactivation, whereas cages with weak Cage-Latch interfaces exhibit strong ‘AND’ activation and leaky ‘NOT’ deactivation. These results show that Cage activity can be tuned for a desired biological function. For example, variants I287A, I287S, and 1269S exhibit greater sensitivity for [Her2 AND EpCAM^{low}] while minimally compromising leakiness in the presence of EGFR, whereas the parental Cage exhibits better deactivation for [Her2 AND EpCAM^{low} NOT EGFR]. b. Decoys can be tuned to reduce the leakiness of ‘NOT’ deactivation. Decoy variants with destabilizing mutations or truncations to weaken the latch were evaluated for the ability to perform [Her2 AND EpCAM NOT EGFR] logic on a mixed population of cells: K562/EpCAM^{low} (gray), K562/EGFR/EpCAM^{low} (yellow), K562/Her2/EpCAM^{high} (purple), and K562/Her2/EpCAM^{high}/EGFR (brown). The strongest Decoys (e.g., G24) exhibit minimal leakiness, but reduce targeting of K562/Her2/EpCAM^{high}, likely due co-localization-independent Key binding; the weakest Decoys (e.g., Box1C1) exhibit the highest targeting of K562/Her2/EpCAM^{high} along with substantial leakiness on K562/Her2/EpCAM^{high}/EGFR. Each bar represents n=1 sample.

FIG. 15a-d. Tuning Cage and Decoy variants to perform [Her2 AND EpCAM NOT EGFR] logic. Different Key and Cage concentrations were tested against 0 nM, 5 nM, or 20 nM of either EGFR_Decoy1 or EGFR_Decoy_G31. The purple “On-target” line corresponds to the desired AND signal for K562/EpCAM^{hi}/Her2 in the absence of Decoy, and the brown “Off-target” line corresponds to the undesired AND signal for K562/EGFR/EpCAM^{hi}/Her2 that the Decoy must abrogate. Using 5 nM EGFR_Decoy_G31 as the NOT gate enhances on-target binding signal, while minimally increasing undesired targeting of K562/EGFR/EpCAM^{hi}/Her2. These results are consistent with the hypothesis that Decoy-Key binding in solution should be minimized to preserve Co-LOCKR signal. a. 5 nM Key_EpCAM, 5 nM Her2_Cage. b. 5 nM Key_EpCAM, 5 nM Her2_Cage_I287A. c. 20 nM Key_EpCAM, 20 nM Her2_Cage. The original condition described in FIG. 3e is annotated. d. 20 nM Key_EpCAM 20 nM Her2_Cage_I287A.

Figure S16a-h. Tuning Co-LOCKR for selective CAR T cell tumor targeting. a. Schematic of a Bim-specific Bcl2 CAR. b. CAR T cell culture methods and flow cytometric analysis of HA tag (CAR expression) and EGFRt (transduction marker) on expanded CAR T cells. Plot is gated on CD8⁺ singlet lymphocytes and is representative of n=4 healthy T cell donors. c. Mean IFN-γ concentrations in supernatant 24 hours after co-culture of CAR T cells and Bim-expressing K562 cells (K562/Bim). Error bars represent SEM for n=2 healthy T cell donors. d,e. IFN-γ concentrations in cell supernatant 24 hours after co-culture of Cage, Key, and Raji cells with CAR T cells (n=1 healthy T cell donor). Responsiveness was tuned by mutating residues in the latch (d) or by deleting the N-terminal three (N3) or seven (N7) amino acids, or the C-terminal seven (C7) amino acids of the Key (e). Marker expression for each cell line and identity of the Cage and Key targeting domains are indicated below each plot. Red highlighting indicates the expected magnitude of signal based on the target cell's relative antigen expression. f. Schematic of cell killing assay in which four Raji cell lines are labeled with Cell Trace dyes and combined together with CAR T cells ±Cage and Key proteins. g. Flow cytometric analysis of cell killing after 48 hours. Plots show all CD5⁻ cells; frequencies of events within a given gate are indicated. h. T cell cytotoxicity was analyzed in a 4-hour Chromium release assay at various effector to target (E:T) ratios.

Figure S17. Co-LOCKR can perform ‘AND’ logic for CAR T cell targeting across a 10-fold concentration range. Her2_Cage and Key_N3_EpCAM concentrations were varied from 0 nM to 80 nM. Using 40 nM or 80 nM Co-LOCKR results in undesired targeting of K562/Her2/EpCAM^{KO} cells. Alternatively, using Cage and Key at <5 nM led to poor targeting of K562/Her2/EpCAM^{lo} but not K562/Her2/EpCAM^{hi}. Graphs show mean IFN-γ production from n=2 experiments performed with unique T cell donors (error bars are SEM)

Figure S18a-h. Co-LOCKR enables ‘AND’ and ‘OR’ logic-gated CAR T cell targeting. a,b. Mean IFN-γ concentration in cell supernatants 24 hours after co-culture of Cage, Key, and Raji (a), tumor cell lines (b) or K562 (c) cells with CAR T cells. Error bars represent SEM of n=4 healthy T cell donors. Marker expression for each cell line and identity of the Cage and Key targeting domains are indicated below each bar plot. Red highlighting indicates the expected magnitude of signal based on the target cell's relative antigen expression. d. CAR T cell proliferation in response to [Her2 AND EGFR] logic. Bar plots show the percent of T cells that have undergone at least one cell division 72 hours after co-culture of CAR T cells, Cage, Key, and target K562 cells. Histograms show flow cytometric analysis of CFSE dye dilution gated on CD8⁺ lymphocytes, and the data are representative of n=3 biological replicates with healthy T cell donors. e. CAR T cell cytotoxicity against mixed populations of Raji cells expressing combinations of Her2 and EGFR. Line graphs show mean frequency of Raji target cells after 0 or 48 hours of co-culture with CAR T cells (n=4 healthy T cell donors; solid lines=with Cage and Key, dotted lines=without Cage and Key). Arrows indicate cell lines targeted by Co-LOCKR. f. Mean frequency of live T cells in the mixed population of CART cells and Raji cells as in e. Error bars represent SEM of n=2 or 4 healthy blood donors. g. Mean IFN-γ concentration in cell supernatants 24 hours after co-culture of Cage, Keys, and K562 cells with CAR T cells as in c. Error bars represent SEM of n=3 healthy T cell donors. h. CAR T cell cytotoxicity against mixed populations of Raji cells expressing combinations of Her2, EGFR, and EpCAM in response to [Ag₁ AND either Ag₂ OR Ag₃] logic as in e. n=4 healthy T cell donors. Two distinct mixed Raji populations with five cell lines each were created because it was difficult to simultaneously distinguish all six cell lines based on Cell Trace staining. Arrows indicate cell lines targeted by Co-LOCKR.

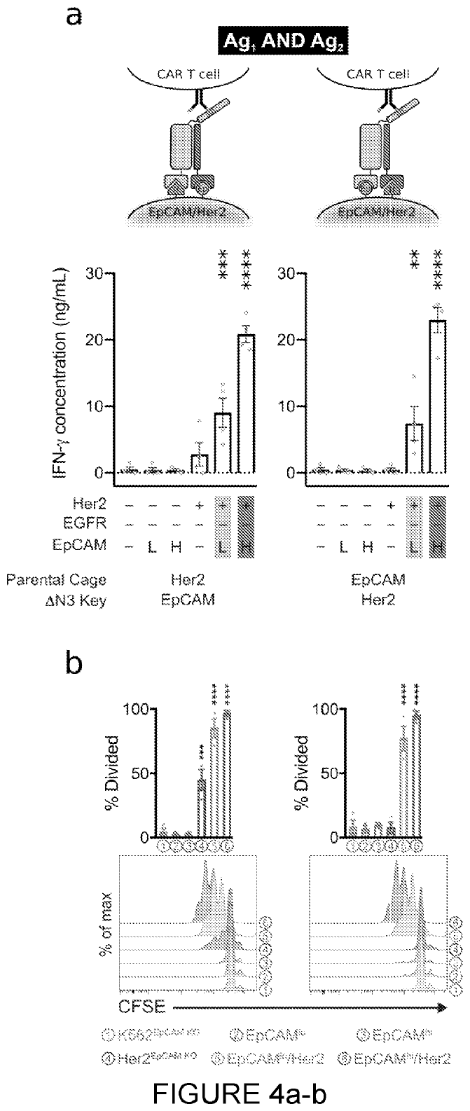

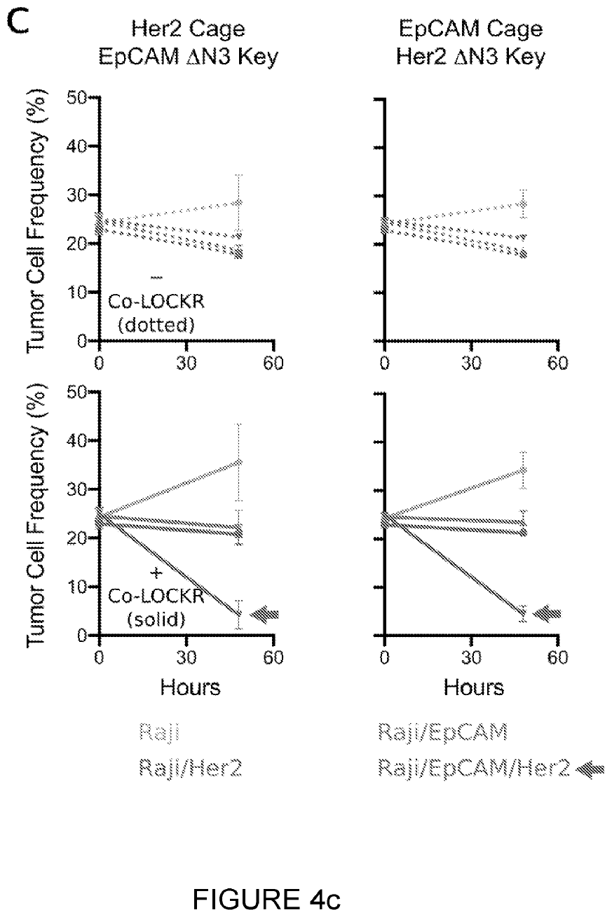

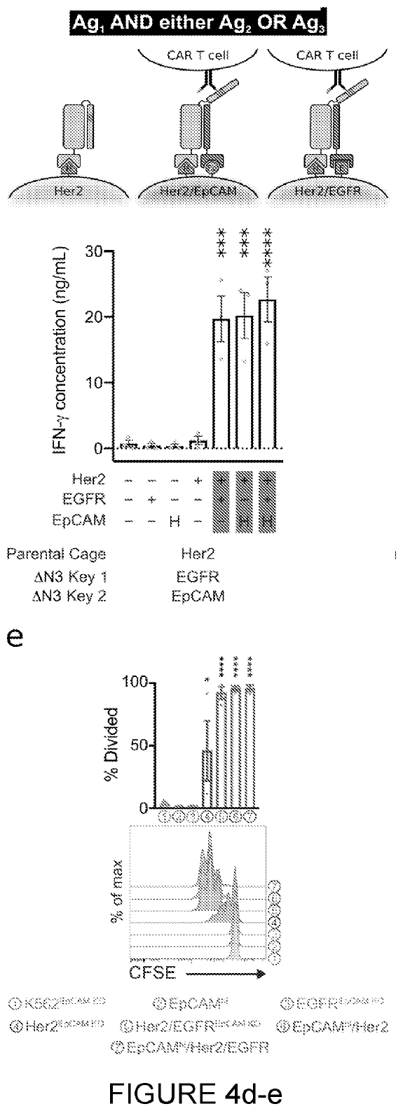

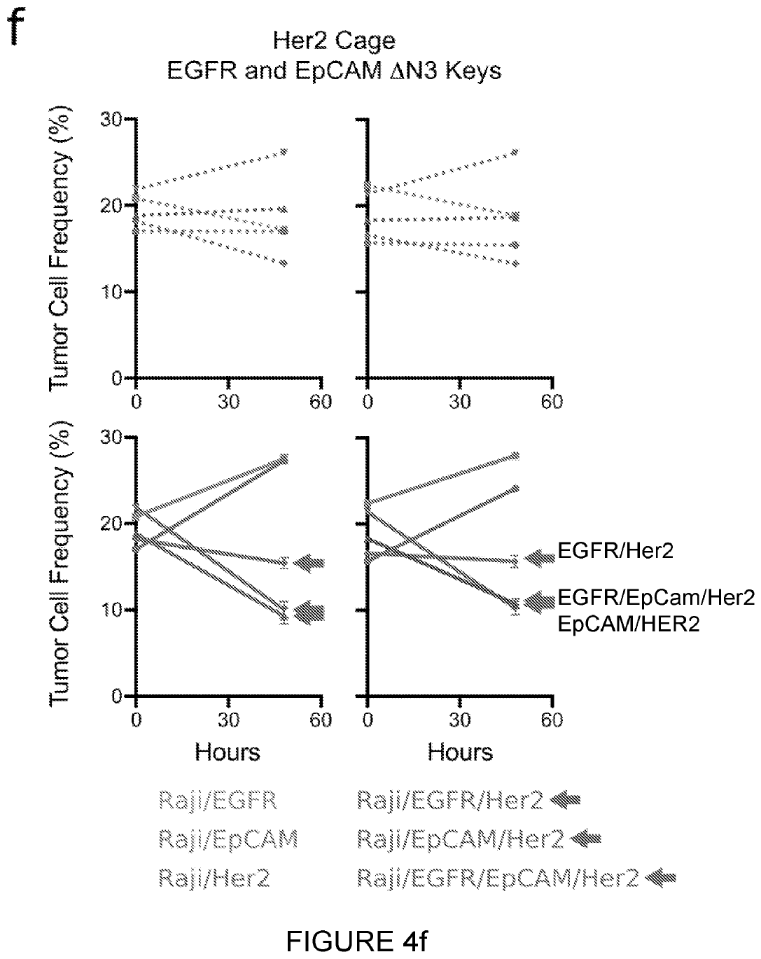

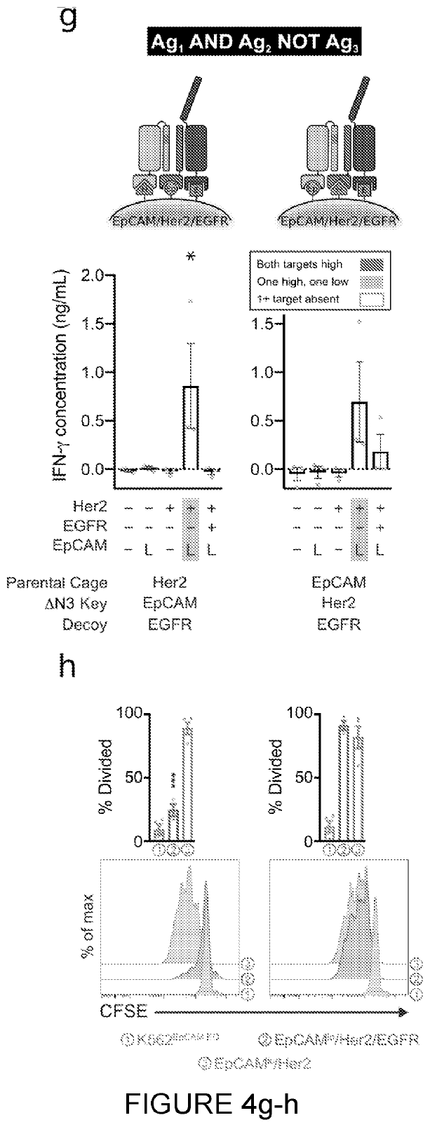

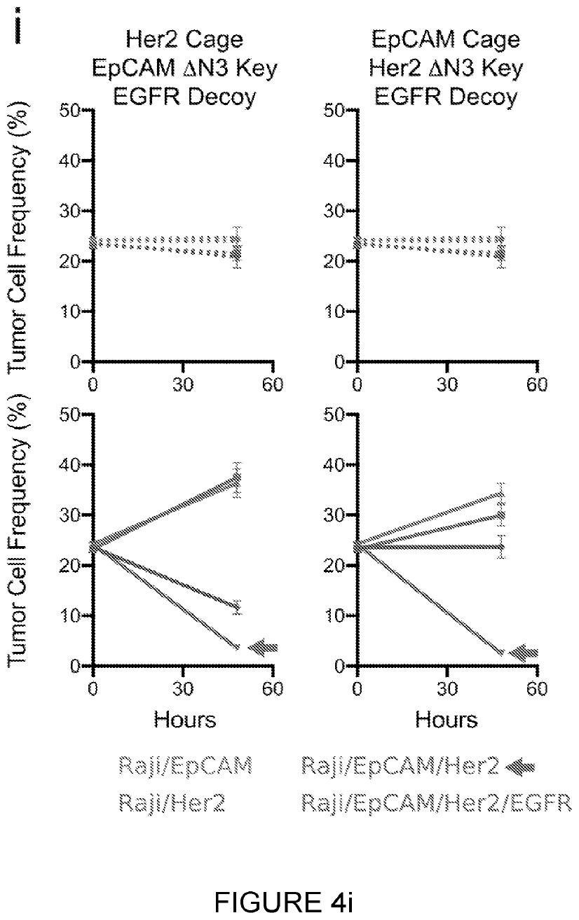

Figure S19a-c. Co-LOCKR ‘NOT’ logic-gated CAR T cell targeting requires that Key antigen is expressed at a lower level than Decoy antigen. a. Mean IFN-γ concentration in cell supernatants 24 hours after co-culture of Cage, Key, and K562 cells with CAR T cells. Error bars represent SEM of n=3 healthy T cell donors. Marker expression for each cell line and identity of the Cage and Key targeting domains are indicated below each bar plot. Red highlighting indicates the expected magnitude of signal based on the target cell's relative antigen expression. b. CART cell proliferation in response to [Ag₁ AND Ag₂ NOT Ag₃] logic. Histograms show flow cytometric analysis of CFSE dye dilution 72 hours after co-culture of Cage, Key, ±Decoy and target K562 cells with CAR T cells. Plots are gated on CD8⁺ lymphocytes, and the data are representative of n=3 biological replicates with healthy T cell donors. Two histograms are copied from FIG. 4h for reference. c. Bar plots show the percent of T cells that have undergone at least one cell division in the corresponding panel of b.

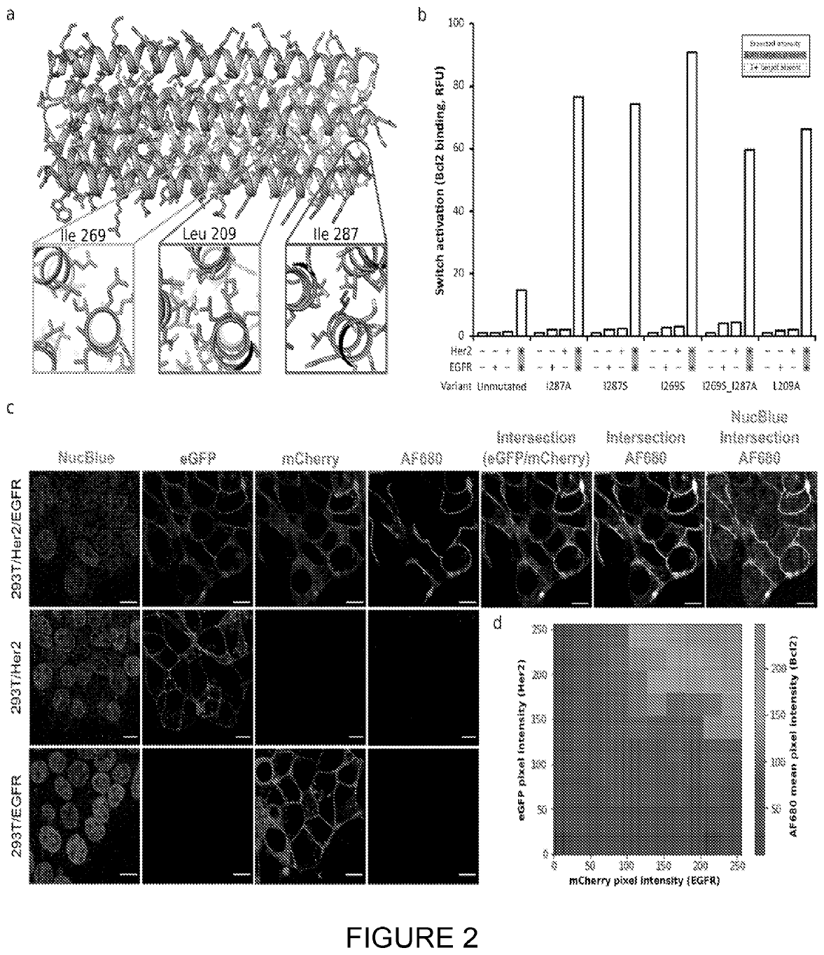

FIG. 20a-c. Uncropped confocal microscopy images of Co-LOCKR targeting HEK293T cells expressing Her2 and EGFR. a. The uncropped 293T/Her2/EGFR image used to generate FIG. 2c-d (green is Her2-eGFP, red is EGFR-mCherry, blue is Bcl2-AF680). b. The uncropped 293T/Her2/EGFR image pseudocolored as in FIG. 2c (white is the intersection of Her2-eGFP and EGFR-mCherry™, blue is NucBlue™, and magenta is Bcl2-AF680). The scale bar for the top panel is 20 μm and for the bottom panel is 10 μm. c. The uncropped images of all cell lines and staining conditions evaluated by confocal microscopy. The scale bars are 20 μm.

FIG. 21. DARPin binder affinity measured by flow cytometry. Anti-Her2 or anti-EGFR DARPins with N-terminal fusions to Bim were pre-complexed with Bcl2-AF594 and serially diluted 3-fold from 300 nM down to 0.4 nM. This dilution series was used to label a mixed population of K562 cells expressing Her2-eGFP, EGFR-iRFP, both, or neither for one hour at room temperature in a 50 μl incubation volume. The cells were then washed in PBS supplemented with 0.1% bovine serum albumin and analyzed on an LSRII flow cytometer. The apparent Kd of the DARPins was roughly 10 nM, consistent with the hypothesis Co-LOCKR activation is limited by DARPin binding affinity.

FIG. 22. Tuning the responsiveness of the Cage can enhance CAR T cell effector function against target cells exhibiting lower antigen expression. Bcl2 CAR T cells, and Co-LOCKR components (20 nM Cage and 20 nM Key_N3_EGFR) were combined with each Raji target cell line and IFN-γ production was evaluated via ELISA. Her2-Cage resulted in poor IFN-γ production against Raji target cells expressing low levels of Her2 and EGFR antigens. Her2_Cage_I269S, which was shown to result in greater activation in FIG. 2, resulted in higher levels of IFN-γ production against the same Raji target cells expressing low levels of Her2 and EGFR antigens. These results show that Co-LOCKR can be turned to target Effector function to target cells expressing different levels of target antigen.

### DETAILED DESCRIPTION

The compositions disclosed herein, also referred to as “Co-LOCKR systems” in the examples that follow, comprise of at least one cage polypeptide and at least one key polypeptide that may be used, for example, as proximity-activated de novo protein switches that perform ‘AND’, ‘OR’, and ‘NOT’ Boolean logic operations and combinations thereof in response to precise combinations of protein-binding events. The switches activate via a onformational change only when all logic conditions are met. The system is demonstrated in the examples to provide for ultraspecific targeting of mammalian cells that are distinguished in a complex cell population only by their precise combination of surface markers. An ‘AND’ gate may be achieved by targeting the cage polypeptide to one antigen and the key polypeptide to a different antigen. A ‘thresholding’ gate may be achieved by targeting the cage polypeptide and key polypeptide to the same antigen (this could be either with binding domains that bind to the same epitope or a different epitope on the same antigen). An ‘OR’ gate may be achieved by targeting the cage polypeptide or the key polypeptide to two different antigens. A ‘NOT’ gate may be achieved by supplementing a decoy cage polypeptide that sequesters the key polypeptide and prevents it from interacting with the cage polypeptide. Additional cage polypeptides, key polypeptides, and decoy cage polypeptides can be included to establish the desired logical operation (e.g., antigen 1 AND antigen 2 NOT antigen 3, antigen 1 AND either antigen 2 OR antigen 3).

Targeting specificity has been a long-standing problem in biomedicine. Despite the long-standing goal to target therapeutic agents against specific cell types, general solutions for targeting precise combinations of antigens that unambiguously identify the desired cell type are lacking. Natural systems capable of multiple-input integration are hard-coded to specific biological outputs that are difficult to modularly reassign. The methods, compositions, and polypeptides disclosed herein are modular because they comprised of de novo designed polypeptides that integrate the co-localization of two target antigens so as to conditionally expose a bioactive peptide that can recruit arbitrary effector functions. Before this work, it was not possible to produce a system that can integrate the co-localization of two or more antigens on the surface of a target cell so as to conditionally expose a bioactive peptide that can modularly recruit arbitrary effector functions. Furthermore, it was not previously possible to design such de novo proteins that can sequester a bioactive peptide in an inactive confirmation until they are co-localized. Finally, it was not previously possible to tune the sensitivity of a protein actuator to recruit the appropriate amount of effector molecule(s).

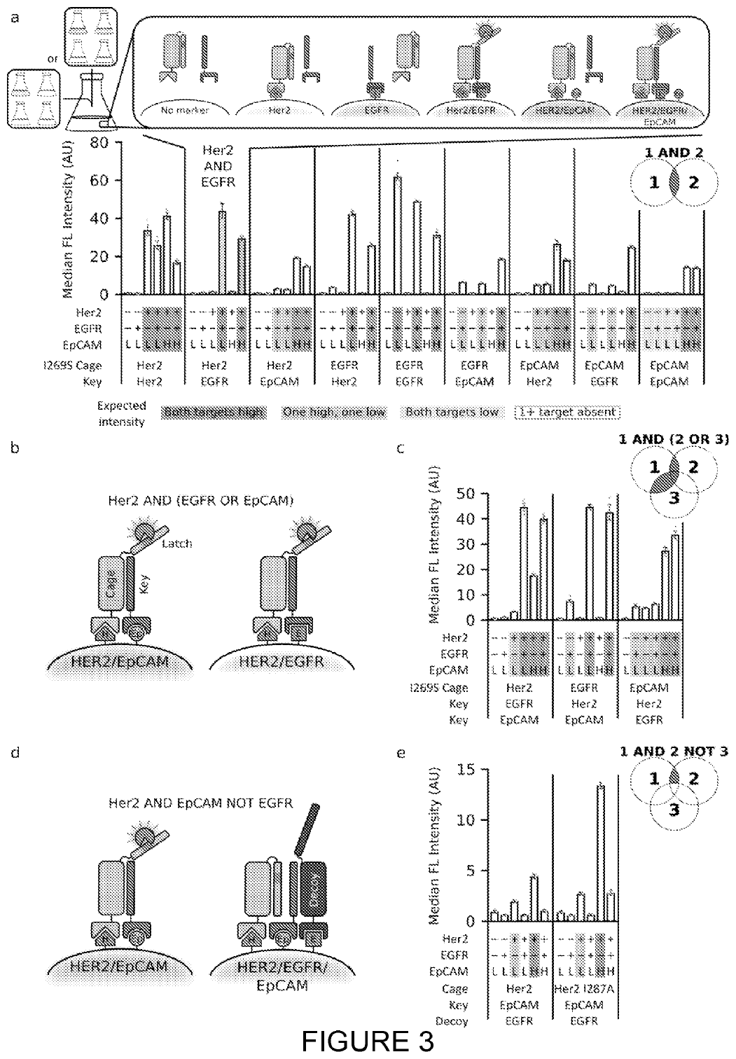

The compositions, fusion proteins, and methods disclosed herein can be used, for example, to specifically target cells of interest such as CART cells. As described in the examples that follow, the methods, fusion proteins, and compositions have been used for ultra-specific CAR T cell targeting, and directing CAR T cell cytotoxicity against certain cells within a complex milieu. The methods disclosed herein compute logic on a single cell expressing precise combinations of antigens in cis, specifically directing cytotoxicity against target cells without harming neighboring off-target cells that only provide a subset of the target antigens (FIG. 4c, f, i). ‘OR’ and ‘NOT’ logic have never been described for CAR T cells in combination with ‘AND’ logic. For example, the ability to implement complex logic (e.g., [Ag₁ AND either Ag₂ OR Ag₃] (FIG. 3c) and [Ag₁ AND Ag₂ NOT Ag₃] (FIG. 3d, FIG. 4g-i)) disclosed herein cannot be achieved with existing technologies.

The methods may comprise use of the fusion proteins, nucleic acids, vectors, cells, and/or compositions of any embodiment or combination of embodiments disclosed herein. In various embodiments, the method comprises the use of AND, OR, and/or NOT logic gates, using any embodiment or combination of embodiments as described in detail above and in the examples.

### I. Definition

All references cited are herein incorporated by reference in their entirety. As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

The “cage polypeptides” as used herein can comprise a helical bundle comprising between 2 and 7 alpha-helices. In various embodiments, the helical bundle comprises 3-7, 4-7, 5-7, 6-7, 2-6, 3-6, 4-6, 5-6, 2-5, 3-5, 4-5, 2-4, 3-4, 2-3, 2, 3, 4, 5, 6, or 7 alpha helices.

Design of the helical bundle cage polypeptides of the disclosure may be carried out by any suitable means. In one non-limiting embodiment, a BundleGridSampler™ in the Rosetta™ program may be used to generate backbone geometry based on the Crick expression for a coiled-coil and allows efficient, parallel sampling of a regular grid of coiled-coil expression parameter values, which correspond to a continuum of peptide backbone conformations. This may be supplemented by design for hydrogen bond networks using any suitable means, followed by Rosetta™ sidechain design. In a further non-limiting embodiment, best scoring designs, based on total score, number of unsatisfied hydrogen bonds, and lack of voids in the core of the protein may be selected for helical bundle cage polypeptide design.

Each alpha helix may be of any suitable length and amino acid composition as appropriate for an intended use. In one embodiment, each helix is independently 18 to 60 amino acids in length. In various embodiments, each helix is independently between 18-60, 18-55, 18-50, 18-45, 22-60, 22-55, 22-50, 22-45, 25-60, 25-55, 25-50, 25-45, 28-60, 28-55, 28-50, 28-45, 32-60, 32-55, 32-50, 32-45, 35-60, 35-55, 35-50, 35-45, 38-60, 38-55, 38-50, 38-45, 40-60, 40-58, 40-55, 40-50, or 40-45 amino acids in length.

As used throughout the present application, the term “polypeptide” is used in its broadest sense to refer to a sequence of subunit amino acids. The polypeptides of the invention may comprise L-amino acids+glycine, D-amino acids+glycine (which are resistant to L-amino acid-specific proteases in vivo), or a combination of D- and L-amino acids+glycine. The polypeptides described herein may be chemically synthesized or recombinantly expressed. The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

The term “linker” as used herein can be used to link one polypeptide, e.g., a structural region, to another polypeptide, e.g., a latch region. In some aspects, a polypeptide disclosed herein comprises a linker. In some aspects, the linker comprises one or more amino acids, e.g., an amino acid linker or a peptide linker. In some aspects, the linker connects a first alpha helix to a second alpha helix. The amino acid linkers connecting each alpha helix can be of any suitable length or amino acid composition as appropriate for an intended use. In one non-limiting embodiment, each amino acid linker is independently between 2 and 10 amino acids in length, not including any further functional sequences that may be fused to the linker. In various non-limiting embodiments, each amino acid linker is independently 3-10, 4-10, 5-10, 6-10, 7-10, 8-10, 9-10, 2-9, 3-9, 4-9, 5-9, 6-9, 7-9, 8-9, 2-8, 3-8, 4-8, 5-8, 6-8, 7-8, 2-7, 3-7, 4-7, 5-7, 6-7, 2-6, 3-6, 4-6, 5-6, 2-5, 3-5, 4-5, 2-4, 3-4, 2-3, 2, 3, 4, 5, 6, 7, 8, 9, or 10 amino acids in length. In all embodiments, the linkers may be structured or flexible (e.g. poly-GS). These linkers may encode further functional sequences, including but not limited to protease cleavage sites or one half of a split intein system.

In some aspects, one or more of the cage polypeptides and the key polypeptides further comprises a linker connecting the cage or key polypeptide and the one or more binding domains. In some aspects, the cage polypeptide comprises a linker connecting the cage polypeptide to the binding domain. In some aspects, the key polypeptide comprises a linker connecting the key polypeptide to the binding domain. Any linker known in the art may be used. In some aspects, the linker comprises one or more amino acids. In some aspects, the linker is cleavable. In some aspect, the linker is any linker disclosed herein.

The cage polypeptides include a region, termed the “latch region”, which may be used for insertion of a bioactive peptide. The cage polypeptide thus comprises a latch region and a structural region (i.e.: the remainder of the cage polypeptide that is not the latch region). When the latch region is modified to include one or more bioactive peptides, the structural region of the cage polypeptide interacts with the latch region to prevent activity of the bioactive peptide. Upon activation by key polypeptide after the cage and key polypeptides are co-localized while the binding domains are bound to their targets (as described below), the latch region dissociates from its interaction with the structural region to expose the bioactive peptide, allowing the peptide to function.

The latch region may be present near either terminus of the cage polypeptide. In one embodiment, the latch region is placed at the C-terminal helix so as to position the bioactive peptide for maximum burial of the functional residues that need to be sequestered to maintain the bioactive peptide in an inactive state while simultaneously burying hydrophobic residues and promoting solvent exposure/compensatory hydrogen bonds of polar residues. In various embodiments, the latch region may comprise a part or all of a single alpha helix in the cage polypeptide at the N-terminal or C-terminal portions. In various other embodiments, the latch region may comprise a part or all of a first, second, third, fourth, fifth, sixth, or seventh alpha helix in the cage polypeptide. In other embodiments, the latch region may comprise all or part of two or more different alpha helices in the cage polypeptide; for example, a C-terminal part of one alpha helix and an N-terminal portion of the next alpha helix, all of two consecutive alpha helices, etc.

As used herein, a “bioactive peptide” is any peptide of any length or amino acid composition that is capable of selectively binding to a defined target (i.e.: capable of binding to an “effector” polypeptide). Such bioactive peptides may comprise peptides of all three types of secondary structure in an inactive conformation: alpha helix, beta strand, and loop. The polypeptides of this aspect can be used to control the activity of a wide range of functional peptides. The ability to harness these biological functions with tight, inducible control is useful, for example, in engineering cells (inducible activation of function, engineering complex logic behavior and circuits, etc.), developing sensors, developing inducible protein-based therapeutics, and creating new biomaterials. Any suitable bioactive peptides and binding domains may be used in the compositions of the disclosure, as appropriate for an intended use. In one embodiment of the compositions of any embodiment or combination of embodiments of the disclosure, the one or more bioactive peptides may comprise one or more bioactive peptide selected from the group consisting of SEQ ID NO:60, 62-64, 66, 27052, 27053, and 27059-27093.

As used herein, the term “chimeric antigen receptor” (CAR) refers to a fusion protein comprising two or more distinct domains that are linked together in an arrangement that does not occur naturally, can function as a receptor when expressed on the surface of a cell, and comprises: an extracellular component comprising an binding domain specific for an antigen, such as the bioactive peptides as contemplated herein; an optional extracellular spacer domain to optimize binding; a hydrophobic portion or transmembrane domain; and an intracellular component comprising an intracellular activation domain (e.g., an immunoreceptor tyrosine-based activation motif (ITAM)-containing T cell activating motif), an intracellular costimulatory domain, or both. In certain embodiments, an intracellular signaling component of a CAR has an ITAM-containing T cell activating domain (e.g., CD3ζ) and an intracellular costimulatory domain (e.g., CD28, 41BB). In certain embodiments, a CAR is synthesized as a single polypeptide chain or is encoded by a nucleic acid molecule as a single chain polypeptide.

As used herein, an “immunoreceptor tyrosine-based activation motif (ITAM) T cell activating domain” refers to an intracellular signaling domain or functional portion thereof which is naturally or endogenously present on an immune cell receptor or a cell surface marker and contains at least one immunoreceptor tyrosine-based activation motif (ITAM). ITAM refers to a conserved motif of YXXL/I-X_{6.8}-YXXL/I, wherein X is any amino acid (i.e., a same or different amino acid over the length of the ITAM). In certain embodiments, an ITAM signaling domain contains one, two, three, four, or more ITAMs. An ITAM signaling domain may initiate T cell activation signaling following antigen binding or ligand engagement. ITAM-signaling domains include, for example, intracellular signaling domains of CD3γ, CD3δ, CD3ε, CD3ζCD79a, CD79b, gamma chain of FcεRI or FcγRI, FcRγ2a, FcRγ2b1, FcRγ2a1, FcRγ2b2, FcRγ3a, FcRγ3b, FcRβ1, FcεR), Natural Killer cell receptor proteins (e.g., DAP12), CD5, CD16a, CD16b, CD22, CD23, CD32, CD64, CD79a, CD79b, CD89, CD278, and CD66d. Exemplary amino acid sequences of these ITAM sequences and those from viruses (e.g., BLV gp30; EBV LMP2A) are described in Paul, Fundamental Immunology 307 (Wolters Kluwer; Lippincott; Wilkins & Wilkins; Seventh Ed., 2008). These ITAMs and functional fragments and variants thereof are also contemplated for use in the presently disclosed chimeric antigen receptor fusion proteins and host cells, and are hereby incorporated by reference.

As used herein, a “costimulatory signaling domain” refers to an intracellular signaling domain, or functional portion thereof, of a costimulatory molecule, which, when activated in conjunction with a primary or classic (e.g., ITAM-driven) activation signal (provided by, for example a CD3ζ intracellular signaling domain), promotes or enhances a T cell response, such as T cell activation, cytokine production, proliferation, differentiation, survival, effector function, or combinations thereof. Costimulatory signaling domains include, for example, CD28, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD226, CD270 (HVEM), CD273 (PD-L2), CD274 (PD-L1), CD278 (ICOS), DAP10, LAT, LFA-1, LIGHT, SLP76, TRIM, ZAP70, CD5, BAFF-R, SLAMF7, NKp80, CD160, B7-H3, a ligand that specifically binds with CD83, or any combination thereof.

An extracellular component of a fusion protein optionally comprises an extracellular, non-signaling spacer or linker region, which, for example, can position the binding domain away from the host cell (e.g., T cell) surface to enable proper cell/cell contact, antigen binding and activation (Patel et al., Gene Therapy 6: 412-419 (1999)). An extracellular spacer region of a fusion binding protein is generally located between a hydrophobic portion or transmembrane domain and the extracellular binding domain. Spacer region length may be varied to maximize antigen recognition (e.g., tumor recognition) based on the selected target molecule, selected binding epitope, or antigen-binding domain size and affinity (see, e.g., Guest et al., J. Immunother. 28:203-11 (2005); PCT Publication No. WO 2014/031687). In certain embodiments, a spacer region comprises an immunoglobulin hinge region. An immunoglobulin hinge region may be a wild-type immunoglobulin hinge region or an altered wild-type immunoglobulin hinge region. In certain embodiments, an immunoglobulin hinge region is a human immunoglobulin hinge region. An immunoglobulin hinge region may be an IgG, IgA, IgD, IgE, or IgM hinge region. An IgG hinge region may be an IgG1, IgG2, IgG3, or IgG4 hinge region. An exemplary altered IgG4 hinge region is described in PCT Publication No. WO 2014/031687, which hinge region, including the amino acid sequence thereof, is incorporated herein by reference in its entirety. In certain embodiments, an altered IgG4 hinge region comprises an amino acid sequence as set forth in SEQ ID NO:12. Other examples of hinge regions used in the fusion binding proteins described herein include the hinge region present in the extracellular regions of type 1 membrane proteins, such as CD8a, CD4, CD28 and CD7, which may be wild-type or variants thereof.

In certain embodiments, an extracellular spacer region comprises all or a portion of an Fc domain selected from: a CH1 domain, a CH2 domain, a CH3 domain, a CH4 domain, or any combination thereof (see, e.g., PCT Publication WO 2014/031687, which spacers are incorporated herein by reference in their entirety). The Fc domain or portion thereof may be wildtype of altered (e.g., to reduce antibody effector function). In certain embodiments, the extracellular component comprises an immunoglobulin hinge region, a CH2 domain, a CH3 domain, or any combination thereof disposed between the binding domain and the hydrophobic portion. In certain embodiments, the extracellular component comprises an IgG1 hinge region, an IgG1 CH2 domain, and an IgG1 CH3 domain. In further embodiments, the IgG1 CH2 domain comprises (i) a N297Q mutation, (ii) substitution of the first six amino acids (APEFLG) with APPVA, or both of (i) and (ii). In certain embodiments, the immunoglobulin hinge region, Fc domain or portion thereof, or both are human.

As used herein, a “hinge region” or a “hinge” refers to (a) an immunoglobulin hinge sequence (made up of, for example, upper and core regions of an immunoglobulin hinge) or a functional fragment or variant thereof, (b) a type II C-lectin interdomain (stalk) region or a functional fragment or variant thereof, or (c) a cluster of differentiation (CD) molecule stalk region or a functional variant thereof. As used herein, a “wild-type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

A “transmembrane domain”, as used herein, is a portion of a transmembrane protein that contains a hydrophobic portion that can insert into or span a cell membrane. Transmembrane components or domains have a three-dimensional structure that is thermodynamically stable in a cell membrane and generally range in length from about 15 amino acids to about 30 amino acids. The structure of a transmembrane component or domain may comprise an alpha helix, a beta barrel, a beta sheet, a beta helix, or any combination thereof. In certain embodiments, a transmembrane component or domain comprises or is derived from a known transmembrane protein (e.g., a CD4 transmembrane domain, a CD8 transmembrane domain, a CD27 transmembrane domain, a CD28 transmembrane domain, or any combination thereof).

A “hydrophobic portion,” as used herein, means any amino acid sequence having a three-dimensional structure that is thermodynamically stable in a cell membrane, and generally ranges in length from about 15 amino acids to about 30 amino acids. The structure of a hydrophobic domain may comprise an alpha helix, a beta barrel, a beta sheet, a beta helix, or any combination thereof. In certain embodiments, a hydrophobic portion is a transmembrane domain, for example, a transmembrane domain derived from a CD8, CD28, or CD27 molecule.

An “effector” is any molecule, nucleic acid, protein, nucleoprotein complex, or cell that carries out a biological activity upon interaction with the bioactive peptide. Exemplary biological activities can include binding, recruitment of fluorophores, recruitment of toxins, recruitment of immunomodulators, proteolysis, enzymatic activity, release of signaling proteins (e.g., cytokines, chemokine), induction of cell death, induction of cell differentiation, nuclear import/export, ubiquitination, and fluorophore/chromophore maturation.

### II. Composition of Disclosure

The present disclosure is directed to a chimeric antigen receptor T cell therapy system that can improve a target cell specificity in vitro, in vivo, or ex vivo. In particular, the system can be within a tumor microenvironment in which a CAR T cell therapy to specifically target a tumor cell is needed. In some aspects, the present composition is capable of increasing selectivity of a cell for a chimeric antigen receptor (CAR) T cell therapy. In some aspects, the composition of the present disclosure is capable of increasing selectivity of cells that are interacting with each other for a chimeric antigen receptor T cell therapy. In some aspects, the present composition is capable of targeting heterogeneous cells (more than two different cell types) for a chimeric antigen receptor T cell therapy, wherein a first cell moiety and a second cell moeity are present on the first cell and a first cell moiety and a third cell moiety are present on the second cell. In some aspects, the composition is also capable of reducing off-target activity for a chimeric antigen receptor T cell therapy. Therefore, in some aspects, the present composition can prepare a subject in need of a CAR T cell therapy so that the subject can respond better to the therapy, the efficacy of the therapy is increased, and/or a toxicity due to non specific binding (or leakiness) is reduced.

Ag1 AND Ag2

In some aspects, the present disclosure is capable of increasing selectivity of a cell that comprises at least two different cell markers (moieties Ag1 AND Ag2) for CAR T cell therapy. By targeting cells that express two different moieties, cells that comprises only one of the moieties (Ag1 OR Ag2) can be de-selected. In some aspects, a composition of the present disclosure comprises:

(a) a polynucleotide encoding a first cage polypeptide fused to a first binding domain, wherein the first cage polypeptide comprises (i) a structural region and (ii) a latch region further comprising one or more bioactive peptides, wherein the structural region interacts with the latch region to prevent activity of the one or more bioactive peptides in the absence of colocalization with a key polypeptide and wherein the first binding domain is capable of binding to a first cell moiety present on or within a cell; and

(b) a polynucleotide encoding a first key polypeptide fused to a second binding domain, wherein upon colocalization with the first cage polypeptide, the first key polypeptide is capable of binding to the cage structural region to activate the one or more bioactive peptides, wherein the second binding domain is capable of binding to a second cell moiety present on or within the cell,

wherein the first cell moiety and the second cell moiety are different or the same and wherein the cell is used for or targeted in a chimeric antigen receptor (CAR) T cell therapy. In some aspects, the polynucleotide encoding the cage polypeptide and the polynucleotide encoding the key polypeptide is on the same vector or on different vectors.

In some aspects, a composition of the present disclosure comprises:

(a) a first cage polypeptide fused to a first binding domain, wherein the first cage polypeptide comprises (i) a structural region and (ii) a latch region further comprising one or more bioactive peptides, wherein the structural region interacts with the latch region to prevent activity of the one or more bioactive peptides in the absence of colocalization with a key polypeptide and wherein the first binding domain is capable of binding to a first cell moiety present on or within a cell; and

(b) a first key polypeptide fused to a second binding domain, wherein upon colocalization with the first cage polypeptide, the first key polypeptide is capable of binding to the cage structural region to activate the one or more bioactive peptides, wherein the second binding domain is capable of binding to a second cell moiety present on or within the cell,

wherein the first cell moiety and the second cell moiety are different or the same and wherein the cell is used for or targeted in a chimeric antigen receptor (CAR) T cell therapy.

For the one or more bioactive peptides are to be activated (e.g., exposed to an effector molecule or capable of transduce its signal downstream), a functional cage polypeptide and a key polypeptide need to be colocalized. The mere expression of the functional cage polypeptide and a key polypeptide is not sufficient. For example, in some aspects, binding of a functional cage polypeptide, e.g., a first cage polypeptide, to a key polypeptide in solution is less efficient to activate the one or more bioactive peptides than binding of the cage and key polypeptides after colocalization. In some aspects, therefore, the colocalization of the first cage polypeptide and the key polypeptide increases selective targeting of a cell that highly expresses the cell moiety.

In some aspects, the colocalization of the first cage polypeptide and the first key polypeptide increases the local concentration of the first cage polypeptide and the first key polypeptide and shifts the binding equilibrium in favor of complex formation between the first cage polypeptide and the first key polypeptide.

In order for two cell moieties to be close enough (e.g., in close proximity) to allow colocalization of a cage polypeptide binding the first cell moiety and a key polypeptide binding to the second cell moiety, the two cell moieties may be colocalized as a result of directly or indirectly forming a complex (e.g., two proteins in the same complex such as a Her2-EGFR heterodimer or CD3ζ in complex with LAT or Zap70; two DNA sequences located in close proximity on a chromosome; two RNA sequences located in close proximity on an mRNA). In this case at least one molecule of the first moiety must be colocalized with at least one molecule of the second moiety to result in colocalization. Alternatively, the two cell moieties may be colocalized by virtue of being expressed in sufficient numbers in the same subcellular compartment (e.g., two transmembrane proteins expressed in the cell membrane such as Her2 and EGFR, Her2 and EpCAM, etc. In some aspects, the cell expresses a first cell moiety and/or the second cell moiety at least about 100 copies per cell, at least about 200 copies per cell, at least about 500 copies per cell, at least about 1000 copies per cell, at least about 1500 copies per cell, at least about 2000 copies per cell, at least about 2500 copies per cell, at least about 3000 copies per cell, at least about 3500 copies per cell, at least about 4000 copies per cell, at least about 4500 copies per cell, at least about 5000 copies per cell, at least about 5500 copies per cell, at least about 6000 copies per cell, at least about 6500 copies per cell, or at least about 7000 copies per cell. In some aspects, the first cell moiety and/or the second cell moiety express about 500 to about 10,000 copies per cell, about 1000 to about 10,000 copies per cell, about 2000 to about 10,0000 copies per cell, about 3000 to about 10,000 copies per cell, about 4000 to about 10,000 copies per cell, about 5000 to about 10,000 copies per cell, about 1000 to about 9,000 copies per cell, about 2000 to about 9,0000 copies per cell, about 3000 to about 9,000 copies per cell, about 4000 to about 9,000 copies per cell, about 5000 to about 9,000 copies per cell, about 1000 to about 8,000 copies per cell, about 2000 to about 8,0000 copies per cell, about 3000 to about 8,000 copies per cell, about 4000 to about 8,000 copies per cell, about 5000 to about 8,000 copies per cell, about 1000 to about 7,000 copies per cell, about 2000 to about 7,0000 copies per cell, about 3000 to about 7,000 copies per cell, about 4000 to about 7,000 copies per cell, about 5000 to about 7,000 copies per cell, about 1000 to about 6,000 copies per cell, about 2000 to about 6,0000 copies per cell, about 3000 to about 6,000 copies per cell, about 4000 to about 6,000 copies per cell, about 5000 to about 6,000 copies per cell. In some aspects, the cell expresses a first cell moiety and/or the second cell moiety at least about 5000 copies up to about 6000 copies, up to about 7000 copies or up to about 8000 copies. In some aspects, the first cage polypeptide and the first key polypeptide are colocalized, thereby forming a complex and activating the one or more bioactive peptides.

In some aspects, the first cell moiety and the second cell moiety are present on the surface of the cell. In some aspects, the first cell moiety and the second cell moiety are present within the cytoplasm of the cell. In some aspects, the first cell moiety and the second cell moiety are present within the nucleus of the cell. In some aspects, the first cell moiety and the second cell moiety are present within the secretory pathway of the cell, including the endoplasmic reticulum (ER) and Golgi apparatus.

Ag1 and (Ag2 or Ag3)

The present disclosure can also target more than two cells at the same time by utilizing various cell markers. For instant, the disclosure can allow a therapy to target heterogenous cell types, more than two (Ag1 AND (Ag2 OR Ag3)), more than three (Ag1 AND (Ag2 OR Ag3 OR Ag4)), more than four (Ag1 AND (Ag2 OR Ag3 OR Ag 4 OR Ag5)), more than five (Ag1 AND (Ag2 OR Ag3 OR Ag 4 OR Ag5 OR Ag6)), etc. for a CAR T cell therapy. In some embodiments, (Ag1 OR Ag2) AND Ag3 can be accomplished by targeting multiple cage polypeptides to multiple cells at the same time with different binding domains and targeting one key polypeptide with a single binding domain to those same cells. In other embodiments, (Ag1 OR Ag2) AND (Ag3 OR Ag4) can be accomplished by targeting multiple cage polypeptides with multiple binding domains and multiple key polypeptides with multiple binding domains.

In some aspects, the composition comprises:

(a) a first cage polypeptide fused to a first binding domain or a polynucleotide encoding the same, wherein the first cage polypeptide comprises (i) a structural region and (ii) a latch region further comprising one or more bioactive peptides, wherein the structural region interacts with the latch region to prevent activity of the one or more bioactive peptides in the absence of colocalization with a key polypeptide and wherein the first binding domain is capable of binding to a first cell moiety present on or within a first cell (Cell Type I, e.g., cell expressing Ag1 AND Ag2);
(b) a first key polypeptide fused to a second binding domain or a polynucleotide encoding the same, wherein upon colocalization with the first cage polypeptide, the first key polypeptide is capable of binding to the cage structural region to activate the one or more bioactive peptides, wherein the second binding domain is capable of binding to a second cell moiety present on or within the first cell; and
(c) a second key polypeptide fused to a third binding domain or a polynucleotide encoding the same, wherein upon colocalization with the first cage polypeptide, the second key polypeptide is capable of binding to the cage structural region to activate the one or more bioactive peptides, wherein the third binding domain is capable of binding to a third cell moiety present on or within a second cell that also comprises the first cell moiety (Cell type II, e.g., cell expressing Ag1 AND Ag3), wherein the first cell moiety, the second cell moiety, and the third cell moiety are different, and wherein the cell is used for or targeted in a CAR T cell therapy.

In some aspects, the first key polypeptide comprises a third binding domain, wherein the second binding domain and/or the third binding domain bind to (i) different moieties than the first binding domain on the surface of the same cell, or (ii) different moieties than the first binding domain at the synapse between two cells that are in contact, wherein upon colocalization with the first cage polypeptide, the first key polypeptide is capable of binding to the cage structural region to activate the one or more bioactive peptides, wherein the third binding domain is capable of binding to a third cell moiety present on or within the cell that also comprises the first cell moiety, wherein the third cell moiety is different from the first cell moiety or the second cell moiety.

In some aspects, the compositions further comprise:

(d) at least a second cage polypeptide comprising (i) a second structural region, (ii) a second latch region further comprising one or more bioactive peptides, and (iii) a sixth binding domain, wherein the second structural region interacts with the second latch region to prevent activity of the one or more bioactive peptides,

wherein the first key and/or the second key polypeptide are capable of binding to the second structural region to activate the one or more bioactive peptides, and

wherein the sixth binding domain and/or the first binding domain bind to (i) different moieties than the second binding domain, third binding domain and/or fourth binding domain on the surface of the same cell, or (ii) different moieties than the second binding domain, third binding domain and/or fourth binding domain at the synapse between two cells that are in contact. Such compositions can be used, for example, to accomplish (Ag1 OR Ag2) AND Ag3 by targeting the two cage polypeptides with different binding domains to multiple cells at the same time and targeting one key polypeptide with a single binding domain to those same cells.

In some aspects, the composition can further comprise multiple key polypeptides, a fourth key polypeptide, a fifth key polypeptide, a sixth key polypeptide, or a seventh key polypeptide, to increase selectivity for the first cell and/or the second cell. For example the composition for the first cell can further comprise additional key polypeptides, a fourth key polypeptide, a fifth key polypeptide, a sixth key polypeptide, or a seventh key polypeptide, that can further increase the selectivity of the first cell. In some aspects, the composition for the second cell further comprises additional key polypeptides, a fourth key polypeptide, a fifth key polypeptide, a sixth key polypeptide, or a seventh key polypeptide, that can further increase the selectivity of the second cell. Each of the additional key polypeptides for the present disclosure can be fused to a binding domain, wherein upon colocalization with the first cage polypeptide, the third key polypeptide is capable of binding to the cage structural region to activate the one or more bioactive peptides, wherein the third binding domain is capable of binding to a cell moiety present on or within the cell that also comprises the first cell moiety. In some aspects, a single key polypeptide can be fused to two or more binding domains such that the same key polypeptide can be targeted to both Cell type I and Cell type II.

### (Ag1 and Ag2) not Ag3

The present disclosure can also direct a therapy to avoid normal (healthy) cells, but only target diseased cells, e.g., tumor cells by utilizing various cell markers, thereby reducing off-target cell specificity or toxicity. Therefore, the disclosure can allow a therapy to avoid targeting normal cell types that express unique cell markers. For example, if normal cells express Ag3 while the diseased cells don't, the composition for the present disclosure can be constructed to avoid the cells expressing Ag3.

In some aspects, the composition comprises:

(a) a first cage polypeptide fused to a first binding domain or a polynucleotide encoding the same, wherein the first cage polypeptide comprises (i) a structural region and (ii) a latch region further comprising one or more bioactive peptides, wherein the structural region interacts with the latch region to prevent activity of the one or more bioactive peptides in the absence of colocalization with a key polypeptide and wherein the first binding domain is capable of binding to a first cell moiety present on or within a cell;
(b) a first key polypeptide fused to a second binding domain or a polynucleotide encoding the same, wherein upon colocalization with the first cage polypeptide, the first key polypeptide is capable of binding to the cage structural region to activate the one or more bioactive peptides, wherein the second binding domain is capable of binding to a second cell moiety present on or within the cell; and
(c) one or more decoy cage polypeptide fused to one or more binding domains (“decoy binding domain”) or a polynucleotide encoding the same, wherein each decoy cage polypeptide comprises a decoy structural region, which upon colocalization with the first key polypeptide and the first cage polypeptide, is capable of preferentially binding to the first key polypeptide and wherein the each decoy binding domain is capable of binding to a cell moiety (“decoy cell moiety”) in the cell that comprises the second cell moiety. In some aspects, the decoy binding domain is capable of binding to a cell moiety (“decoy cell moiety”) in the cell that comprises the first cell moiety and the second cell moiety. In some aspects, the decoy cell moiety is present only on a healthy cell. In some aspects, the decoy cage polypeptide, upon colocalization with the first key polypeptide, binds to the first key polypeptide such that the first key polypeptide does not bind to the first cage polypeptide and wherein the one or more bioactive peptides in the first cage polypeptide are not activated.

Any first cage polypeptide can serve as a decoy polypeptide for any second cage polypeptide, provided that the first cage polypeptide has a higher affinity for the key polypeptide than does the second cage polypeptide.

The compositions and methods of all aspects described herein may comprise use of a single decoy cage polypeptide comprising multiple binding domains, or multiple decoy cage polypeptides each with one (or more) binding domains to avoid cells with different decoy cell moieties (e.g., 1 AND 2 NOT (3 OR 4) logic).

In some aspects, the binding affinity of the decoy cage polypeptide to a key polypeptide (e.g., K_{D}) is stronger (e.g., lower) than the binding affinity of the first cage polypeptide to a key polypeptide (e.g., K_{D}), e.g., by at least about 1.1 fold, at least about 1.5 fold, at least about 2 fold, at least about 3 fold, at least about 4 fold, at least about 5 fold, at least about 6 fold, at least about 7 fold, at least about 8 fold, at least about 9 fold, at least about 10 fold, at least about 20 fold, at least about 30 fold, at least about 40 fold, at least about 50 fold, at least about 60 fold, at least about 70 fold, at least about 80 fold, at least about 90 fold, at least about 100 fold, at least about 150 fold, at least about 200 fold, at least about 300 fold, at least about 400 fold, at least about 500 fold, at least about 600 fold, at least about 700 fold, at least about 800 fold, at least about 900 fold, or at least about 1000 fold. In some aspects, the decoy cage polypeptide comprises at least one alpha helix, at least two alpha helices, at least three alpha helices, at least four alpha helices, or at least five alpha helices. In some aspects, the decoy cage polypeptide further comprises a decoy latch region. In some aspects, the decoy latch region is not functional. In some aspects, the decoy latch region does not comprise any bioactive peptide. In some aspects, the decoy latch region is not present. In some aspects, the decoy latch region comprises a non-functional bioactive peptide. In some aspects, the decoy latch region comprises a functional bioactive peptide with a distinct biological function. By way of non-limiting example, the cage polypeptide may comprise a bioactive peptide with immunostimulatory function and the decoy cage polypeptide comprises a bioactive peptide with immunoinhibitory function.

Exemplary Co-LOCKR Systems

In a first aspect, the disclosure provides compositions comprising

(a) a first cage polypeptide comprising (i) a structural region, (ii) a latch region further comprising one or more bioactive peptides, and (iii) a first binding domain wherein the structural region interacts with the latch region to prevent activity of the one or more bioactive peptides;

(b) a first key polypeptide capable of binding to the cage structural region to activate the one or more bioactive peptides, wherein the key polypeptide comprises a second binding domain,

wherein the first binding domain and the second binding domain bind to (i) different moieties on the surface of the same cell, (ii) the same moiety on the surface of the same cell, (iii) different moieties at the synapse between two cells that are in contact, or (iv) the same moiety at the synapse between two cells that are in contact; and

(c) cells comprising one or more chimeric antigen receptor(s) that bind to the one or more bioactive peptides when the one or more bioactive peptides are activated.

In any of the embodiments described herein, the chimeric antigen receptor may further comprise a self-cleaving polypeptide, wherein a polynucleotide encoding the self-cleaving polypeptide is located between the polynucleotide encoding the fusion protein and the polynucleotide encoding the transduction marker. In certain embodiments, a self-cleaving polypeptide comprises a 2A peptide from porcine teschovirus-1 (P2A), Thosea asigna virus (T2A), equine rhinitis A virus (E2A), foot-and-mouth disease virus (F2A), or variant thereof. Further exemplary nucleic acid and amino acid sequences of 2A peptides are set forth in, for example, Kim et al. (PLOS One 6:e18556 (2011), which 2A nucleic acid and amino acid sequences are incorporated herein by reference in their entirety).

The cells may be any suitable cell comprising the chimeric antigen receptor, including but not limited to T cells.

As used herein, a “synapse” is a junction between two interacting cells, typically involving protein-protein contacts across the junction. An immunological synapse is the interface between an antigen-presenting cell or target cell and a lymphocyte such as a TB cell or Natural Killer cell. A neuronal synapse is a junction between two nerve cells, consisting of a minute gap across which impulses pass by diffusion of a neurotransmitter. This embodiment is particularly useful, for example, when detecting cells that are in contact with each other, but not cells that are not. For example, one could identify only T cells that are interacting with a specified target cell but avoid all non-interacting T cells.

Thus, in one embodiment the first binding domain and the second binding domain bind to (i) different moieties on the surface of the same cell, or (iii) different moieties at the synapse between two cells that are in contact. In this embodiment, the composition can be used to establish an AND gate.

In another embodiment, the first binding domain and the second binding domain bind to (ii) the same moiety on the surface of the same cell, or (iv) the same moiety at the synapse between two cells that are in contact. In this embodiment, the composition can be used to establish a thresholding gate.

In one embodiment, (c) the first key polypeptide comprises a third binding domain, wherein the second binding domain and/or the third binding domain bind to (i) different moieties than the first binding domain on the surface of the same cell, or (ii) different moieties than the first binding domain at the synapse between two cells that are in contact. In a further embodiment, the second binding domain and the third binding domain bind to different moieties on the surface of different cells. In these embodiments, the composition can be used to establish a 1 AND either 2 OR 3 logic gate, provided the moiety bound by the first binding domain is present on one of those cells.

In another embodiment, the composition further comprises (d) at least a second key polypeptide capable of binding to the first cage structural region, wherein the key polypeptide comprises a fourth binding domain, wherein the second binding domain and/or the fourth binding domain bind to (i) different moieties than the first binding domain on the surface of the same cell, or (ii) different moieties than the first binding domain at the synapse between two cells that are in contact. In one embodiment, the second binding domain and the fourth binding domain bind to (i) different moieties on the surface of the same cell, or (ii) different moieties at the synapse between two cells that are in contact. In a further embodiment, the second binding domain and the fourth binding domain bind to different moieties on the surface of different cells. In these embodiments, the composition can be used to establish a 1 AND either 2 OR 3 logic gate, provided the moiety bound by the first binding domain is present on one of those cells.

In a further embodiment, the first cage polypeptide further comprises a fifth binding domain, wherein the fifth binding domain and/or the first binding domain bind to (i) different moieties than the second binding domain, third binding domain and/or fourth binding domain on the surface of the same cell, or (ii) different moieties than the second binding domain, third binding domain and/or fourth binding domain at the synapse between two cells that are in contact. In one embodiment, the fifth binding domain and the first binding domain bind to (i) different moieties on the surface of the same cell, or (ii) different moieties at the synapse between two cells that are in contact. In this embodiment, the composition can be used to establish an OR logic gate, specifically the [(1 OR 5) AND (2 OR 3)] logic gate, based on the additional binding domain present on a single cage polypeptide.

In one embodiment, the composition further comprises (e) at least a second cage polypeptide comprising (i) a second structural region, (ii) a second latch region further comprising one or more bioactive peptides, and (iii) a sixth binding domain, wherein the second structural region interacts with the second latch region to prevent activity of the one or more bioactive peptides, wherein the first key and/or the second key polypeptide are capable of binding to the second structural region to activate the one or more bioactive peptides, and wherein the sixth binding domain and/or the first binding domain bind to (i) different moieties than the second binding domain, third binding domain and/or fourth binding domain on the surface of the same cell, or (ii) different moieties than the second binding domain, third binding domain and/or fourth binding domain at the synapse between two cells that are in contact. In one embodiment, the sixth binding domain and the first binding domain bind to (i) different moieties on the surface of different cells, or (ii) different moieties at the synapse between two cells that are in contact. In these embodiments, the composition can be used to establish an OR logic gate based on the additional binding domain present on a second cage polypeptide. In one such embodiment, there may be two separate but identical cage polypeptides be each attached to one different binding domain. In another such embodiment, the two cage polypeptides may be different cage polypeptides that both are activated by the same key polypeptide and are each attached to one different binding domain.

In another embodiment, the composition further comprises (f) a decoy cage polypeptide comprising (i) a decoy structural region, (ii) a decoy latch region optionally further comprising one or more bioactive peptides, and (iii) a seventh binding domain, wherein the decoy structural region interacts with the first key polypeptide and/or the second key polypeptide to prevent them from binding to the first and/or the second cage polypeptides, and wherein the seventh binding domain binds to a moiety on the surface of the same cell as the second binding domain, third binding domain, and/or fourth binding domain. In one embodiment, the seventh binding domain binds to a moiety that is present on the cell at an equal or higher level than the moieties to which the second binding domain, the third binding domain, and/or the fourth binding domain bind to. In this embodiment, the composition can be used to establish a NOT logic gate based on the decoy cage polypeptide binding to a different target on the same cell as the target of the key polypeptide. In this embodiment, the composition can be used, for example, to establish a 1 AND 2 NOT 7 logic, provided the moieties bound by the first and second binding domains are present the same cell. In one embodiment, the decoy cage polypeptide does not comprise a bioactive peptide. This embodiment can be used, for example, to establish a 3 AND 4 NOT 7 logic (provided that the moieties bound by the third and fourth binding domains are present on the same cell), or a 5 AND 6 NOT 7 logic (provided that the moieties bound by the fifth and sixth binding domains are present on the same cell. Such AND/NOT embodiments require at least one cage polypeptide, at least one key polypeptide, and at least one decoy cage polypeptide.

In one embodiment of all these embodiments of the composition, the first binding domain, the second binding domain, the third binding domain (when present), the fourth binding domain (when present), the fifth binding domain (when present), the sixth binding domain (when present), and/or the seventh binding domain (when present) comprise polypeptides capable of binding moieties present on the cell surface, including proteins, saccharides, and lipids. In one embodiment, the one or more binding proteins comprise cell surface protein binding polypeptides.

All of the compositions above are described as polypeptide compositions. The disclosure also provides compositions comprising expression vectors and/or cells that express the cage polypeptides and key polypeptides as described in the compositions above, and thus can be used for the same purposes (for example, in establishing the same logic gates as for the corresponding polypeptide compositions described above). Thus, in a fifth aspect, the disclosure provides compositions comprising:

(a) one or more expression vectors encoding and/or cells expressing:
(i) a first cage polypeptide comprising (i) a structural region, (ii) a latch region further comprising one or more bioactive peptides, and (iii) a first binding domain wherein the structural region interacts with the latch region to prevent activity of the one or more bioactive peptides; and
(ii) a first key polypeptide capable of binding to the cage structural region to activate the one or more bioactive peptides, wherein the key polypeptide comprises a second binding domain,

wherein the first binding domain and the second binding domain bind to (i) different moieties on the surface of the same cell, (ii) the same moiety on the surface of the same cell, (iii) different moieties at the synapse between two cells that are in contact, or (iv) the same moiety at the synapse between two cells that are in contact; and

(b) (i) cells comprising one or more chimeric antigen receptor(s) that bind to the one or more bioactive peptides when the one or more bioactive peptides are activated; and/or (ii) one or more the fusion protein, the nucleic acid encoding the fusion protein, the vector comprising the fusion protein encoding nucleic acid, and/or the cell comprising the fusion protein, the nucleic acid encoding the fusion protein, and/or the vector comprising the fusion protein encoding nucleic acid as described herein.

The one or more expression vectors may comprise a separate expression vector encoding each separate polypeptide, may comprise an expression vector encoding two or more of the separate polypeptides, or any combination thereof as suitable for an intended use. The expression vector may comprise any suitable expression vector that operatively links a nucleic acid coding region for the cited polypeptide(s) to any control sequences capable of effecting expression of the gene product. Similarly, the cells may be any prokaryotic or eukaryotic cell capable of expressing the recited polypeptide(s); the cells may comprise a single cell capable of expressing all of the recited polypeptides, separate cells capable of expressing each individual polypeptide, or any combination thereof.

In one embodiment the first key polypeptide comprises a third binding domain, wherein the second binding domain and/or the third binding domain bind to (i) different moieties than the first binding domain on the surface of the same cell, or (ii) different moieties than the first binding domain at the synapse between two cells that are in contact. In another embodiment, the second binding domain and the third binding domain bind to different moieties on the surface of different target cells.

In one embodiment, the composition further comprises (c) an expression vector encoding and/or a cell expressing at least a second key polypeptide capable of binding to the first cage structural region, wherein the key polypeptide comprises a fourth binding domain, wherein the second binding domain and/or the fourth binding domain bind to (i) different moieties than the first binding domain on the surface of the same cell, or (ii) different moieties than the first binding domain at the synapse between two cells that are in contact. In another embodiment wherein the second binding domain and the fourth binding domain bind to (i) different moieties on the surface of the same cell, or (ii) different moieties at the synapse between two cells that are in contact.

In another embodiment, the first cage polypeptide further comprises a fifth binding domain, wherein the fifth binding domain and/or the first binding domain bind to (i) different moieties than the second binding domain, third binding domain, and/or fourth binding domain on the surface of the same cell, or (ii) different moieties than the second binding domain, third binding domain, and/or fourth binding domain at the synapse between two cells that are in contact. In one embodiment, the fifth binding domain and the first binding domain bind to (i) different moieties on the surface of the same cell, or (ii) different moieties at the synapse between two cells that are in contact.

In a further embodiment, the composition further comprises (d) an expression vector encoding and/or a cell expressing at least a second cage polypeptide comprising (i) a second structural region, (ii) a second latch region further comprising one or more bioactive peptides, and (iii) a sixth binding domain, wherein the second structural region interacts with the second latch region to prevent activity of the one or more bioactive peptides,

wherein the first key and/or the second key polypeptide are capable of binding to the second structural region to activate the one or more bioactive peptides, and

wherein the sixth binding domain and/or the first binding domain bind to (i) different moieties than the second binding domain, third binding domain, and/or fourth binding domain on the surface of the same cell, or (ii) different moieties than the second binding domain, third binding domain, and/or fourth binding domain at the synapse between two cells that are in contact. In one embodiment, the sixth binding domain and the first binding domain bind to (i) different moieties on the surface of different cells, or (ii) different moieties at the synapse between two cells that are in contact.

In another embodiment, the composition further comprises (e) an expression vector encoding and/or a cell expressing a decoy cage polypeptide comprising (i) a decoy structural region, (ii) a decoy latch region optionally further comprising one or more bioactive peptides, and (iii) a seventh binding domain, wherein the decoy structural region interacts with the first key polypeptide and/or the second key polypeptide to prevent them from binding to the first and/or the second cage polypeptides, and wherein the seventh binding domain binds to a moiety on the surface of the same cell as the second binding domain, third binding domain, and/or fourth binding domain. In one embodiment, the seventh binding domain and the first binding domain and/or second binding domain bind to (i) different moieties on the surface of the same cell, or (ii) different moieties at the synapse between two cells that are in contact. In another embodiment, the seventh binding domain binds to a moiety that is present on the cell at an equal or higher level than the moieties to which the second binding domain, the third binding domain, and/or the fourth binding domain bind to.

In one embodiment of all of the compositions of the disclosure, the first binding domain, the second binding domain, the third binding domain (when present), the fourth binding domain (when present), the fifth binding domain (when present), the sixth binding domain (when present), and/or the seventh binding domain (when present) comprise polypeptides capable of binding moieties present on the cell surface, including proteins, saccharides, and lipids. In one embodiment, the one or more binding proteins comprise cell surface protein binding polypeptides.

In some embodiments, the compositions do not include an effector molecule, as the proximity-dependent binding even may be detectable without an effector protein. In one embodiment of the compositions of any embodiment of the of the disclosure, the effector molecule(s) is/are present. Any effector molecule suitable for an intended use may be used. In one embodiment, the effector molecule(s) are selected from the non-limiting group comprising Bcl2, GFP1-10, small molecules, antibodies, antibody drug conjugates, immunogenic peptides, proteases, T cell receptors, cytotoxic agents, fluorophores, fluorescent proteins, cell adhesion molecules, endocytic receptors, phagocytic receptors, magnetic beads, and gel filtration resin, and polypeptides having at least 40%, 45%, 50%, 55%, 60%, 65%, 70%, 75%, 80%, 85%, 90%, 91%, 92%, 93%, 94%, 95%, 96%, 97%, 98%, 99%, or 100% sequence to the amino acid sequence selected from the group consisting of SEQ ID NOS: 27,460-27,469.

Cage and Key Polypeptides

The polypeptides disclosed herein can be used as cage polypeptides that sequester a bioactive peptide in an inactive state (until activated by a key polypeptide binding to the cage polypeptide, as described herein), and wherein the binding domain can serve to target the polypeptide to the entity to which the binding domain binds. In one embodiment, the polypeptides are part of a “protein switch” (together with appropriate key polypeptide(s)), wherein the cage polypeptide and the key polypeptide comprise binding domains that bind to different targets, and the key polypeptide binds to the cage polypeptide and triggers activation of the bioactive peptide only when the different targets are closely associated so that the cage and key polypeptides are co-localized while bound to their targets.

In some aspects, the cage polypeptide comprises a helical bundle, comprising between 2 and 7 alpha-helices; wherein the helical bundle is fused to one or more binding domain; wherein the one or more binding domain and the helical bundle are not both present in the same naturally occurring polypeptide.

In each embodiment, the N-terminal and/or C-terminal 60 amino acids of each cage polypeptides may be optional, as the terminal 60 amino acid residues may comprise a latch region that can be modified, such as by replacing all or a portion of a latch with a bioactive peptide. In one embodiment, the N-terminal 60 amino acid residues are optional; in another embodiment, the C-terminal 60 amino acid residues are optional; in a further embodiment, each of the N-terminal 60 amino acid residues and the C-terminal 60 amino acid residues are optional. In one embodiment, these optional N-terminal and/or C-terminal 60 residues are not included in determining the percent sequence identity. In another embodiment, the optional residues may be included in determining percent sequence identity.

In some aspects, the first cage polypeptide comprises no more than 5 alpha helices, no more than 4 alpha helices, no more than 3 alpha helices, or no more than 2 alpha helices, wherein the structural region comprises at least one alpha helices and the latch region comprises at least one alpha helices. In some aspects, the structural region of the first cage polypeptide comprises one alpha helix. In some aspects, the structural region of the first cage polypeptide comprises two alpha helices. In some aspects, the structural region of the first cage polypeptide comprises three alpha helices.

In some aspects, the first cage polypeptide, the first key polypeptide, the second key polypeptide, and/or the decoy polypeptide are further modified to change (i) hydrophobicity, (ii) a hydrogen bond network, (iii) a binding affinity to each, and/or (iv) any combination thereof. In some aspects, the cage polypeptide and/or the key polypeptide are modified to reduce hydrophobicity. In some aspects, the latch region is mutated to reduce the hydrophobicity. For example, hydrophobic amino acids are known: glycine (Gly), alanine (Ala), valine (Val), leucine (Leu), isoleucine (Ile), proline (Pro), phenylalanine (Phe), methionine (Met), and tryptophan (Trp). In some aspects, one or more hydrophobic amino acids are replaced with a polar amino acid, e.g., serine (Ser), threonine (Thr), cysteine (Cys), asparagine (Asn), glutamine (Gln), and tyrosine (Tyr). In some aspects, an interface between the latch region and the structural region of the first cage polypeptide includes a hydrophobic amino acid to polar amino acid residue ratio of between 1:1 and 10:1, e.g., 1:1, 2:1, 3:1, 4:1, 5:1, 6:1, 7:1, 8:1, 9:1, or 10:1. In some aspects, an interface between the latch region and the structural region includes a hydrophobic amino acid to polar amino acid residue ratio of 1:1. In some aspects, an interface between the latch region and the structural region includes a hydrophobic amino acid to polar amino acid residue ratio of 2:1. In some aspects, an interface between the latch region and the structural region includes a hydrophobic amino acid to polar amino acid residue ratio of 3:1. In some aspects, an interface between the latch region and the structural region includes a hydrophobic amino acid to polar amino acid residue ratio of 4:1. In some aspects, an interface between the latch region and the structural region includes a hydrophobic amino acid to polar amino acid residue ratio of 5:1. In some aspects, an interface between the latch region and the structural region includes a hydrophobic amino acid to polar amino acid residue ratio of 6:1. In some aspects, an interface between the latch region and the structural region includes a hydrophobic amino acid to polar amino acid residue ratio of 7:1. In some aspects, an interface between the latch region and the structural region includes a hydrophobic amino acid to polar amino acid residue ratio of 8:1. In some aspects, an interface between the latch region and the structural region includes a hydrophobic amino acid to polar amino acid residue ratio of 9:1. In some aspects, an interface between the latch region and the structural region includes a hydrophobic amino acid to polar amino acid residue ratio of 10:1.

In some aspects, 1, 2, 3, or more large hydrophobic residues in the latch region, e.g., isoleucine, valine, or leucine, are mutated to serine, threonine, or a smaller hydrophobic amino acid residue, e.g., valine (if the starting amino acid is isoleucine or leucine) or alanine.

In some aspects, the first cage polypeptide comprises buried amino acid residues at the interface between the latch region and the structural region of the first cage polypeptide, wherein the buried amino acid residues at the interface have side chains comprising nitrogen or oxygen atoms involved in hydrogen bonding.

In another embodiment of the compositions of any aspect and embodiment of the disclosure, the first cage polypeptide, the second cage polypeptide, and/or the decoy cage polypeptide comprise:

(a) a polypeptide comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence of a cage polypeptide disclosed herein, or selected from the group consisting SEQ IDS NOS: 27359-27392, 1-49, 51-52, 54-59, 61, 65, 67-14317, 27094-27117, 27120-27125, and 27278-27321 not including optional amino acid residues; or cage polypeptides listed in Table 7, Table 8, or Table 9, wherein the N-terminal and/or C-terminal 60 amino acids of the polypeptides are optional; and

(b) one or more first, fifth, sixth, or seventh binding domains.

In another embodiment, the first cage polypeptide, the second cage polypeptide, and/or the decoy cage polypeptide comprise:

(a) a polypeptide comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence of a cage polypeptide disclosed herein, or selected from the group consisting SEQ IDS NOS: 27359-27392, not including optional amino acid residues; and

(b) one or more first, fifth, sixth, or seventh binding domains.

In one embodiment, the first cage polypeptide, the second cage polypeptide, and/or the decoy cage polypeptide comprise an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical along its length to the amino acid sequence of a cage polypeptide disclosed herein, or selected from the group consisting SEQ IDS NOS: 27359-27392, including optional amino acid residues

In another embodiment, the first key polypeptide and/or the second key polypeptide comprise:

(a) a polypeptide comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NOS: 27393-27398, 14318-26601, 26602-27015, 27016-27050, 27,322-27,358, and key polypeptides listed in Table 7, Table 8, and/or Table 9; and

(b) one or more second, third, or fourth binding domains.

In a further embodiment, the first key polypeptide and/or the second key polypeptide comprise:

(a) a polypeptide comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27393-27398, or SEQ ID NOS: 27394-27395, not including optional residues, or including optional residues; and

(b) one or more second, third, or fourth binding domains.

As disclosed herein, bioactive peptides to be sequestered by the polypeptides of the disclosure are located within the latch region. The latch region is denoted by brackets in the sequence of each cage polypeptide. The bioactive peptide may be added to the latch region without removing any residues of the latch region, or may replace one or more (1, 2, 3, 4, 5, 6, 7, 8, 9, 10, or more) amino acid residues in the cage scaffold latch region to produce the final polypeptide. Thus, the latch region may be significantly modified upon inclusion of the bioactive peptide. In one embodiment, the optional residues are not included in determining percent sequence identity. In another embodiment, the latch region residues may be included in determining percent sequence identity. In a further embodiment, each of the optional residues and the latch residues may are not included in determining percent sequence identity.

Exemplary cage and key polypeptides of the disclosure have been identified and subjected to mutational analysis. Furthermore, different designs starting from the same exemplary polypeptides yield different amino acid sequences while maintaining the same intended function. In various embodiments, a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that the desired activity is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In one embodiment of cage polypeptides, interface residues between the latch and structural regions are primarily (i.e.: 50%, 60%, 70%, 75%, 80%, 85%, 90%, or greater) hydrophobic residues. In one embodiment, interface residues are primarily valine, leucine, isoleucine, and alanine residues. In a further embodiment an interface between a latch region and a structural region of the polypeptide includes a hydrophobic amino acid to polar amino acid residue ratio of between 1:1 and 10:1. The cage polypeptides may be “tuned” to modify strength of the interaction between the latch region and structural region as deemed appropriate for an intended use. In one embodiment 1, 2, 3, or more large hydrophobic residues in the latch region, including but not limited to isoleucine, valine or leucine, are mutated to serine, threonine, or a smaller hydrophobic amino acid residue including but not limited to valine (if the starting amino acid is isoleucine or leucine) or alanine. In this embodiment, the tuning weakens structural region-latch affinity. In another embodiment, buried amino acid residues at the interface have side chains comprising nitrogen or oxygen atoms involved in hydrogen bonding. Tuning can include increasing or decreasing the number of hydrogen bonds present at the interface. Based on the teachings herein, those of skill in the art will understand that such tuning may take any number of forms depending on the desired structural region-latch region affinity.

In one embodiment of the compositions of any embodiment or combination of embodiments of the disclosure, (i) the first cage polypeptide, the second cage polypeptide, and/or the decoy cage polypeptide; and (ii) the first and/or second key polypeptide, comprise at least one cage polypeptide and at least one key polypeptide comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence of a cage polypeptide and a key polypeptide, respectively, in the same row or one of 7, 8, or 9 (i.e.: each cage polypeptide in row 2 column 1 of the table can be used with each key polypeptide in row 2 column 1 of the table, and so on), with the proviso that each cage polypeptide and each key polypeptide further comprise one or more binding domain.

In one embodiment, the first cage polypeptide, the second cage polypeptide, and/or the decoy cage polypeptide comprise:

(a) an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the non-limiting group consisting of SEQ ID NOS: 27359-27392, either including optional amino acid residues or not including optional amino acid residues; and

(b) a binding domain comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:27,399-27,403.

In another embodiment, the first key polypeptide and/or the second key polypeptide comprise:

(a) an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27393-27398 or 27394-27395, either including optional amino acid residues or not including optional amino acid residues; and

(b) a binding domain comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical the amino acid sequence selected from the group consisting of SEQ ID NOS: 27,399-27,403.

In another embodiment, the first cage polypeptide, the second cage polypeptide, and/or the decoy cage polypeptide comprise an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27404-27446. In another embodiment, the first key polypeptide and/or the second key polypeptide comprise an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27448-27459. In a further embodiment, (i) the first cage polypeptide, the second cage polypeptide, and/or the decoy cage polypeptide comprise an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27404-27446; and (ii) the first key polypeptide and/or the second key polypeptide comprise an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27448-27459.

TABLE 1
Modular Co-LOCKR Cage domains and Decoy domains
(parentheses are optional sequences of which a portion can be deleted to
tune latch affinity)
(underlined amino acids can be changed to any other amino acid to tune
latch affinity)
(bold is bioactive peptide; other bioactive peptides can be substituted for
Bim or GFP11)
Versions without bioactive peptides are decoy cage polypeptides,
noting that any cage polypeptide can serve as a decoy polypeptide by
exclusion of the bioactive peptide, and any first cage polypeptide can
serve as a decoy polypeptide for any second cage polypeptide, provided that
the first cage polypeptide has a higher affinity for the key polypeptide
than does the second cage polypeptide.

>Cage_Bim_A
SEQ ID NO: 27359
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASELTDEIWIAQELRRIGDEF(NAYYADAERLIREAAAASEKISREAERLIR)
>Decoy_GFP11_A
SEQ ID NO: 27360
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(RDHMVLHEYVNAAGITFNAYYADAERLIREAAAASEKISREAG)
>Decoy_GFP11_C
SEQ ID NO: 27361
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(RDHMVLHEYVNAAGITFNAYYADAERLIREAAAASEKISREAERLIR)
>Decoy_AYYA_A
SEQ ID NO: 27362
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(TDPDEARKAIARVKRESNAYYADAERLIREAAAASEKISREAERLIR)
>Decoy_AYYA_B
SEQ ID NO: 27363
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASELG(TDPDEARKAIARVKRESNAYYADAERLIREAAAASEKISREAERLIR)
>Decoy_GFP11_B
SEQ ID NO: 27364
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(RDHMVLHEYVNAAGITFNAYYADAERLIREAAAASEKISREAG)
SEQ ID NO: 27365
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(TDEIWIAQELRRIGDEFNAYYADAERLIREAAAASEKISREAERLAR)
SEQ ID NO: 27366
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(TDEIWIAQELRRIGDEFNAYYADAERLSREAAAASEKISREAERLIR)
Anti-Her2-scFv_Cage_I269S Tuned Cage targeted to Her2 by anti-her2 scfv
SEQ ID NO: 27367
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASELTDEIWIAQELRRIGDEFNAYYADAERLSREAAAASEKISREAERLIR
SEQ ID NO: 27368
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(TDEIWIAQELRRIGDEFNAYYADAERLIREAAAASEKISREAERLIR)
SEQ ID NO: 27369
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(RDHMVLHEYVNAAGITFNAYYADAERLIREAAAASEKISREAER)
SEQ ID NO: 27370
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(TDPDEARKAIARVKRESNAYYADAERLSREAAAASEKISREAERLIR)
SEQ ID NO: 27371
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(TDPDEARKAIARVKRESNAYYADAERLIREAAAASEKISREAERLIR)
>Her2_Cage_I287S Tuned Cage targeted to Her2 by DARPin (I287S)
SEQ ID NO: 27372
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(TDEIWIAQELRRIGDEFNAYYADAERLIREAAAASEKISREAERLSR)
>Her2_Cage_I269S_I287A Tuned Cage targeted to Her2 by DARPin (I269S, I287A)
SEQ ID NO: 27373
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(TDEIWIAQELRRIGDEFNAYYADAERLSREAAAASEKISREAERLAR)
>Her2_Cage_I269S_I287S Tuned Cage targeted to Her2 by DARPin (I269S, I287A)
SEQ ID NO: 27374
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(TDEIWIAQELRRIGDEFNAYYADAERLSREAAAASEKISREAERLSR)
>Her2_Cage_L209A_L216A Tuned Cage targeted to Her2 by DARPin (L209A)
SEQ ID NO: 27375
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(TDEIWIAQELRRIGDEFNAYYADAERLIREAAAASEKISREAERLIR)
>EGFR_Decoyl Original decoy targeted to EGFR by DARPin (for NOT logic)
SEQ ID NO: 27376
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(RDHMVLHEYVNAAGITFNAYYADAERLIREAAAASEKISREAG)
>EGFR_Decoy_G24 Tuned Decoy targeted to EGFR by DARPin
SEQ ID NO: 27377
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(GRDHMVLHEYVNAAGITFNAYYADAERLIREAAAASEKISREAG)
>EGFR_Decoy_G25 Tuned Decoy targeted to EGFR by DARPin
SEQ ID NO: 27378
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(RDHMVLHEYVNAAGITFNAYYADAERLSREAAAASEKISREAG)
>EGFR_Decoy_G33 Tuned Decoy targeted to EGFR by DARPin
SEQ ID NO: 27379
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(GTDPDEARKAIARVKRESNAYYADAERLSREAAAASEKISREAG)
>EGFR_Decoy_G34 Tuned Decoy targeted to EGFR by DARPin
SEQ ID NO: 27380
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(GTDPDEARKAIARVKRESNAYYADAERLSREAAAASEKISREAER)
>EGFR_Decoy_G35 Tuned Decoy targeted to EGFR by DARPin
SEQ ID NO: 27381
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(GTDPDEARKAIARVKRESNAYYADAERLSREAAAASEKISREAERLI)
>EGFR_Decoy_G7(1A7) Tuned Decoy targeted to EGFR by DARPin
SEQ ID NO: 27382
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(GTDPDEARKAIARVKRESNAYYADAERLSREAAAASEKISREAERLIR)
>EGFR_Decoy_Box1C1 Tuned Decoy targeted to EGFR by DARPin
SEQ ID NO: 27383
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(GTDPDEARKAIARVKRESNAYYADAERLIREAAAASEKISREAERLIR)
>EGFR_Decoy3 Tuned Decoy targeted to EGFR by DARPin
SEQ ID NO: 27384
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(GTDPDEARKAIARVKRESNAYYADAERLSREAAAASEKISREAERLAR)
>EGFR_Decoy5 Tuned Decoy targeted to EGFR by DARPin
SEQ ID NO: 27385
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(GTDPDEARKAIARVKRESNAYYADAERLIREAAAASEKISREAERLAR)
>EGFR_Decoy7 Tuned Decoy targeted to EGFR by DARPin
SEQ ID NO: 27386
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(TDPDEARKAIARVKRESNAYYADAERLSREAAAASEKISREAERLAR)
>EGFR_Decoy10 Tuned Decoy targeted to EGFR by DARPin
SEQ ID NO: 27387
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(GSE(D/N)LYFQGSTDPDEARKAIARVKRESNAYYADAERLSREAAAASEKISREAER
LAR)
>EGFR_Decoy11 Tuned Decoy targeted to EGFR by DARPin
SEQ ID NO: 27388
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(GSE(D/N)LYFQGSTDPDEARKAIARVKRESNAYYADAERLSREAAAASEKISREAER
LIR)
>EGFR_Decoy12 Tuned Decoy targeted to EGFR by DARPin
SEQ ID NO: 27389
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(GSE(D/N)LYFQGSTDPDEARKAIARVKRESNAYYADAERLIREAAAASEKISREAER
LAR)
>EGFR_Decoy13 Tuned Decoy targeted to EGFR by DARPin
SEQ ID NO: 27390
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(GSE(D/N)LYFQGSTDPDEARKAIARVKRESNAYYADAERLIREAAAASEKISREAER
LIR)
>EGFR_Decoy_G30 Tuned Decoy targeted to EGFR by DARPin
SEQ ID NO: 27391
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(GTDPDEARKAIARVKRESNAYYADAERLSREAAAASEKISREAERLSR)
>EGFR_Decoy_G32 Tuned Decoy targeted to EGFR by DARPin
SEQ ID NO: 27392
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASEL(GTDPDEARKAIARVKRESNAYYADAERLSREAAAASEKISREAERLIR)

TABLE 2
Other exemplary cage polypeptides (see also SEQ ID NOS: 92-14317,
27094-27117, 27120-27125, 27,728-27321, and cage polypeptides listed in Table 7,
Table 8, and/or Table 9):

1) Exemplary reference cage polypeptides; latch regions denoted by brackets [ ]
6His-MBP-TEV, 6His-TEV, and flexible linker sequences are underlined text
fused functional domains (DARPins, componants of the split intein, and fluorescent
proteins) are bolded text
Functional peptide is italicized underlined text
Exemplary positions that have been mutated to any amino acid to tune responsiveness
are underlined bolded text. These positions are exemplary, and not an exhaustive list
of residues able to tune responsiveness.
C-terminal sequences that can be removed to tune responsiveness are contained
within brackets. A range from one (1) to all residues encompassed within the
brackets may be removed, starting from the C-terminus and removing successive
residues therein.
All sequences in parentheses are optional
>SB76L (SEQ ID NO: 1)
(MGSSHHHHHHSSGLVPRGSHM)SKEAVTKLQALNIKLAEKLLEALARLQELNIALVYLAVELTDPKRIADEIKKVKDKSKEI
VERAEEEIARAAAESKKILDEGSGSGSDAVAELQALNLKLAELLLEAIAKLQELNIKLVELLTKLTDPATIREAIRKVKEDSE
RIVAEAERLIAAAKAESERIIREGSGSGDPDVARLQELNIELARELLRAAAELQELNIKLVELASELTDP[DEARKAIARVKR
ESKRIVEDAERLIREAAAASEKISRE]
>SB76L_17 (SEQ ID NO: 2)
(MGSSHHHHHHSSGLVPRGSHM)GSKEAVTKLMALNLKLAEKLLEAIARLQELNIALVYLATELTDPERIREEIRKVKEESAR
IVEEAEEEIRRAAARSEDILREGSGSGSDAVAELQRLNLELAELLLRAAAKLQELNIDLVRLLTELTDPKTIRDAIERVKAES
ERIVREAERLIREAKADSERILREGSGSGDPDVARLQELFIELARELLEALARLQELNIDLVRLASELTDP[DTIRDAIRRVK
EESARIVEDARRLIKEAAEEAEKISRE]
>SB76L_18 (SEQ ID NO: 3)
(MGSSHHHHHHSSGLVPRGSHM)GSKRAVTELQKLNIELARKLLRALAELMELNIALVYLAVELTDPRRIREEIRKVKEKSDE
IVKRAEDEIRKAAAESEKILREGSGSGSDAVAELQRLNLELAKLLLEAIAKLQALNIDLVRLLTELTDPETIRRAIKRVKDES
ARIVEEAEKLIRAAKDKAREIIDKGSGSGDPDVARLQELNIELARELLEAAARLQELFIDLVRLASELTDP[DEARKAIERVK
REAERIVREAERLIREAKRASKEISDE]
>LOCKR_extend5 (SEQ ID NO: 4)
(MGSSHHHHHHSSGLVPRGSHM)KLLEAVTKLQALNIKLAEKLLEALARLQELNIALVYLAVELTDPKRIADEIKKVKDKSKE
IVERAEEEIARAAAESKKILDEAEEEGSGSGSELLLEAVAELQALNLKLAELLLEAIAKLQELNIKLVELLTKLTDPATIREA
IRKVKEDSERIVAEAERLIAAAKAESERIIREAERLAGSGSGSRELLRDVARLQELNIELARELLRAAAELQELNIKLVELAS
ELTDP[DEARKAIARVKRESKRIVEDAERLIREAAAASEKISREAERLI]
>LOCKR_extend9 (SEQ ID NO: 5)
(MGSSHHHHHHSSGLVPRGSHM)KLAEKLLEAVTKLQALNIKLAEKLLEALARLQELNIALVYLAVELTDPKRIADEIKKVKD
KSKEIVERAEEEIARAAAESKKILDEAEEEIARAGSGSGSLKLAELLLEAVAELQALNLKLAELLLEAIAKLQELNIKLVELL
TKLTDPATIREAIRKVKEDSERIVAEAERLIAAAKAESERIIREAERLIAAAAGSGSGSIELARELLRDVARLQELNIELARE
LLRAAAELQELNIKLVELASELTDP[DEARKAIARVKRESKRIVEDAERLIREAAAASEKISREAERLIREAA]
>LOCKR_extend18 (SEQ ID NO: 6)
(MGSSHHHHHHSSGLVPRGSHM)SKEAVTKLQALNIKLAEKLLEAVTKLQALNIKLAEKLLEALARLQELNIALVYLAVELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARLQELNIELARELLRDVARLQELNIELARELLRAAAELQELNIKLVELASELTDP[DEARK
AIARVKRESKRIVEDAERLIREAAAASEKISREAERLIREAAAASEKISRE]
>LOCKRb (SEQ ID NO: 7)
(MGSSHHHHHHSSGLVPRGSHM)SHAAVIKLSDLNIRLLDKLLQAVIKLTELNAELNRKLIEALQRLFDLNVALVHLAAELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSNDPQVAQNQETFIELARDALRLVAENQEAFIEVARLTLRAAALAQEVAIKAVEAASEGGSGSG[NKE
EIEKLAKEAREKLKKAEKEHKEIHDKLRKKNKKAREDLKKKADELRETNKRVN]
>LOCKRc (SEQ ID NO: 8)
(MGSSHHHHHHSSGLVPRGSHM)SLEAVLKLAELNLKLSDKLAEAVQKLAALLNKLLEKLSEALQRLFELNVALVTLAIELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSNDPLVARLQELLIEHARELLRLVATSQEIFIELARAFLANAAQLQEAAIKAVEAASENGSGSG[SSE
KVRRELKESLKENHKQNQKLLKDHKRAQEKLNRELEELKKKHKKTLDDIRRES]
>LOCKRd (SEQ ID NO: 9)
(MGSSHHHHHHSSGLVPRGSHM)SLEAVLKLFELNHKLSEKLLEAVLKLHALNQKLSQKLLEALARLLELNVALVELAIELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPEVARLQEAFIEQAREILRNVAAAQEALIEQARRLLALAALAQEAAIKAVELASEHGSGSG[DTV
KRILEELRRRFEKLAKDLDDIARKLLEDHKKHNKELKDKQRKIKKEADDAARS]
>LOCKRe (SEQ ID NO: 10)
(MGSSHHHHHHSSGLVPRGSHM)SLEAVLKLQDLNSKLSEKLSEAQLKLQALNNKLLRKLLEALLRLQDLNQALVNLALQLTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVAKSQEHLIEHARELLRQVAKSQELFIELARQLLRLAAKSQELAIKAVELASEAGSGSG[DDV
ERRLRKANKESKKEAEELTEEAKKANEKTKEDSKELTKENRKTNKTIKDEARS]
>LOCKRf (SEQ ID NO: 11)
(MGSSHHHHHHSSGLVPRGSHM)SREAVEKLAELNHKLSHKLQQAQQKLQALNLKLLQKLLEALDRLQDLNNALVKLAQRLTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARQQETLIEQARRLLRNVAESQELFIEAARTVLRLAAKLQEINIKQVELASEAGSGSG[DDE
ERRSEKTVQDAKREIKKVEDDLQRLNEEQKKKVKKQEDENQKTLKKHKDDARS]
>miniLOCKRa_1 (SEQ ID NO: 12)
(MGSSHHHHHHSSGLVPRGSHM)NKEDATEAQKKAIRAAEELLKDVTRIQERAIREAEKALERLARVQEEAIRRVYEAVESKN
KEELKKVKEEIEELLRRLKRELDELEREIRELLKEIKEKADRLEKEIRDLIERIRRDRNASDEVVTRLARLNEELIRELREDV
RRLAELNKELLRELERAARELARLNEKLLELADRVETE[EEARKAIARVKRESKRIVEDAERLIREAAAASEKISREAERLIR
EAAAASEKISRE]
>miniLOCKRa_2 (SEQ ID NO: 13)
(MGSSHHHHHHSSGLVPRGSHM)DERLKRLNERLADELDKDLERLLRLNEELARELTRAAEELRELNEKLVELAKKLQGGRSR
EVAERAEKEREKIRRKLEEIKKEIKEDADRIKKRADELRRRLEKTLEDAARELEKLKREPRTEELKRKATELQKEAIRRAEEL
LKEVTDVQRRAIERAEELLEKLARLQEEAIRTVYLLVELNKV[DRARKAIARVKRESKRIVEDAERLIREAAAASEKISREAE
RLIREAAAASEKISRE]
>miniLOCKRc_1 (SEQ ID NO: 14)
(MGSSHHHHHHSSGLVPRGSHM)LIERLTRLEKEHVRELKRLLDTSLEILRRLVEAFETNLRQLKEALKRALEAANLHNEEVE
EVLRKLEEDLRRLEEELRKTLDDVRKEVKRLKEELDKRIKEVEDELRKIKEKLKKGDKNEKRVLEEILRLAEDVLKKSDKLAK
DVQERARELNEILEELSRKLQELFERVVEEVTRNVPT[TERIEKVRRELKESLKENHKQNQKLLKDHKRAQEKLNRELEELKK
KHKKTLDDIRRES]
>miniLOCKRc_2 (SEQ ID NO: 15)
(MGSSHHHHHHSSGLVPRGSHM)SEERVLELAEEALRLSDEAAKEIQELARRLNEELEKLSKELQDLFERIVETVTRLIDADE
ETLKRAAEEIKKRLEDARKKAKEAADKAREELDRARKKLKELVDEIRKKAKDALEKAGADEELVARLLRLLEEHARELERLLR
TSARIIERLLDAFRRNLEQLKEAADKAVEAAEEAVRRVED[VRVWSEKVRRELKESLKENHKQNQKLLKDHKRAQEKLNRELE
ELKKKHKKTLDDIRRES]
>1fix-short-noBim-t0 (SEQ ID NO: 16)
(MGSHHHHHHGSGSENLYFQGSGG)SELARKLLEASTKLQRLNIRLARALLEAIARLQELNLELVYLAVELTDPKRIRDEIKE
VKDKSKEIIRRAEKEIDDAAKESEKILEEAREAISGSGSELAKLLLKAIAETQDLNLRAAKAFLEAAAKLQELNIRAVELLVK
LTDPATIREALEHAKRRSKEIIDEAERAIRAAKRESERIIEEARRLIEKGSGSGS[ELARELLRAHAQLQRLNLELLRELLRA
LAQLQELNLDLLRLASELTDPDEARKAIARVKRESKRIVEDAERLIREAAAASEKISREAERLIR]
>1fix-short-noBim(AYYA)-t0 (SEQ ID NO: 17)
(MGSHHHHHHGSGSENLYFQGSGG)SELARKLLEASTKLQRLNIRLARALLEAIARLQELNLELVYLAVELTDPKRIRDEIKE
VKDKSKEIIRRAEKEIDDAAKESEKILEEAREAISGSGSELAKLLLKAIAETQDLNLRAAKAFLEAAAKLQELNIRAVELLVK
LTDPATIREALEHAKRRSKEIIDEAERAIRAAKRESERIIEEARRLIEKGSGSGS[ELARELLRAHAQLQRLNLELLRELLRA
LAQLQELNLDLLRLASELTDPDEARKAIARVKRESNAYYADAERLIREAAAASEKISREAERLIR]
“(3) Functional LOCKR Cage designs with bioactive peptides encoded into the Latch”,
>aBc12LOCKR (SEQ ID NO: 18)
(MGSSHHHHHHSSGLVPRGSHM)GSKEAVTKLQALNIKLAEKLLEALARLQELNIALVYLAVELTDPKRIADEIKKVKDKSKE
IVERAEEEIARAAAESKKILDEGSGSGSDAVAELQALNLKLAELLLEAIAKLQELNIKLVELLTKLTDPATIREAIRKVKEDS
ERIVAEAERLIAAAKAESERIIREGSGSGDPDVARLQELNIELARELLRAAAELQELNIKLVELASELT(GSGSGSG)[DPKM
AQEL [symbol] DE [symbol] RAASLQI [symbol] GDAFYA [symbol] LRALAASEKLSKE]
>pBimLOCKR (SEQ ID NO: 19)
(MGSSHHHHHHSSGLVPRGSHM)KEAVTKLQALNIKLAEKLLEALARLQELNIALVYLAVELTDPKRIADEIKKVKDKSKEIV
ERAEEEIARAAAESKKILDEGSGSGSDAVAELQALNLKLAELLLEAIAKLQELNIKLVELLTKLTDPATIREAIRKVKEDSER
IVAEAERLIAAAKAESERIIREGSGSGDPDVARLQELNIELARELLRAAAELQELNIKLVELASEGSGSGS[EIAEALRAIGD
VFNESYRIVEDAERLIREAAAASEKISRE]
>BimLOCKR_extend5 (SEQ ID NO: 20)
(MGSSHHHHHHSSGLVPRGSHM)KLLEAVTKLQALNIKLAEKLLEALARLQELNIALVYLAVELTDPKRIADEIKKVKDKSKE
IVERAEEEIARAAAESKKILDEAEEEGSGSGSELLLEAVAELQALNLKLAELLLEAIAKLQELNIKLVELLTKLTDPATIREA
IRKVKEDSERIVAEAERLIAAAKAESERIIREAERLAGSGSGSRELLRDVARLQELNIELARELLRAAAELQELNIKLVELAS
ELTD[EIWIAQELRRIGDEFNAYYADAERLIREAAAASEKISREAERLI]
>BimLOCKR_extend9 (SEQ ID NO: 21)
(MGSSHHHHHHSSGLVPRGSHM)KLAEKLLEAVTKLQALNIKLAEKLLEALARLQELNIALVYLAVELTDPKRIADEIKKVKD
KSKEIVERAEEEIARAAAESKKILDEAEEEIARAGSGSGSLKLAELLLEAVAELQALNLKLAELLLEAIAKLQELNIKLVELL
TKLTDPATIREAIRKVKEDSERIVAEAERLIAAAKAESERIIREAERLIAAAAGSGSGSIELARELLRDVARLQELNIELARE
LLRAAAELQELNIKLVELASELTD[EIWIAQELRRIGDEFNAYYADAERLIREAAAASEKISREAERLIREAA]
>BimLOCKR_extend18 (SEQ ID NO: 22)
(MGSSHHHHHHSSGLVPRGSHM)SKEAVTKLQALNIKLAEKLLEAVTKLQALNIKLAEKLLEALARLQELNIALVYLAVELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARLQELNIELARELLRDVARLQELNIELARELLRAAAELQELNIKLVELASELTD[EIWIAQ
ELRRIGDEFNAYYADAERLIREAAAASEKISREAERLIREAAAASEKISRE]
>BimLOCKRb (SEQ ID NO: 23)
(MGSSHHHHHHSSGLVPRGSHM)SHAAVIKLSDLNIRLLDKLLQAVIKLTELNAELNRKLIEALQRLFDLNVALVHLAAELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSNDPQVAQNQETFIELARDALRLVAENQEAFIEVARLTLRAAALAQEVAIKAVEAASEGGSGSG[NEI
WIAQELRRIGDEFNAYYAEHKEIHDKLRKKNKKAREDLKKKADELRETNKRVN]
>BimLOCKRc (SEQ ID NO: 24)
(MGSSHHHHHHSSGLVPRGSHM)SLEAVLKLAELNLKLSDKLAEAVQKLAALLNKLLEKLSEALQRLFELNVALVTLAIELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSNDPLVARLQELLIEHARELLRLVATSQEIFIELARAFLANAAQLQEAAIKAVEAASENGSG[EIWIA
QELRRIGDEFNAYYAQNQKLLKDHKRAQEKLNRELEELKKKHKKTLDDIRRES]
>BimLOCKRd (SEQ ID NO: 25)
(MGSSHHHHHHSSGLVPRGSHM)SLEAVLKLFELNHKLSEKLLEAVLKLHALNQKLSQKLLEALARLLELNVALVELAIELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPEVARLQEAFIEQAREILRNVAAAQEALIEQARRLLALAALAQEAAIKAVELASEHGSGS[EIWI
AQELRRIGDEFNAYYADLDDIARKLLEDHKKHNKELKDKQRKIKTIKDEARS]
>StrepLOCKRa_300 (SEQ ID NO: 26)
(MGSSHHHHHHSSGLVPRGSHM)SKEAVTKLQALNIKLAEKLLEAVTKLQALNIKLAEKLLEALARLQELNIALVYLAVELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARLQELNIELARELLRDVARLQELNIELARELLRAAAELQELNIKLVELAS(GG)[NWSHPQ
FEKKAIARVKRESKRIVEDAERLIREAAAASEKISREAERLIREAAAASEKISRE]
>strepLOCKRa_306 (SEQ ID NO: 27)
(MGSSHHHHHHSSGLVPRGSHM)SKEAVTKLQALNIKLAEKLLEAVTKLQALNIKLAEKLLEALARLQELNIALVYLAVELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARLQELNIELARELLRDVARLQELNIELARELLRAAAELQELNIKLVELASELTDPD[ENWS
HPQFEKRESKRIVEDAERLIREAAAASEKISREAERLIREAAAASEKISRE]
>strepLOCKRa_309 (SEQ ID NO: 28)
(MGSSHHHHHHSSGLVPRGSHM)SKEAVTKLQALNIKLAEKLLEAVTKLQALNIKLAEKLLEALARLQELNIALVYLAVELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARLQELNIELARELLRDVARLQELNIELARELLRAAAELQELNIKLVELASELTDPD[EARK
NWSHPQFEKKRIVEDAERLIREAAAASEKISREAERLIREAAAASEKISRE]
>strepLOCKRa_312 (SEQ ID NO: 29)
(MGSSHHHHHHSSGLVPRGSHM)SKEAVTKLQALNIKLAEKLLEAVTKLQALNIKLAEKLLEALARLQELNIALVYLAVELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARLQELNIELARELLRDVARLQELNIELARELLRAAAELQELNIKLVELASELTDPD[EARK
AIANWSHPQFEKVEDAERLIREAAAASEKISREAERLIREAAAASEKISRE]
>strepLOCKRa_313 (SEQ ID NO: 30)
(MGSSHHHHHHSSGLVPRGSHM)SKEAVTKLQALNIKLAEKLLEAVTKLQALNIKLAEKLLEALARLQELNIALVYLAVELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARLQELNIELARELLRDVARLQELNIELARELLRAAAELQELNIKLVELASELTDPD[EARK
AIARNWSHPQFEKEDAERLIREAAAASEKISREAERLIREAAAASEKISRE]
>strepLOCKRa_317 (SEQ ID NO: 31)
(MGSSHHHHHHSSGLVPRGSHM)SKEAVTKLQALNIKLAEKLLEAVTKLQALNIKLAEKLLEALARLQELNIALVYLAVELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARLQELNIELARELLRDVARLQELNIELARELLRAAAELQELNIKLVELASELTDPD[EARK
AIARVKRENWSHPQFEKRLIREAAAASEKISREAERLIREAAAASEKISRE]
>strepLOCKRa_320 (SEQ ID NO: 32)
(MGSSHHHHHHSSGLVPRGSHM)SKEAVTKLQALNIKLAEKLLEAVTKLQALNIKLAEKLLEALARLQELNIALVYLAVELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARLQELNIELARELLRDVARLQELNIELARELLRAAAELQELNIKLVELASELTDPD[EARK
AIARVKRESKRNWSHPQFEKREAAAASEKISREAERLIREAAAASEKISRE]
>strepLOCKRa_323 (SEQ ID NO: 33)
(MGSSHHHHHHSSGLVPRGSHM)SKEAVTKLQALNIKLAEKLLEAVTKLQALNIKLAEKLLEALARLQELNIALVYLAVELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARLQELNIELARELLRDVARLQELNIELARELLRAAAELQELNIKLVELASELTDPD[EARK
AIARVKRESKRIVENWSHPQFEKAAASEKISREAERLIREAAAASEKISRE]
>strepLOCKRa_329 (SEQ ID NO: 34)
(MGSSHHHHHHSSGLVPRGSHM)SKEAVTKLQALNIKLAEKLLEAVTKLQALNIKLAEKLLEALARLQELNIALVYLAVELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARLQELNIELARELLRDVARLQELNIELARELLRAAAELQELNIKLVELASELTDPD[EARK
AIARVKRESKRIVEDAERLINWSHPQFEKISREAERLIREAAAASEKISRE]
>SB13_LOCKR (SEQ ID NO: 35)
(MGSSHHHHHHSSGLVPRGSHM)GSKEAVTKLQALNIKLAEKLLEALARLQELNIALVYLAVELTDPKRIADEIKKVKDKSKE
IVERAEEEIARAAAESKKILDEGSGSGSDAVAELQALNLKLAELLLEAIAKLQELNIKLVELLTKLTDPATIREAIRKVKEDS
ERIVAEAERLIAAAKAESERIIREGSGSGDPDVARLQELNIELARELLRAAAELQELNIKLVELASEGSGSGSGYELRRALE
ELEKALRELEKSLDEL [symbol] RSLEEL [symbol] KNPSEDALVENNRLNVENNKIIVEVLRIIAEVLKINAKS]
>ZCX12_LOCKR (SEQ ID NO: 36)
(MGSSHHHHHHSSGLVPRGSHM)GSKEAVTKLQALNIKLAEKLLEALARLQELNIALVYLAVELTDPKRIADEIKKVKDKSKE
IVERAEEEIARAAAESKKILDEGSGSGSDAVAELQALNLKLAELLLEAIAKLQELNIKLVELLTKLTDPATIREAIRKVKEDS
ERIVAEAERLIAAAKAESERIIREGSGSGDPDVARLQELNIELARELLRAAAELQELNIKLVELASEGSGSGSGKKLVEEVE
RALRELLKTSEDLVRKV [symbol] KALRELLELIRRGGTKDKIEEKIRRVLEEIKRELERQKRKIEDVLRQIKEELYRS]
>SB13_LOCKR_extend18 (SEQ ID NO: 37)
(MGSSHHHHHHSSGLVPRGSHM)SKEAVTKLQALNIKLAEKLLEAVTKLQALNIKLAEKLLEALARLQELNIALVYLAVELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARLQELNIELARELLRDVARLQELNIELARELLRAAAELQELNIKLVELASEGSGSGSGYE
LRRALEELEKALRELKKSLDELERSLEELEKNPSEDALVENNRLNVENNKIIVEVLRIIAEVLKINAKS]
>ZCX12_LOCKR_extend18 (SEQ ID NO: 38)
(MGSSHHHHHHSSGLVPRGSHM)SKEAVTKLQALNIKLAEKLLEAVTKLQALNIKLAEKLLEALARLQELNIALVYLAVELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARLQELNIELARELLRDVARLQELNIELARELLRAAAELQELNIKLVELASEGSGSGSGKK
LVEEVERALRELLKTSEDLVRKVEKALRELLELIRRGGTKDKIEEKIRRVLEEIKRELERQKRKIEDVLRQIKEELYRS
>fretLOCKRa (SEQ ID NO: 39)
(GHHHHHHHHHHGVSKGEELFTGVVPILVELDGDVNGHKFSVSGEGEGDATYGKLTLKFICTTGKLPVPWPTLVTTLSWGVQC
FARYPDHMKQHDFFKSAMPEGYVQERTIFFKDDGNYKTRAEVKFEGDTLVNRIELKGIDFKEDGNILGHKLEYNYFSDNVYIT
ADKQKNGIKANFKIRHNIEDGGVQLADHYQQNTPIGDGPVLLPDNHYLSTQSKLSKDPNEKRDHMVLLEFVTAAGITLGMDEL
YKGSGCSLQGM)SKEAVTKLQALNIKLAEKLLEAVTKLQALNIKLAEKLLEALARLQELNIALVYLAVELTDPKRIADEIKKV
KDKSKEIVERAEEEIARAAAESKKILDEAEEEIARAAAESKKILDEGSGSGSDAVAELQALNLKLAELLLEAVAELQALNLKL
AELLLEAIAKLQELNIKLVELLTKLTDPATIREAIRKVKEDSERIVAEAERLIAAAKAESERIIREAERLIAAAKAESERIIR
EGSGSGDPDVARLQELNIELARELLRDVARLQELNIELARELLRAAAELQELNIKLVELASELTDPD[EARKAIARVKRESKR
IVEDAERLIREAAAASEKISREAERLIREAAAASEKISRE](VSKGEELFTGVVPILVELDGDVNGHKFSVSGEGEGDATYGK
LTLKLICTTGKLPVPWPTLVTTLGYGVQCFARYPDHMKQHDFFKSAMPEGYVQERTIFFKDDGNYKTRAEVKFEGDTLVNRIE
LKGIDFKEDGNILGHKLEYNYNSHNVYITADKQKNGIKANFKIRHNIEDGGVQLADHYQQNTPIGDGPVLLPDNHYLSYQSKL
SKDPNEKRDHMVLLEFVTAAGITLGMDELYK)
>fretLOCKRb (SEQ ID NO: 40)
(MGHHHHHHHHHHGVSKGEELFTGVVPILVELDGDVNGHKFSVSGEGEGDATYGKLTLKFICTTGKLPVPWPTLVTTLSWGVQ
CFARYPDHMKQHDFFKSAMPEGYVQERTIFFKDDGNYKTRAEVKFEGDTLVNRIELKGIDFKEDGNILGHKLEYNYFSDNVYI
TADKQKNGIKANFKIRHNIEDGGVQLADHYQQNTPIGDGPVLLPDNHYLSTQSKLSKDPNEKRDHMVLLEFVTAAGITLGMDE
LYKGSGCSLQGM)SHAAVIKLSDLNIRLLDKLLQAVIKLTELNAELNRKLIEALQRLFDLNVALVHLAAELTDPKRIADEIKK
VKDKSKEIVERAEEEIARAAAESKKILDEAEEEIARAAAESKKILDEGSGSGSDAVAELQALNLKLAELLLEAVAELQALNLK
LAELLLEAIAKLQELNIKLVELLTKLTDPATIREAIRKVKEDSERIVAEAERLIAAAKAESERIIREAERLIAAAKAESERII
REGSGSNDPQVAQNQETFIELARDALRLVAENQEAFIEVARLTLRAAALAQEVAIKAVEAASEGGSGSG[NKEEIEKLAKEAR
EKLKKAEKEHKEIHDKLRKKNKKAREDLKKKADELRETNKRVN](VSKGEELFTGVVPILVELDGDVNGHKFSVSGEGEGDAT
YGKLTLKLICTTGKLPVPWPTLVTTLGYGVQCFARYPDHMKQHDFFKSAMPEGYVQERTIFFKDDGNYKTRAEVKFEGDTLVN
RIELKGIDFKEDGNILGHKLEYNYNSHNVYITADKQKNGIKANFKIRHNIEDGGVQLADHYQQNTPIGDGPVLLPDNHYLSYQ
SKLSKDPNEKRDHMVLLEFVTAAGITLGMDELYK)
>fretLOCKRc (SEQ ID NO: 41)
(GHHHHHHHHHHGVSKGEELFTGVVPILVELDGDVNGHKFSVSGEGEGDATYGKLTLKFICTTGKLPVPWPTLVTTLSWGVQC
FARYPDHMKQHDFFKSAMPEGYVQERTIFFKDDGNYKTRAEVKFEGDTLVNRIELKGIDFKEDGNILGHKLEYNYFSDNVYIT
ADKQKNGIKANFKIRHNIEDGGVQLADHYQQNTPIGDGPVLLPDNHYLSTQSKLSKDPNEKRDHMVLLEFVTAAGITLGMDEL
YKGSGCSLQGM)SLEAVLKLAELNLKLSDKLAEAVQKLAALLNKLLEKLSEALQRLFELNVALVTLAIELTDPKRIADEIKKV
KDKSKEIVERAEEEIARAAAESKKILDEAEEEIARAAAESKKILDEGSGSGSDAVAELQALNLKLAELLLEAVAELQALNLKL
AELLLEAIAKLQELNIKLVELLTKLTDPATIREAIRKVKEDSERIVAEAERLIAAAKAESERIIREAERLIAAAKAESERIIR
EGSGSNDPLVARLQELLIEHARELLRLVATSQEIFIELARAFLANAAQLQEAAIKAVEAASENGSGSGS[SEKVRRELKESLK
ENHKQNQKLLKDHKRAQEKLNRELEELKKKHKKTLDDIRRES](VSKGEELFTGVVPILVELDGDVNGHKFSVSGEGEGDATY
GKLTLKLICTTGKLPVPWPTLVTTLGYGVQCFARYPDHMKQHDFFKSAMPEGYVQERTIFFKDDGNYKTRAEVKFEGDTLVNR
IELKGIDFKEDGNILGHKLEYNYNSHNVYITADKQKNGIKANFKIRHNIEDGGVQLADHYQQNTPIGDGPVLLPDNHYLSYQS
KLSKDPNEKRDHMVLLEFVTAAGITLGMDELYK)
>fretLOCKRd (SEQ ID NO: 42)
(GHHHHHHHHHHGVSKGEELFTGVVPILVELDGDVNGHKFSVSGEGEGDATYGKLTLKFICTTGKLPVPWPTLVTTLSWGVQC
FARYPDHMKQHDFFKSAMPEGYVQERTIFFKDDGNYKTRAEVKFEGDTLVNRIELKGIDFKEDGNILGHKLEYNYFSDNVYIT
ADKQKNGIKANFKIRHNIEDGGVQLADHYQQNTPIGDGPVLLPDNHYLSTQSKLSKDPNEKRDHMVLLEFVTAAGITLGMDEL
YKGSGCSLQGM)SLEAVLKLFELNHKLSEKLLEAVLKLHALNQKLSQKLLEALARLLELNVALVELAIELTDPKRIADEIKKV
KDKSKEIVERAEEEIARAAAESKKILDEAEEEIARAAAESKKILDEGSGSGSDAVAELQALNLKLAELLLEAVAELQALNLKL
AELLLEAIAKLQELNIKLVELLTKLTDPATIREAIRKVKEDSERIVAEAERLIAAAKAESERIIREAERLIAAAKAESERIIR
EGSGSGDPEVARLQEAFIEQAREILRNVAAAQEALIEQARRLLALAALAQEAAIKAVELASEHGSGSG[DTVKRILEELRRRF
EKLAKDLDDIARKLLEDHKKHNKELKDKQRKIKKEADDAARS](VSKGEELFTGVVPILVELDGDVNGHKFSVSGEGEGDATY
GKLTLKLICTTGKLPVPWPTLVTTLGYGVQCFARYPDHMKQHDFFKSAMPEGYVQERTIFFKDDGNYKTRAEVKFEGDTLVNR
IELKGIDFKEDGNILGHKLEYNYNSHNVYITADKQKNGIKANFKIRHNIEDGGVQLADHYQQNTPIGDGPVLLPDNHYLSYQS
KLSKDPNEKRDHMVLLEFVTAAGITLGMDELYK)
>tevLCCKR (SEQ ID NO: 43)
(MGSSHHHHHHSSGLVPRGSHM)SKEAVTKLQALNIKLAEKLLEAVTKLQALNIKLAEKLLEALARLQELNIALVYLAVELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARLQELNIELARELLRDVARLQELNIELARELLRAAAELQELNIKLVELASELTDPD[EARK
AIARVKRESKRIVEDAEENLYFQGAASEKISREAERLIREAAAASEKISRE]
>spyLCCKR (SEQ ID NO: 44)
(MGSSHHHHHHSSGLVPRGSHM)SKEAVTKLQALNIKLAEKLLEAVTKLQALNIKLAEKLLEALARLQELNIALVYLAVELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARLQELNIELARELLRDVARLQELNIELARELLRAAAELQELNIKLVELASELTDPD[EARA
HIVMVDAYKKRIVEDAERLIREAAAASEKISREAERLIREAAAASEKISRE]
>1_nesLOCKR (SEQ ID NO: 45)
SKEAVTKLQALNIKLAEKLLEAVTKLQALNIKLAEKLLEALARLQELNIALVYLAVELTDPKRIADEIKKVKDKSKEIVERAE
EEIARAAAESKKILDEAEEEIARAAAESKKILDEGSGSGSDAVAELQALNLKLAELLLEAVAELQALNLKLAELLLEAIAKLQ
ELNIKLVELLTKLTDPATIREAIRKVKEDSERIVAEAERLIAAAKAESERIIREAERLIAAAKAESERIIREGSGSGDPDVAR
LQELNIELARELLRDVARLQELNIELARELLRAAAELQELNIKLVELASELTDPD[EARKAIARVKRESKRIVEDLALKLAGL
DINSEKISREAERLIREAAAASEKISRE]
>2_nesLOCKR (SEQ ID NO: 46)
SKEAVTKLQALNIKLAEKLLEAVTKLQALNIKLAEKLLEALARLQELNIALVYLAVELTDPKRIADEIKKVKDKSKEIVERAE
EEIARAAAESKKILDEAEEEIARAAAESKKILDEGSGSGSDAVAELQALNLKLAELLLEAVAELQALNLKLAELLLEAIAKLQ
ELNIKLVELLTKLTDPATIREAIRKVKEDSERIVAEAERLIAAAKAESERIIREAERLIAAAKAESERIIREGSGSGDPDVAR
LQELNIELARELLRDVARLQELNIELARELLRAAAELQELNIKLVELASELTDPD[EARKAIARVKRESKRIVEDAERLIREL
AEKLAGLDINAERLIREAAAASEKISRE]
>3_nesLOCKR (SEQ ID NO: 47)
SKEAVTKLQALNIKLAEKLLEAVTKLQALNIKLAEKLLEALARLQELNIALVYLAVELTDPKRIADEIKKVKDKSKEIVERAE
EEIARAAAESKKILDEAEEEIARAAAESKKILDEGSGSGSDAVAELQALNLKLAELLLEAVAELQALNLKLAELLLEAIAKLQ
ELNIKLVELLTKLTDPATIREAIRKVKEDSERIVAEAERLIAAAKAESERIIREAERLIAAAKAESERIIREGSGSGDPDVAR
LQELNIELARELLRDVARLQELNIELARELLRAAAELQELNIKLVELASELTDPD[EARKAIARVKRESKELAEKLRAGLDLN
AAASEKISREAERLIREAAAASEKISRE]
>nlsLOCKR (SEQ ID NO: 48)
SKEAVTKLQALNIKLAEKLLEAVTKLQALNIKLAEKLLEALARLQELNIALVYLAVELTDPKRIADEIKKVKDKSKEIVERAE
EEIARAAAESKKILDEAEEEIARAAAESKKILDEGSGSGSDAVAELQALNLKLAELLLEAVAELQALNLKLAELLLEAIAKLQ
ELNIKLVELLTKLTDPATIREAIRKVKEDSERIVAEAERLIAAAKAESERIIREAERLIAAAKAESERIIREGSGSGDPDVAR
LQELNIELARELLRDVARLQELNIELARELLRAAAELQELNIKLVELASELTDPD[EARKAIARVKRESKAAARRARTSIREA
AAASEKISREAERLIREAAAASEKISRE]
>ezh2LOCKR (SEQ ID NO: 49)
(MGSSHHHHHHSSGLVPRGSHM)SKEAVTKLQALNIKLAEKLLEAVTKLQALNIKLAEKLLEALARLQELNIALVYLAVELTD
PKRIADEIKKVKDKSKEIVERAEEEIARAAAESKKILDEAEEEIARAAAESKKILDEGSGSGSDAVAELQALNLKLAELLLEA
VAELQALNLKLAELLLEAIAKLQELNIKLVELLTKLTDPATIREAIRKVKEDSERIVAEAERLIAAAKAESERIIREAERLIA
AAKAESERIIREGSGSGDPDVARLQELNIELARELLRDVARLQELNIELARELLRAAAELQELNIKLVELASELTDPD[EARK
AIARVKTMFSSNRQKILERTETLNQEWKQRRIQAERLIREAAAASEKISRE]
>1fix_VMAc_C_BIMlatcht9 (SEQ ID NO: 51)
(MGSHHHHHHGSGSENLYFQG)SKEAAKKLQDLNIELARKLLEASTKLQRLNIRLAEALLEAIARLQELNLELVYLAVELTDP
KRIRDEIKEVKDKSKEIIRRAEKEIDDAAKESKKILEEARKAIRDAAEESRKILEEGSGSGSDALDELQKLNLELAKLLLKAI
AETQDLNLRAAKAFLEAAAKLQELNIRAVELLVKLTDPATIRRALEHAKRRSKEIIDEAERAIRAAKRESERIIEEARRLIEK
AKEESERIIREGSGSGDPDIKKLQDLNIELARELLRAHAQLQRLNLELLRELLRALAQLQELNLDLLRLASELTGGSGGSGGS
(VLLNVLSKCAGSKKFRPAPAAAFARECRGFYFELQELKEDDYYGITLSDDSDHQFLLANQVVVHNC)GGSGGS[DEIWIAQE
LRRIGDEFNAYYADAERLIREAAAASEKISREAERLIREAA]
>sfGFP_VMAn_1fix_BIM_t0_latch (SEQ ID NO: 52)
(MGSHHHHHHGSGSENLYFQG)HMSKGEELFTGVVPILVELDGDVNGHKFSVRGEGEGDATNGKLTLKFICTTGKLPVPWPTL
VTTLTYGVQCFARYPDHMKQHDFFKSAMPEGYVQERTISFKDDGTYKTRAEVKFEGDTLVNRIELKGIDFKEDGNILGHKLEY
NFNSHNVYITADKQKNGIKANFKIRHNVEDGSVQLADHYQQNTPIGDGPVLLPDNHYLSTQSVLSKDPNEKRDHMVLLEFVTA
AGITHGMDELYKSGSGSGCFAKGTNVLMADGSIECIENIEVGNKVMGKDGRPREVIKLPRGRETMYSVVQKSQHRAHKSDSSR
EVPELLKFTCNATHELVVRTPRSVRRLSRTIKGVEYFEVITFEMGQKKAPDGRIVELVKEVSKSYPISEGPERANELVESYRK
ASNKAYFEWTIEARDLSLLGSHVRKATYQTYAPILYGGSGGSGGGGSGGSGSKEAAKKLQDLNIELARKLLEASTKLQRLNIR
LAEALLEAIARLQELNLELVYLAVELTDPKRIRDEIKEVKDKSKEIIRRAEKEIDDAAKESKKILEEARKAIRDAAEESRKIL
EEGSGSGSDALDELQKLNLELAKLLLKAIAETQDLNLRAAKAFLEAAAKLQELNIRAVELLVKLTDPATIRRALEHAKRRSKE
IIDEAERAIRAAKRESERIIEEARRLIEKAKEESERIIREGSGSGDPDIKKLQDLNIELARELLRAHAQLQRLNLELLRELLR
ALAQLQELNLDLLRLASELT[DEIWIAQELRRIGDEFNAYYADAERLSREAAAASEKISREAERSIREAAAASEKISRE]
Asymmetrized functional Cages encoding Bim and GFP11 (i.e.: bioactive peptides)
(6His-MBP-TEV, 6His-TEV, and flexible linker sequences are underlined text)
(Co-localization domain is bolded text)
(Functional peptide is italicized underlined text)
(Positions that can be mutated to any amino acid to tune responsiveness are
underlined bolded text)
(C-terminal sequences that can be removed to tune responsiveness are italicized
text)
(all sequences in parentheses are optional)
>1fix-long-BIM-t0 (SEQ ID NO: 54)
(MGSHHHHHHGSGSENLYFQG)SKEAAKKLQDLNIELARKLLEASTKLQRLNIRLAEALLEAIARLQELNLELVYLAVELTDP
KRIRDEIKEVKDKSKEIIRRAEKEIDDAAKESKKILEEARKAIRDAAEESRKILEEGSGSGSDALDELQKLNLELAKLLLKAI
AETQDLNLRAAKAFLEAAAKLQELNIRAVELLVKLTDPATIRRALEHAKRRSKEIIDEAERAIRAAKRESERIIEEARRLIEK
AKEESERIIREGSGSGDPDIKKLQDLNIELARELLRAHAQLQRLNLELLRELLRALAQLQELNLDLLRLASEL(TD[EIWIA]
ELRRIGDEFNAYYA)DAERLIREAAAASEKISREAERLIREAAAASEKISRE]
>1fix-long-GFP-t0 (SEQ ID NO: 55)
(MGSHHHHHHGSGSENLYFQG)SKEAAKKLQDLNIELARKLLEASTKLQRLNIRLAEALLEAIARLQELNLELVYLAVELTDP
KRIRDEIKEVKDKSKEIIRRAEKEIDDAAKESKKILEEARKAIRDAAEESRKILEEGSGSGSDALDELQKLNLELAKLLLKAI
AETQDLNLRAAKAFLEAAAKLQELNIRAVELLVKLTDPATIRRALEHAKRRSKEIIDEAERAIRAAKRESERIIEEARRLIEK
AKEESERIIREGSGSGDPDIKKLQDLNIELARELLRAHAQLQRLNLELLRELLRALAQLQELNLDLLRLASEL[(RDHMVLHE
YVNAAGITFNAYYA)DAERLIREAAAASEKISREAERLIREAAAASEKISRE]
>1fix-short-BIM-t0 (SEQ ID NO: 56)
(MGSHHHHHHGSGSENLYFQGSGG)SELARKLLEASTKLQRLNIRLARALLEAIARLQELNLELVYLAVELTDPKRIRDEIKE
VKDKSKEIIRRAEKEIDDAAKESEKILEEAREAISGSGSELAKLLLKAIAETQDLNLRAAKAFLEAAAKLQELNIRAVELLVK
LTDPATIREALEHAKRRSKEIIDEAERAIRAAKRESERIIEEARRLIEKGSGSGSELARELLRAHAQLQRLNLELLRELLRAL
AQLQELNLDLLRLASEL(TD[EIWIA]ELRRIGDEFNAYYA)DAERLIREAAAASEKISREAERLIR]
>1fix-short-GFP-t0 (SEQ ID NO: 57)
(MGSHHHHHHGSGSENLYFQGSGG)SELARKLLEASTKLQRLNIRLARALLEAIARLQELNLELVYLAVELTDPKRIRDEIKE
VKDKSKEIIRRAEKEIDDAAKESEKILEEAREAISGSGSELAKLLLKAIAETQDLNLRAAKAFLEAAAKLQELNIRAVELLVK
LTDPATIREALEHAKRRSKEIIDEAERAIRAAKRESERIIEEARRLIEKGSGSGSELARELLRAHAQLQRLNLELLRELLRAL
AQLQELNLDLLRLASEL[(RDHMVLHEYVNAAGITFNAYYA)DAERLIREAAAASEKISREAERLIR]
>Spycatcher-1fix-long-GFP-t0 (SEQ ID NO: 58)
(MGSHHHHHHGSGSENLYFQGS)AMVDTLSGLSSEQGQSGDMTIEEDSATHIKESKRDEDGKELAGATMELRDSSGETISTWI
SDGQVKDFYLYPGKYTFVETAAPDGYEVATAITFTVNEQGQVTVNGKATEGSGGSKEAAKKLQDLNIELARKLLEASTKLQRL
NIRLARALLEAIARLQELNLELVYLAVELTDPKRIRDEIKEVKDKSKEIIRRAEKEIDDAAKESKKILEEARKAIRDAAEESR
KILEEGSGSGSDALDELQKLNLELAKLLLKAIAETQDLNLRAAKAFLEAAAKLQELNIRAVELLVKLTDPATIRRALEHAKRR
SKEIIDEAERAIRAAKRESERIIEEARRLIEKAKEESERIIREGSGSGDPDIKKLQDLNIELARELLRAHAQLQRLNLELLRE
LLRALAQLQELNLDLLRLASEL[(RDHMVLHEYVNAAGITFNAYYA)DAERLIREAAAASEKISREAERLIREAAAASEKISR
E]
>Spycatcher-1fix-short-GFP-t0 (SEQ ID NO: 59)
(MGSHHHHHHGSGSENLYFQGS)AMVDTLSGLSSEQGQSGDMTIEEDSATHIKESKRDEDGKELAGATMELRDSSGETISTWI
SDGQVKDFYLYPGKYTFVETAAPDGYEVATAITFTVNEQGQVTVNGKATKGSGGSELARKLLEASTKLQRLNIRLAEALLEAI
ARLQELNLELVYLAVELTDPKRIRDEIKEVKDKSKEIIRRAEKEIDDAAKESEKILEEAREAISGSGSELAKLLLKAIAETQD
LNLRAAKAFLEAAAKLQELNIRAVELLVKLTDPATIREALEHAKRRSKEIIDEAERAIRAAKRESERIIEEARRLIEKGSGSG
SELARELLRAHAQLQRLNLELLRELLRALAQLQELNLDLLRLASEL[(RDHMVLHEYVNAAGITFNAYYA)DAERLIREAAAA
SEKISREAERLIR]
>1fix-latch_Mad1SID_t0_1 (SEQ ID NO: 61)
(MGSHHHHHHGSGSENLYFQG)SKEAAKKLQDLNIELARKLLEASTKLQRLNIRLAEALLEAIARLQELNLELVYLAVELTDP
KRIRDEIKEVKDKSKEIIRRAEKEIDDAAKESKKILEEARKAIRDAAEESRKILEEGSGSGSDALDELQKLNLELAKLLLKAI
AETQDLNLRAAKAFLEAAAKLQELNIRAVELLVKLTDPATIRRALEHAKRRSKEIIDEAERAIRAAKRESERIIEEARRLIEK
AKEESERIIREGSGSGDPDIKKLQDLNIELARELLRAHAQLQRLNLELLRELLRALAQLQELNLDLLRLASELT[(NIQMLLE
AADYLE)RESKRIVEDAERLIREAAAASEKISREAERSIREAAAASEKISRE]
>1fix-latch_Mad1SID_T0_2 (SEQ ID NO: 65)
(MGSHHHHHHGSGSENLYFQG)SKEAAKKLQDLNIELARKLLEASTKLQRLNIRLAEALLEAIARLQELNLELVYLAVELTDP
KRIRDEIKEVKDKSKEIIRRAEKEIDDAAKESKKILEEARKAIRDAAEESRKILEEGSGSGSDALDELQKLNLELAKLLLKAI
AETQDLNLRAAKAFLEAAAKLQELNIRAVELLVKLTDPATIRRALEHAKRRSKEIIDEAERAIRAAKRESERIIEEARRLIEK
AKEESERIIREGSGSGDPDIKKLQDLNIELARELLRAHAQLQRLNLELLRELLRALAQLQELNLDLLRLASELTDP[DEARK
(NIQMLLEAADYLE)EDAERLIREAAAASEKISREAERLIREAASEKISRE]
>1fix-short-Bim-t0-relooped (SEQ ID NO: 67)
[MDEARKAIARVKRESKRI(EIWIAQELRRIGDEFNAYYA)EAEKLAT]DELWHRLLEASTKLQRLNIRLARALLEAIARLQE
LNLELVYLAVELTDPKRIRDEIKEVKDKSKEIIRRAEKEIDDAAKESEKILEEAREAISGSGSELAKLLLKAIAETQDLNLRA
AKAFLEAAAKLQELNIRAVELLVKLTDPATIREALEHAKRRSKEIIDEAERAIRAAKRESERIIEEARRLIEKGSGSGSELAR
ELLRAHAQLQRLNLELLRELLRALAQLQELNLDLLRLASE
>1fix-short-spytag-t0_2 (SEQ ID NO: 68)
(MGSSHHHHHHSSGLVPRGSHM)SELARKLLEASTKLQRLNIRLARALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLRELLRALAQ
LQELNLDLLRLASELTDPD[EAR(AHIVMVDAYK)KRIVEDAERLIREAAAASEKISREAERLIR]
>1fix-short-spytag-t0_8 (SEQ ID NO: 69)
(MGSSHHHHHHSSGLVPRGSHM)SELARKLLEASTKLQRLNIRLARALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLRELLRALAQ
LQELNLDLLRLASELTDPD[EARKAIARVKRESK(AHIVMVDAYK)REAAAASEKISREAERLIR]
>1fix-short-TEV-t0_1 (SEQ ID NO: 70)
(MGSSHHHHHHSSGLVPRGSHM)SELARKLLEASTKLQRLNIRLARALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLRELLRALAQ
LQELNLDLLRLASELTDP[DEAR(ENLYFQGS)ESKRIVEDAERLIREAAAASEKISREAERLIR]
>1fix-short-TEV-t0_6 (SEQ ID NO: 71)
(MGSSHHHHHHSSGLVPRGSHM)SELARKLLEASTKLQRLNIRLARALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLRELLRALAQ
LQELNLDLLRLASELTDP[DEARKAIARVKRESKRIV(ENLYFQGS)EAAAASEKISREAERLIR]
>1fix-short-nanoBit-t0_1 (SEQ ID NO: 72)
(MGSSHHHHHHSSGLVPRGSHM)SELARKLLEASTKLQRLNIRLARALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLRELLRALAQ
LQELNLDLLRLASELTDP[DEAR(VSGWRLFRKIS)RIVEDAERLIREAAAASEKISREAERLIR]
>1fix-short-nanoBit-t0_3 (SEQ ID NO: 73)
(MGSSHHHHHHSSGLVPRGSHM)SELARKLLEASTKLQRLNIRLARALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLRELLRALAQ
LQELNLDLLRLASELTDP[DEARKAIARVKRESK(VSGWRLFKKIS)EAAAASEKISREAERLIR]
>1fix-short-RHIM-t0_8 (SEQ ID NO: 74)
(MGSSHHHHHHSSGLVPRGSHM)SELARKLLEASTKLQRLNIRLARALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLRELLRALAQ
LQELNLDLLRLASELTDP[DEARKAI(IQIG)RESKRIVEDAERLIREAAAASEKIS(VQLG)RLIR]
>lfix-short-RHIM-t0_19 (SEQ ID NO: 75)
(MGSSHHHHHHSSGLVPRGSHM)SELARKLLEASTKLQRLNIRLARALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLRELLRALAQ
LQELNLDLLRLASELTDP[DEARKAIARVKRESKRIV(IQIG)RLI(VQLG)AASEKISREAERLIR]
>1fix-short-RHIM-t0_22 (SEQ ID NO: 76)
(MGSSHHHHHHSSGLVPRGSHM)SELARKLLEASTKLQRLNIRLARALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLRELLRALAQ
LQELNLDLLRLASELTDP[DEARKAIARVKRESKRIV(IQIG)RLIREAAAASEKIS(VQLG)RLIR]
>1fix-short-gcn4-t0_4 (SEQ ID NO: 77)
(MGSSHHHHHHSSGLVPRGSHM)SELARKLLEASTKLQRLNIRLARALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLRELLRALAQ
LQELNLDLLRLASELTDP[DESVKE(LEDKVEELLSKNYHLENEVARLKKLVGER)SREAERLIR]
>1fix-short-ccDi-t0_6 (SEQ ID NO: 78)
(MGSSHHHHHHSSGLVPRGSHM)SELARKLLEASTKLQRLNIRLARALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLRELLRALAQ
LQELNLDLLRLASELTDP[DEARKAIA(GEIAALKQEIAALKKENAALKWEIAALKQG)AERLIR]
>1fix-short-cc-a-t0_6 (SEQ ID NO: 79)
(MGSSHHHHHHSSGLVPRGSHM)SELARKLLEASTKLQRLNIRLARALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLRELLRALAQ
LQELNLDLLRLASELTDP[DEARKAIARVKR(GLEQEIAALEKENAALEWEIAALEQGG)ERLIR]
>1fix-short-cc-b-t0_6 (SEQ ID NO: 80)
(MGSSHHHHHHSSGLVPRGSHM)SELARKLLEASTKLQRLNIRLARALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLRELLRALAQ
LQELNLDLLRLASELTDP[DEARKAIARVKR(GLKQKIAALKYRNAALKEKIAALKQGG)ERLIR
STREPII-LOCKR functional Cages:
>STREPII-2plus1_LOCK_1 (SEQ ID NO: 81)
SRVEEIIEDLRRLLEEIRKENADSIRASKELLDRVKEINDTIIAELERLLKDIEKEVREKGSESEEVKKALRRVLEELEKLLR
RVAEINEEVLRRNSKLVEEDARRNAEVLKELKRLVEELMREIGDED[KVRKVAEVAEKVLRDIDKLDR(WSHPQFEK)TNGEI
SKLDEDTRRVAERVKKAIEDLAK]
>STREPII-2plus1_LOCK_2 (SEQ ID NO: 82)
[SEVDEIIADNERALDEVRREVEEIDKENAERLGE(WSHPQFEK)GDRLAKALEEIRK]GVRSRLVDELERAIREVEEVIRRV
LERVRRLIEEVSKIITDVLREVERLHEEVTKELRKVEDGNSREALDALRRLIEKVVEDSARLIKKVDEALKAVNKEIEDLSRE
VADLVRAVAEELDARVK
>STREPII-2plus1_LOCK_3 (SEQ ID NO: 83)
SSDEVLKEIEEIIRRLEAEVRRVNAEVNASTEDLAREVEEVLRATNELIEELERRVTGTEELKRVIDELRDRDRKVRRRVERV
IEESAKRDDESRKRLTRAVEKLRADLKKLADDGVPE[EALSKAIKDVRDIVKKVKDELKE(WSHPQFEK)VDRLSEELKEWLK
DVERVLKELTDKDR]
>STREPII-2plus1_LOCK_4C (SEQ ID NO: 84)
SDAEELLKRVADLLKASLESLEKILRDSKELMDRWRKKLEDLLRESEELVDRAEKILRRGGSDKEVLDKIAEEVRRTNDDSRR
LDEELHRLSRDTLRKLEENLRRTEKEVREMDKRAAERG[VDERVREELKKLLTRVE(WSHPQFEK)GDKKILKEAHKESKEVN
DRDRELLERLEESVR]
>STREPII-2plus1_LOCK_4N (SEQ ID NO: 85)
[SDAEELLKRVADLLKASLESLEKILRDSKELMDR(WSHPQFEK)LGESEELVDRAEKILRR]GGSDKEVLDKIAEEVRRTND
DSRRLDEELHRLSRDTLRKLEENLRRTEKEVREMDKRAAERGVDERVREELKKLLTRVEEEHRKVLETDKKILKEAHKESKEV
NDRDRELLERLEESVR
>STREPII-3plus1_LOCK1 (SEQ ID NO: 86)
SEAEDLLERVKRVLDELIEIVDRNHELNARVVETSARLVERLLEEVERALETLEREIPGRELLDKAIKDLRDVLRRVAEKVKR
SIEELKEVLEESRRVLEEVVRALAEVIDRVRRLVEKGVDLRDLIRELKRVLEEAVSLIERLVRLNTRAAEKDNESLRELVRAI
KEALKRAVDMVRADGL[DSRLVKKLDEIVKEVAKKLEDVVRANEEL(WSHPQFEK)GSSVARLREAVERVARDLEETAR]
>STREPII-3plus1_LOCK_2 (SEQ ID NO: 87)
[SDEERLEKVVKDVIEKVRRILEK(WSHPQFEK)GSELRRILEEWEKIIREVLDKVRR]GSGSADALVEVLEEILRLAEELSK
RVEEVLREILKLAKALSDELVKVLAEIVEAAKRISRDDELRKAVEDVARELEDLAAKDRKILDDVREALERIAKEDKDILREA
EETLRRLADEMRRSGVDERLLKRVVDILARLLELNATTIERLLRILEELLKLNKELAERVIRVLEKLLEEIKR
>STREPII-3plus1_LOCK_3 (SEQ ID NO: 88)
SVLETVKKALEDSSEKIERIVEEDERVAKESSDRIRRLVEEDKRVADEILDLIEKGGDTDTLAKLVEEWSRTSKKLLDDVLKL
HKDWSDDSRRLLEEILRVHEELIRAVKEILDRGGKPEEVVRELEKVLKESLDTLEEIIRRLDEANARTVKRVADVIRELEDAN
AKVLEEIERKGD[DKDAVIKVIEELIRANAAV(WSHPQFEK)GDLVRVNKTVWKELLRVNEKLARDLERVVK]
>STREPII-3plus1_LOCK_4 (SEQ ID NO: 89)
[SLVDELRKSLERNVRVSEEVARRLKEALGR(WSHPQFEK)GGDLIRLNEDVVRVVEKV]GVDESAIERVRRIIEELNRALDA
VLKKNEDLVRRLTELLDKLLEENRRLVEELDEDLKRRGGTEEVIDTILELIERSIERLKRLLDELLRIVREALKDNARVADEN
LKALKEILDELRKDGVSDEELKRVLEKAADLHARLKDAHRKLLEDLERIIRELKKKLDEVVEENKRSVDELKR
>STREPII-3plusl_LOCK_3-relooped (SEQ ID NO: 90)
[MKDAVIKVIEELIRANAAV(WSHPQFEK)GDLVRVNKTVWKELLRVNEKLARDLERAL]DERDVSAWETVKKALEDSSEKIE
RIVEEDERVAKESSDRIRRLVEEDKRVADEILDLIEKGGDTDTLAKLVEEWSRTSKKLLDDVLKLHKDWSDDSRRLLEEILRV
HEELIRAVKEILDRGGAPEEVVRELEKVLKESLDTLEEIIRRLDEANARTVKRVADVIRELEDANAKVLEEIERK
>STREPII-2plusl_LOCK_3-relooped (SEQ ID NO: 91)
[MEEAASKAIKDVRDIVKKVKDELKE(WSHPQFEK)VDRLSEELKEWLKDVERVLKELT]DREEASEEELKRVIDELRDRDRK
VRRRVERVIEESAKRDDESRKRLTRAVEKLRADLKKLSVEGASDEVLKEIEEIIRRLEAEVRRVNAEVNASTEDLAREVEEVL
RATNELIEELERR
>BimLOCKR_a_short_Nterm (SEQ ID NO: 27094)
[MDEARKAIARVKRESKRI(EIWIAQELRRIGDEFNAYYA)EAEKLATDEL]WHRLLEASTKLQRLNIRLARALLEAIARLQE
LNLELVYLAVELTDPKRIRDEIKEVKDKSKEIIRRAEKEIDDAAKESEKILEEAREAISGSGSELAKLLLKAIAETQDLNLRA
AKAFLEAAAKLQELNIRAVELLVKLTDPATIREALEHAKRRSKEIIDEAERAIRAAKRESERIIEEARRLIEKGSGSGSELAR
ELLRAHAQLQRLNLELLRELLRALAQLQELNLDLLRLASE
>BimLOCKR_g (SEQ ID NO: 27095)
[MSLVDEL(EIWIAQELRRIGDEFNAYYA)ALKRWVDVVRKVVEDLIRLNEDVVRVVEKV]GVDESAIERVRRIIEELNRALD
AVLKKNEDLVRRLTELLDKLLEENRRLVEELDEDLKRRGGTEEVIDTILELIERSIERLKRLLDELLRIVREALKDNARVADE
NLKALKEILDELRKDGVSDEELKRVLEKAADLHARLKDAHRKLLEDLERIIRELKKKLDEVVEENKRSVDELKR
>reloop_strepLOCKRh (SEQ ID NO: 27096)
[MKDAVIKVIEELIRANAAV(WSHPQFEK)GDLVRVNKTVWKELLRVNEKLARDLERALDEMDVSAWETVKKALEDSSEKIE
RIVEEDERVAKESSDRIRRLVEEDKRVADEILDLIEKGGDTDTLAKLVEEWSRTSKKLLDDVLKLHKDWSDDSRRLLEEILRV
HEELIRAVKEILDRGGAPEEVVRELEKVLKESLDTLEEIIRRLDEANARTVKRVADVIRELEDANAKVLEEIERK
>reloop_strepLOCKRi (SEQ ID NO: 27097)
[MEEAASKAIKDVRDIVKKVKDELKE(WSHPQFEK)VDRLSEELKEWLKDVERVLKELTDREEA]SEEELKRVIDELRDRDRK
VRRRVERVIEESAKRDDESRKRLTRAVEKLRADLKKLSVEGASDEVLKEIEEIIRRLEAEVRRVNAEVNASTEDLAREVEEVL
RATNELIEELERR
>spyLOCKRa_2 (SEQ ID NO: 27098)
MSELARKLLEASTKLQRLNIRLAEALLEAIARLQELNLELVYLAVELTDPKRIRDEIKEVKDKSKEIIRRAEKEIDDAAKESE
KILEEAREAISGSGSELAKLLLKAIAETQDLNLRAAKAFLEAAAKLQELNIRAVELLVKLTDPATIREALEHAKRRSKEIIDE
AERAIRAAKRESERIIEEARRLIEKGSGSGSELARELLRAHAQLQRLNLELLRELLRALAQLQELNLDLLRLASELTDP[DEA
R(AHIVMVDAYK)KRIVEDAERLIREAAAASEKISREAERLIR]
>spyLOCKRa_8 (SEQ ID NO: 27099)
MSELARKLLEASTKLQRLNIRLAEALLEAIARLQELNLELVYLAVELTDPKRIRDEIKEVKDKSKEIIRRAEKEIDDAAKESE
KILEEAREAISGSGSELAKLLLKAIAETQDLNLRAAKAFLEAAAKLQELNIRAVELLVKLTDPATIREALEHAKRRSKEIIDE
AERAIRAAKRESERIIEEARRLIEKGSGSGSELARELLRAHAQLQRLNLELLRELLRALAQLQELNLDLLRLASELTDPDEAR
KAIARVKRESK(AHIVMVDAYK)REAAAASEKISREAERLIR]
>tevLOCKRa_1 (SEQ ID NO: 27100)
MSELARKLLEASTKLQRLNIRLAEALLEAIARLQELNLELVYLAVELTDPKRIRDEIKEVKDKSKEIIRRAEKEIDDAAKESE
KILEEAREAISGSGSELAKLLLKAIAETQDLNLRAAKAFLEAAAKLQELNIRAVELLVKLTDPATIREALEHAKRRSKEIIDE
AERAIRAAKRESERIIEEARRLIEKGSGSGSELARELLRAHAQLQRLNLELLRELLRALAQLQELNLDLLRLASELTDP[DEA
R(ENLYFQGS)ESKRIVEDAERLIREAAAASEKISREAERLIR]
>tevLOCKRa_6 (SEQ ID NO: 27101)
MSELARKLLEASTKLQRLNIRLAEALLEAIARLQELNLELVYLAVELTDPKRIRDEIKEVKDKSKEIIRRAEKEIDDAAKESE
KILEEAREAISGSGSELAKLLLKAIAETQDLNLRAAKAFLEAAAKLQELNIRAVELLVKLTDPATIREALEHAKRRSKEIIDE
AERAIRAAKRESERIIEEARRLIEKGSGSGSELARELLRAHAQLQRLNLELLRELLRALAQLQELNLDLLRLASELTDP[DEA
RKAIARVKRESKRIV(ENLYFQGS)EAAAASEKISREAERLIR]
>lucLOCKRa_1 (SEQ ID NO: 27102)
MSELARKLLEASTKLQRLNIRLAEALLEAIARLQELNLELVYLAVELTDPKRIRDEIKEVKDKSKEIIRRAEKEIDDAAKESE
KILEEAREAISGSGSELAKLLLKAIAETQDLNLRAAKAFLEAAAKLQELNIRAVELLVKLTDPATIREALEHAKRRSKEIIDE
AERAIRAAKRESERIIEEARRLIEKGSGSGSELARELLRAHAQLQRLNLELLRELLRALAQLQELNLDLLRLASELTDP[DEA
R(VSGWRLFKKIS)RIVEDAERLIREAAAASEKISREAERLIR]
>lucLOCKRa_3 (SEQ ID NO: 27103)
MSELARKLLEASTKLQRLNIRLAEALLEAIARLQELNLELVYLAVELTDPKRIRDEIKEVKDKSKEIIRRAEKEIDDAAKESE
KILEEAREAISGSGSELAKLLLKAIAETQDLNLRAAKAFLEAAAKLQELNIRAVELLVKLTDPATIREALEHAKRRSKEIIDE
AERAIRAAKRESERIIEEARRLIEKGSGSGSELARELLRAHAQLQRLNLELLRELLRALAQLQELNLDLLRLASELTDP[DEA
RKAIARVKRESK(VSGWRLFKKIS)EAAAASEKISREAERLIR]
>rhimLOCKRa_8 (SEQ ID NO: 27104)
MSELARKLLEASTKLQRLNIRLAEALLEAIARLQELNLELVYLAVELTDPKRIRDEIKEVKDKSKEIIRRAEKEIDDAAKESE
KILEEAREAISGSGSELAKLLLKAIAETQDLNLRAAKAFLEAAAKLQELNIRAVELLVKLTDPATIREALEHAKRRSKEIIDE
AERAIRAAKRESERIIEEARRLIEKGSGSGSELARELLRAHAQLQRLNLELLRELLRALAQLQELNLDLLRLASELTDP[DEA
RKAI(IQIG)RESKRIVEDAERLIREAAAASEKIS(VQLG)RLIR]
>rhimLOCKRa_19 (SEQ ID NO: 27105)
MSELARKLLEASTKLQRLNIRLAEALLEAIARLQELNLELVYLAVELTDPKRIRDEIKEVKDKSKEIIRRAEKEIDDAAKESE
KILEEAREAISGSGSELAKLLLKAIAETQDLNLRAAKAFLEAAAKLQELNIRAVELLVKLTDPATIREALEHAKRRSKEIIDE
AERAIRAAKRESERIIEEARRLIEKGSGSGSELARELLRAHAQLQRLNLELLRELLRALAQLQELNLDLLRLASELTDP[DEA
RKAIARVKRESKRIV(IQIG)RLI(VQLG)AASEKISREAERLIR]
>rhimLOCKRa_22 (SEQ ID NO: 27106)
MSELARKLLEASTKLQRLNIRLAEALLEAIARLQELNLELVYLAVELTDPKRIRDEIKEVKDKSKEIIRRAEKEIDDAAKESE
KILEEAREAISGSGSELAKLLLKAIAETQDLNLRAAKAFLEAAAKLQELNIRAVELLVKLTDPATIREALEHAKRRSKEIIDE
AERAIRAAKRESERIIEEARRLIEKGSGSGSELARELLRAHAQLQRLNLELLRELLRALAQLQELNLDLLRLASELTDP[DEA
RKAIARVKRESKRIV(IQIG)RLIREAAAASEKIS(VQLG)RLIR]
>gcn4LOCKRa_4 (SEQ ID NO: 27107)
MSELARKLLEASTKLQRLNIRLAEALLEAIARLQELNLELVYLAVELTDPKRIRDEIKEVKDKSKEIIRRAEKEIDDAAKESE
KILEEAREAISGSGSELAKLLLKAIAETQDLNLRAAKAFLEAAAKLQELNIRAVELLVKLTDPATIREALEHAKRRSKEIIDE
AERAIRAAKRESERIIEEARRLIEKGSGSGSELARELLRAHAQLQRLNLELLRELLRALAQLQELNLDLLRLASELTDP[DES
VKE(LEDEVEELLSKNYHLENEVARLKKLVGER)SREAERLIR]
>cc-DiLOCKRa_6 (SEQ ID NO: 27108)
MSELARKLLEASTKLQRLNIRLAEALLEAIARLQELNLELVYLAVELTDPKRIRDEIKEVKDKSKEIIRRAEKEIDDAAKESE
KILEEAREAISGSGSELAKLLLKAIAETQDLNLRAAKAFLEAAAKLQELNIRAVELLVKLTDPATIREALEHAKRRSKEIIDE
AERAIRAAKRESERIIEEARRLIEKGSGSGSELARELLRAHAQLQRLNLELLRELLRALAQLQELNLDLLRLASELTDP[DEA
RKAIA(GEIAALKQEIAALKKENAALKWEIAALKQG)AERLIR]
>cc-aLOCKRa_6 (SEQ ID NO: 27109)
MSELARKLLEASTKLQRLNIRLAEALLEAIARLQELNLELVYLAVELTDPKRIRDEIKEVKDKSKEIIRRAEKEIDDAAKESE
KILEEAREAISGSGSELAKLLLKAIAETQDLNLRAAKAFLEAAAKLQELNIRAVELLVKLTDPATIREALEHAKRRSKEIIDE
AERAIRAAKRESERIIEEARRLIEKGSGSGSELARELLRAHAQLQRLNLELLRELLRALAQLQELNLDLLRLASELTDP[DEA
RKAIARVKR(GLEQEIAALEKENAALEWEIAALEQGG)ERLIR]
>cc-bLOCKRa_6 (SEQ ID NO: 27110)
MSELARKLLEASTKLQRLNIRLAEALLEAIARLQELNLELVYLAVELTDPKRIRDEIKEVKDKSKEIIRRAEKEIDDAAKESE
KILEEAREAISGSGSELAKLLLKAIAETQDLNLRAAKAFLEAAAKLQELNIRAVELLVKLTDPATIREALEHAKRRSKEIIDE
AERAIRAAKRESERIIEEARRLIEKGSGSGSELARELLRAHAQLQRLNLELLRELLRALAQLQELNLDLLRLASELTDP[DEA
RKAIARVKR(GLKQKIAALKYRNAALKKKIAALKQGG)ERLIR]
>tev-spyLOCKRa_short_40 (SEQ ID NO: 27111)
SELARKLLEASTKLQRLNIRLAEALLEAIARLQELNLELVYLAVELTDPKRIRDEIKEVKDKSKEIIRRAEKEIDDAAKESEK
ILEEAREAISGSGSELAKLLLKAIAETQDLNLRAAKAFLEAAAKLQELNIRAVELLVKLTDPATIREALEHAKRRSKEIIDEA
ERAIRAAKRESERIIEEARRLIEKGSGSGSELARELLRAHAQLQRLNLELLRELLRALAQLQELNLDLLRLASELTDP[DEAR
KAI(ENLYFQGS)RIVEDAE(AHIVMVDAYK)EKISREAERLIR]
>tev-spyLOCKRa_short_57 (SEQ ID NO: 27112)
SELARKLLEASTKLQRLNIRLAEALLEAIARLQELNLELVYLAVELTDPKRIRDEIKEVKDKSKEIIRRAEKEIDDAAKESEK
ILEEAREAISGSGSELAKLLLKAIAETQDLNLRAAKAFLEAAAKLQELNIRAVELLVKLTDPATIREALEHAKRRSKEIIDEA
ERAIRAAKRESERIIEEARRLIEKGSGSGSELARELLRAHAQLQRLNLELLRELLRALAQLQELNLDLLRLASELTDP[DEAR
KAIARV(ENLYFQGS)EDAERLIREA(AHIVMVDAYK)AERLIR]
>tev-spyLOCKRa_short_63 (SEQ ID NO: 27113)
SELARKLLEASTKLQRLNIRLAEALLEAIARLQELNLELVYLAVELTDPKRIRDEIKEVKDKSKEIIRRAEKEIDDAAKESEK
ILEEAREAISGSGSELAKLLLKAIAETQDLNLRAAKAFLEAAAKLQELNIRAVELLVKLTDPATIREALEHAKRRSKEIIDEA
ERAIRAAKRESERIIEEARRLIEKGSGSGSELARELLRAHAQLQRLNLELLRELLRALAQLQELNLDLLRLASELTDP[DEAR
KAIARVK(ENLYFQGS)DAERLIREA(AHIVMVDAYK)AERLIR]
>tev-spyLOCKRa_29 (SEQ ID NO: 27114)
SKEAAKKLQDLNIELARKLLEASTKLQRLNIRLAEALLEAIARLQELNLELVYLAVELTDPKRIRDEIKEVKDKSKEIIRRAE
KEIDDAAKESKKILEEARKAIRDAAEESRKILEEGSGSGSDALDELQKLNLELAKLLLKAIAETQDLNLRAAKAFLEAAAKLQ
ELNIRAVELLVKLTDPATIRRALEHAKRRSKEIIDEAERAIRAAKRESERIIERARRLIEKAKEESERIIREGSGSGDP[DIK
KLQDLNIELARELLRAHAQLQRLNLELLRELLRALAQLQELNLDLLRLASELTDPDEARKAIARVK(ENLYFQGS)DAERLIR
EAAAASE(AHIVMVDAYK)REAAAASEKISRE]
>tev-spyLOCKRa_32 (SEQ ID NO: 27115)
SKEAAKKLQDLNIELARKLLEASTKLQRLNIRLAEALLEAIARLQELNLELVYLAVELTDPKRIRDEIKEVKDKSKEIIRRAE
KEIDDAAKESKKILEEARKAIRDAAEESRKILEEGSGSGSDALDELQKLNLELAKLLLKAIAETQDLNLRAAKAFLEAAAKLQ
ELNIRAVELLVKLTDPATIRRALEHAKRRSKEIIDEAERAIRAAKRESERIIERARRLIEKAKEESERIIREGSGSGDP[DIK
KLQDLNIELARELLRAHAQLQRLNLELLRELLRALAQLQELNLDLLRLASELTDPDEARKAIARVK(ENLYFQGS)DAERLIR
EAAAASEKISREAE(AHIVMVDAYK)EKISRE]
>Bim-fretLOCKRa_short (SEQ ID NO: 27116)
(VSKGEELFTGVVPILVELDGDVNGHKFSVSGEGEGDATYGKLTLKFICTTGKLPVPWPTLVTTLSWGVQCFARYPDHMKQHD
FFKSAMPEGYVQERTIFFKDDGNYKTRAEVKFEGDTLVNRIELKGIDFKEDGNILGHKLEYNYFSDNVYITADKQKNGIKANF
KIRHNIEDGGVQLADHYQQNTPIGDGPVLLPDNHYLSTQSKLSKDPNEKRDHMVLLEFVTAAGITLE)LARKLLEASTKLQRL
NIRLARALLEAIARLQELNLELVYLAVELTDPKRIRDEIKEVKDKSKEIIRRAEKEIDDAAKESEKILEEAREAISGSGSELA
KLLLKAIAETQDLNLRAAKAFLEAAAKLQELNIRAVELLVKLTDPATIREALEHAKRRSKEIIDEAERAIRAAKRESERIIEE
ARRLIEKGSGSGSELARELLRAHAQLQRLNLELLRELLRALAQLQELNLDLLRLASELT[D(EIWIAQELRRIGDEFNAYYA)
DAERLIREAAAASEKISREAERLIR](VSKGEELFTGVVPILVELDGDVNGHKFSVSGEGEGDATYGKLTLKLICTTGKLPVP
WPTLVTTLGYGVQCFARYPDHMKQHDFFKSAMPEGYVQERTIFFKDDGNYKTRAEVKFEGDTLVNRIELKGIDFKEDGNILGH
KLEYNYNSHNVYITADKQKNGIKANFKIRHNIEDGGVQLADHYQQNTPIGDGPVLLPDNHYLSYQSKLSKDPNEKRDHMVLLE
FVTAAGITLGMDELYKGSGC)
>fretLOCKRa_short (SEQ ID NO: 27117)
(VSKGEELFTGVVPILVELDGDVNGHKFSVSGEGEGDATYGKLTLKFICTTGKLPVPWPTLVTTLSWGVQCFARYPDHMKQHD
FFKSAMPEGYVQERTIFFKDDGNYKTRAEVKFEGDTLVNRIELKGIDFKEDGNILGHKLEYNYFSDNVYITADKQKNGIKANF
KIRHNIEDGGVQLADHYQQNTPIGDGPVLLPDNHYLSTQSKLSKDPNEKRDHMVLLEFVTAAGITL)ELARKLLEASTKLQRL
NIRLARALLEAIARLQELNLELVYLAVELTDPKRIRDEIKEVKDKSKEIIRRAEKEIDDAAKESEKILEEAREAISGSGSELA
KLLLKAIAETQDLNLRAAKAFLEAAAKLQELNIRAVELLVKLTDPATIREALEHAKRRSKEIIDEAERAIRAAKRESERIIEE
ARRLIEKGSGSGSELARELLRAHAQLQRLNLELLRELLRALAQLQELNLDLLRLASELTDP[DEARKAIARVKRESNAYYADA
ERLIREAAAASEK](VSKGEELFTGVVPILVELDGDVNGHKFSVSGEGEGDATYGKLTLKLICTTGKLPVPWPTLVTTLGYGV
QCFARYPDHMKQHDFFKSAMPEGYVQERTIFFKDDGNYKTRAEVKFEGDTLVNRIELKGIDFKEDGNILGHKLEYNYNSHNVY
ITADKQKNGIKANFKIRHNIEDGGVQLADHYQQNTPIGDGPVLLPDNHYLSYQSKLSKDPNEKRDHMVLLEFVTAAGITLGMD
ELYKGSGC)
E18_KRAB_full (SEQ ID NO: 27120)
MSKEAVTKLQALNIKLAEKLLEAVTKLQALNIKLAEKLLEALARLQELNIALVYLAVELTDPKRIADEIKKVKDKSKEIVERA
EEEIARAAAESKKILDEAEEEIARAAAESKKILDEGSGSGSDAVAELQALNLKLAELLLEAVAELQALNLKLAELLLEAIAKL
QELNIKLVELLTKLTDPATIREAIRKVKEDSERIVAEAERLIAAAKAESERIIREAERLIAAAKAESERIIREGSGSGDPDVA
RLQELNIELARELLRDVARLQELNIELARELLRAAAELQELNIKLVELASELTGS[(RTLVTFEDVEVDFTREEWKLLDTAQQ
IVYRNVMLENYENLVSLGYG)SDEARKAIARVKRESKRIVEDAERLIREAAAASEKISREAERLIREAAAASEKISRE]
E18_KRAB_N13t (SEQ ID NO: 27121)
MSKEAVTKLQALNIKLAEKLLEAVTKLQALNIKLAEKLLEALARLQELNIALVYLAVELTDPKRIADEIKKVKDKSKEIVERA
EEEIARAAAESKKILDEAEEEIARAAAESKKILDEGSGSGSDAVAELQALNLKLAELLLEAVAELQALNLKLAELLLEAIAKL
QELNIKLVELLTKLTDPATIREAIRKVKEDSERIVAEAERLIAAAKAESERIIREAERLIAAAKAESERIIREGSGSGDPDVA
RLQELNIELARELLRDVARLQELNIELARELLRAAAELQELNIKLVELASELTGS[(RTLVTFKDVFVDFTREEWKLLDTAQQ
IVYRNVMLENYKNLVSLGY)GSSKRIVEDAERLIREAAAASEKISREAERLIREAAAASEKISRE]
E18_KRAB_C9t (SEQ ID NO: 27122)
MSKEAVTKLQALNIKLAEKLLEAVTKLQALNIKLAEKLLEALARLQELNIALVYLAVELTDPKRIADEIKKVKDKSKEIVERA
EEEIARAAAESKKILDEAEEEIARAAAESKKILDEGSGSGSDAVAELQALNLKLAELLLEAVAELQALNLKLAELLLEAIAKL
QELNIKLVELLTKLTDPATIREAIRKVKEDSERIVAEAERLIAAAKAESERIIREAERLIAAAKAESERIIREGSGSGDPDVA
RLQELNIELARELLRDVARLQELNIELARELLRAAAELQELNIKLVELASELTGS[(RTLVTFKDVFVDFTREEWKLLDTAQQ
IVYRNVMLENYKNLVSLGY)GSDEARKAIARVKRESKRIVEDAERLIREAAAASEKISREAERLIREAA]
E18_KRAB_Cterm1 (SEQ ID NO: 27123)
MSKEAVTKLQALNIKLAEKLLEAVTKLQALNIKLAEKLLEALARLQELNIALVYLAVELTDPKRIADEIKKVKDKSKEIVERA
EEEIARAAAESKKILDEAEEEIARAAAESKKILDEGSGSGSDAVAELQALNLKLAELLLEAVAELQALNLKLAELLLEAIAKL
QELNIKLVELLTKLTDPATIREAIRKVKEDSERIVAEAERLIAAAKAESERIIREAERLIAAAKAESERIIREGSGSGDPDVA
RLQELNIELARELLRDVARLQELNIELARELLRAAAELQELNIKLVELASELT[DEARKAIARVKRESKRIVEDAE(RTLVTF
KDVFVDFTREEWKLLDTAQQIVYRNVMLENYENLVSLGY)]
E18_KRAB_Cterm2 (SEQ ID NO: 27124)
MSKEAVTKLQALNIKLAEKLLEAVTKLQALNIKLAEKLLEALARLQELNIALVYLAVELTDPKRIADEIKKVKDKSKEIVERA
EEEIARAAAESKKILDEAEEEIARAAAESKKILDEGSGSGSDAVAELQALNLKLAELLLEAVAELQALNLKLAELLLEAIAKL
QELNIKLVELLTKLTDPATIREAIRKVKEDSERIVAEAERLIAAAKAESERIIREAERLIAAAKAESERIIREGSGSGDPDVA
RLQELNIELARELLRDVARLQELNIELARELLRAAAELQELNIKLVELASELT[DEARKAIARVKRESKRIVEDAERLI(RTL
VTFKDVFVDFTREEWKLLDIAQQIVYRNVMLENYKNLVSLGY)]
E18_KRAB_Cterm3 (SEQ ID NO: 27125)
MSKEAVTKLQALNIKLAEKLLEAVTKLQALNIKLAEKLLEALARLQELNIALVYLAVELTDPKRIADEIKKVKDKSKEIVERA
EEEIARAAAESKKILDEAEEEIARAAAESKKILDEGSGSGSDAVAELQALNLKLAELLLEAVAELQALNLKLAELLLEAIAKL
QELNIKLVELLTKLTDPATIREAIRKVKEDSERIVAEAERLIAAAKAESERIIREAERLIAAAKAESERIIREGSGSGDPDVA
RLQELNIELARELLRDVARLQELNIELARELLRAAAELQELNIKLVELASELT[DEARKAIARVKRESKRIVEDAERLIREAA
AASEKISRTLVTFKDVFVDFTREEWKLLDTAQQIVYRNVMLENYKNLVSLGY)]
>3plus1_Cage_Nterm_GFP11_668 (SEQ ID NO: 27,278)
DEAKELLDEIRKAVKESEDRLEKLLRDYEKELRRDHMVLHEYVNAAGITLEELRRGSLDAKELLKTLEDLLREVLEVARRVVE
TLKELNRRVLEVVREDIEANERLLRRVLDTLRRGGVDERRIKDLERLIRESLKKAEEVLREAAEKSREIVDEIREVLKRADEA
LKRIIKKIRETRGADALSRLLEELLRVVDDLIRVLKELIDKSRKVIEELLELLKRINEENLKVLAEIIK
>3plus1_Cage_Cterm_GFP11_668 (SEQ ID NO: 27,279)
DEAKELLDEIRKAVKESEDRLEKLLRDYEKELRRLEKELRDLKRRIEEKLEELRRGSLDAKELLKTLEDLLREVLEVARRVVE
TLKELNRRVLEVVREDIERNERLLRRVLDTLRRGGVDERRIKDLERLIRESLKKAEEVLREAAEKSREIVDEIREVLKRADEA
LKRIIKKIRETRGADADHMVLHEYVNAAGITIRVLKELIDKSRKVIEELLELLKRINEENLKVLAEIIK
>3plus1_Cage_Cterm_GFP11_668 (SEQ ID NO: 27,280)
DEAKELLDEIRKAVKESEDRLEKLLRDYEKELRRLEKELRDLKRRIEEKLEELRRGSLDAKELLKTLEDLLREVLEVARRVVE
TLKELNRRVLEVVREDIERNERLLRRVLDTLRRGGVDERRIKDLERLIRESLKKAEEVLREAAEKSREIVDEIREVLKRADEA
LKRIIKKIRETRGADARDHMVLHEYVNAAGITRVLKELIDKSRKVIEELLELLKRINEENLKVLAEIIK
>3plus1_Cage_Cterm_GFP11_668 (SEQ ID NO: 27,281)
DEAKELLDEIRKAVKESEDRLEKLLRDYEKELRRLEKELRDLKRRIEEKLEELRRGSLDAKELLKTLEDLLREVLEVARRVVE
TLKELNRRVLEVVREDIERNERLLRRVLDTLRRGGVDERRIKDLERLIRESLKKAEEVLREAAEKSREIVDEIREVLKRADEA
LKRIIKKIRETRGADALSRDHMVLHEYVNAAGITLKELIDKSRKVIEELLELLKRINEENLKVLAEIIK
>3plus1_Cage_Cterm_GFP11_668 (SEQ ID NO: 27,282)
DEAKELLDEIRKAVKESEDRLEKLLRDYEKELRRLEKELRDLKRRIEEKLEELRRGSLDAKELLKTLEDLLREVLEVARRVVE
TLKELNRRVLEVVREDIERNERLLRRVLDTLRRGGVDERRIKDLERLIRESLKKAEEVLREAAEKSREIVDEIREVLKRADEA
LKRIIKKIRDHMVLHEYVNAAGITLRVVDDLIRVLKELIDKSRKVIEELLELLKRINEENLKVLAEIIK
>3plus1_Cage_Nterm_GFP11_669 (SEQ ID NO: 27,283)
SEKEKLLKESEEEVRRLRRTLEELLRKYREVLERLRDHMVLHEYVNAAGITRLKEVLDRSGLDIDTIIKEVEDLLKTVLDRLR
ELLDKIARLTKEAIEVVREIIERIVRHAERVKDELRKGGADKRKLDRVDRLIKENTRHLKEILDRIEDLVRRSEKKLRDIIRE
VRRLIEELRKKAEEIKKDPDERLVKTLIEDVERVIKRILELITRVAEDNERVLERIIRELTDNLERHLKIVREIVK
>3plus1_Cage_Nterm_GFP11_670 (SEQ ID NO: 27,284)
SEKEDLARKLRKLVEELTREYEELVKKLERLIEEIERDHMVLHEYVNAAGITISEEVRKLGTDERVLKRLLERLRRIIEEDHE
LNTELLKRLLDLLKEILDTSRELLKRLLDILRKGVRDEEVLRDLERTLREVLEENERAIEEAERVLRKVLEDSERAVRDARRV
LAEVDKSPTGDEALRKLVELLVEVLRRLIRVNRELVKLLREVLERLLRILRESVKKLKRLIEKVIKDAT
>3plus1_Cage_Cterm_GFP11_670 (SEQ ID NO: 27,285)
SEKEDLARKLRKLVEELTREYEELVKKLERLIEEIEKVSEESVRKLEKLLREISEEVRKLGTDERVLKRLLERLRRIIEEDHE
LNTELLKRLLDLLKEILDTSRELLKRLLDILRKGVRDEEVLRDLERTLREVLEENERAIEEAERVLRKVLEDSERAVRDARRV
LAEVDKSPTGDEARDHMVLHEYVNAAGITRVNRELVKLLREVLERLLRILRESVKKLKRLIEKVIKDAT
>3plus1_Cage_Cterm_GFP11_670 (SEQ ID NO: 27,286)
SEKEDLARKLRKLVEELTREYEELVKKLERLIEEIEKVSEESVRKLEKLLREISEEVRKLGTDERVLKRLLERLRRIIEEDHE
LNTELLKRLLDLLKEILDTSRELLKRLLDILRKGVRDEEVLRDLERTLREVLEENERAIEEAERVLRKVLEDSERAVRDARRV
LAEVDKSPTGDERDHMVLHEYVNAAGITIRVNRELVKLLREVLERLLRILRESVKKLKRLIEKVIKDAT
>3plus1_Cage_Cterm_GFP11_670 (SEQ ID NO: 27,287)
SEKEDLARKLRKLVEELTREYEELVKKLERLIEEIEKVSEESVRKLEKLLREISEEVRKLGTDERVLKRLLERLRRIIEEDHE
LNTELLKRLLDLLKEILDTSRELLKRLLDILRKGVRDEEVLRDLERTLREVLEENERAIEEAERVLRKVLEDSERAVRDARRV
LAEVDKSPTGRDHMVLHEYVNAAGITRLIRVNRELVKLLREVLERLLRILRESVKKLKRLIEKVIKDAT
>3plus1_Cage_Nterm_GFP11_670 (SEQ ID NO: 27,288)
SEKEDLARKLRKLVEELTREYEELVKKLERLIEEIRDHMVLHEYVNAAGITEISEEVRKLGTDERVLKRLLERLRRIIEEDHE
LNTELLKRLLDLLKEILDTSRELLKRLLDILRKGVRDEEVLRDLERTLREVLEENERAIEEAERVLRKVLEDSERAVRDARRV
LAEVDKSPTGDEALRKLVELLVEVLRRLIRVNRELVKLLREVLERLLRILRESVKKLKRLIEKVIKDAT
>3plus1_Cage_Cterm_GFP11_670 (SEQ ID NO: 27,289)
SEKEDLARKLRKLVEELTREYEELVKKLERLIEEIEKVSEESVRKLEKLLREISEEVRKLGTDERVLKRLLERLRRIIEEDHE
LNTELLKRLLDLLKEILDTSRELLKRLLDILRKGVRDEEVLRDLERTLREVLEENERAIEEAERVLRKVLEDSERAVRDARRV
LAEVDKSPTRDHMVLHEYVNAAGITRRLIRVNRELVKLLREVLERLLRILRESVKKLKRLIEKVIKDAT
>3plus1_Cage_Nterm_GFP11_670 (SEQ ID NO: 27,290)
SEKEDLARKLRKLVEELTREYEELVKKLERLIERDHMVLHEYVNAAGITLREISEEVRKLGTDERVLKRLLERLRRIIEEDHE
LNTELLKRLLDLLKEILDTSRELLKRLLDILRKGVRDEEVLRDLERTLREVLEENERAIEEAERVLRKVLEDSERAVRDARRV
LAEVDKSPTGDEALRKLVELLVEVLRRLIRVNRELVKLLREVLERLLRILRESVKKLKRLIEKVIKDAT
>3plus1_Cage_Nterm_GFP11_670 (SEQ ID NO: 27,291)
SEKEDLARKLRKLVEELTREYEELVKKLERLIEEIEKRDHMVLHEYVNAAGITSEEVRKLGTDERVLKRLLERLRRIIEEDHE
LNTELLKRLLDLLKEILDTSRELLKRLLDILRKGVRDEEVLRDLERTLREVLEENERAIEEAERVLRKVLEDSERAVRDARRV
LAEVDKSPTGDEALRKLVELLVEVLRRLIRVNRELVKLLREVLERLLRILRESVKKLKRLIEKVIKDAT
>3plus1_Cage_Nterm_GFP11_670 (SEQ ID NO: 27,292)
SEKEDLARKLRKLVEELTREYEELVKKLERLIEEIEKVSEESRDHMVLHEYVNAAGITKLGTDERVLKRLLERLRRIIEEDHE
LNTELLKRLLDLLKEILDTSRELLKRLLDILRKGVRDEEVLRDLERTLREVLEENERAIEEAERVLRKVLEDSERAVRDARRV
LAEVDKSPTGDEALRKLVELLVEVLRRLIRVNRELVKLLREVLERLLRILRESVKKLKRLIEKVIKDAT
>3plus1_Cage_Nterm_GFP11_670 (SEQ ID NO: 27,293)
SEKEDLARKLRKLVEELTREYEELVKKLERLIEEIEKVSEESVRDHMVLHEYVNAAGITLGTDERVLKRLLERLRRIIEEDHE
LNTELLKRLLDLLKEILDTSRELLKRLLDILRKGVRDEEVLRDLERTLREVLEENERAIEEAERVLRKVLEDSERAVRDARRV
LAEVDKSPTGDEALRKLVELLVEVLRRLIRVNRELVKLLREVLERLLRILRESVKKLKRLIEKVIKDAT
>3plus1_Cage_Cterm_GFP11_671 (SEQ ID NO: 27,294)
SEEEDLLERVKRVLDELIEIVDRNHELNRRVVETSAALVERLLEEVERALETLEREIPGSSLLDKAIKDLRDVLRRVKEKVKR
SIEELKEVLEESRRVLEEVVRKLREVIDRVRRLVEKGVDLRDLIRELKRVLEEAVKLIERLVRLNTRAAEKDNESLRELVRAI
KEALKRAVDAVRKGGLDSRAVKKLDRDHMVLHEYVNAAGITNEELWRALVELNKESVRRLREIVERVARDLEETAR
>3plus1_Cage_Cterm_GFP11_671 (SEQ ID NO: 27,295)
SEEEDLLERVKRVLDELIEIVDRNHELNRRVVETSAALVERLLEEVERALETLEREIPGSSLLDKAIKDLRDVLRRVKEKVKR
SIEELKEVLEESRRVLEEVVRKLREVIDRVRRLVEKGVDLRDLIRELKRVLEEAVKLIERLVRLNTRAAEKDNESLRELVRAI
KEALKRAVDAVRKGGLDSRARDHMVLHEYVNAAGITDVVRRNEELWRALVELNKESVRRLREIVERVARDLEETAR
>3plus1_Cage_Cterm_GFP11_671 (SEQ ID NO: 27,296)
SEEEDLLERVKRVLDELIEIVDRNHELNRRVVETSAALVERLLEEVERALETLEREIPGSSLLDKAIKDLRDVLRRVKEKVKR
SIEELKEVLEESRRVLEEVVRKLREVIDRVRRLVEKGVDLRDLIRELKRVLEEAVKLIERLVRLNTRAAEKDNESLRELVRAI
KEALKRAVDAVRKGGLDSRRDHMVLHEYVNAAGITEDVVRRNEELWRALVELNKESVRRLREIVERVARDLEETAR
>3plus1_Cage_Cterm_GFP11_671 (SEQ ID NO: 27,297)
SEEEDLLERVKRVLDELIEIVDRNHELNRRVVETSAALVERLLEEVERALETLEREIPGSSLLDKAIKDLRDVLRRVKEKVKR
SIEELKEVLEESRRVLEEVVRKLREVIDRVRRLVEKGVDLRDLIRELKRVLEEAVKLIERLVRLNTRAAEKDNESLRELVRAI
KEALKRAVDAVRKGGLRDHMVLHEYVNAAGITKKLEDVVRRNEELWRALVELNKESVRRLREIVERVARDLEETAR
>3plus1_Cage_Cterm_GFP11_672 (SEQ ID NO: 27,298)
DETEEVIKKIAKLIDELIETLKKIVEDSAKLIKELIDLLEEIIKRSADLIRELEKVSKRGDRDRLKKLIDRMRRETERALEEL
RRVIEELKEATERALKTIEDLIREIEEAIRRELGDEDLAREILEVIRRILELLREDMELNLRALEEILRLLEEVIKLNKDAID
EILELLKRDHMVLHEYVNAAGITESKKLVRDVVEEAKRLLKENRDSTRKIIEDIRRLLRKIEDSTR
>3plus1_Cage_Cterm_GFP11_672 (SEQ ID NO: 27,299)
DETEEVIKKIAKLIDELIETLKKIVEDSAKLIKELIDLLEEIIKRSADLIRELEKVSKRGDRDRLKKLIDRMRRETERALEEL
RRVIEELKEATERALKTIEDLIREIEEAIRRELGDEDLAREILEVIRRILELLREDMELNLRALEEILRLLEEVIKLNKDAID
EILELLKGRDHMVLHEYVNAAGITSKKLVRDVVEEAKRLLKENRDSTRKIIEDIRRLLRKIEDSTR
>3plus1_Cage_Nterm_GFP11_67>3 (SEQ ID NO: 27,300)
SKEETLRKEAEDLLRRLEELTRRLEKKARELLERAKKLSRDLARDHMVLHEYVNAAGITEELIKVLEEVAKTSIDLNKEVIDV
IRRVIEESLELNKRVLDVLRRVLDIAARKGRDKKAIRDVVREAARVIKEVIDEDEKVNRDLKRVIERVIDDDRKALERLRKAV
EEVASRGDDEAAKRLVEELLKAVTDLSRKNKRILEELLKAIETLSDENKKILTEILDALRRLVEKIEK
>3plus1_Cage_Nterm_GFP11_67>3 (SEQ ID NO: 27,301)
SKEETLRKEAEDLLRRLEELTRRLEKKARELLERAKKLSRRDHMVLHEYVNAAGITGVPEELIKVLEEVAKTSIDLNKEVIDV
IRRVIEESLELNKRVLDVLRRVLDIAARKGRDKKAIRDVVREAARVIKEVIDEDEKVNRDLKRVIERVIDDDRKALERLRKAV
EEVASRGDDEAAKRLVEELLKAVTDLSRKNKRILEELLKAIETLSDENKKILTEILDALRRLVEKIEK
>3plus1_Cage_Cterm_GFP11_67>3 (SEQ ID NO: 27,302)
SKEETLRKEAEDLLRRLEELTRRLEKKARELLERAKKLSRDLAEELKRLLKELREKGVPEELIKVLEEVAKTSIDLNKEVIDV
IRRVIEESLELNKRVLDVLRRVLDIAARKGRDKKAIRDVVREAARVIKEVIDEDEKVNRDLKRVIERVIDDDRKALERLRKAV
EEVASRGDDEARDHMVLHEYVNAAGITRKNKRILEELLKAIETLSDENKKILTEILDALRRLVEKIEK
>3plus1_Cage_Nterm_GFP11_67>3 (SEQ ID NO: 27,303)
SKEETLRKEAEDLLRRLEELTRRLEKKARELLERARDHMVLHEYVNAAGITELREKGVPEELIKVLEEVAKTSIDLNKEVIDV
IRRVIEESLELNKRVLDVLRRVLDIAARKGRDKKAIRDVVREAARVIKEVIDEDEKVNRDLKRVIERVIDDDRKALERLRKAV
EEVASRGDDEAAKRLVEELLKAVTDLSRKNKRILEELLKAIETLSDENKKILTEILDALRRLVEKIEK
>3plus1_Cage_Nterm_GFP11_67>3 (SEQ ID NO: 27,304)
SKEETLRKEAEDLLRRLEELTRRLEKKARELLERAKKLSRDHMVLHEYVNAAGITKGVPEELIKVLEEVAKTSIDLNKEVIDV
IRRVIEESLELNKRVLDVLRRVLDIAARKGRDKKAIRDVVREAARVIKEVIDEDEKVNRDLKRVIERVIDDDRKALERLRKAV
EEVASRGDDEAAKRLVEELLKAVTDLSRKNKRILEELLKAIETLSDENKKILTEILDALRRLVEKIEK
>3plus1_Cage_Nterm_GFP11_67>3 (SEQ ID NO: 27,305)
SKEETLRKEAEDLLRRLEELTRRLEKKARELLERAKKLRDHMVLHEYVNAAGITEKGVPEELIKVLEEVAKTSIDLNKEVIDV
IRRVIEESLELNKRVLDVLRRVLDIAARKGRDKKAIRDVVREAARVIKEVIDEDEKVNRDLKRVIERVIDDDRKALERLRKAV
EEVASRGDDEAAKRLVEELLKAVTDLSRKNKRILEELLKAIETLSDENKKILTEILDALRRLVEKIEK
>3plus1_Cage_Nterm_GFP11_67>3 (SEQ ID NO: 27,306)
SKEETLRKEAEDLLRRLEELTRRLEKKARELLERAKKLSRDLRDHMVLHEYVNAAGITPEELIKVLEEVAKTSIDLNKEVIDV
IRRVIEESLELNKRVLDVLRRVLDIAARKGRDKKAIRDVVREAARVIKEVIDEDEKVNRDLKRVIERVIDDDRKALERLRKAV
EEVASRGDDEAAKRLVEELLKAVTDLSRKNKRILEELLKAIETLSDENKKILTEILDALRRLVEKIEK
>3plus1_Cage_Nterm_GFP11_674 (SEQ ID NO: 27,307)
SEREEVKEKLDRLLEEVEKTVRELKREHDELLKEVEKLVRDLKRDHMVLHEYVNAAGITKEILDVIREHVRTNKEILDRVLEV
VEEHLRRNKEILDKLLDDIRKVVEDAKRILGIGDDETLRRAVRRILEELRKLVEDIRKKVKDSLETLERALEEAEERIRRSLE
DLKRVLKEAKDKTKDKDRLDKVEELVKKLLEDTKRTVDRVRELVRKILKKSRETLEELERLIEKILRELEKDAR
>3plus1_Cage_Cterm_GFP11_674 (SEQ ID NO: 27,308)
SEREEVKEKLDRLLEEVEKTVRELKREHDELLKEVEKLVRDLKKEHDELLKKVKDDGVPKEILDVIREHVRTNKEILDRVLEV
VEEHLRRNKEILDKLLDDIRKVVEDAKRILGIGDDETLRRAVRRILEELRKLVEDIRKKVKDSLETLERALEEAEERIRRSLE
DLKRVLKEAKDKTKDKDRDHMVLHEYVNAAGITKRTVDRVRELVRKILKKSRETLEELERLIEKILRELEKDAR
>3plus1_Cage_Nterm_GFP11_675 (SEQ ID NO: 27,309)
SERETVKRRLEELLKEVKRTLDKLKEEHDRLLEDVRRVVEELRDHMVLHEYVNAAGITPEELLRVIAKVLETNKRILDDLLRV
VKKHVDLNKEILDRILEMIKEIVERVKRVLGDGDEKTLRDKIRDIIRRLEDAAREAEERVRRSLEELKKAVEKIRKKIEDSLR
ELEEALKRVRDKEEDDKRLEDISRLVKRLLDESRRVLRELEETIRKRAEESKRVLEEVKRLVEKLIRELRKEAE
>3plus1_Cage_Nterm_GFP11_676 (SEQ ID NO: 27,310)
SEDEIIKKIIEDLRRVLKEVEEIHKEVEERLDKRDHMVLHEYVNAAGITDRVLDEVKRIGDVETVLRLAIEAVRRALEIVRKV
TEDLIRVVRKLLEIVDAVNKRVKKAVEDVLKLLRDTLKGSKDVERVLDEARRLLEKLHRDSRRILKRIAELLDELKELSERIL
ERIKRLLEEVERRGSDDERLLEELIEELKDLIRRHIELLRRLIERLKELLKKHIEDLRRLLEEIEKLLEEARK
>3plus1_Cage_Nterm_GFP11_677 (SEQ ID NO: 27,311)
SREEVLRELEEVIEDNRRLLEELIEKSKKVLDESLKLIDELLRRDHMVLHEYVNAAGITSKGDDEETARRIVTKIRKELEDVL
DEVRRIVKEILDRDRRVAEDLKRVVDENLEKLKKVLDELDKKLGDRRLVEILRDIVRLLIEINERALRELLKLHEEHTRIVAE
LLRELERVVEELRRLVELIARGEISEDDLKRVVDEVEKKLRELKRRYKEALERIKEKIKELKDRYERAVREVVAELRKTTK
>3plus1_Cage_Cterm_GFP11_677 (SEQ ID NO: 27,312)
SREEVLRELEEVIEDNRRLLEELIEKSKKVLDESLKLIDELLRRLEEVLERVLRLLEELSKGDDEETARRIVTKIRKELEDVL
DEVRRIVKEILDRDRRVAEDLKRVVDENLEKLKKVLDELDKKLGDRRLVEILRDIVRLLIEINERALRELLKLHEEHTRIVAE
LLRELERVVEELRRLVELIARGEISEDDRDHMVLHEYVNAAGITRRYKEALERIKEKIKELKDRYERAVREVVAELRKTTK
>3plus1_Cage_Cterm_GFP11_677 (SEQ ID NO: 27,313)
SREEVLRELEEVIEDNRRLLEELIEKSKKVLDESLKLIDELLRRLEEVLERVLRLLEELSKGDDEETARRIVTKIRKELEDVL
DEVRRIVKEILDRDRRVAEDLKRVVDENLEKLKKVLDELDKKLGDRRLVEILRDIVRLLIEINERALRELLKLHEEHTRIVAE
LLRELERVVEELRRLVELIARGERDHMVLHEYVNAAGITLRELKRRYKEALERIKEKIKELKDRYERAVREVVAELRKTTK
>3plus1_Cage_Nterm_GFP11_678 (SEQ ID NO: 27,314)
SKAEEIAEKLDRLLEENRRALEEITTRLDDLLRRDHMVLHEYVNAAGITLDDLRRGGIREELLKRIADVIRDIMRLLKELHDH
TAEVIKTIKKLLKELHDINKEIIERLKRLKDGNVPKEELLKRVEELVRTSARLTTEVLKTVEKLIRDDKRLSEEILKRVKELI
EELKRGVDSERVKEILERILRVVEEAVRLNEESLRRILDVVRKAVKLDRESLKKILDVVEEAVR
>3plus1_Cage_Cterm_GFP11_678 (SEQ ID NO: 27,315)
SKAEEIAEKLDRLLEENRRALEEITTRLDDLLRRNKDALRKVMEKLKRLLDDLRRGGIREELLKRIADVIRDIMRLLKELHDH
TAEVIKTIKKLLKELHDINKEIIERLKRLKDGNVPKEELLKRVEELVRTSARLTTEVLKTVEKLIRDDKRLSEEILKRVKELI
EELRDHMVLHEYVNAAGITLRVVEEAVRLNEESLRRILDVVRKAVKLDRESLKKILDVVEEAVR
>3plus1_Cage_Nterm_GFP11_678 (SEQ ID NO: 27,316)
SKAEEIAEKLDRLLEENRRALEEITTRLDDLLRRNKDRDHMVLHEYVNAAGITRRGGIREELLKRIADVIRDIMRLLKELHDH
TAEVIKTIKKLLKELHDINKEIIERLKRLKDGNVPKEELLKRVEELVRTSARLTTEVLKTVEKLIRDDKRLSEEILKRVKELI
EELKRGVDSERVKEILERILRVVEEAVRLNEESLRRILDVVRKAVKLDRESLKKILDVVEEAVR
>3plus1_Cage_Cterm_GFP11_678 (SEQ ID NO: 27,317)
SKAEEIAEKLDRLLEENRRALEEITTRLDDLLRRNKDALRKVMEKLKRLLDDLRRGGIREELLKRIADVIRDIMRLLKELHDH
TAEVIKTIKKLLKELHDINKEIIERLKRLKDGNVPKEELLKRVEELVRTSARLTTEVLKTVEKLIRDDKRLSEEILKRVKELI
EELKRGRDHMVLHEYVNAAGITVEEAVRLNEESLRRILDVVRKAVKLDRESLKKILDVVEEAVR
>3plus1_Cage_Nterm_GFP11_678 (SEQ ID NO: 27,318)
SKAEEIAEKLDRLLEENRRALEEITTRLDDLLRRNKRDHMVLHEYVNAAGITLRRGGIREELLKRIADVIRDIMRLLKELHDH
TAEVIKTIKKLLKELHDINKEIIERLKRLKDGNVPKEELLKRVEELVRTSARLTTEVLKTVEKLIRDDKRLSEEILKRVKELI
EELKRGVDSERVKEILERILRVVEEAVRLNEESLRRILDVVRKAVKLDRESLKKILDVVEEAVR
>3plus1_Cage_Nterm_GFP11_678 (SEQ ID NO: 27,319)
SKAEEIAEKLDRLLEENRRALEEITTRLDDLLRRNKDALRDHMVLHEYVNAAGITGGIREELLKRIADVIRDIMRLLKELHDH
TAEVIKTIKKLLKELHDINKEIIERLKRLKDGNVPKEELLKRVEELVRTSARLTTEVLKTVEKLIRDDKRLSEEILKRVKELI
EELKRGVDSERVKEILERILRVVEEAVRLNEESLRRILDVVRKAVKLDRESLKKILDVVEEAVR
>3plus1_Cage_Cterm_GFP11_679 (SEQ ID NO: 27,320)
SRVEELKKLIEDILRISREVVERIKRVAEDIHRINRRVLDDLRKLIEDILRTVEEILARKVGDTEIAERLRDTIARVVDEIAK
LLEEHEKRSRELLEEIRKLLEDILRRSERAVEEIRELLKKGVSTKDVLRIIEEILREHLELLERVVRRIEEILRELLKTIEEI
VKRIKEILEELKEVLKRGRVKDDEVERDHMVLHEYVNAAGITYRRLLEEIKRKLEEILRRVEELHRRLRRKLEEIDR
>3plus1_Cage_Nterm_GFP11_679 (SEQ ID NO: 27,321)
SRVEELKKLIEDILRISREVVERIKRVAEDIHRINRRVRDHMVLHEYVNAAGITEILARKVGDTEIAERLRDTIARVVDEIAK
LLEEHEKRSRELLEEIRKLLEDILRRSERAVEEIRELLKKGVSTKDVLRIIEEILREHLELLERVVRRIEEILRELLKTIEEI
VKRIKEILEELKEVLKRGRVKDDEVEREIRRVKEDLDRILEEYRRLLEEIKRKLEEILRRVEELHRRLRRKLEEIDR
>SB76L (SEQ ID NO: 1)
(MGSSHHHHHHSSGLVPRGSHM)SKEAVIKLQALNIKLAEKLLEALARLQELNIALVYLAVELTDPKRIADEIKK
VKDKSKEIVERAEEEIARAAAESKKILDEGSGSGSDAVAELQALNLKLAELLLEAIAKLQELNIKLVELLTKLID
PATIREAIRKVKEDSERIVAEAERLIAAAKAESERIIREGSGSGDPDVARLQELNIELARELLRAAAELQELNIK
LVELASELTDP[DEARKAIARVKRESKRIVEDAERLIREAAAASEKISRE]
>SB76L_17 (SEQ ID NO: 2)
(MGSSHHHHHHSSGLVPRGSHM)GSKEAVIKLMALNLKLAEKLLEAIARLQELNIALVYLATELTDPERIREEIR
KVKEESARIVEEAEEEIRRAAARSEDILREGSGSGSDAVAELQRLNLELAELLLRAAAKLQELNIDLVRLLTELT
DPKTIRDAIERVKAESERIVREAERLIREAKADSERILREGSGSGDPDVARLQELFIELARELLEALARLQELNI
DLVRLASELTDP[DTIRDAIRRVKEESARIVEDARRLIKEAAEEAEKISRE]
>SB76L_18 (SEQ ID NO: 3)
(MGSSHHHHHHSSGLVPRGSHM)GSKRAVTELQKLNIELARKLLRALAELMELNIALVYLAVELTDPRRIREEIR
KVKEKSDEIVKRAEDEIRKAAAESEKILREGSGSGSDAVAELQRLNLELAKLLLEAIAKLQALNIDLVRLLTELT
DPETIRRAIKRVKDESARIVEEAEKLIRAAKDKAREIIDKGSGSGDPDVARLQELNIELARELLEAAARLQELFI
DLVRLASELTDP[DEARKAIERVKREAERIVREAERLIREAKRASKEISDE]
>LOCKR_extend5 (SEQ ID NO: 4)
(MGSSHHHHHHSSGLVPRGSHM)KLLEAVIKLQALNIKLAEKLLEALARLQELNIALVYLAVELTDPKRIADEIK
KVKDKSKEIVERAEEEIARAAAESKKILDEAEEEGSGSGSELLLEAVAELQALNLKLAELLLEAIAKLQELNIKL
VELLTKLTDPATIREAIRKVKEDSERIVAEAERLIAAAKAESERIIREAERLAGSGSGSRELLRDVARLQELNIE
LARELLRAAAELQELNIKLVELASELTDP[DEARKAIARVKRESKRIVEDAERLIREAAAASEKISREAERLI]
>LOCKR_extend9 (SEQ ID NO: 5)
(MGSSHHHHHHSSGLVPRGSHM)KLAEKLLEAVIKLQALNIKLAEKLLEALARLQELNIALVYLAVELTDPKRIA
DEIKKVKDKSKEIVERAEEEIARAAAESKKILDEAEEEIARAGSGSGSLKLAELLLEAVAELQALNLKLAELLLE
AIAKLQELNIKLVELLTKLTDPATIREAIRKVKEDSERIVAEAERLIAAAKAESERIIREAERLIAAAAGSGSGS
IELARELLRDVARLQELNIELARELLRAAAELQELNIKLVELASELTDP[DEARKAIARVKRESKRIVEDAERLI
REAAAASEKISREAERLIREAA]
>LOCKR_extend18 (SEQ ID NO: 6)
(MGSSHHHHHHSSGLVPRGSHM)SKEAVIKLQALNIKLAEKLLEAVIKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDP[DEARKAIARVKRESKRIVEDAERLIREAAAASEKISREAERLIREAAA
ASEKISRE]
>LOCKRb (SEQ ID NO: 7)
(MGSSHHHHHHSSGLVPRGSHM)SHAAVIKLSDLNIRLLDKLLQAVIKLTELNAELNRKLIEALQRLFDLNVALV
HLAAELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSNDPQVAQNQETFIELARDALRLVAENQEAFIEVAR
LTLRAAALAQEVAIKAVEAASEGGSGSG[NKEEIEKLAKEAREKLKKAEKEHKEIHDKLRKKNKKAREDLKKKAD
ELRETNKRVN]
>LOCKRc (SEQ ID NO: 8)
(MGSSHHHHHHSSGLVPRGSHM)SLEAVLKLAELNLKLSDKLAEAVQKLAALLNKLLEKLSEALQRLFELNVALV
TLAIELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSNDPLVARLQELLIEHARELLRLVATSQEIFIELAR
AFLANAAQLQEAAIKAVEAASENGSGSG[SSEKVRRELKESLKENHKQNQKLLKDHKRAQEKLNRELEELKKKHK
KTLDDIRRES]
>LOCKRd (SEQ ID NO: 9)
(MGSSHHHHHHSSGLVPRGSHM)SLEAVLKLFELNHKLSEKLLEAVLKLHALNQKLSQKLLEALARLLELNVALV
ELAIELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPEVARLQEAFIEQAREILRNVAAAQEALIEQAR
RLLALAALAQEAAIKAVELASEHGSGSG[DTVKRILEELRRRFEKLAKDLDDIARKLLEDHKKHNKELKDKQRKI
KKEADDAARS]
>LOCKRe (SEQ ID NO: 10)
(MGSSHHHHHHSSGLVPRGSHM)SLEAVLKLQDLNSKLSEKLSEAQLKLQALNNKLLRKLLEALLRLQDLNQALV
NLALQLTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVAKSQEHLIEHARELLRQVAKSQELFIELAR
QLLRLAAKSQELAIKAVELASEAGSGSG[DDVERRLRKANKESKKEAEELTEEAKKANEKTKEDSKELTKENRKT
NKTIKDEARS]
>LOCKRf (SEQ ID NO: 11)
(MGSSHHHHHHSSGLVPRGSHM)SREAVEKLAELNHKLSHKLQQAQQKLQALNLKLLQKLLEALDRLQDLNNALV
KLAQRLTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARQQETLIEQARRLLRNVAESQELFIEAAR
TVLRLAAKLQEINIKQVELASEAGSGSG[DDEERRSEKTVQDAKREIKKVEDDLQRLNEEQKKKVKKQEDENQKT
LKKHKDDARS]
>miniLOCKRa_1 (SEQ ID NO: 12)
(MGSSHHHHHHSSGLVPRGSHM)NKEDATEAQKKAIRAAEELLKDVTRIQERAIREAEKALERLARVQEEAIRRV
YEAVESKNKEELKKVKEEIEELLRRLKRELDELEREIRELLKEIKEKADRLEKEIRDLIERIRRDRNASDEVVIR
LARLNEELIRELREDVRRLAELNKELLRELERAARELARLNEKLLELADRVETE[EEARKAIARVKRESKRIVED
AERLIREAAAASEKISREAERLIREAAAASEKISRE]
>miniLOCKRa_2 (SEQ ID NO: 13)
(MGSSHHHHHHSSGLVPRGSHM)DERLKRLNERLADELDKDLERLLRLNEELARELTRAAEELRELNEKLVELAK
KLQGGRSREVAERAEKEREKIRRKLEEIKKEIKEDADRIKKRADELRRRLEKTLEDAARELEKLKREPRTEELKR
KATELQKEAIRRAEELLKEVIDVQRRAIERAEELLEKLARLQEEAIRTVYLLVELNKV[DRARKAIARVKRESKR
IVEDAERLIREAAAASEKISREAERLIREAAAASEKISRE]
>miniLOCKRc_1 (SEQ ID NO: 14)
(MGSSHHHHHHSSGLVPRGSHM)LIERLTRLEKEHVRELKRLLDTSLEILRRLVEAFETNLRQLKEALKRALEAA
NLHNEEVEEVLRKLEEDLRRLEEELRKILDDVRKEVKRLKEELDKRIKEVEDELRKIKEKLKKGDKNEKRVLEEI
LRLAEDVLKKSDKLAKDVQERARELNEILEELSRKLQELFERVVEEVIRNVPT[TERIEKVRRELKESLKENHKQ
NQKLLKDHKRAQEKLNRELEELKKKHKKTLDDIRRES]
>miniLOCKRc 2 (SEQ ID NO: 15)
(MGSSHHHHHHSSGLVPRGSHM)SEERVLELAEEALRLSDEAAKEIQELARRLNEELEKLSKELQDLFERIVETV
TRLIDADEETLKRAAEEIKKRLEDARKKAKEAADKAREELDRARKKLKELVDEIRKKAKDALEKAGADEELVARL
LRLLEEHARELERLLRTSARIIERLLDAFRRNLEQLKEAADKAVEAAEEAVRRVED[VRVWSEKVRRELKESLKE
NHKQNQKLLKDHKRAQEKLNRELEELKKKHKKTLDDIRRES]
>1fix-short-noBim-t0 (SEQ ID NO: 16)
(MGSHHHHHHGSGSENLYFQGSGG)SELARKLLEASTKLQRLNIRLAEALLEAIARLQELNLELVYLAVELTDPK
RIRDEIKEVKDKSKEIIRRAEKEIDDAAKESEKILEEAREAISGSGSELAKLLLKAIAETQDLNLRAAKAFLEAA
AKLQELNIRAVELLVKLTDPATIREALEHAKRRSKEIIDEAERAIRAAKRESERIIEEARRLIEKGSGSGS[ELA
RELLRAHAQLQRLNLELLRELLRALAQLQELNLDLLRLASELTDPDEARKAIARVKRESKRIVEDAERLIREAAA
ASEKISREAERLIR]
>1fix-short-noBim(AYYA)-t0 (SEQ ID NO: 17)
(MGSHHHHHHGSGSENLYFQGSGG)SELARKLLEASTKLQRLNIRLAEALLEAIARLQELNLELVYLAVELTDPK
RIRDEIKEVKDKSKEIIRRAEKEIDDAAKESEKILEEAREAISGSGSELAKLLLKAIAETQDLNLRAAKAFLEAA
AKLQELNIRAVELLVKLTDPATIREALEHAKRRSKEIIDEAERAIRAAKRESERIIEEARRLIEKGSGSGS[ELA
RELLRAHAQLQRLNLELLRELLRALAQLQELNLDLLRLASELTDPDEARKAIARVKRESNAYYADAERLIREAAA
ASEKISREAERLIR]
“(3) Functional LOCKR Cage designs with bioactive peptides encoded into the Latch”,
>aBc12LOCKR (SEQ ID NO: 18)
(MGSSHHHHHHSSGLVPRGSHM)GSKEAVIKLQALNIKLAEKLLEALARLQELNIALVYLAVELTDPKRIADEIK
KVKDKSKEIVERAEEEIARAAAESKKILDEGSGSGSDAVAELQALNLKLAELLLEAIAKLQELNIKLVELLTKLT
DPATIREAIRKVKEDSERIVAEAERLIAAAKAESERIIREGSGSGDPDVARLQELNIELARELLRAAAELQELNI
KLVELASELT(GSGSGSG)[DPKMAQEL [symbol] DK [symbol] RAASLQI [symbol] GDAFYA [symbol] LRALAASEKLSKE]
>pBimLOCKR (SEQ ID NO: 19)
(MGSSHHHHHHSSGLVPRGSHM)KEAVIKLQALNIKLAEKLLEALARLQELNIALVYLAVELTDPKRIADEIKKV
KDKSKEIVERAEEEIARAAAESKKILDEGSGSGSDAVAELQALNLKLAELLLEAIAKLQELNIKLVELLTKLTDP
ATIREAIRKVKEDSERIVAEAERLIAAAKAESERIIREGSGSGDPDVARLQELNIELARELLRAAAELQELNIKL
VELASEGSGSGS[EIAEALRAIGDVFNESYRIVEDAERLIREAAAASEKISRE]
>BimLOCKR_extend5 (SEQ ID NO: 20)
(MGSSHHHHHHSSGLVPRGSHM)KLLEAVIKLQALNIKLAEKLLEALARLQELNIALVYLAVELTDPKRIADEIK
KVKDKSKEIVERAEEEIARAAAESKKILDEAEEEGSGSGSELLLEAVAELQALNLKLAELLLEAIAKLQELNIKL
VELLTKLTDPATIREAIRKVKEDSERIVAEAERLIAAAKAESERIIREAERLAGSGSGSRELLRDVARLQELNIE
LARELLRAAAELQELNIKLVELASELTD[EIWIAQELRRIGDEFNAYYADAERLIREAAAASEKISREAERLI]
>BimLOCKR_extend9 (SEQ ID NO: 21)
(MGSSHHHHHHSSGLVPRGSHM)KLAEKLLEAVIKLQALNIKLAEKLLEALARLQELNIALVYLAVELTDPKRIA
DEIKKVKDKSKEIVERAEEEIARAAAESKKILDEAEEEIARAGSGSGSLKLAELLLEAVAELQALNLKLAELLLE
AIAKLQELNIKLVELLTKLTDPATIREAIRKVKEDSERIVAEAERLIAAAKAESERIIREAERLIAAAAGSGSGS
IELARELLRDVARLQELNIELARELLRAAAELQELNIKLVELASELTD[EIWIAQELRRIGDEFNAYYADAERLI
REAAAASEKISREAERLIREAA]
>BimLOCKR_extend18 (SEQ ID NO: 22)
(MGSSHHHHHHSSGLVPRGSHM)SKEAVIKLQALNIKLAEKLLEAVIKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTD[EIWIAQELRRIGDEFNAYYADAERLIREAAAASEKISREAERLIREAAA
ASEKISRE]
>BimLOCKRb (SEQ ID NO: 23)
(MGSSHHHHHHSSGLVPRGSHM)SHAAVIKLSDLNIRLLDKLLQAVIKLTELNAELNRKLIEALQRLFDLNVALV
HLAAELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSNDPQVAQNQETFIELARDALRLVAENQEAFIEVAR
LTLRAAALAQEVAIKAVEAASEGGSGSG[NEIWIAQELRRIGDEFNAYYAEHKEIHDKLRKKNKKAREDLKKKAD
ELRETNKRVN]
>BimLOCKRc (SEQ ID NO: 24)
(MGSSHHHHHHSSGLVPRGSHM)SLEAVLKLAELNLKLSDKLAEAVQKLAALLNKLLEKLSEALQRLFELNVALV
TLAIELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSNDPLVARLQELLIEHARELLRLVATSQEIFIELAR
AFLANAAQLQEAAIKAVEAASENGSG[EIWIAQELRRIGDEFNAYYAQNQKLLKDHKRAQEKLNRELEELKKKHK
KTLDDIRRES]
>BimLOCKRd (SEQ ID NO: 25)
(MGSSHHHHHHSSGLVPRGSHM)SLEAVLKLFELNHKLSEKLLEAVLKLHALNQKLSQKLLEALARLLELNVALV
ELAIELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPEVARLQEAFIEQAREILRNVAAAQEALIEQAR
RLLALAALAQEAAIKAVELASEHGSGS[EIWIAQELRRIGDEFNAYYADLDDIARKLLEDHKKHNKELKDKQRKI
KTIKDEARS]
>StrepLOCKRa_300 (SEQ ID NO: 26)
(MGSSHHHHHHSSGLVPRGSHM)SKEAVIKLQALNIKLAEKLLEAVIKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELAS(GG)[NWSHPQFEKKAIARVKRESKRIVEDAERLIREAAAASEKISREAERLIR
EAAAASEKISRE]
>strepLOCKRa_306 (SEQ ID NO: 27)
(MGSSHHHHHHSSGLVPRGSHM)SKEAVIKLQALNIKLAEKLLEAVIKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[ENWSHPQFEKRESKRIVEDAERLIREAAAASEKISREAERLIREAAA
ASEKISRE]
>strepLOCKRa_309 (SEQ ID NO: 28)
(MGSSHHHHHHSSGLVPRGSHM)SKEAVIKLQALNIKLAEKLLEAVIKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKNWSHPQFEKRIVEDAERLIREAAAASEKISREAERLIREAAA
ASEKISRE]
>strepLOCKRa_312 (SEQ ID NO: 29)
(MGSSHHHHHHSSGLVPRGSHM)SKEAVIKLQALNIKLAEKLLEAVIKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKAIANWSHPQFEKVEDAERLIREAAAASEKISREAERLIREAAA
ASEKISRE]
>strepLOCKRa_313 (SEQ ID NO: 30)
(MGSSHHHHHHSSGLVPRGSHM)SKEAVIKLQALNIKLAEKLLEAVIKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKAIARNWSHPQFEKEDAERLIREAAAASEKISREAERLIREAAA
ASEKISRE]
>strepLOCKRa_317 (SEQ ID NO: 31)
(MGSSHHHHHHSSGLVPRGSHM)SKEAVIKLQALNIKLAEKLLEAVIKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKAIARVKRENWSHPQFEKRLIREAAAASEKISREAERLIREAAA
ASEKISRE]
>strepLOCKRa_320 (SEQ ID NO: 32)
(MGSSHHHHHHSSGLVPRGSHM)SKEAVIKLQALNIKLAEKLLEAVIKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKAIARVKRESKRNWSHPQFEKREAAAASEKISREAERLIREAAA
ASEKISRE]
>strepLOCKRa_323 (SEQ ID NO: 33)
(MGSSHHHHHHSSGLVPRGSHM)SKEAVIKLQALNIKLAEKLLEAVIKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKAIARVKRESKRIVENWSHPQFEKAAASEKISREAERLIREAAA
ASEKISRE]
>strepLOCKRa_329 (SEQ ID NO: 34)
(MGSSHHHHHHSSGLVPRGSHM)SKEAVIKLQALNIKLAEKLLEAVIKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKAIARVKRESKRIVEDAERLINWSHPQFEKISREAERLIREAAA
ASEKISRE]
>SB13_LOCKR (SEQ ID NO: 35)
(MGSSHHHHHHSSGLVPRGSHM)GSKEAVIKLQALNIKLAEKLLEALARLQELNIALVYLAVELTDPKRIADEIK
KVKDKSKEIVERAEEEIARAAAESKKILDEGSGSGSDAVAELQALNLKLAELLLEAIAKLQELNIKLVELLTKLT
DPATIREAIRKVKEDSERIVAEAERLIAAAKAESERIIREGSGSGDPDVARLQELNIELARELLRAAAELQELNI
KLVELASEGSGSGSG[YELRRALEELEKALRELKKSLDEL [symbol] RSLEEL [symbol] KNPSEDALVENNRLNVENNKIIVEVLR
IIAEVLEINAKS]
>ZCX12_LOCKR (SEQ ID NO: 36)
(MGSSHHHHHHSSGLVPRGSHM)GSKEAVIKLQALNIKLAEKLLEALARLQELNIALVYLAVELTDPKRIADEIK
KVKDKSKEIVERAEEEIARAAAESKKILDEGSGSGSDAVAELQALNLKLAELLLEAIAKLQELNIKLVELLTKLT
DPATIREAIRKVKEDSERIVAEAERLIAAAKAESERIIREGSGSGDPDVARLQELNIELARELLRAAAELQELNI
KLVELASEGSGSGSG[KKLVEEVERALRELLKTSEDLVRKV [symbol] KALRELLELIRRGGTKDKIEERIRRVLEEIKRE
LERQKRKIEDVLRQIKEELYRS]
>SB13_LOCKR_extend18 (SEQ ID NO: 37)
(MGSSHHHHHHSSGLVPRGSHM)SKEAVIKLQALNIKLAEKLLEAVIKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASEGSGSGSG[YELRRALEELEKALRELKKSLDELERSLEELEKNPSEDALVENNR
LNVENNKIIVEVLRIIAEVLEINAKS]
>ZCX12_LOCKR_extend18 (SEQ ID NO: 38)
(MGSSHHHHHHSSGLVPRGSHM)SKEAVIKLQALNIKLAEKLLEAVIKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASEGSGSGSG[KKLVEEVERALRELLKTSEDLVRKVERALRELLELIRRGGTKDKI
EEKIRRVLEEIKRELERQKRKIEDVLRQIKEELYRS
>fretLOCKRa (SEQ ID NO: 39)
(GHHHHHHHHHHGVSKGEELFTGVVPILVELDGDVNGHKFSVSGEGEGDATYGKLTLKFICTTGKLPVPWPTLVT
TLSWGVQCFARYPDHMKQHDFFKSAMPEGYVQERTIFFKDDGNYKTRAEVKFEGDTLVNRIELKGIDFKEDGNIL
GHKLEYNYFSDNVYITADKQKNGIKANFKIRHNIEDGGVQLADHYQQNTPIGDGPVLLPDNHYLSTQSKLSKDPN
EKRDHMVLLEFVTAAGITLGMDELYKGSGCSLQGM)SKEAVTKLQALNIKLAEKLLEAVTKLQALNIKLAEKLLE
ALARLQELNIALVYLAVELTDPKRIADEIKKVKDKSKEIVERAEEEIARAAAESKKILDEAEEEIARAAAESKKI
LDEGSGSGSDAVAELQALNLKLAELLLEAVAELQALNLKLAELLLEAIAKLQELNIKLVELLTKLTDPATIREAI
RKVKEDSERIVAEAERLIAAAKAESERIIREAERLIAAAKAESERIIREGSGSGDPDVARLQELNIELARELLRD
VARLQELNIELARELLRAAAELQELNIKLVELASELTDPD[EARKAIARVKRESKRIVEDAERLIREAAAASEKI
SREAERLIREAAAASEKISRE](VSKGEELFTGVVPILVELDGDVNGHKFSVSGEGEGDATYGKLTLKLICTTGK
LPVPWPTLVTTLGYGVQCFARYPDHMKQHDFFKSAMPEGYVQERTIFFKDDGNYKTRAEVKFEGDTLVNRIELKG
IDFKEDGNILGHKLEYNYNSHNVYITADKQKNGIKANFKIRHNIEDGGVQLADHYQQNTPIGDGPVLLPDNHYLS
YQSKLSKDPNEKRDHMVLLEFVTAAGITLGMDELYK)
>fretLOCKRb (SEQ ID NO: 40)
(MGHHHHHHHHHHGVSKGEELFTGVVPILVELDGDVNGHKFSVSGEGEGDATYGKLTLKFICTTGKLPVPWPTLV
TTLSWGVQCFARYPDHMKQHDFFKSAMPEGYVQERTIFFKDDGNYKTRAEVKFEGDTLVNRIELKGIDFKEDGNI
LGHKLEYNYFSDNVYITADKQKNGIKANFKIRHNIEDGGVQLADHYQQNTPIGDGPVLLPDNHYLSTQSKLSKDP
NEKRDHMVLLEFVTAAGITLGMDELYKGSGCSLQGM)SHAAVIKLSDLNIRLLDKLLQAVIKLTELNAELNRKLI
EALQRLFDLNVALVHLAAELTDPKRIADEIKKVKDKSKEIVERAEEEIARAAAESKKILDEAEEEIARAAAESKK
ILDEGSGSGSDAVAELQALNLKLAELLLEAVAELQALNLKLAELLLEAIAKLQELNIKLVELLTKLTDPATIREA
IRKVKEDSERIVAEAERLIAAAKAESERIIREAERLIAAAKAESERIIREGSGSNDPQVAQNQETFIELARDALR
LVAENQEAFIEVARLTLRAAALAQEVAIKAVEAASEGGSGSG[NKEEIEKLAKEAREKLKKAEKEHKEIHDKLRK
KNKKAREDLKKKADELRETNKRVN](VSKGEELFTGVVPILVELDGDVNGHKFSVSGEGEGDATYGKLTLKLICT
TGKLPVPWPTLVTTLGYGVQCFARYPDHMKQHDFFKSAMPEGYVQERTIFFKDDGNYKTRAEVKFEGDTLVNRIE
LKGIDEKEDGNILGHKLEYNYNSHNVYITADKQKNGIKANFKIRHNIEDGGVQLADHYQQNTPIGDGPVLLPDNH
YLSYQSKLSKDPNEKRDHMVLLEFVTAAGITLGMDELYK)
>fretLOCKRc (SEQ ID NO: 41)
(GHHHHHHHHHHGVSKGEELFTGVVPILVELDGDVNGHKFSVSGEGEGDATYGKLTLKFICTTGKLPVPWPTLVT
TLSWGVQCFARYPDHMKQHDFFKSAMPEGYVQERTIFFKDDGNYKTRAEVKFEGDTLVNRIELKGIDEKEDGNIL
GHKLEYNYFSDNVYITADKQKNGIKANFKIRHNIEDGGVQLADHYQQNTPIGDGPVLLPDNHYLSTQSKLSKDPN
EKRDHMVLLEFVTAAGITLGMDELYKGSGCSLQGM)SLEAVLKLAELNLKLSDKLAEAVQKLAALLNKLLEKLSE
ALQRLFELNVALVTLAIELTDPKRIADEIKKVKDKSKEIVERAEEEIARAAAESKKILDEAEEEIARAAAESKKI
LDEGSGSGSDAVAELQALNLKLAELLLEAVAELQALNLKLAELLLEAIAKLQELNIKLVELLTKLTDPATIREAI
RKVKEDSERIVAEAERLIAAAKAESERIIREAERLIAAAKAESERIIREGSGSNDPLVARLQELLIEHARELLRL
VATSQEIFIELARAFLANAAQLQEAAIKAVEAASENGSGSGS[SEKVRRELKESLKENHKQNQKLLKDHKRAQEK
LNRELEELKKKHKKTLDDIRRES](VSKGEELFTGVVPILVELDGDVNGHKFSVSGEGEGDATYGKLTLKLICTT
GKLPVPWPTLVTTLGYGVQCFARYPDHMKQHDFFKSAMPEGYVQERTIFFKDDGNYKTRAEVKFEGDTLVNRIEL
KGIDFKEDGNILGHKLEYNYNSHNVYITADKQKNGIKANFKIRHNIEDGGVQLADHYQQNTPIGDGPVLLPDNHY
LSYQSKLSKDPNEKRDHMVLLEFVTAAGITLGMDELYK)
>fretLOCKRd (SEQ ID NO: 42)
(GHHHHHHHHHHGVSKGEELFTGVVPILVELDGDVNGHKFSVSGEGEGDATYGKLTLKFICTTGKLPVPWPTLVT
TLSWGVQCFARYPDHMKQHDFFKSAMPEGYVQERTIFFKDDGNYKTRAEVKFEGDTLVNRIELKGIDFKEDGNIL
GHKLEYNYFSDNVYITADKQKNGIKANFKIRHNIEDGGVQLADHYQQNTPIGDGPVLLPDNHYLSTQSKLSKDPN
EKRDHMVLLEFVTAAGITLGMDELYKGSGCSLQGM)SLEAVLKLFELNHKLSEKLLEAVLKLHALNQKLSQKLLE
ALARLLELNVALVELAIELTDPKRIADEIKKVKDKSKEIVERAEEEIARAAAESKKILDEAEEEIARAAAESKKI
LDEGSGSGSDAVAELQALNLKLAELLLEAVAELQALNLKLAELLLEAIAKLQELNIKLVELLTKLTDPATIREAI
RKVKEDSERIVAEAERLIAAAKAESERIIREAERLIAAAKAESERIIREGSGSGDPEVARLQEAFIEQAREILRN
VAAAQEALIEQARRLLALAALAQEAAIKAVELASEHGSGSG[DTVKRILEELRRRFEKLAKDLDDIARKLLEDHK
KHNKELKDKQRKIKKEADDAARS](VSKGEELFTGVVPILVELDGDVNGHKFSVSGEGEGDATYGKLTLKLICTT
GKLPVPWPTLVTTLGYGVQCFARYPDHMKQHDFFKSAMPEGYVQERTIFFKDDGNYKTRAEVKFEGDTLVNRIEL
KGIDFKEDGNILGHKLEYNYNSHNVYITADKQKNGIKANFKIRHNIEDGGVQLADHYQQNTPIGDGPVLLPDNHY
LSYQSKLSKDPNEKRDHMVLLEFVTAAGITLGMDELYK)
>tevLOCKR (SEQ ID NO: 43)
(MGSSHHHHHHSSGLVPRGSHM)SKEAVTKLQALNIKLAEKLLEAVTKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKAIARVKRESKRIVEDAEENLYFQGAASEKISREAERLIREAAA
ASEKISRE]
>spyLOCKR (SEQ ID NO: 44)
(MGSSHHHHHHSSGLVPRGSHM)SKEAVIKLQALNIKLAEKLLEAVIKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARAHIVMVDAYKKRIVEDAERLIREAAAASEKISREAERLIREAAA
ASEKISRE]
>1_nesLOCKR (SEQ ID NO: 45)
SKEAVTKLQALNIKLAEKLLEAVTKLQALNIKLAEKLLEALARLQELNIALVYLAVELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPDVARLQELNIELARELLRDVARLQELNIELARELLRAAAELQELNIKLVELASEL
TDPD[EARKAIARVKRESKRIVEDLALKLAGLDINSEKISREAERLIREAAAASEKISRE]
>2_nesLOCKR (SEQ ID NO: 46)
SKEAVTKLQALNIKLAEKLLEAVTKLQALNIKLAEKLLEALARLQELNIALVYLAVELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPDVARLQELNIELARELLRDVARLQELNIELARELLRAAAELQELNIKLVELASEL
TDPD[EARKAIARVKRESKRIVEDAERLIRELAEKLAGLDINAERLIREAAAASEKISRE]
>3_nesLOCKR (SEQ ID NO: 47)
SKEAVTKLQALNIKLAEKLLEAVTKLQALNIKLAEKLLEALARLQELNIALVYLAVELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPDVARLQELNIELARELLRDVARLQELNIELARELLRAAAELQELNIKLVELASEL
TDPD[EARKAIARVKRESKELAEKLRAGLDLNAAASEKISREAERLIREAAAASEKISRE]
>nlsLOCKR (SEQ ID NO: 48)
SKEAVTKLQALNIKLAEKLLEAVTKLQALNIKLAEKLLEALARLQELNIALVYLAVELTDPKRIADEIKKVKDKS
KEIVERAEEEIARAAAESKKILDEAEEEIARAAAESKKILDEGSGSGSDAVAELQALNLKLAELLLEAVAELQAL
NLKLAELLLEAIAKLQELNIKLVELLTKLTDPATIREAIRKVKEDSERIVAEAERLIAAAKAESERIIREAERLI
AAAKAESERIIREGSGSGDPDVARLQELNIELARELLRDVARLQELNIELARELLRAAAELQELNIKLVELASEL
TDPD[EARKAIARVKRESKAAAKRARTSIREAAAASEKISREAERLIREAAAASEKISRE]
>ezh2LOCKR (SEQ ID NO: 49)
(MGSSHHHHHHSSGLVPRGSHM)SKEAVTKLQALNIKLAEKLLEAVTKLQALNIKLAEKLLEALARLQELNIALV
YLAVELTDPKRIADEIKKVKDKSKEIVERAEEEIARAAAESKKILDEAEEEIARAAAESKKILDEGSGSGSDAVA
ELQALNLKLAELLLEAVAELQALNLKLAELLLEAIAKLQELNIKLVELLTKLTDPATIREAIRKVKEDSERIVAE
AERLIAAAKAESERIIREAERLIAAAKAESERIIREGSGSGDPDVARLQELNIELARELLRDVARLQELNIELAR
ELLRAAAELQELNIKLVELASELTDPD[EARKAIARVKTMFSSNRQKILERTETLNQEWKQRRIQAERLIREAAA
ASEKISRE]
>1fix_VMAc_C_BIMlatcht9 (SEQ ID NO: 51)
(MGSHHHHHHGSGSENLYFQG)SKEAAKKLQDLNIELARKLLEASTKLQRLNIRLAEALLEAIARLQELNLELVY
LAVELTDPKRIRDEIKEVKDKSKEIIRRAEKEIDDAAKESKKILEEARKAIRDAAEESRKILEEGSGSGSDALDE
LQKLNLELAKLLLKAIAETQDLNLRAAKAFLEAAAKLQELNIRAVELLVKLTDPATIRRALEHAKRRSKEIIDEA
ERAIRAAKRESERIIEEARRLIEKAKEESERIIREGSGSGDPDIKKLQDLNIELARELLRAHAQLQRLNLELLRE
LLRALAQLQELNLDLLRLASELTGGSGGSGGS(VLLNVLSKCAGSKKFRPAPAAAFARECRGFYFELQELKEDDY
YGITLSDDSDHQFLLANQVVVHNC)GGSGGS[DEIWIAQELRRIGDEFNAYYADAERLIREAAAASEKISREAER
LIREAA]
>sfGFP_VMAn_1fix_BIM_t0_latch (SEQ ID NO: 52)
(MGSHHHHHHGSGSENLYFQG)HMSKGEELFTGVVPILVELDGDVNGHKFSVRGEGEGDATNGKLILKFICTTGK
LPVPWPTLVTTLTYGVQCFARYPDHMKQHDFFKSAMPEGYVQERTISFKDDGTYKTRAEVKFEGDTLVNRIELKG
IDFKEDGNILGHKLEYNFNSHNVYITADKQKNGIKANFKIRHNVEDGSVQLADHYQQNTPIGDGPVLLPDNHYLS
TQSVLSKDPNEKRDHMVLLEFVTAAGITHGMDELYKSGSGSGCFAKGTNVLMADGSIECIENIEVGNKVMGKDGR
PREVIKLPRGRETMYSVVQKSQHRAHKSDSSREVPELLKFTCNATHELVVRTPRSVRRLSRTIKGVEYFEVITFE
MGQKKAPDGRIVELVKEVSKSYPISEGPERANELVESYRKASNKAYFEWTIEARDLSLLGSHVRKATYQTYAPIL
YGGSGGSGGGGSGGSGSKEAAKKLQDLNIELARKLLEASTKLQRLNIRLAEALLEAIARLQELNLELVYLAVELT
DPKRIRDEIKEVKDKSKEIIRRAEKEIDDAAKESKKILEEARKAIRDAAEESRKILEEGSGSGSDALDELQKLNL
ELAKLLLKAIAETQDLNLRAAKAFLEAAAKLQELNIRAVELLVKLTDPATIRRALEHAKRRSKEIIDEAERAIRA
AKRESERIIEEARRLIEKAKEESERIIREGSGSGDPDIKKLQDLNIELARELLRAHAQLQRLNLELLRELLRALA
QLQELNLDLLRLASELT[DEIWIAQELRRIGDEFNAYYADAERLSREAAAASEKISREAERSIREAAAASEKISR
E]
Asymmetrized functional Cages encoding Bim and GFP11 (i.e.: bioactive
peptides)
(6His-MBP-TEV, 6His-TEV, and flexible linker sequences are underlined text)
(Co-localization domain is bolded text)
(Functional peptide is italicized underlined text)
(Positions that can be mutated to any amino acid to tune responsiveness are
underlined bolded text)
(C-terminal sequences that can be removed to tune responsiveness are
italicized text)
(all sequences in parentheses are optional)
>1fix-long-BIM-t0 (SEQ ID NO: 54)
(MGSHHHHHHGSGSENLYFQG)SKEAAKKLQDLNIELARKLLEASTKLQRLNIRLAEALLEAIARLQELNLELVY
LAVELTDPKRIRDEIKEVKDKSKEIIRRAEKEIDDAAKESKKILEEARKAIRDAAEESRKILEEGSGSGSDALDE
LQKLNLELAKLLLKAIAETQDLNLRAAKAFLEAAAKLQELNIRAVELLVKLTDPATIRRALEHAKRRSKEIIDEA
ERAIRAAKRESERIIEEARRLIEKAKEESERIIREGSGSGDPDIKKLQDLNIELARELLRAHAQLQRLNLELLRE
LLRALAQLQELNLDLLRLASEL(TD[EIWIAQELRRIGDEFNAYYA)DAERLIREAAAASEKISREAERLIREAA
AASEKISRE]
>1fix-long-GFP-t0 (SEQ ID NO: 55)
(MGSHHHHHHGSGSENLYFQG)SKEAAKKLQDLNIELARKLLEASTKLQRLNIRLAEALLEAIARLQELNLELVY
LAVELTDPKRIRDEIKEVKDKSKEIIRRAEKEIDDAAKESKKILEEARKAIRDAAEESRKILEEGSGSGSDALDE
LQKLNLELAKLLLKAIAETQDLNLRAAKAFLEAAAKLQELNIRAVELLVKLTDPATIRRALEHAKRRSKEIIDEA
ERAIRAAKRESERIIEEARRLIEKAKEESERIIREGSGSGDPDIKKLQDLNIELARELLRAHAQLQRLNLELLRE
LLRALAQLQELNLDLLRLASEL[(RDHMVLHEYVNAAGITFNAYYA)DAERLIREAAAASEKISREAERLIREAA
AASEKISRE]
>1fix-short-BIM-t0 (SEQ ID NO: 56)
(MGSHHHHHHGSGSENLYFQGSGG)SELARKLLEASTKLQRLNIRLAEALLEAIARLQELNLELVYLAVELTDPK
RIRDEIKEVKDKSKEIIRRAEKEIDDAAKESEKILEEAREAISGSGSELAKLLLKAIAETQDLNLRAAKAFLEAA
AKLQELNIRAVELLVKLTDPATIREALEHAKRRSKEIIDEAERAIRAAKRESERIIEEARRLIEKGSGSGSELAR
ELLRAHAQLQRLNLELLRELLRALAQLQELNLDLLRLASEL(TD[EIWIAQELRRIGDEFNAYYA)DAERLIREA
AAASEKISREAERLIR]
>1fix-short-GFP-t0 (SEQ ID NO: 57)
(MGSHHHHHHGSGSENLYFQGSGG)SELARKLLEASTKLQRLNIRLAEALLEAIARLQELNLELVYLAVELTDPK
RIRDEIKEVKDKSKEIIRRAEKEIDDAAKESEKILEEAREAISGSGSELAKLLLKAIAETQDLNLRAAKAFLEAA
AKLQELNIRAVELLVKLTDPATIREALEHAKRRSKEIIDEAERAIRAAKRESERIIEEARRLIEKGSGSGSELAR
ELLRAHAQLQRLNLELLRELLRALAQLQELNLDLLRLASEL[(RDHMVLHEYVNAAGITFNAYYA)DAERLIREA
AAASEKISREAERLIR]
>Spycatcher-1fix-long-GFP-t0 (SEQ ID NO: 58)
(MGSHHHHHHGSGSENLYFQGS)AMVDTLSGLSSEQGQSGDMTIEEDSATHIKFSKRDEDGKELAGATMELRDSS
GKTISTWISDGQVKDFYLYPGKYTFVETAAPDGYEVATAITFTVNEQGQVTVNGKATKGSGGSKEAAKKLQDLNI
ELARKLLEASTKLQRLNIRLAEALLEAIARLQELNLELVYLAVELTDPKRIRDEIKEVKDKSKEIIRRAEKEIDD
AAKESKKILEEARKAIRDAAEESRKILEEGSGSGSDALDELQKLNLELAKLLLKAIAETQDLNLRAAKAFLEAAA
KLQELNIRAVELLVKLTDPATIRRALEHAKRRSKEIIDEAERAIRAAKRESERIIEEARRLIEKAKEESERIIRE
GSGSGDPDIKKLQDLNIELARELLRAHAQLQRLNLELLRELLRALAQLQELNLDLLRLASEL[(RDHMVLHEYVN
AAGITFNAYYA)DAERLIREAAAASEKISREAERLIREAAAASEKISRE]
>Spycatcher-1fix-short-GFP-t0 (SEQ ID NO: 59)
(MGSHHHHHHGSGSENLYFQGS)AMVDTLSGLSSEQGQSGDMTIEEDSATHIKESKRDEDGKELAGATMELRDSS
GKTISTWISDGQVKDFYLYPGKYTFVETAAPDGYEVATAITFTVNEQGQVTVNGKATKGSGGSELARKLLEASTK
LQRLNIRLAEALLEAIARLQELNLELVYLAVELTDPKRIRDEIKEVKDKSKEIIRRAEKEIDDAAKESEKILEEA
REAISGSGSELAKLLLKAIAETQDLNLRAAKAFLEAAAKLQELNIRAVELLVKLTDPATIREALEHAKRRSKEII
DEAERAIRAAKRESERIIEEARRLIEKGSGSGSELARELLRAHAQLQRLNLELLRELLRALAQLQELNLDLLRLA
SEL[(RDHMVLHEYVNAAGITFNAYYA)DAERLIREAAAASEKISREAERLIR]
>1fix-latch_Mad1SID_t0_1 (SEQ ID NO: 61)
(MGSHHHHHHGSGSENLYFQG)SKEAAKKLQDLNIELARKLLEASTKLQRLNIRLAEALLEAIARLQELNLELVY
LAVELTDPKRIRDEIKEVKDKSKEIIRRAEKEIDDAAKESKKILEEARKAIRDAAEESRKILEEGSGSGSDALDE
LQKLNLELAKLLLKAIAETQDLNLRAAKAFLEAAAKLQELNIRAVELLVKLTDPATIRRALEHAKRRSKEIIDEA
ERAIRAAKRESERIIEEARRLIEKAKEESERIIREGSGSGDPDIKKLQDLNIELARELLRAHAQLQRLNLELLRE
LLRALAQLQELNLDLLRLASELT[(NIQMLLEAADYLE)RESKRIVEDAERLIREAAAASEKISREAERSIREAA
AASEKISRE]
>1fix-1atch_Mad1SID_T0_2 (SEQ ID NO: 65)
(MGSHHHHHHGSGSENLYFQG)SKEAAKKLQDLNIELARKLLEASTKLQRLNIRLAEALLEAIARLQELNLELVY
LAVELTDPKRIRDEIKEVKDKSKEIIRRAEKEIDDAAKESKKILEEARKAIRDAAEESRKILEEGSGSGSDALDE
LQKLNLELAKLLLKAIAETQDLNLRAAKAFLEAAAKLQELNIRAVELLVKLTDPATIRRALEHAKRRSKEIIDEA
ERAIRAAKRESERIIEEARRLIEKAKEESERIIREGSGSGDPDIKKLQDLNIELARELLRAHAQLQRLNLELLRE
LLRALAQLQELNLDLLRLASELTDP[DEARK(NIQMLLEAADYLE)EDAERLIREAAAASEKISREAERLIREAA
SEKISRE]
>1fix-short-Bim-t0-relooped (SEQ ID NO: 67)
[MDEARKAIARVKRESKRI(EIWIAQELRRIGDEFNAYYA)EAEKLAT]DELWHRLLEASTKLQRLNIRLAEALL
EAIARLQELNLELVYLAVELTDPKRIRDEIKEVKDKSKEIIRRAEKEIDDAAKESEKILEEAREAISGSGSELAK
LLLKAIAETQDLNLRAAKAFLEAAAKLQELNIRAVELLVKLTDPATIREALEHAKRRSKEIIDEAERAIRAAKRE
SERIIEEARRLIEKGSGSGSELARELLRAHAQLQRLNLELLRELLRALAQLQELNLDLLRLASE
>1fix-short-spytag-t0_2 (SEQ ID NO: 68)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDPD[EAR(AHIVMVDAYK)KRIVEDAERLIREAAA
ASEKISREAERLIR]
>1fix-short-spytag-t0_8 (SEQ ID NO: 69)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDPD[EARKAIARVKRESK(AHIVMVDAYK)REAAA
ASEKISREAERLIR]
>1fix-short-TEV-t0_1 (SEQ ID NO: 70)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEAR(ENLYFQGS)ESKRIVEDAERLIREAAA
ASEKISREAERLIR]
>1fix-short-TEV-t0_6 (SEQ ID NO: 71)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEARKAIARVKRESKRIV(ENLYFQGS)EAAA
ASEKISREAERLIR]
>1fix-short-nanoBit-t0_1 (SEQ ID NO: 72)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEAR(VSGWRLFKKIS)RIVEDAERLIREAAA
ASEKISREAERLIR]
>1fix-short-nanoBit-t0_3 (SEQ ID NO: 73)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEARKAIARVKRESK(VSGWRLFKKIS)EAAA
ASEKISREAERLIR]
>1fix-short-RHIM-t0_8 (SEQ ID NO: 74)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEARKAI(IQIG)RESKRIVEDAERLIREAAA
ASEKIS(VQLG)RLIR]
>1fix-short-RHIM-t0_19 (SEQ ID NO: 75)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEARKAIARVKRESKRIV(IQIG)RLI
(VQLG)AASEKISREAERLIR]
>1fix-short-RHIM-t0_22 (SEQ ID NO: 76)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEARKAIARVKRESKRIV(IQIG)RLIREAAA
ASEKIS(VQLG)RLIR]
>1fix-short-gcn4-t0_4 (SEQ ID NO: 77)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DESVKE(LEDKVEELLSKNYHLENEVARLKKL
VGER)SREAERLIR]
>1fix-short-ccDi-t0_6 (SEQ ID NO: 78)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEARKAIA(GEIAALKQEIAALKKENAALKWE
IAALKQG)AERLIR]
>1fix-short-cc-a-t0_6 (SEQ ID NO: 79)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEARKAIARVKR(GLEQEIAALEKENAALEWE
IAALEQGG)ERLIR]
>1fix-short-cc-b-t0_6 (SEQ ID NO: 80)
(MGSSHHHHHHSSGLVPRGSHM)SELARKLLEASTKLQRLNIRLAEALLEAIARLQELNLELVYLAVELTDPKRI
RDEIKEVKDKSKEIIRRAEKEIDDAAKESEKILEEAREAISGSGSELAKLLLKAIAETQDLNLRAAKAFLEAAAK
LQELNIRAVELLVKLTDPATIREALEHAKRRSKEIIDEAERAIRAAKRESERIIEEARRLIEKGSGSGSELAREL
LRAHAQLQRLNLELLRELLRALAQLQELNLDLLRLASELTDP[DEARKAIARVKR(GLKQKIAALKYKNAALKKK
IAALKQGG)ERLIR
STREPII-LOCKR functional Cages:
>STREPII-2plus1_LOCK_1 (SEQ ID NO: 81)
SRVEEIIEDLRRLLEEIRKENADSIRASKELLDRVKEINDTIIAELERLLKDIEKEVREKGSESEEVKKALRRVL
EELEKLLRRVAEINEEVLRRNSKLVEEDARRNAEVLKELKRLVEELMREIGDED[KVRKVAEVAEKVLRDIDKLD
R(WSHPQFEK)TNGEISKLDEDIRRVAERVKKAIEDLAK]
>STREPII-2plus1_LOCK_2 (SEQ ID NO: 82)
[SEVDEIIADNERALDEVRREVEEIDKENAERLGE(WSHPQFEK)GDRLAKALEEIRK]GVRSRLVDELERAIRE
VEEVIRRVLERVRRLIEEVSKIITDVLREVERLHEEVTKELRKVEDGNSREALDALRRLIEKVVEDSARLIKKVD
EALKAVNKEIEDLSREVADLVRAVAEELDARVK
>STREPII-2plus1_LOCK_3 (SEQ ID NO: 83)
SSDEVLKEIEEIIRRLEAEVRRVNAEVNASTEDLAREVEEVLRATNELIEELERRVIGTEELKRVIDELRDRDRK
VRRRVERVIEESAKRDDESRKRLTRAVEKLRADLKKLADDGVPE[EALSKAIKDVRDIVKKVKDELKE(WSHPQF
EK)VDRLSEELKEWLKDVERVLKELTDKDR]
>STREPII-2plus1_LOCK_4C (SEQ ID NO: 84)
SDAEELLKRVADLLKASLESLEKILRDSKELMDRWRKKLEDLLRESEELVDRAEKILRRGGSDKEVLDKIAEEVR
RTNDDSRRLDEELHRLSRDTLRKLEENLRRTEKEVREMDKRAAERG[VDERVREELKKLLTRVE(WSHPQFEK)G
DKKILKEAHKESKEVNDRDRELLERLEESVR]
>STREPII-2plus1_LOCK_4N (SEQ ID NO: 85)
[SDAEELLKRVADLLKASLESLEKILRDSKELMDR(WSHPQFEK)LGESEELVDRAEKILRR]GGSDKEVLDKIA
EEVRRTNDDSRRLDEELHRLSRDTLRKLEENLRRTEKEVREMDKRAAERGVDERVREELKKLLTRVEEEHRKVLE
TDKKILKEAHKESKEVNDRDRELLERLEESVR
>STREPII-3plus1_LOCK_1 (SEQ ID NO: 86)
SEAEDLLERVKRVLDELIEIVDRNHELNARVVETSARLVERLLEEVERALETLEREIPGRELLDKAIKDLRDVLR
RVAEKVKRSIEELKEVLEESRRVLEEVVRALAEVIDRVRRLVEKGVDLRDLIRELKRVLEEAVSLIERLVRLNIR
AAEKDNESLRELVRAIKEALKRAVDMVRADGL[DSRLVKKLDEIVKEVAKKLEDVVRANEEL(WSHPQFEK)GSS
VARLREAVERVARDLEETAR]
>STREPII-3plus1_LOCK_2 (SEQ ID NO: 87)
[SDEERLEKVVKDVIEKVRRILEK(WSHPQFEK)GSELRRILEEWEKIIREVLDKVRR]GSGSADALVEVLEEIL
RLAEELSKRVEEVLREILKLAKALSDELVKVLAEIVEAAKRISRDDELRKAVEDVARELEDLAAKDRKILDDVRE
ALERIAKEDKDILREAEETLRRLADEMRRSGVDERLLKRVVDILARLLELNATTIERLLRILEELLKLNKELAER
VIRVLEKLLEEIKR
>STREPII-3plus1_LOCK_3 (SEQ ID NO: 88)
SVLETVKKALEDSSEKIERIVEEDERVAKESSDRIRRLVEEDKRVADEILDLIEKGGDIDTLAKLVEEWSRTSKK
LLDDVLKLHKDWSDDSRRLLEEILRVHEELIRAVKEILDRGGKPEEVVRELEKVLKESLDTLEEIIRRLDEANAR
TVKRVADVIRELEDANAKVLEEIERKGD[DKDAVIKVIEELIRANAAV(WSHPQFEK)GDLVRVNKTVWKELLRV
NEKLARDLERVVK]
>STREPII-3plus1_LOCK_4 (SEQ ID NO: 89)
[SLVDELRKSLERNVRVSEEVARRLKEALGR(WSHPQFEK)GGDLIRLNEDVVRVVEKV]GVDESAIERVRRIIE
ELNRALDAVLKKNEDLVRRLTELLDKLLEENRRLVEELDEDLKRRGGTEEVIDTILELIERSIERLKRLLDELLR
IVREALKDNARVADENLKALKEILDELRKDGVSDEELKRVLEKAADLHARLKDAHRKLLEDLERIIRELKKKLDE
VVEENKRSVDELKR
>STREPII-3plus1_LOCK_3-relooped (SEQ ID NO: 90)
[MKDAVIKVIEELIRANAAV(WSHPQFEK)GDLVRVNKTVWKELLRVNEKLARDLERAL]DERDVSAWETVKKAL
EDSSEKIERIVEEDERVAKESSDRIRRLVEEDKRVADEILDLIEKGGDTDTLAKLVEEWSRTSKKLLDDVLKLHK
DWSDDSRRLLEEILRVHEELIRAVKEILDRGGAPEEVVRELEKVLKESLDTLEEIIRRLDEANARTVKRVADVIR
ELEDANAKVLEEIERK
>STREPII-2plus1_LOCK_3-relooped (SEQ ID NO: 91)
[MEEAASKAIKDVRDIVKKVKDELKE(WSHPQFEK)VDRLSEELKEWLKDVERVLKELT]DREEASEEELKRVID
ELRDRDRKVRRRVERVIEESAKRDDESRKRLTRAVEKLRADLKKLSVEGASDEVLKEIEEIIRRLEAEVRRVNAE
VNASTEDLAREVEEVLRATNELIEELERR
>BimLOCKR a_short_Nterm (SEQ ID NO: 27094)
[MDEARKAIARVKRESKRI(EIWIAQELRRIGDEFNAYYA)EAEKLATDEL]WHRLLEASTKLQRLNIRLAEALL
EAIARLQELNLELVYLAVELTDPKRIRDEIKEVKDKSKEIIRRAEKEIDDAAKESEKILEEAREAISGSGSELAK
LLLKAIAETQDLNLRAAKAFLEAAAKLQELNIRAVELLVKLTDPATIREALEHAKRRSKEIIDEAERAIRAAKRE
SERIIEEARRLIEKGSGSGSELARELLRAHAQLQRLNLELLRELLRALAQLQELNLDLLRLASE
>BimLOCKR_g (SEQ ID NO: 27095)
[MSLVDEL(EIWIAQELRRIGDEFNAYYA)ALKRWVDVVRKVVEDLIRLNEDVVRVVEKV]GVDESAIERVRRII
EELNRALDAVLKKNEDLVRRLTELLDKLLEENRRLVEELDEDLKRRGGTEEVIDTILELIERSIERLKRLLDELL
RIVREALKDNARVADENLKALKEILDELRKDGVSDEELKRVLEKAADLHARLKDAHRKLLEDLERIIRELKKKLD
EVVEENKRSVDELKR
>reloop_strepLOCKRh (SEQ ID NO: 27096)
[MKDAVIKVIEELIRANAAV(WSHPQFEK)GDLVRVNKTVWKELLRVNEKLARDLERALDER]DVSAWETVKKAL
EDSSEKIERIVEEDERVAKESSDRIRRLVEEDKRVADEILDLIEKGGDTDTLAKLVEEWSRTSKKLLDDVLKLHK
DWSDDSRRLLEEILRVHEELIRAVKEILDRGGAPEEVVRELEKVLKESLDTLEEIIRRLDEANARTVKRVADVIR
ELEDANAKVLEEIERK
>reloop_strepLOCKRi (SEQ ID NO: 27097)
[MEEAASKAIKDVRDIVKKVKDELKE(WSHPQFEK)VDRLSEELKEWLKDVERVLKELTDREEA]SEEELKRVID
ELRDRDRKVRRRVERVIEESAKRDDESRKRLTRAVEKLRADLKKLSVEGASDEVLKEIEEIIRRLEAEVRRVNAE
VNASTEDLAREVEEVLRATNELIEELERR
>spyLOCKRa_2 (SEQ ID NO: 27098)
MSELARKLLEASTKLQRLNIRLAEALLEAIARLQELNLELVYLAVELTDPKRIRDEIKEVKDKSKEIIRPAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEAR(AHIVMVDAYK)KRIVEDAERLIREAAAASEKISREAERLIR]
>spyLOCKRa_8 (SEQ ID NO: 27099)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDPDEARKAIARVKRESK(AHIVMVDAYK)REAAAASEKISREAERLIR]
>tevLOCKRa_1 (SEQ ID NO: 27100)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEAR(ENLYFQGS)ESKRIVEDAERLIREAAAASEKISREAERLIR]
>tevLOCKRa_6 (SEQ ID NO: 27101)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEARKAIARVKRESKRIV(ENLYFQGS)EAAAASEKISREAERLIR]
>lucLOCKRa_1 (SEQ ID NO: 27102)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEAR(VSGWRLFKKIS)RIVEDAERLIREAAAASEKISREAERLIR]
>lucLOCKRa_3 (SEQ ID NO: 27103)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEARKAIARVKRESK(VSGWRLFKKIS)EAAAASEKISREAERLIR]
>rhimLOCKRa_8 (SEQ ID NO: 27104)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEARKAI(IQIG)RESKRIVEDAERLIREAAAASEKIS(VQLG)RLIR]
>rhimLOCKRa_19 (SEQ ID NO: 27105)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEARKAIARVKRESKRIV(IQIG)RLI(VQLG)AASEKISREAERLIR]
>rhimLOCKRa_22 (SEQ ID NO: 27106)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEARKAIARVKRESKRIV(IQIG)RLIREAAAASEKIS(VQLG)RLIR]
>gcn4LOCKRa_4 (SEQ ID NO: 27107)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DESVKE(LEDKVEELLSKNYHLENEVARLKKLVGER)SREAERLIR]
>cc-DiLOCKRa_6 (SEQ ID NO: 27108)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEARKAIA(GEIAALKQEIAALKKENAALKWEIAALKQG)AERLIR]
>cc-aLOCKRa_6 (SEQ ID NO: 27109)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEARKAIARVKR(GLEQEIAALEKENAALEWEIAALEQGG)ERLIR]
>cc-bLOCKRa_6 (SEQ ID NO: 27110)
MSELARKLLEASTKLQRLNIRLAEALLEAIARLQELNLELVYLAVELTDPKRIRDEIKEVKDKSKEIIRRAEKEI
DDAAKESEKILEEAREAISGSGSELAKLLLKAIAETQDLNLRAAKAFLEAAAKLQELNIRAVELLVKLTDPATIR
EALEHAKRRSKEIIDEAERAIRAAKRESERIIEEARRLIEKGSGSGSELARELLRAHAQLQRLNLELLRELLRAL
AQLQELNLDLLRLASELTDP[DEARKAIARVKR(GLKQEIAALRYKNAALKKLIAALKQGG)ERLIR]
>tev-spyLOCKRa_short_40 (SEQ ID NO: 27111)
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASELTDP[DEARKAI(ENLYFQGS)RIVEDAE(AHIVMVDAYK)EKISREAERLIR]
>tev-spyLOCKRa_short_57 (SEQ ID NO: 27112)
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASELTDP[DEARKAIARV(ENLYFQGS)EDAERLIREA(AHIVMVDAYK)AERLIR]
>tev-spyLOCKRa_short_63 (SEQ ID NO: 27113)
SELARKLLEASTKLQRLNIRLAEALLEAIARLQELNLELVYLAVELTDPKRIRDEIKEVKDKSKEIIRRAEKEID
DAAKESEKILEEAREAISGSGSELAKLLLKAIAETQDLNLRAAKAFLEAAAKLQELNIRAVELLVKLTDPATIRE
ALEHAKRRSKEIIDEAERAIRAAKRESERIIEEARRLIEKGSGSGSELARELLRAHAQLQRLNLELLRELLRALA
QLQELNLDLLRLASELTDP[DEARKAIARVK(ENLYFQGS)DAERLIREA(AHIVMVDAYK)AERLIR]
>tev-spyLOCKRa_29 (SEQ ID NO: 27114)
SKEAAKKLQDLNIELARKLLEASTKLQRLNIRLAEALLEAIARLQELNLELVYLAVELTDPKRIRDEIKEVKDKS
KEIIRRAEKEIDDAAKESKKILEEARKAIRDAAEESRKILEEGSGSGSDALDELQKLNLELAKLLLKAIAETQDL
NLRAAKAFLEAAAKLQELNIRAVELLVKLTDPATIRRALEHAKRRSKEIIDEAERAIRAAKRESERIIEEARRLI
EKAKEESERIIREGSGSGDP[DIKKLQDLNIELARELLRAHAQLQRLNLELLRELLRALAQLQELNLDLLRLASE
LTDPDEARKAIARVK(ENLYFQGS)DAERLIREAAAASE(AHIVMVDAYK)REAAAASEKISRE]
>tev-spyLOCKRa_32 (SEQ ID NO: 27115)
SKEAAKKLQDLNIELARKLLEASTKLQRLNIRLAEALLEAIARLQELNLELVYLAVELTDPKRIRDEIKEVKDKS
KEIIRRAEKEIDDAAKESKKILEEARKAIRDAAEESRKILEEGSGSGSDALDELQKLNLELAKLLLKAIAETQDL
NLRAAKAFLEAAAKLQELNIRAVELLVKLTDPATIRRALEHAKRRSKEIIDEAERAIRAAKRESERIIEEARRLI
EKAKEESERIIREGSGSGDP[DIKKLQDLNIELARELLRAHAQLQRLNLELLRELLRALAQLQELNLDLLRLASE
LTDPDEARKAIARVK(ENLYFQGS)DAERLIREAAAASEKISREAE(AHIVMVDAYK)EKISRE]
>Bim-fretLOCKRa_short (SEQ ID NO: 27116)
(VSKGEELFTGVVPILVELDGDVNGHKFSVSGEGEGDATYGKLILKFICTTGKLPVPWPTLVTTLSWGVQCFARY
PDHMKQHDFFKSAMPEGYVQERTIFFKDDGNYKTRAEVKFEGDTLVNRIELKGIDFKEDGNILGHKLEYNYFSDN
VYITADKQKNGIKANFKIRHNIEDGGVQLADHYQQNTPIGDGPVLLPDNHYLSTQSKLSKDPNEKRDHMVLLEFV
TAAGITLE)LARKLLEASTKLQRLNIRLAEALLEAIARLQELNLELVYLAVELTDPKRIRDEIKEVKDKSKEIIR
RAEKEIDDAAKESEKILEEAREAISGSGSELAKLLLKAIAETQDLNLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLR
ELLRALAQLQELNLDLLRLASELT[D(EIWIAQELRRIGDEFNAYYA)DAERLIREAAAASEKISREAERLIR]
(VSKGEELFTGVVPILVELDGDVNGHKFSVSGEGEGDATYGKLILKLICTTGKLPVPWPTLVTTLGYGVQCFARYP
DHMKQHDFFKSAMPEGYVQERTIFFKDDGNYKTRAEVKFEGDTLVNRIELKGIDFKEDGNILGHKLEYNYNSHNV
YITADKQKNGIKANFKIRHNIEDGGVQLADHYQQNTPIGDGPVLLPDNHYLSYQSKLSKDPNEKRDHMVLLEFVT
AAGITLGMDELYKGSGC)
>fretLOCKRa_short (SEQ ID NO: 27117)
(VSKGEELFTGVVPILVELDGDVNGHKFSVSGEGEGDATYGKLTLKFICTTGKLPVPWPTLVTTLSWGVQCFARY
PDHMKQHDFFKSAMPEGYVQERTIFFKDDGNYKTRAEVKFEGDILVNRIELKGIDFKEDGNILGHKLEYNYFSDN
VYITADKQKNGIKANFKIRHNIEDGGVQLADHYQQNTPIGDGPVLLPDNHYLSTQSKLSKDPNEKRDHMVLLEFV
TAAGITL)ELARKLLEASTKLQRLNIRLAEALLEAIARLQELNLELVYLAVELTDPKRIRDEIKEVKDKSKEIIR
RAEKEIDDAAKESEKILEEAREAISGSGSELAKLLLKAIAETQDLNLRAAKAFLEAAAKLQELNIRAVELLVKLT
DPATIREALEHAKRRSKEIIDEAERAIRAAKRESERIIEEARRLIEKGSGSGSELARELLRAHAQLQRLNLELLR
ELLRALAQLQELNLDLLRLASELTDP[DEARKAIARVKRESNAYYADAERLIREAAAASEK](VSKGEELFTGVV
PILVELDGDVNGHKFSVSGEGEGDATYGKLTLKLICTTGKLPVPWPTLVTTLGYGVQCFARYPDHMKQHDFFKSA
MPEGYVQERTIFFKDDGNYKTRAEVKFEGDTLVNRIELKGIDFKEDGNILGHKLEYNYNSHNVYITADKQKNGIK
ANFKIRHNIEDGGVQLADHYQQNTPIGDGPVLLPDNHYLSYQSKLSKDPNEKRDHMVLLEFVTAAGITLGMDELY
KGSGC)
E18_KRAB_full (SEQ ID NO: 27120)
MSKEAVTKLQALNIKLAEKLLEAVTKLQALNIKLAEKLLEALARLQELNIALVYLAVELTDPKRIADEIKKVKDK
SKEIVERAEEEIARAAAESKKILDEAEEEIARAAAESKKILDEGSGSGSDAVAELQALNLKLAELLLEAVAELQA
LNLKLAELLLEAIAKLQELNIKLVELLTKLTDPATIREAIRKVKEDSERIVAEAERLIAAAKAESERIIREAERL
IAAAKAESERIIREGSGSGDPDVARLQELNIELARELLRDVARLQELNIELARELLRAAAELQELNIKLVELASE
LTGS[(RTLVTFKDVFVDFTREEWKLLDTAQQIVYRNVMLENYKNLVSLGYG)SDEARKAIARVKRESKRIVEDA
ERLIREAAAASEKISREAERLIREAAAASEKISRE]
E18_KRAB_N13t (SEQ ID NO: 27121)
MSKEAVTKLQALNIKLAEKLLEAVTKLQALNIKLAEKLLEALARLQELNIALVYLAVELTDPKRIADEIKKVKDK
SKEIVERAEEEIARAAAESKKILDEAEEEIARAAAESKKILDEGSGSGSDAVAELQALNLKLAELLLEAVAELQA
LNLKLAELLLEAIAKLQELNIKLVELLTKLTDPATIREAIRKVKEDSERIVAEAERLIAAAKAESERIIREAERL
IAAAKAESERIIREGSGSGDPDVARLQELNIELARELLRDVARLQELNIELARELLRAAAELQELNIKLVELASE
LTGS[(RTLVTFKDVFVDFTREEWKLLDTAQQIVYRNVMLENYKNLVSLGY)GSSKRIVEDAERLIREAAAASEK
ISREAERLIREAAAASEKISRE]
E18_KRAB_C9t (SEQ ID NO: 27122)
MSKEAVTKLQALNIKLAEKLLEAVTKLQALNIKLAEKLLEALARLQELNIALVYLAVELTDPKRIADEIKKVKDK
SKEIVERAEEEIARAAAESKKILDEAEEEIARAAAESKKILDEGSGSGSDAVAELQALNLKLAELLLEAVAELQA
LNLKLAELLLEAIAKLQELNIKLVELLTKLTDPATIREAIRKVKEDSERIVAEAERLIAAAKAESERIIREAERL
IAAAKAESERIIREGSGSGDPDVARLQELNIELARELLRDVARLQELNIELARELLRAAAELQELNIKLVELASE
LTGS[(RTLVTFKDVFVDFTREEWKLLDTAQQIVYRNVMLENYKNLVSLGY)GSDEARKAIARVKRESKRIVEDA
ERLIREAAAASEKISREAERLIREAA]
E18_KRAB_Cterm1 (SEQ ID NO: 27123)
MSKEAVTKLQALNIKLAEKLLEAVTKLQALNIKLAEKLLEALARLQELNIALVYLAVELTDPKRIADEIKKVKDK
SKEIVERAEEEIARAAAESKKILDEAEEEIARAAAESKKILDEGSGSGSDAVAELQALNLKLAELLLEAVAELQA
LNLKLAELLLEAIAKLQELNIKLVELLTKLTDPATIREAIRKVKEDSERIVAEAERLIAAAKAESERIIREAERL
IAAAKAESERIIREGSGSGDPDVARLQELNIELARELLRDVARLQELNIELARELLRAAAELQELNIKLVELASE
LT[DEARKAIARVKRESKRIVEDAE(RTLVTFKDVFVDFTREEWKLLDTAQQIVYRNVMLENYKNLVSLGY)]
E18_KRAB_Cterm2 (SEQ ID NO: 27124)
MSKEAVTKLQALNIKLAEKLLEAVTKLQALNIKLAEKLLEALARLQELNIALVYLAVELTDPKRIADEIKKVKDK
SKEIVERAEEEIARAAAESKKILDEAEEEIARAAAESKKILDEGSGSGSDAVAELQALNLKLAELLLEAVAELQA
LNLKLAELLLEAIAKLQELNIKLVELLTKLTDPATIREAIRKVKEDSERIVAEAERLIAAAKAESERIIREAERL
IAAAKAESERIIREGSGSGDPDVARLQELNIELARELLRDVARLQELNIELARELLRAAAELQELNIKLVELASE
LT[DEARKAIARVKRESKRIVEDAERLI(RTLVTFKDVFVDFTREEWKLLDTAQQIVYRNVMLENYKNLVSLGY)]
E18_KRAB_Cterm3 (SEQ ID NO: 27125)
MSKEAVTKLQALNIKLAEKLLEAVTKLQALNIKLAEKLLEALARLQELNIALVYLAVELTDPKRIADEIKKVKDK
SKEIVERAEEEIARAAAESKKILDEAEEEIARAAAESKKILDEGSGSGSDAVAELQALNLKLAELLLEAVAELQA
LNLKLAELLLEAIAKLQELNIKLVELLTKLTDPATIREAIRKVKEDSERIVAEAERLIAAAKAESERIIREAERL
IAAAKAESERIIREGSGSGDPDVARLQELNIELARELLRDVARLQELNIELARELLRAAAELQELNIKLVELASE
LT[DEARKAIARVKRESKRIVEDAERLIREAAAASEKISRTLVTFKDVFVDFTREEWKLLDTAQQIVYRNVMLEN
YKNLVSLGY)]
>3plus1_Cage_Nterm_GFP11_668 (SEQ ID NO: 27,278)
DEAKELLDEIRKAVKESEDRLEKLLRDYEKELRRDHMVLHEYVNAAGITLEELRRGSLDAKELLKTLEDLLREVL
EVARRVVETLKELNRRVLEVVREDIEANERLLRRVLDTLRRGGVDERRIKDLERLIRESLKKAEEVLREAAEKSR
EIVDEIREVLKRADEALKRIIKKIRETRGADALSRLLEELLRVVDDLIRVLKELIDKSRKVIEELLELLKRINEE
NLKVLAEIIK
>3plus1_Cage_Cterm_GFP11_668 (SEQ ID NO: 27,279)
DEAKELLDEIRKAVKESEDRLEKLLRDYEKELRRLEKELRDLKRRIEEKLEELRRGSLDAKELLKTLEDLLREVL
EVARRVVETLKELNRRVLEVVREDIERNERLLRRVLDTLRRGGVDERRIKDLERLIRESLKKAEEVLREAAEKSR
EIVDEIREVLKRADEALKRIIKKIRETRGADADHMVLHEYVNAAGITIRVLKELIDKSRKVIEELLELLKRINEE
NLKVLAEIIK
>3plus1_Cage_Cterm_GFP11_668 (SEQ ID NO: 27,280)
DEAKELLDEIRKAVKESEDRLEKLLRDYEKELRRLEKELRDLKRRIEEKLEELRRGSLDAKELLKTLEDLLREVL
EVARRVVETLKELNRRVLEVVREDIERNERLLRRVLDTLRRGGVDERRIKDLERLIRESLKKAEEVLREAAEKSR
EIVDEIREVLKRADEALKRIIKKIRETRGADARDHMVLHEYVNAAGITRVLKELIDKSRKVIEELLELLKRINEE
NLKVLAEIIK
>3plus1_Cage_Cterm_GFP11_668 (SEQ ID NO: 27,281)
DEAKELLDEIRKAVKESEDRLEKLLRDYEKELRRLEKELRDLKRRIEEKLEELRRGSLDAKELLKTLEDLLREVL
EVARRVVETLKELNRRVLEVVREDIERNERLLRRVLDTLRRGGVDERRIKDLERLIRESLKKAEEVLREAAEKSR
EIVDEIREVLKRADEALKRIIKKIRETRGADALSRDHMVLHEYVNAAGITLKELIDKSRKVIEELLELLKRINEE
NLKVLAEIIK
>3plus1_Cage_Cterm_GFP11_668 (SEQ ID NO: 27,282)
DEAKELLDEIRKAVKESEDRLEKLLRDYEKELRRLEKELRDLKRRIEEKLEELRRGSLDAKELLKTLEDLLREVL
EVARRVVETLKELNRRVLEVVREDIERNERLLRRVLDTLRRGGVDERRIKDLERLIRESLKKAEEVLREAAEKSR
EIVDEIREVLKRADEALKRIIKKIRDHMVLHEYVNAAGITLRVVDDLIRVLKELIDKSRKVIEELLELLKRINEE
NLKVLAEIIK
>3plus1_Cage_Nterm_GFP11_669 (SEQ ID NO: 27,283)
SEKEKLLKESEEEVRRLRRTLEELLRKYREVLERLRDHMVLHEYVNAAGITRLKEVLDRSGLDIDTIIKEVEDLL
KTVLDRLRELLDKIARLIKEAIEVVREIIERIVRHAERVKDELRKGGADKRKLDRVDRLIKENTRHLKEILDRIE
DLVRRSEKKLRDIIREVRRLIEELRKKAEEIKKDPDERLVKILIEDVERVIKRILELITRVAEDNERVLERIIRE
LTDNLERHLKIVREIVK
>3plus1_Cage_Nterm_GFP11_670 (SEQ ID NO: 27,284)
SEKEDLARKLRKLVEELTREYEELVKKLERLIEEIERDHMVLHEYVNAAGITISEEVRKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPIGDEALRKLVELLVEVLRRLIRVNRELVKLLREVLERLLRILRESVKKLKR
LIEKVIKDAT
>3plus1_Cage_Cterm_GFP11_670 (SEQ ID NO: 27,285)
SEKEDLARKLRKLVEELTREYEELVKKLERLIEEIEKVSEESVRKLEKLLREISEEVRKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPIGDEARDHMVLHEYVNAAGITRVNRELVKLLREVLERLLRILRESVKKLKR
LIEKVIKDAT
>3plus1_Cage_Cterm_GFP11_670 (SEQ ID NO: 27,286)
SEKEDLARKLRKLVEELTREYEELVKKLERLIEEIEKVSEESVRKLEKLLREISEEVRKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPIGDERDHMVLHEYVNAAGITIRVNRELVKLLREVLERLLRILRESVKKLKR
LIEKVIKDAT
>3plus1_Cage_Cterm_GFP11_670 (SEQ ID NO: 27,287)
SEKEDLARKLRKLVEELTREYEELVKKLERLIEEIEKVSEESVRKLEKLLREISEEVRKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPIGRDHMVLHEYVNAAGITRLIRVNRELVKLLREVLERLLRILRESVKKLKR
LIEKVIKDAT
>3plus1_Cage_Nterm_GFP11_670 (SEQ ID NO: 27,288)
SEKEDLARKLRKLVEELTREYEELVKKLERLIEEIRDHMVLHEYVNAAGITEISEEVRKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPIGDEALRKLVELLVEVLRRLIRVNRELVKLLREVLERLLRILRESVKKLKR
LIEKVIKDAT
>3plus1_Cage_Cterm_GFP11_670 (SEQ ID NO: 27,289)
SEKEDLARKLRKLVEELTREYEELVKKLERLIEEIEKVSEESVRKLEKLLREISEEVRKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPIRDHMVLHEYVNAAGITRRLIRVNRELVKLLREVLERLLRILRESVKKLKR
LIEKVIKDAT
>3plus1_Cage_Nterm_GFP11_670 (SEQ ID NO: 27,290)
SEKEDLARKLRKLVEELTREYEELVKKLERLIERDHMVLHEYVNAAGITLREISEEVRKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPIGDEALRKLVELLVEVLRRLIRVNRELVKLLREVLERLLRILRESVKKLKR
LIEKVIKDAT
>3plus1_Cage_Nterm_GFP11_670 (SEQ ID NO: 27,291)
SEKEDLARKLRKLVEELTREYEELVKKLERLIEEIEKRDHMVLHEYVNAAGITSEEVRKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPIGDEALRKLVELLVEVLRRLIRVNRELVKLLREVLERLLRILRESVKKLKR
LIEKVIKDAT
>3plus1_Cage_Nterm_GFP11_670 (SEQ ID NO: 27,292)
SEKEDLARKLRKLVEELTREYEELVKKLERLIEEIEKVSEESRDHMVLHEYVNAAGITKLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPIGDEALRKLVELLVEVLRRLIRVNRELVKLLREVLERLLRILRESVKKLKR
LIEKVIKDAT
>3plus1_Cage_Nterm_GFP11_670 (SEQ ID NO: 27,293)
SEKEDLARKLRKLVEELTREYEELVKKLERLIEEIEKVSEESVRDHMVLHEYVNAAGITLGTDERVLKRLLERLR
RIIEEDHELNTELLKRLLDLLKEILDTSRELLKRLLDILRKGVRDEEVLRDLERTLREVLEENERAIEEAERVLR
KVLEDSERAVRDARRVLAEVDKSPIGDEALRKLVELLVEVLRRLIRVNRELVKLLREVLERLLRILRESVKKLKR
LIEKVIKDAT
>3plus1_Cage_Cterm_GFP11_671 (SEQ ID NO: 27,294)
SEEEDLLERVKRVLDELIEIVDRNHELNRRVVETSAALVERLLEEVERALETLEREIPGSSLLDKAIKDLRDVLR
RVKEKVKRSIEELKEVLEESRRVLEEVVRKLREVIDRVRRLVEKGVDLRDLIRELKRVLEEAVKLIERLVRLNIR
AAEKDNESLRELVRAIKEALKRAVDAVRKGGLDSRAVKKLDRDHMVLHEYVNAAGITNEELWRALVELNKESVRR
LREIVERVARDLEETAR
>3plus1_Cage_Cterm_GFP11_671 (SEQ ID NO: 27,295)
SEEEDLLERVKRVLDELIEIVDRNHELNRRVVETSAALVERLLEEVERALETLEREIPGSSLLDKAIKDLRDVLR
RVKEKVKRSIEELKEVLEESRRVLEEVVRKLREVIDRVRRLVEKGVDLRDLIRELKRVLEEAVKLIERLVRLNIR
AAEKDNESLRELVRAIKEALKRAVDAVRKGGLDSRARDHMVLHEYVNAAGITDVVRRNEELWRALVELNKESVRR
LREIVERVARDLEETAR
>3plus1_Cage_Cterm_GFP11_671 (SEQ ID NO: 27,296)
SEEEDLLERVKRVLDELIEIVDRNHELNRRVVETSAALVERLLEEVERALETLEREIPGSSLLDKAIKDLRDVLR
RVKEKVKRSIEELKEVLEESRRVLEEVVRKLREVIDRVRRLVEKGVDLRDLIRELKRVLEEAVKLIERLVRLNIR
AAEKDNESLRELVRAIKEALKRAVDAVRKGGLDSRRDHMVLHEYVNAAGITEDVVRRNEELWRALVELNKESVRR
LREIVERVARDLEETAR
>3plus1_Cage_Cterm_GFP11_671 (SEQ ID NO: 27,297)
SEEEDLLERVKRVLDELIEIVDRNHELNRRVVETSAALVERLLEEVERALETLEREIPGSSLLDKAIKDLRDVLR
RVKEKVKRSIEELKEVLEESRRVLEEVVRKLREVIDRVRRLVEKGVDLRDLIRELKRVLEEAVKLIERLVRLNIR
AAEKDNESLRELVRAIKEALKRAVDAVRKGGLRDHMVLHEYVNAAGITKKLEDVVRRNEELWRALVELNKESVRR
LREIVERVARDLEETAR
>3plus1_Cage_Cterm_GFP11_672 (SEQ ID NO: 27,298)
DETEEVIKKIAKLIDELIETLKKIVEDSAKLIKELIDLLEEIIKRSADLIRELEKVSKRGDRDRLKKLIDRMRRE
TERALEELRRVIEELKEATERALKTIEDLIREIEEAIRRELGDEDLAREILEVIRRILELLREDMELNLRALEEI
LRLLEEVIKLNKDAIDEILELLKRDHMVLHEYVNAAGITESKKLVRDVVEEAKRLLKENRDSTRKIIEDIRRLLR
KIEDSTR
>3plus1_Cage_Cterm_GFP11_672 (SEQ ID NO: 27,299)
DETEEVIKKIAKLIDELIETLKKIVEDSAKLIKELIDLLEEIIKRSADLIRELEKVSKRGDRDRLKKLIDRMRRE
TERALEELRRVIEELKEATERALKTIEDLIREIEEAIRRELGDEDLAREILEVIRRILELLREDMELNLRALEEI
LRLLEEVIKLNKDAIDEILELLKGRDHMVLHEYVNAAGITSKKLVRDVVEEAKRLLKENRDSTRKIIEDIRRLLR
KIEDSTR
>3plus1_Cage_Nterm_GFP11_67>3 (SEQ ID NO: 27,300)
SKEETLRKEAEDLLRRLEELTRRLEKKARELLERAKKLSRDLARDHMVLHEYVNAAGITEELIKVLEEVAKTSID
LNKEVIDVIRRVIEESLELNKRVLDVLRRVLDIAARKGRDKKAIRDVVREAARVIKEVIDEDEKVNRDLKRVIER
VIDDDRKALERLRKAVEEVASRGDDEAAKRLVEELLKAVIDLSRKNKRILEELLKAIETLSDENKKILTEILDAL
RRLVEKIEK
>3plus1_Cage_Nterm_GFP11_67>3 (SEQ ID NO: 27,301)
SKEETLRKEAEDLLRRLEELTRRLEKKARELLERAKKLSRRDHMVLHEYVNAAGITGVPEELIKVLEEVAKTSID
LNKEVIDVIRRVIEESLELNKRVLDVLRRVLDIAARKGRDKKAIRDVVREAARVIKEVIDEDEKVNRDLKRVIER
VIDDDRKALERLRKAVEEVASRGDDEAAKRLVEELLKAVIDLSRKNKRILEELLKAIETLSDENKKILTEILDAL
RRLVEKIEK
>3plus1_Cage_Cterm_GFP11_67>3 (SEQ ID NO: 27,302)
SKEETLRKEAEDLLRRLEELTRRLEKKARELLERAKKLSRDLAEELKRLLKELREKGVPEELIKVLEEVAKTSID
LNKEVIDVIRRVIEESLELNKRVLDVLRRVLDIAARKGRDKKAIRDVVREAARVIKEVIDEDEKVNRDLKRVIER
VIDDDRKALERLRKAVEEVASRGDDEARDHMVLHEYVNAAGITRKNKRILEELLKAIETLSDENKKILTEILDAL
RRLVEKIEK
>3plus1_Cage_Nterm_GFP11_67>3 (SEQ ID NO: 27,303)
SKEETLRKEAEDLLRRLEELTRRLEKKARELLERARDHMVLHEYVNAAGITELREKGVPEELIKVLEEVAKTSID
LNKEVIDVIRRVIEESLELNKRVLDVLRRVLDIAARKGRDKKAIRDVVREAARVIKEVIDEDEKVNRDLKRVIER
VIDDDRKALERLRKAVEEVASRGDDEAAKRLVEELLKAVIDLSRKNKRILEELLKAIETLSDENKKILTEILDAL
RRLVEKIEK
>3plus1_Cage_Nterm_GFP11_67>3 (SEQ ID NO: 27,304)
SKEETLRKEAEDLLRRLEELTRRLEKKARELLERAKKLSRDHMVLHEYVNAAGITKGVPEELIKVLEEVAKTSID
LNKEVIDVIRRVIEESLELNKRVLDVLRRVLDIAARKGRDKKAIRDVVREAARVIKEVIDEDEKVNRDLKRVIER
VIDDDRKALERLRKAVEEVASRGDDEAAKRLVEELLKAVIDLSRKNKRILEELLKAIETLSDENKKILTEILDAL
RRLVEKIEK
>3plus1_Cage_Nterm_GFP11_67>3 (SEQ ID NO: 27,305)
SKEETLRKEAEDLLRRLEELTRRLEKKARELLERAKKLRDHMVLHEYVNAAGITEKGVPEELIKVLEEVAKTSID
LNKEVIDVIRRVIEESLELNKRVLDVLRRVLDIAARKGRDKKAIRDVVREAARVIKEVIDEDEKVNRDLKRVIER
VIDDDRKALERLRKAVEEVASRGDDEAAKRLVEELLKAVIDLSRKNKRILEELLKAIETLSDENKKILTEILDAL
RRLVEKIEK
>3plus1_Cage_Nterm_GFP11_67>3 (SEQ ID NO: 27,306)
SKEETLRKEAEDLLRRLEELTRRLEKKARELLERAKKLSRDLRDHMVLHEYVNAAGITPEELIKVLEEVAKTSID
LNKEVIDVIRRVIEESLELNKRVLDVLRRVLDIAARKGRDKKAIRDVVREAARVIKEVIDEDEKVNRDLKRVIER
VIDDDRKALERLRKAVEEVASRGDDEAAKRLVEELLKAVIDLSRKNKRILEELLKAIETLSDENKKILTEILDAL
RRLVEKIEK
>3plus1_Cage_Nterm_GFP11_674 (SEQ ID NO: 27,307)
SEREEVKEKLDRLLEEVEKTVRELKREHDELLKEVEKLVRDLKRDHMVLHEYVNAAGITKEILDVIREHVRINKE
ILDRVLEVVEEHLRRNKEILDKLLDDIRKVVEDAKRILGIGDDETLRRAVRRILEELRKLVEDIRKKVKDSLETL
ERALEEAEERIRRSLEDLKRVLKEAKDKIKDKDRLDKVEELVKKLLEDTKRTVDRVRELVRKILKKSRETLEELE
RLIEKILRELEKDAR
>3plus1_Cage_Cterm_GFP11_674 (SEQ ID NO: 27,308)
SEREEVKEKLDRLLEEVEKTVRELKREHDELLKEVEKLVRDLKKEHDELLKKVKDDGVPKEILDVIREHVRINKE
ILDRVLEVVEEHLRRNKEILDKLLDDIRKVVEDAKRILGIGDDETLRRAVRRILEELRKLVEDIRKKVKDSLETL
ERALEEAEERIRRSLEDLKRVLKEAKDKIKDKDRDHMVLHEYVNAAGITKRTVDRVRELVRKILKKSRETLEELE
RLIEKILRELEKDAR
>3plus1_Cage_Nterm_GFP11_675 (SEQ ID NO: 27,309)
SERETVKRRLEELLKEVKRILDKLKEEHDRLLEDVRRVVEELRDHMVLHEYVNAAGITPEELLRVIAKVLEINKR
ILDDLLRVVKKHVDLNKEILDRILEMIKEIVERVKRVLGDGDEKTLRDKIRDIIRRLEDAAREAEERVRRSLEEL
KKAVEKIRKKIEDSLRELEEALKRVRDKEEDDKRLEDISRLVKRLLDESRRVLRELEETIRKRAEESKRVLEEVK
RLVEKLIRELRKEAE
>3plus1_Cage_Nterm_GFP11_676 (SEQ ID NO: 27,310)
SEDEIIKKIIEDLRRVLKEVEEIHKEVEERLDKRDHMVLHEYVNAAGITDRVLDEVKRIGDVETVLRLAIEAVRR
ALEIVRKVIEDLIRVVRKLLEIVDAVNKRVKKAVEDVLKLLRDILKGSKDVERVLDEARRLLEKLHRDSRRILKR
IAELLDELKELSERILERIKRLLEEVERRGSDDERLLEELIEELKDLIRRHIELLRRLIERLKELLKKHIEDLRR
LLEEIEKLLEEARK
>3plus1_Cage_Nterm_GFP11_677 (SEQ ID NO: 27,311)
SREEVLRELEEVIEDNRRLLEELIEKSKKVLDESLKLIDELLRRDHMVLHEYVNAAGITSKGDDEETARRIVIKI
RKELEDVLDEVRRIVKEILDRDRRVAEDLKRVVDENLEKLKKVLDELDKKLGDRRLVEILRDIVRLLIEINERAL
RELLKLHEEHTRIVAELLRELERVVEELRRLVELIARGEISEDDLKRVVDEVEKKLRELKRRYKEALERIKEKIK
ELKDRYERAVREVVAELRKTTK
>3plus1_Cage_Cterm_GFP11_677 (SEQ ID NO: 27,312)
SREEVLRELEEVIEDNRRLLEELIEKSKKVLDESLKLIDELLRRLEEVLERVLRLLEELSKGDDEETARRIVIKI
RKELEDVLDEVRRIVKEILDRDRRVAEDLKRVVDENLEKLKKVLDELDKKLGDRRLVEILRDIVRLLIEINERAL
RELLKLHEEHTRIVAELLRELERVVEELRRLVELIARGEISEDDRDHMVLHEYVNAAGITRRYKEALERIKEKIK
ELKDRYERAVREVVAELRKTTK
>3plus1_Cage_Cterm_GFP11_677 (SEQ ID NO: 27,313)
SREEVLRELEEVIEDNRRLLEELIEKSKKVLDESLKLIDELLRRLEEVLERVLRLLEELSKGDDEETARRIVIKI
RKELEDVLDEVRRIVKEILDRDRRVAEDLKRVVDENLEKLKKVLDELDKKLGDRRLVEILRDIVRLLIEINERAL
RELLKLHEEHTRIVAELLRELERVVEELRRLVELIARGERDHMVLHEYVNAAGITLRELKRRYKEALERIKEKIK
ELKDRYERAVREVVAELRKTTK
>3plus1_Cage_Nterm_GFP11_678 (SEQ ID NO: 27,314)
SKAEEIAEKLDRLLEENRRALEEITTRLDDLLRRDHMVLHEYVNAAGITLDDLRRGGIREELLKRIADVIRDIMR
LLKELHDHTAEVIKTIKKLLKELHDINKEIIERLKRLKDGNVPKEELLKRVEELVRTSARLITEVLKTVEKLIRD
DKRLSEEILKRVKELIEELKRGVDSERVKEILERILRVVEEAVRLNEESLRRILDVVRKAVKLDRESLKKILDVV
EEAVR
>3plus1_Cage_Cterm_GFP11_678 (SEQ ID NO: 27,315)
SKAEEIAEKLDRLLEENRRALEEITTRLDDLLRRNKDALRKVMEKLKRLLDDLRRGGIREELLKRIADVIRDIMR
LLKELHDHTAEVIKTIKKLLKELHDINKEIIERLKRLKDGNVPKEELLKRVEELVRTSARLITEVLKTVEKLIRD
DKRLSEEILKRVKELIEELRDHMVLHEYVNAAGITLRVVEEAVRLNEESLRRILDVVRKAVKLDRESLKKILDVV
EEAVR
>3plus1_Cage_Nterm_GFP11_678 (SEQ ID NO: 27,316)
SKAEEIAEKLDRLLEENRRALEEITTRLDDLLRRNKDRDHMVLHEYVNAAGITRRGGIREELLKRIADVIRDIMR
LLKELHDHTAEVIKTIKKLLKELHDINKEIIERLKRLKDGNVPKEELLKRVEELVRTSARLITEVLKTVEKLIRD
DKRLSEEILKRVKELIEELKRGVDSERVKEILERILRVVEEAVRLNEESLRRILDVVRKAVKLDRESLKKILDVV
EEAVR
>3plus1_Cage_Cterm_GFP11_678 (SEQ ID NO: 27,317)
SKAEEIAEKLDRLLEENRRALEEITTRLDDLLRRNKDALRKVMEKLKRLLDDLRRGGIREELLKRIADVIRDIMR
LLKELHDHTAEVIKTIKKLLKELHDINKEIIERLKRLKDGNVPKEELLKRVEELVRTSARLITEVLKTVEKLIRD
DKRLSEEILKRVKELIEELKRGRDHMVLHEYVNAAGITVEEAVRLNEESLRRILDVVRKAVKLDRESLKKILDVV
EEAVR
>3plus1_Cage_Nterm_GFP11_678 (SEQ ID NO: 27,318)
SKAEEIAEKLDRLLEENRRALEEITTRLDDLLRRNKRDHMVLHEYVNAAGITLRRGGIREELLKRIADVIRDIMR
LLKELHDHTAEVIKTIKKLLKELHDINKEIIERLKRLKDGNVPKEELLKRVEELVRTSARLITEVLKTVEKLIRD
DKRLSEEILKRVKELIEELKRGVDSERVKEILERILRVVEEAVRLNEESLRRILDVVRKAVKLDRESLKKILDVV
EEAVR
>3plus1_Cage_Nterm_GFP11_678 (SEQ ID NO: 27,319)
SKAEEIAEKLDRLLEENRRALEEITTRLDDLLRRNKDALRDHMVLHEYVNAAGITGGIREELLKRIADVIRDIMR
LLKELHDHTAEVIKTIKKLLKELHDINKEIIERLKRLKDGNVPKEELLKRVEELVRTSARLITEVLKTVEKLIRD
DKRLSEEILKRVKELIEELKRGVDSERVKEILERILRVVEEAVRLNEESLRRILDVVRKAVKLDRESLKKILDVV
EEAVR
>3plus1_Cage_Cterm_GFP11_679 (SEQ ID NO: 27,320)
SRVEELKKLIEDILRISREVVERIKRVAEDIHRINRRVLDDLRKLIEDILRIVEEILARKVGDTEIAERLRDTIA
RVVDEIAKLLEEHEKRSRELLEEIRKLLEDILRRSERAVEEIRELLKKGVSTKDVLRIIEEILREHLELLERVVR
RIEEILRELLKTIEEIVKRIKEILEELKEVLKRGRVKDDEVERDHMVLHEYVNAAGITYRRLLEEIKRKLEEILR
RVEELHRRLRRKLEEIDR
>3plus1_Cage_Nterm_GFP11_679 (SEQ ID NO: 27,321)
SRVEELKKLIEDILRISREVVERIKRVAEDIHRINRRVRDHMVLHEYVNAAGITEILARKVGDTEIAERLRDTIA
RVVDEIAKLLEEHEKRSRELLEEIRKLLEDILRRSERAVEEIRELLKKGVSTKDVLRIIEEILREHLELLERVVR
RIEEILRELLKTIEEIVKRIKEILEELKEVLKRGRVKDDEVEREIRRVKEDLDRILEEYRRLLEEIKRKLEEILR
RVEELHRRLRRKLEEIDR

TABLE 3
Exemplary Bioactive peptides

GFP11 fluorescence peptide and binding peptide to GFP1-10: RDHMVLHEYVNAAGIT
(SEQ ID NO: 27052)
BIM binding peptide and apoptotic peptide to BCL-2: IxxxLRxIGDxFxxxY (SEQ
ID NO: 50), where x is any amino acid; in one embodiment, the peptide is
EIWIAQELRRIGDEFNAYYA (SEQ ID NO: 60)
Designed peptide for binding to BCL-2: KMAQELIDKVRAASLQINGDAFYAILRAL (SEQ
ID NO: 62)
StreptagII binding peptide to streptactin or an antibody: (N)WSHPQFEK (SEQ
ID NO: 63)
TEV protease cleavage site: ENLYFQ (G)-X (SEQ ID NO: 64), wherein (G) can
also be S, last position, -X can be anything except Proline
Thrombin protease cleavage site: LVPRGS (SEQ ID NO: 66)
Cathepsin cleavage site: RLVGFE (SEQ ID NO: 27053)
EZH2 binding peptide to recruit DNA-methylases:
TMFSSNRQKILERTETLNQEWKQRRIQ (SEQ ID NO: 27059)
MDM2 binding peptide to recruit p53: ETFSDLWKLL (SEQ ID NO: 27060)
CP5 binding peptide: GELDELVYLLDGPGYDPIHSDVVTRGGSHLFNF (SEQ ID NO: 27061)
9aaTAD1 for transcriptional activation: TMDDVYNYLFDD (SEQ ID NO: 27062)
9aaTAD2 for transcriptional activation: LLTGLFVQYLFDD (SEQ ID NO: 27063)
9aaTAD3 for transcriptional activation: DDAVVESFFSS (SEQ ID NO: 27064)
9aaTAD4 for transcriptional activation: GDFLSDLFD (SEQ ID NO: 27065)
9aaTAD5 for transcriptional activation: GDVLSDLVD (SEQ ID NO: 27066)
Mad1-SID - epigenetic modification: NIQMLLEAADYLE (SEQ ID NO: 27067)
Mad1-SID (3A mutant) - epigenetic modification: NIAMLLAAAAYLE (SEQ ID
NO: 27068)
RHIM Domain 1 from ZBP1: IQIG (SEQ ID NO: 27069)
RHIM Domain 2 from ZBP1: VQLG (SEQ ID NO: 27070)
nanoBit Split Luciferase: VSGWRLFKKIS (SEQ ID NO: 27071)
CC-A: GLEQEIAALEKENAALEWEIAALEQGG (SEQ ID NO: 27072)
CC-B: GLKQKIAALKYKNAALKKKIAALKQGG (SEQ ID NO: 27073)
GCN4: RMKQLEDKVEELLSKNYHLENEVARLKKLVGER (SEQ ID NO: 27074)
CC-Di: GEIAALKQEIAALKKENAALKWEIAALKQG (SEQ ID NO: 27075)
Membrane-disrupting/cell-penetrating peptides:
GALA for membrane disruption: WEAALAEALAEALAEHLAEALAEALEALAA (SEQ ID
NO: 27076)
Aurein 1.2: GLFDIIKKIAESF (SEQ ID NO: 27077)
Magainin-1: GIGKFLHSAGKFGKAFVGEIMKS (SEQ ID NO: 27078)
Magainin-2: GIGKFLHSAKKFGKAFVGEIMNS (SEQ ID NO: 27079)
Melittin: GIGAVLKVLTTGLPALISWIKRKRQQ (SEQ ID NO: 27080)
Mastoparan X: INWKGIAAMAKKLL (SEQ ID NO: 27081)
Cecropin A: KWKLFKKIEKVGQNIRDGIIKAGPAVAVVGQATQIAK (SEQ ID NO: 27082)
Cecropin P1: SWLSKTAKKLENSAKKRISEGIAIAIQGGPR (SEQ ID NO: 27083)
Citropin 1.1: GLFDVIKKVASVIGGL (SEQ ID NO: 27084)
Temporin-1Lb: NFLGTLINLAKKIL (SEQ ID NO: 27085)
HPV33 L2 peptide: SYFILRRRRKRFPYFFTDVRVAA (SEQ ID NO: 27086)
Adenovirus pVI membrane fusion domain: AFSWGSLWSGIKNFGSTVKNY (SEQ ID
NO: 27087)
Gamma-1 peptide from flock house virus: ASMWERVKSIIKSSLAAASNI (SEQ ID
NO: 27088)
Poliovirus 2B pore-forming peptide:
VTSTITEKLLKNLIKIISSLVIITRNYEDTTTVLATLALLGCDASPWQWL (SEQ ID NO: 27089)
Rhinovirus pore-forming peptide: IAQNPVENYIDEVLNEVLVVPNIN (SEQ ID
NO: 27090)
Influenza HA2 pore-forming peptide: FLGIAEAIDIGNGWEGMEFG (SEQ ID
NO: 27091)
Influenza HA2 derivative: GLFGAIAGFIENGWEGMIDG (SEQ ID NO: 27092)
HA-derived INF6: GLFGAIAGFIENGWEGMIDGWYG. (SEQ ID NO: 27093)

```
TABLE 4
Modular Co-LOCKR Key domains
  (parentheses are optional sequences of which a
portion can be deleted to tune Key affinity)
  (underlined amino acids can be changed to any
other amino acid to tune latch affinity)
```

>Key SEQ ID NO: 27393
(DEARKAIAR)VKRESKRIVEDAERLIREAAAASEKISR (EAERLIR)
>Key_B SEQ ID NO: 27394
DEAIARVKRESKRIVEDAERLIREAAAASEKISREAERLIR
>Key_C SEQ ID NO: 27395
DEVKRESKRIVEDAERLIREAAAASEKISREAERLIR
>Key_D SEQ ID NO: 27396
DEARKAIARVKRESKRIVEDAERLIREAAAASEKISREAER
>Key_E SEQ ID NO: 27397
DEARKAIARVKRESKRIVEDAERLIREAAAASEKISR
>Key_F SEQ ID NO: 27398
DEARKAIARVKRESKRIVEDAERLIREAAAASEKSSREAERLAR

In another embodiment, non-naturally occurring polypeptides comprising a polypeptide comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence of a key polypeptide selected from the group consisting of SEQ ID NOS: 26602-27050, and 27,322 to 27,358, as detailed below.
Key sequences are normal text
6His-MBP-TEV, 6His-TEV, and flexible linker sequences are underlined text sequence in bold, italics, are optional residues necessary for biotinylation of MBP_key
all sequences in parentheses are optional
Any number of consecutive amino acids from the N or C terminus in the non-optional key sequence may be removed to tune responsiveness

TABLE 5
Other Exemplary Key polypeptides without binding domains

>SB76_C-helix (SEQ ID NO: 27016)
DEARKAIARVKRESKRIVEDAERLIREAAAASEKIS
>SB76_C-helix-biotin (SEQ ID NO: 27017)
DEARKAIARVKRESKRIVEDAERLIREAAAASEKISGSGK-Biotin
>p5_MBP (SEQ ID NO: 27018)
(MDP)DEARKAIARVKRESKRIVEDAERLIREAAAASEKISREA(SSGLVPRGSHMKIEEGKLVIWINGDKGYNG
LAEVGKKFEKDTGIKVTVEHPDKLEEKFPQVAATGDGPDIIFWAHDRFGGYAQSGLLAEITPDKAFQDKLYPFTW
DAVRYNGKLIAYPIAVEALSLIYNKDLLPNPPKTWEEIPALDKELKAKGKSALMFNLQEPYFTWPLIAADGGYAF
KYENGKYDIKDVGVDNAGAKAGLTFLVDLIKNKHMNADTDYSIAEAAFNKGETAMTINGPWAWSNIDTSKVNYGV
TVLPTFKGQPSKPFVGVLSAGINAASPNKELAKEFLENYLLTDEGLEAVNKDKPLGAVALKSYEEELVKDPRIAA
TMENAQKGEIMPNIPQMSAFWYAVRTAVINAASGRQTVDEALKDAQTNSSGSGLNDIFEAQ [symbol] IEWHELEHHHHHH)
>p9_MBP (SEQ ID NO: 27019)
(MDP)DEARKAIARVKRESKRIVEDAERLIREAAAASEKISREAERLIREAA(SSGLVPRGSHMKIEEGKLVIWI
NGDKGYNGLAEVGKKFEKDTGIKVTVEHPDKLEEKFPQVAATGDGPDIIFWAHDREGGYAQSGLLAEITPDKAFQ
DKLYPFTWDAVRYNGKLIAYPIAVEALSLIYNKDLLPNPPKTWEEIPALDKELKAKGKSALMFNLQEPYFTWPLI
AADGGYAFKYENGKYDIKDVGVDNAGAKAGLTFLVDLIKNKHMNADTDYSIAEAAFNKGETAMTINGPWAWSNID
TSKVNYGVTVLPTFKGQPSKPFVGVLSAGINAASPNKELAKEFLENYLLTDEGLEAVNKDKPLGAVALKSYEEEL
VKDPRIAATMENAQKGEIMPNIPQMSAFWYAVRTAVINAASGRQTVDEALKDAQINSSGSGLNDIFEAQ [symbol] IEWHE
LEHHHHHH)
>p18_MBP (SEQ ID NO: 27020)
(MDP)DEARKAIARVKRESKRIVEDAERLIREAAAASEKISREAERLIREAAAASEKISRE(SSGLVPRGSHMKI
EEGKLVIWINGDKGYNGLAEVGKKFEKDTGIKVTVEHPDKLEEKFPQVAATGDGPDIIFWAHDRFGGYAQSGLLA
EITPDKAFQDKLYPFTWDAVRYNGKLIAYPIAVEALSLIYNKDLLPNPPKTWEEIPALDKELKAKGKSALMFNLQ
EPYFTWPLIAADGGYAFKYENGKYDIKDVGVDNAGAKAGLTFLVDLIKNKHMNADTDYSIAEAAFNKGETAMTIN
GPWAWSNIDTSKVNYGVTVLPTFKGQPSKPFVGVLSAGINAASPNKELAKEFLENYLLTDEGLEAVNKDKPLGAV
ALKSYEEELVKDPRIAATMENAQKGEIMPNIPQMSAFWYAVRTAVINAASGRQTVDEALKDAQTNSSGSGLNDIF
EAQ [symbol] IEWHELEHHHHHH)
>MBP_p18 (aka. p76) (SEQ ID NO: 27021)
(MGSSHHHHHHSSGLVPRGSHMKIEEGKLVIWINGDKGYNGLAEVGKKFEKDTGIKVTVEHPDKLEEKFPQVAAT
GDGPDIIFWAHDRFGGYAQSGLLAEITPDKAFQDKLYPFTWDAVRYNGKLIAYPIAVEALSLIYNKDLLPNPPKT
WEEIPALDKELKAKGKSALMFNLQEPYFTWPLIAADGGYAFKYENGKYDIKDVGVDNAGAKAGLIFLVDLIKNKH
MNADTDYSIAEAAFNKGETAMTINGPWAWSNIDTSKVNYGVTVLPTFKGQPSKPFVGVLSAGINAASPNKELAKE
FLENYLLTDEGLEAVNKDKPLGAVALKSYEEELVKDPRIAATMENAQKGEIMPNIPQMSAFWYAVRTAVINAASG
RQTVDEALKDAQTNSGSGSGENLYFQ)DEARKAIARVKRESKRIVEDAERLIREAAAASEKISRE(AERLIREAA
AASERISRE)
>key_b (SEQ ID NO: 27022)
(M)NKEEIEKLAKEAREKLKKAEKEHKEIHDKLRKKNKKAREDLKKKADELRETNKRVN(GSENLYFQGSGSGKI
EEGKLVIWINGDKGYNGLAEVGKKFEKDTGIKVTVEHPDKLEEKFPQVAATGDGPDIIFWAHDRFGGYAQSGLLA
EITPDKAFQDKLYPFTWDAVRYNGKLIAYPIAVEALSLIYNKDLLPNPPKTWEEIPALDKELKAKGKSALMFNLQ
EPYFTWPLIAADGGYAFKYENGKYDIKDVGVDNAGAKAGLTFLVDLIKNKHMNADTDYSIAEAAFNKGETAMTIN
GPWAWSNIDTSKVNYGVTVLPTFKGQPSKPFVGVLSAGINAASPNKELAKEFLENYLLTDEGLEAVNKDKPLGAV
ALKSYEEELVKDPRIAATMENAQKGEIMPNIPQMSAFWYAVRTAVINAASGRQTVDEALKDAQINLEHHHHHH)
>key_c (SEQ ID NO: 27023)
(M)SSEKVRRELKESLKENHKQNQKLLKDHKRAQEKLNRELEELKKKHKKTLDDIRRES(GSENLYFQGSGSGKI
EEGKLVIWINGDKGYNGLAEVGKKFEKDTGIKVTVEHPDKLEEKFPQVAATGDGPDIIFWAHDRFGGYAQSGLLA
EITPDKAFQDKLYPFTWDAVRYNGKLIAYPIAVEALSLIYNKDLLPNPPKTWEEIPALDKELKAKGKSALMENLQ
EPYFTWPLIAADGGYAFKYENGKYDIKDVGVDNAGAKAGLTFLVDLIKNKHMNADTDYSIAEAAFNKGETAMTIN
GPWAWSNIDTSKVNYGVTVLPTFKGQPSKPFVGVLSAGINAASPNKELAKEFLENYLLTDEGLEAVNKDKPLGAV
ALKSYEEELVKDPRIAATMENAQKGEIMPNIPQMSAFWYAVRTAVINAASGRQTVDEALKDAQTNLEHHHHHH)
>key_d (SEQ ID NO: 27024)
(M)DTVKRILEELRRRFEKLAKDLDDIARKLLEDHKKHNKELKDKQRKIKKEADDAARS(GSENLYFQGSGSGKI
EEGKLVIWINGDKGYNGLAEVGKKFEKDTGIKVTVEHPDKLEEKFPQVAATGDGPDIIFWAHDRFGGYAQSGLLA
EITPDKAFQDKLYPFTWDAVRYNGKLIAYPIAVEALSLIYNKDLLPNPPKTWEEIPALDKELKAKGKSALMFNLQ
EPYFTWPLIAADGGYAFKYENGKYDIKDVGVDNAGAKAGLTFLVDLIKNKHMNADTDYSIAEAAFNKGETAMTIN
GPWAWSNIDTSKVNYGVTVLPTFKGQPSKPFVGVLSAGINAASPNKELAKEFLENYLLTDEGLEAVNKDKPLGAV
ALKSYEEELVKDPRIAATMENAQKGEIMPNIPQMSAFWYAVRTAVINAASGRQTVDEALKDAQTNLEHHHHHH)
>key_e (SEQ ID NO: 27025)
(M)DDVERRLRKANKESKKEAEELTEEAKKANEKTKEDSKELTKENRKINKTIKDEARS(GSENLYFQGSGSGKI
EEGKLVIWINGDKGYNGLAEVGKKFEKDTGIKVTVEHPDKLEEKFPQVAATGDGPDIIFWAHDRFGGYAQSGLLA
EITPDKAFQDKLYPFTWDAVRYNGKLIAYPIAVEALSLIYNKDLLPNPPKTWEEIPALDKELKAKGKSALMFNLQ
EPYFTWPLIAADGGYAFKYENGKYDIKDVGVDNAGAKAGLTFLVDLIKNKHMNADTDYSIAEAAFNKGETAMTIN
GPWAWSNIDTSKVNYGVTVLPTFKGQPSKPFVGVLSAGINAASPNKELAKEFLENYLLTDEGLEAVNKDKPLGAV
ALKSYEEELVKDPRIAATMENAQKGEIMPNIPQMSAFWYAVRTAVINAASGRQTVDEALKDAQTNLEHHHHHH)
>key_f (SEQ ID NO: 27026)
(M)DDEERRSEKTVQDAKREIKKVEDDLQRLNEEQKKKVKKQEDENQKTLKKHKDDARS(GSENLYFQGSGSGKI
EEGKLVIWINGDKGYNGLAEVGKKFEKDTGIKVTVEHPDKLEEKFPQVAATGDGPDIIFWAHDRFGGYAQSGLLA
EITPDKAFQDKLYPFTWDAVRYNGKLIAYPIAVEALSLIYNKDLLPNPPKTWEEIPALDKELKAKGKSALMFNLQ
EPYFTWPLIAADGGYAFKYENGKYDIKDVGVDNAGAKAGLTFLVDLIKNKHMNADTDYSIAEAAFNKGETAMTIN
GPWAWSNIDTSKVNYGVTVLPTFKGQPSKPFVGVLSAGINAASPNKELAKEFLENYLLTDEGLEAVNKDKPLGAV
ALKSYEEELVKDPRIAATMENAQKGEIMPNIPQMSAFWYAVRTAVINAASGRQTVDEALKDAQTNLEHHHHHH)
Additional Keys:
Key sequences are normal text
(6His-MBP-TEV, 6His-TEV, and flexible linker sequences are underlined text)
(Co-localization domain is bolded text)
(Positions that can be mutated to any amino acid to tune responsiveness are
underlined bolded text. These are exemplary but not exhaustive.)
(Any number of consecutive amino acids from the N or C terminus in the
non-optional key sequence may be removed to tune responsiveness)
(all sequences in parentheses are optional)
>p76-long (SEQ ID NO: 27027)
(MGSSHHHHHHSSGLVPRGSHMKIEEGKLVIWINGDKGYNGLAEVGKKFEKDTGIKVTVEHPDKLEEKFPQVAAT
GDGPDIIFWAHDRFGGYAQSGLLAEITPDKAFQDKLYPFTWDAVRYNGKLIAYPIAVEALSLIYNKDLLPNPPKT
WEEIPALDKELKAKGKSALMFNLQEPYFTWPLIAADGGYAFKYENGKYDIKDVGVDNAGAKAGLTFLVDLIKNKH
MNADTDYSIAEAAFNKGETAMTINGPWAWSNIDTSKVNYGVTVLPTFKGQPSKPFVGVLSAGINAASPNKELAKE
FLENYLLTDEGLEAVNKDKPLGAVALKSYEEELVKDPRIAATMENAQKGEIMPNIPQMSAFWYAVRTAVINAASG
RQTVDEALKDAQTNSGSGSGENLYFQ)DEARKAIARVKRESKRIVEDAERLIREAAAASEKISREAERL [symbol] REAAA
ASEK [symbol] SRE
>p76-short (SEQ ID NO: 27028)
(MGSSHHHHHHSSGLVPRGSHMKIEEGKLVIWINGDKGYNGLAEVGKKFEKDTGIKVTVEHPDKLEEKFPQVAAT
GDGPDIIFWAHDRFGGYAQSGLLAEITPDKAFQDKLYPFTWDAVRYNGKLIAYPIAVEALSLIYNKDLLPNPPKT
WEEIPALDKELKAKGKSALMFNLQEPYFTWPLIAADGGYAFKYENGKYDIKDVGVDNAGAKAGLTFLVDLIKNKH
MNADTDYSIAEAAFNKGETAMTINGPWAWSNIDTSKVNYGVTVLPTFKGQPSKPFVGVLSAGINAASPNKELAKE
FLENYLLTDEGLEAVNKDKPLGAVALKSYEEELVKDPRIAATMENAQKGEIMPNIPQMSAFWYAVRTAVINAASG
RQTVDEALKDAQTNSGSGSGENLYFQ)DEARKAIARVKRESKRIVEDAERLIREAAAASEK [symbol] SREAERL [symbol] R
>k76-long (SEQ ID NO: 27029)
(MGSSHHHHHHSSGLVPRGSHMKIEEGKLVIWINGDKGYNGLAEVGKKFEKDTGIKVTVEHPDKLEEKFPQVAAT
GDGPDIIFWAHDRFGGYAQSGLLAEITPDKAFQDKLYPFTWDAVRYNGKLIAYPIAVEALSLIYNKDLLPNPPKT
WEEIPALDKELKAKGKSALMFNLQEPYFTWPLIAADGGYAFKYENGKYDIKDVGVDNAGAKAGLTFLVDLIKNKH
MNADTDYSIAEAAFNKGETAMTINGPWAWSNIDTSKVNYGVTVLPTFKGQPSKPFVGVLSAGINAASPNKELAKE
FLENYLLTDEGLEAVNKDKPLGAVALKSYEEELVKDPRIAATMENAQKGEIMPNIPQMSAFWYAVRTAVINAASG
RQTVDEALKDAQTNSGSGSGENLYFQ)DEARKAIARVKRESKRIVEDAERLIREAAQASEKISREAREL [symbol] ERAAQ
ASEK [symbol] SRE
>k76-short (SEQ ID NO: 27030)
(MGSSHHHHHHSSGLVPRGSHMKIEEGKLVIWINGDKGYNGLAEVGKKFEKDTGIKVTVEHPDKLEEKFPQVAAT
GDGPDIIFWAHDRFGGYAQSGLLAEITPDKAFQDKLYPFTWDAVRYNGKLIAYPIAVEALSLIYNKDLLPNPPKT
WEEIPALDKELKAKGKSALMFNLQEPYFTWPLIAADGGYAFKYENGKYDIKDVGVDNAGAKAGLTFLVDLIKNKH
MNADTDYSIAEAAFNKGETAMTINGPWAWSNIDTSKVNYGVTVLPTFKGQPSKPFVGVLSAGINAASPNKELAKE
FLENYLLTDEGLEAVNKDKPLGAVALKSYEEELVKDPRIAATMENAQKGEIMPNIPQMSAFWYAVRTAVINAASG
RQTVDEALKDAQTNSGSGSGENLYFQ)DEARKAIARVKRESKRIVEDAERLIREAAQASEK [symbol] SREAERL [symbol] R
>p76_GLISE (SEQ ID NO: 27031)
(MGSHHHHHHGSGSENLYFQGSGGS)DEARKAIARVKRESKRIVEDAEGLISEAAAASEKISREAERLIREAAAA
SEKISRE
>p76_GSSEKIS (SEQ ID NO: 27032)
(MGSHHHHHHGSGSENLYFQGSGGS)DEARKAIARVKRESKRIVEDAERLIREAAGSSEKISREAERLIREAAAA
SEKISRE
>p76_R26G (SEQ ID NO: 27033)
(MGSHHHHHHGSGSENLYFQGSGGS)DEARKAIARVKRESKRIVEDAERLIGEAAAASEKISREAERLIREAAAA
SEKISRE
>p76-short_E19G (SEQ ID NO: 27034)
(MGSHHHHHHGSGSENLYFQGSGGS)DEARKAIARVKRESKRIVGDAERLIREAAAASEKISREAERLIR
>p76-short_GLISE_E01_EGFR (SEQ ID NO: 27035)
(MGSHHHHHHGSGSENLYFQGSGGS)DEARKAIARVKRESKRIVEDAEGLISEAAAASEKISREAERLIR
>p76-short_AE_EGFR (SEQ ID NO: 27036)
(MGSHHHHHHGSGSENLYFQGSGGS)DEARKAIARVAEESKRIVEDAERLIREAAAASEKISREAERLIR
>p76-short_AAE_EGFR (SEQ ID NO: 27037)
(MGSHHHHHHGSGSENLYFQGSGGS)DEAAKAIARVAEESKRIVEDAERLIREAAAASEKISREAERLIR
>p76-short_EE_EGFR (SEQ ID NO: 27038)
(MGSHHHHHHGSGSENLYFQGSGGS)DEARKAIARVKRESKRIVEDAERLIREAAEASEEISREAERLIR
>p76-spytag (SEQ ID NO: 27039)
(MGSHHHHHHGSGSENLYFQGSGGSMKIEEGKLVIWINGDKGYNGLAEVGKKFEKDTGIKVTVEHPDKLEEKFPQ
VAATGDGPDIIFWAHDRFGGYAQSGLLAEITPDKAFQDKLYPFTWDAVRYNGKLIAYPIAVEALSLIYNKDLLPN
PPKTWEEIPALDKELKAKGKSALMFNLQEPYFTWPLIAADGGYAFKYENGKYDIKDVGVDNAGAKAGLTFLVDLI
KNKHMNADTDYSIAEAAFNKGETAMTINGPWAWSNIDTSKVNYGVTVLPTFKGQPSKPFVGVLSAGINAASPNKE
LAKEFLENYLLTDEGLEAVNKDKPLGAVALKSYEEELVKDPRIAATMENAQKGEIMPNIPQMSAFWYAVRTAVIN
AASGRQTVDEALKDAQTNSGSGSGENLYFQ)DEARKAIARVKRESKRIVEDAERLIREAAAASEKISREAERL [symbol] R
EAAAASEK [symbol] SRE(GGGSGSGSGSGKPGQASGS)AHIVMVDAYKPTK
>p76-short-spytag (SEQ ID NO: 27040)
(MGSHHHHHHGSGSENLYFQGSGGSMKIEEGKLVIWINGDKGYNGLAEVGKKFEKDTGIKVTVEHPDKLEEKFPQ
VAATGDGPDIIFWAHDRFGGYAQSGLLAEITPDKAFQDKLYPFTWDAVRYNGKLIAYPIAVEALSLIYNKDLLPN
PPKTWEEIPALDKELKAKGKSALMFNLQEPYFTWPLIAADGGYAFKYENGKYDIKDVGVDNAGAKAGLTFLVDLI
KNKHMNADTDYSIAEAAFNKGETAMTINGPWAWSNIDTSKVNYGVTVLPTFKGQPSKPFVGVLSAGINAASPNKE
LAKEFLENYLLTDEGLEAVNKDKPLGAVALKSYEEELVKDPRIAATMENAQKGEIMPNIPQMSAFWYAVRTAVIN
AASGRQTVDEALKDAQTNSGSGSGENLYFQ)DEARKAIARVKRESKRIVEDAERLIREAAAASEK [symbol] SREAERL [symbol] R
(GGGSGSGSGSGKPGQASGS)AHIVMVDAYKPTK
>sfGFP_VMAn_p18 (SEQ ID NO: 27041)
(MGSSHHHHHHSSGLVPRGSHMSKGEELFTGVVPILVELDGDVNGHKFSVRGEGEGDATNGKLTLKFICTTGKLP
VPWPTLVTTLTYGVQCFARYPDHMKQHDFFKSAMPEGYVQERTISFKDDGTYKTRAEVKFEGDTLVNRIELKGID
FKEDGNILGHKLEYNFNSHNVYITADKQKNGIKANFKIRHNVEDGSVQLADHYQQNTPIGDGPVLLPDNHYLSTQ
SVLSKDPNEKRDHMVLLEFVTAAGITHGMDELYKSGSGSGCFAKGTNVLMADGSIECIENIEVGNKVMGKDGRPR
EVIKLPRGRETMYSVVQKSQHRAHKSDSSREVPELLKFTCNATHELVVRTPRSVRRLSRTIKGVEYFEVITFEMG
QKKAPDGRIVELVKEVSKSYPISEGPERANELVESYRKASNKAYFEWTIEARDLSLLGSHVRKATYQTYAPILYG
GSGGS)DEARKAIARVKRESKRIVEDAERLIREAAAASEKISREAERLIREAAAASEKISRE
>p18_VMAc_mCherry (SEQ ID NO: 27042)
(MGSHHHHHHGSGSENLYFQGSG)DEARKAIARVKRESKRIVEDAERLIREAAAASEKISREAERLIREAAAASE
KISRE(GSGGSGSGGGVLLNVLSKCAGSKKFRPAPAAAFARECRGFYFELQELKEDDYYGITLSDDSDHQFLLAN
QVVVHNCGSGGSVSKGEEDNMAIIKEFMRFKVHMEGSVNGHEFEIEGEGEGRPYEGTQTAKLKVTKGGPLPFAWD
ILSPQFMYGSKAYVKHPADIPDYLKLSFPEGFKWERVMNFEDGGVVTVTQDSSLQDGEFIYKVKLRGTNFPSDGP
VMQKKTMGWEASSERMYPEDGALKGEIKQRLKLKDGGHYDAEVKTTYKAKKPVQLPGAYNVNIKLDITSHNEDYT
IVEQYERAEGRHSTGGMDELYK)
(Cognate Keys for 2plus1 and 3plus1 STREPII-LOCKR functional Cage designs):
>2plus1_KEY_100000.fasta alt_STREP_2plus1_1 (SEQ ID NO: 27043)
DKVRKVAEVAEKVLRDIDKLDRESKEAFRRTNGEISKLDEDTRRVAERVKKAIEDLAK
>2plus1_KEY_2 (SEQ ID NO: 27044)
SEVDEIIADNERALDEVRREVEEIDKENAERLKEWVEEAREILDRLAKALEEIR
>2plus1_KEY_3 (SEQ ID NO: 27045)
PEEALSKAIKDVRDIVKKVKDELKEWRDRNKELVDRLSEELKEWLKDVERVLKELTDKDR
>2plus1_KEY_4 (SEQ ID NO: 27046)
ERVREELKKLLTRVEEEHRKVLETDKKILKEAHKESKEVNDRDRELLERLEESVR
>3plus1_KEY_1 (SEQ ID NO: 27047)
SRLVKKLDEIVKEVAKKLEDVVRANEELWRKLVELNKESVARLREAVERVARDLEETAR
>3plus1_KEY_2 (SEQ ID NO: 27048)
DEERLEKVVKDVIEKVRRILEKWKKDIDKVVKELRRILEEWEKIIREVLDKVR
>3plus1_KEY_3 (SEQ ID NO: 27049)
KDAVIKVIEKLIRANAAVWDALLKINEDLVRVNKTVWKELLRVNEKLARDLERVVK
>3plus1_KEY_4 (SEQ ID NO: 27050)
SLVDELRKSLERNVRVSEEVARRLKEALKRWVDVVRKVVEDLIRLNEDVVRVVEK
SEQ ID NOs: 26,602-27,015:
>3plus1_GFP11_Key_Cterm_1 (SEQ ID NO: 26602)
SGSKEVLDILERAVEVVRRVIKALKEVLERHVDATREVIERVKRVNKRLLEAVREVVT
>3plus1_GFP11_Key_Cterm_2 (SEQ ID NO: 26603)
GVPEEIDRELKRVVEELRRLHEEIKERLDDVARRSEEELRRIIKKLKEVVKEIRKKLK
>3plus1_GFP11_Key_Cterm_3 (SEQ ID NO: 26604)
DLLRKLEEELRRIKEKLRKALEELEREHRELEKELDKLHDESRKEHERIEEELRR
>3plus1_GFP11_Key_Cterm_4 (SEQ ID NO: 26605)
DEDLLEKIKRVIREHIKALEKLARDLKEILRRHIEALKELARDLAEVIRKLLEDVKR
>3plus1_GFP11_Key_Cterm_5 (SEQ ID NO: 26606)
DLERLRRKVEELEDRLRRLLEKLARDSAELMRELERILDRYARESEELDRRLAE
>3plus1_GFP11_Key_Cterm_6 (SEQ ID NO: 26607)
DLEDILRKNLDRLRKLLERLREILRENLEALKKTLKRLEDVVREILEDLKRERK
>3plus1_GFP11_Key_Cterm_7 (SEQ ID NO: 26608)
DLERLRRKVEELEDRLRRLLEKLARDSAELMRELERILDRYARESEELDRRLAE
>3plus1_GFP11_Key_Cterm_8 (SEQ ID NO: 26609)
SGSKEVLDILERAVEVVRRVIKALKEVLERHVDATREVIERVKRVNKRLLEAVREVVT
>3plus1_GFP11_Key_Cterm_9 (SEQ ID NO: 26610)
DLERLRRKVEELEDRLRRLLEKLARDSAELMRELERILDRYARESEELDRRLAE
>3plus1_GFP11_Key_Cterm_10 (SEQ ID NO: 26611)
RLIEEVVRLLRENLDVVRRILEALAKLIKELLEALEEVLRRNKELIRELLRVLDEALK
>3plus1_GFP11_Key_Cterm_11 (SEQ ID NO: 26612)
DIVRAMEEVIRRLIEILRRDVELNLDVAKKLLELLKEDSKLNLDVARELLELLDR
>3plus1_GFP11_Key_Cterm_12 (SEQ ID NO: 26613)
DIVRAMEEVIRRLIEILRRDVELNLDVAKKLLELLKEDSKLNLDVARELLELLDR
>3plus1_GFP11_Key_Cterm_13 (SEQ ID NO: 26614)
RLIEEVVRLLRENLDVVRRILEALAKLIKELLEALEEVLRRNKELIRELLRVLDEALK
>3plus1_GFP11_Key_Cterm_14 (SEQ ID NO: 26615)
RLIEEVVRLLRENLDVVRRILEALAKLIKELLEALEEVLRRNKELIRELLRVLDEALK
>3plus1_GFP11_Key_Cterm_15 (SEQ ID NO: 26616)
DLLRKLEEELRRIKEKLRKALEELEREHRELEKELDKLHDESRKEHERIEEELRR
>3plus1_GFP11_Key_Cterm_16 (SEQ ID NO: 26617)
DLLRKLEEELRRIKEKLRKALEELEREHRELEKELDKLHDESRKEHERIEEELRR
>3plus1_GFP11_Key_Cterm_17 (SEQ ID NO: 26618)
ELAREVERVIKELLDKSKEILERIERAIDELLKVSEEILKLSEDASEELLKILREFAK
>3plus1_GFP11_Key_Cterm_18 (SEQ ID NO: 26619)
DVKDIIRTILEVARDLLRLLEEDSRTNSEVVKRLLDLLREDSKANSEVVKRLLDVLRE
>3plus1_GFP11_Key_Cterm_19 (SEQ ID NO: 26620)
DLERLRRKVEELEDRLRRLLEKLARDSAELMRELERILDRYARESEELDRRLAE
>3plus1_GFP11_Key_Cterm_20 (SEQ ID NO: 26621)
DLERLRRKVEELEDRLRRLLEKLARDSAELMRELERILDRYARESEELDRRLAE
>3plus1_GFP11_Key_Cterm_21 (SEQ ID NO: 26622)
RLIEEVVRLLRENLDVVRRILEALAKLIKELLEALEEVLRRNKELIRELLRVLDEALK
>3plus1_GFP11_Key_Cterm_22 (SEQ ID NO: 26623)
DLEDILRKNLDRLRKLLERLREILRENLEALKKTLKRLEDVVREILEDLKRERK
>3plus1_GFP11_Key_Cterm_23 (SEQ ID NO: 26624)
DLLRKLEEELRRIKEKLRKALEELEREHRELEKELDKLHDESRKEHERIEEELRR
>3plus1_GFP11_Key_Cterm_24 (SEQ ID NO: 26625)
DEDLLEKIKRVIREHIKALEKLARDLKEILRRHIEALKELARDLAEVIRKLLEDVKR
>3plus1_GFP11_Key_Cterm_25 (SEQ ID NO: 26626)
ELVRIAIEVLKRLLEIIEELVRLNNEILERLLKIVRELHKDNIKILEDLLRIIEEVLR
>3plus1_GFP11_Key_Cterm_26 (SEQ ID NO: 26627)
ELVRIAIEVLKRLLEIIEELVRLNNEILERLLKIVRELHKDNIKILEDLLRIIEEVLR
>3plus1_GFP11_Key_Cterm_27 (SEQ ID NO: 26628)
RLARLLKALADKLIRVLEEILKINEELNRKIIKFARENLERNRRVNKKVIEVLREAAR
>3plus1_GFP11_Key_Cterm_28 (SEQ ID NO: 26628)
DLERLRRKVEELEDRLRRLLEKLARDSAELMRELERILDRYARESEELDRRLAE
>3plus1_GFP11_Key_Cterm_29 (SEQ ID NO: 26630)
ELVRIAIEVLKRLLEIIEELVRLNNEILERLLKIVRELHKDNIKILEDLLRIIEEVLR
>3plus1_GFP11_Key_Cterm_30 (SEQ ID NO: 26631)
ELVRIAIEVLKRLLEIIEELVRLNNEILERLLKIVRELHKDNIKILEDLLRIIEEVLR
>3plus1_GFP11_Key_Cterm_31 (SEQ ID NO: 26632)
DIVRAMEEVIRRLIEILRRDVELNLDVAKKLLELLKEDSKLNLDVARELLELLDR
>3plus1_GFP11_Key_Cterm_32 (SEQ ID NO: 26633)
RKIAKIIEELKRLLEDLARDIRRVIEEAKRLLKEWRDRNKEVADTLKKLLEDLIRKIR
>3plus1_GFP11_Key_Cterm_33 (SEQ ID NO: 26634)
DLLRKLEEELRRIKEKLRKALEELEREHRELEKELDKLHDESRKEHERIEEELRR
>3plus1_GFP11_Key_Cterm_34 (SEQ ID NO: 26635)
DLLRKLEEELRRIKEKLRKALEELEREHRELEKELDKLHDESRKEHERIEEELRR
>3plus1_GFP11_Key_Cterm_35 (SEQ ID NO: 26636)
RKIAKIIEELKRLLEDLARDTRRVIEEAKRLLKEWRDRNKEVADTLKKLLEDLIRKIR
>3plus1_GFP11_Key_Cterm_36 (SEQ ID NO: 26637)
ELVRIAIEVLKRLLEIIEELVRLNNEILERLLKIVRELHKDNIKILEDLLRIIEEVLR
>3plus1_GFP11_Key_Cterm_37 (SEQ ID NO: 26638)
TVRRLREALKKLEDDLRKIERDAEREYKKLKDELEELTERYRREIRKLKEELKADRK
>3plus1_GFP11_Key_Cterm_38 (SEQ ID NO: 26639)
DEAERRRRELKDKLDRLREEHEEVKRRLEEELTRLRETHKKIEKELREALKRVRDRST
>3plus1_GFP11_Key_Cterm_39 (SEQ ID NO: 26640)
DEAERRRRELKDKLDRLREEHEEVKRRLEEELTRLRETHKKIEKELREALKRVRDRST
>3plus1_GFP11_Key_Cterm_40 (SEQ ID NO: 26641)
DLEDILRKNLDRLRKLLERLREILRENLEALKKTLKRLEDVVREILEDLKRERK
>3plus1_GFP11_Key_Cterm_41 (SEQ ID NO: 26642)
DLERLRRKVEELEDRLRRLLEKLARDSAELMRELERILDRYARESEELDRRLAE
>3plus1_GFP11_Key_Cterm_42 (SEQ ID NO: 26643)
DEAERRRRELKDKLDRLREEHEEVKRRLEEELTRLRETHKKIEKELREALKRVRDRST
>3plus1_GFP11_Key_Cterm_43 (SEQ ID NO: 26644)
DEAERRRRELKDKLDRLREEHEEVKRRLEEELTRLRETHKKIEKELREALKRVRDRST
>3plus1_GFP11_Key_Cterm_44 (SEQ ID NO: 26645)
DEAERRRRELKDKLDRLREEHEEVKRRLEEELTRLRETHKKIEKELREALKRVRDRST
>3plus1_GFP11_Key_Cterm_45 (SEQ ID NO: 26646)
DKAVEELEKALEEIKRRLKEVIDRYEDELRKLRKEYKEKIDKYERKLEEIERRERT
>3plus1_GFP11_Key_Cterm_46 (SEQ ID NO: 26647)
DVKRALEELVSRLRKLLEDVKKASEDIVREVERIVRELAKRSDEILKKLEDIVEKLRE
>3plus1_GFP11_Key_Cterm_47 (SEQ ID NO: 26648)
DVKRALEELVSRLRKLLEDVKKASEDIVREVERIVRELAKRSDEILKKLEDIVEKLRE
>3plus1_GFP11_Key_Cterm_48 (SEQ ID NO: 26649)
DEAERRRRELKDKLDRLREEHEEVKRRLEEELTRLRETHKKIEKELREALKRVRDRST
>3plus1_GFP11_Key_Cterm_49 (SEQ ID NO: 26650)
DVKRALEELVSRLRKLLEDVKKASEDIVREVERIVRELAKRSDEILKKLEDIVEKLRE
>3plus1_GFP11_Key_Cterm_50 (SEQ ID NO: 26651)
EVKRRLEEKERRIRTRYEELRRRLRKRVKDYEDKLREIEKKVRRDAERIEEELERAKK
>3plus1_GFP11_Key_Cterm_51 (SEQ ID NO: 26652)
DEAERRRRELKDKLDRLREEHEEVKRRLEEELTRLRETHKKIEKELREALKRVRDRST
>3plus1_GFP11_Key_Cterm_52 (SEQ ID NO: 26653)
KIAEEIERELEELRRMIKRLHEDLERKLKESEDELREIEARLEEKIRRLEEKLERKRR
>3plus1_GFP11_Key_Cterm_53 (SEQ ID NO: 26654)
KIAEEIERELEELRRMIKRLHEDLERKLKESEDELREIEARLEEKIRRLEEKLERKRR
>3plus1_GFP11_Key_Cterm_54 (SEQ ID NO: 26655)
DKAVEELEKALEEIKRRLKEVIDRYEDELRKLRKEYKEKIDKYERKLEEIERRERT
>3plus1_GFP11_Key_Cterm_55 (SEQ ID NO: 26656)
KIAEEIERELEELRRMIKRLHEDLERKLKESEDELREIEARLEEKIRRLEEKLERKRR
>3plus1_GFP11_Key_Cterm_56 (SEQ ID NO: 26657)
ELVRIAIEVLKRLLEIIEELVRLNNEILERLLKIVRELHKDNIKILEDLLRIIEEVLR
>3plus1_GFP11_Key_Cterm_57 (SEQ ID NO: 26658)
DEVEREIRRVKEDLDRILEEYRRLLEEIKRKLEEILRRVEELHRRLRRKLEEIDR
>3plus1_GFP11_Key_Cterm_58 (SEQ ID NO: 26659)
DVKRALEELVSRLRKLLEDVKKASEDIVREVERIVRELAKRSDEILKKLEDIVEKLRE
>3plus1_GFP11_Key_Cterm_59 (SEQ ID NO: 26660)
DEAERRRRELKDKLDRLREEHEEVKRRLEEELTRLRETHKKIEKELREALKRVRDRST
>3plus1_GFP11_Key_Cterm_60 (SEQ ID NO: 26661)
DEAERRRRELKDKLDRLREEHEEVKRRLEEELTRLRETHKKIEKELREALKRVRDRST
>3plus1_GFP11_Key_Cterm_61 (SEQ ID NO: 26662)
TLREVVRKVLEEAKRLLDELEEVHKRVKKELEDIIEENRRVVKRVRDELREIKRELDE
>3plus1_GFP11_Key_Cterm_62 (SEQ ID NO: 26663)
DVKRALEELVSRLRKLLEDVKKASEDIVREVERIVRELAKRSDEILKKLEDIVEKLRE
>3plus1_GFP11_Key_Cterm_63 (SEQ ID NO: 26664)
DVKRALEELVSRLRKLLEDVKKASEDIVREVERIVRELAKRSDEILKKLEDIVEKLRE
>3plus1_GFP11_Key_Cterm_64 (SEQ ID NO: 26665)
DEAERRRRELKDKLDRLREEHEEVKRRLEEELTRLRETHKKIEKELREALKRVRDRST
>3plus1_GFP11_Key_Cterm_65 (SEQ ID NO: 26666)
DEAERRRRELKDKLDRLREEHEEVKRRLEEELTRLRETHKKIEKELREALKRVRDRST
>3plus1_GFP11_Key_Cterm_66 (SEQ ID NO: 26667)
KIAEEIERELEELRRMIKRLHEDLERKLKESEDELREIEARLEEKIRRLEEKLERKRR
>3plus1_GFP11_Key_Cterm_67 (SEQ ID NO: 26668)
DVKRALEELVSRLRKLLEDVKKASEDIVREVERIVRELAKRSDEILKKLEDIVEKLRE
>3plus1_GFP11_Key_Nterm_68 (SEQ ID NO: 26669)
SEAERLADEVRKAVKKSEEDNETLVREVEKAVRELKKNNKTWVDEVRKLMKRLVDLLR
>3plus1_GFP11_Key_Nterm_69 (SEQ ID NO: 26670)
SEAERLADEVRKAVKKSEEDNETLVREVEKAVRELKKNNKTWVDEVRKLMKRLVDLLR
>3plus1_GFP11_Key_Nterm_70 (SEQ ID NO: 26671)
DKDKRLEELLKRLKELNDKTFEELERILEELKRANEASLREAERILEELRARIEGGNL
>3plus1_GFP11_Key_Nterm_71 (SEQ ID NO: 26672)
SEAEDLEELIKELAELLKDVIRKLEKINRRLVKILEDIIRRLKEISKEAEEELRKGTV
>3plus1_GFP11_Key_Nterm_72 (SEQ ID NO: 26673)
SDKEEIKRRVEKTARDLETEHDKIKKRLEDTVRDIKRELDELLEKYERVLRKIEKTLR
>3plus1_GFP11_Key_Nterm_73 (SEQ ID NO: 26674)
SEAEKIREALETNLRLLEELIKRLKEILDTHNELLRRVIETLERLLKELLELLEEGGL
>3plus1_GFP11_Key_Nterm_74 (SEQ ID NO: 26675)
SEAEKIREALETNLRLLEELIKRLKEILDTHNELLRRVIETLERLLKELLELLEEGGL
>3plus1_GFP11_Key_Nterm_75 (SEQ ID NO: 26676)
SKEERLREVAEKHKKDLEDIVKRVDEAAKETARRLEEILKRLEEVLKKILDDLEKGPD
>3plus1_GFP11_Key_Nterm_76 (SEQ ID NO: 26677)
SLEEITKRLLELVEENLARHEEILRELLELAKRLAKEDRDILEEVLKLIEELLKLLED
>3plus1_GFP11_Key_Nterm_77 (SEQ ID NO: 26678)
SKEETLKRLLDELEKRNRETVERLERLLKELEDRNRASLEELEAVLEELERKIEESGL
>3plus1_GFP11_Key_Nterm_78 (SEQ ID NO: 26679)
SKEETLKRLLDELEKRNRETVERLERLLKELEDRNRASLEELEAVLEELERKIEESGL
>3plus1_GFP11_Key_Nterm_79 (SEQ ID NO: 26680)
SKEETLKRLLDELEKRNRETVERLERLLKELEDRNRASLEELEAVLEELERKIEESGL
>3plus1_GFP11_Key_Nterm_80 (SEQ ID NO: 26681)
STREKAKKVLDTLRADNEDMKRVVEKILRALKRTNERAEKLAREITEEIKRILKEVGV
>3plus1_GFP11_Key_Nterm_81 (SEQ ID NO: 26682)
DAEEVVKRLADVLRENDETIRKVVEDLVRIAEENDRLWKKLVEDIAEILRRIVELLRR
>3plus1_GFP11_Key_Nterm_82 (SEQ ID NO: 26683)
SKEETLKRLLDELEKRNRETVERLERLLKELEDRNRASLEELEAVLEELERKIEESGL
>3plus1_GFP11_Key_Nterm_83 (SEQ ID NO: 26684)
STREKAKKVLDTLRADNEDMKRVVEKILRALKRTNERAEKLAREITEEIKRILKEVGV
>3plus1_GFP11_Key_Nterm_84 (SEQ ID NO: 26685)
SKEEEVEKVLRKWEEILRRLIEENKRANDKIRREYEELVKEIRRVLEEIKEVAERLGV
>3plus1_GFP11_Key_Nterm_85 (SEQ ID NO: 26686)
DREKSVRDIEEDLKRVLDKLRRRVETSKEELKKVLKADKENADELEKTLRDVVRELDR
>3plus1_GFP11_Key_Nterm_86 (SEQ ID NO: 26687)
SDKEEIKRRVEKTARDLETEHDKIKKRLEDTVRDIKRELDELLEKYERVLRKIEKTLR
>3plus1_GFP11_Key_Nterm_87 (SEQ ID NO: 26688)
STREKAKKVLDTLRADNEDMKRVVEKILRALKRTNERAEKLAREITEEIKRILKEVGV
>3plus1_GFP11_Key_Nterm_88 (SEQ ID NO: 26689)
SKDEELARLLEELVERWRKIVEDLERDHRRLVKEIRELVERIRKKLEELVDRIRKNGI
>3plus1_GFP11_Key_Nterm_89 (SEQ ID NO: 26690)
SEAERLADEVRKAVKKSEEDNETLVREVEKAVRELKKNNKTWVDEVRKLMKRLVDLLR
>3plus1_GFP11_Key_Nterm_90 (SEQ ID NO: 26691)
SKDEELARLLEELVERWRKIVEDLERDHRRLVKEIRELVERIRKKLEELVDRIRKNGI
>3plus1_GFP11_Key_Nterm_91 (SEQ ID NO: 26692)
KEIEETLKELEDLNREMVETNRRVLEETRRLNKETVDRVKATLDELAKMLKKLVDDVR
>3plus1_GFP11_Key_Nterm_92 (SEQ ID NO: 26693)
SEAERLADEVRKAVKKSEEDNETLVREVEKAVRELKKNNKTWVDEVRKLMKRLVDLLR
>3plus1_GFP11_Key_Nterm_93 (SEQ ID NO: 26694)
SKEETLKRLLDELEKRNRETVERLERLLKELEDRNRASLEELEAVLEELERKIEESGL
>3plus1_GFP11_Key_Nterm_94 (SEQ ID NO: 26695)
DKAEVLREALKLLKDLLEELIKIHEESLKRILDLIDTLVKVHEDALRALKELLERSGL
>3plus1_GFP11_Key_Nterm_95 (SEQ ID NO: 26696)
SKEEEVEKVLRKWEEILRRLIEENKRANDKIRREYEELVKEIRRVLEEIKEVAERLGV
>3plus1_GFP11_Key_Nterm_96 (SEQ ID NO: 26697)
SKEETLKRLLDELEKRNRETVERLERLLKELEDRNRASLEELEAVLEELERKIEESGL
>3plus1_GFP11_Key_Cterm_97 (SEQ ID NO: 26698)
SERVKEILERILRVVEEAVRLNEESLRRILDVVRKAVKLDRESLKKILDVVEEAVR
>3plus1_GFP11_Key_Cterm_98 (SEQ ID NO: 26699)
SERVKEILERILRVVEEAVRLNEESLRRILDVVRKAVKLDRESLKKILDVVEEAVR
>3plus1_GFP11_Key_Cterm_99 (SEQ ID NO: 26700)
DERRIAERIRELLRESKKLVRDVVEEAKRLLKENRDSTRKIIEDIRRLLRKIEDSTR
>3plus1_GFP11_Key_Cterm_100 (SEQ ID NO: 26701)
DALSRLLEELLRVVDDLIRVLKELIDKSRKVIEELLELLKRINEENLKVLAEIIK
>3plus1_GFP11_Key_Cterm_101 (SEQ ID NO: 26702)
DERRIAERIRELLRESKKLVRDVVEEAKRLLKENRDSTRKIIEDIRRLLRKIEDSTR
>3plus1_GFP11_Key_Cterm_102 (SEQ ID NO: 26703)
EALRKLVELLVEVLRRLIRVNRELVKLLREVLERLLRILRESVKKLKRLIEKVIKDAT
>3plus1_GFP11_Key_Cterm_103 (SEQ ID NO: 26704)
EALRKLVELLVEVLRRLIRVNRELVKLLREVLERLLRILRESVKKLKRLIEKVIKDAT
>3plus1_GFP11_Key_Cterm_104 (SEQ ID NO: 26705)
AAKRLVEELLKAVTDLSRKNKRILEELLKAIETLSDENKKILTEILDALRRLVEKIEK
>3plus1_GFP11_Key_Cterm_105 (SEQ ID NO: 26706)
EALRKLVELLVEVLRRLIRVNRELVKLLREVLERLLRILRESVKKLKRLIEKVIKDAT
>3plus1_GFP11_Key_Cterm_106 (SEQ ID NO: 26707)
EALRKLVELLVEVLRRLIRVNRELVKLLREVLERLLRILRESVKKLKRLIEKVIKDAT
>3plus1_GFP11_Key_Cterm_107 (SEQ ID NO: 26708)
DALSRLLEELLRVVDDLIRVLKELIDKSRKVIEELLELLKRINEENLKVLAEIIK
>3plus1_GFP11_Key_Cterm_108 (SEQ ID NO: 26709)
DALSRLLEELLRVVDDLIRVLKELIDKSRKVIEELLELLKRINEENLKVLAEIIK
>3plus1_GFP11_Key_Cterm_109 (SEQ ID NO: 26710)
RAVKKLDEIVKEVAKKLEDVVRANEELWRALVELNKESVRRLREIVERVARDLEETAR
>3plus1_GFP11_Key_Cterm_110 (SEQ ID NO: 26711)
DRLDKVEELVKKLLEDTKRTVDRVRELVRKILKKSRETLEELERLIEKILRELEKDAR
>3plus1_GFP11_Key_Cterm_111 (SEQ ID NO: 26712)
DALSRLLEELLRVVDDLIRVLKELIDKSRKVIEELLELLKRINEENLKVLAEIIK
>3plus1_GFP11_Key_Cterm_112 (SEQ ID NO: 26713)
RAVKKLDEIVKEVAKKLEDVVRANEELWRALVELNKESVRRLREIVERVARDLEETAR
>3plus1_GFP11_Key_Cterm_113 (SEQ ID NO: 26714)
RAVKKLDEIVKEVAKKLEDVVRANEELWRALVELNKESVRRLREIVERVARDLEETAR
>3plus1_GFP11_Key_Cterm_114 (SEQ ID NO: 26715)
SEDDLKRVVDEVEKKLRELKRRYAEALERIKEKIKELKDRYERAVREVVAELRKTTK
>3plus1_GFP11_Key_Cterm_115 (SEQ ID NO: 26716)
RAVKKLDEIVKEVAKKLEDVVRANEELWRALVELNKESVRRLREIVERVARDLEETAR
>3plus1_GFP11_Key_Cterm_116 (SEQ ID NO: 26717)
DEVEREIRRVKEDLDRILEEYRRLLEEIKRKLEEILRRVEELHRRLRRKLEEIDR
>3plus1_GFP11_Key_Cterm_117 (SEQ ID NO: 26718)
SEDDLKRVVDEVEKKLRELKRRYAEALERIKEKIKELKDRYERAVREVVAELRKTTK
>3plus1_GFP11_Key_Nterm_118 (SEQ ID NO: 26719)
DEAKELLDEIRKAVKESEDRLEKLLRDYEKELRRLEKELRDLKRRIEEKLEELRRGSL
>3plus1_GFP11_Key_Nterm_119 (SEQ ID NO: 26720)
SEKEDAARKLRKLVEELTREYEELVKKLERLIEEIEKVSEESVRKLEKLLAEISEEVR
>3plus1_GFP11_Key_Nterm_120 (SEQ ID NO: 26721)
SEDEIIKKIIEDLRRVLKEVEEIHKEVEERLDKVLKEAEEMHKEVLKELDRVLDEVKR
>3plus1_GFP11_Key_Nterm_121 (SEQ ID NO: 26722)
SKAEEIAEKLDRLLEENRRALEEITTRLDDLLRRNKDALRKVMEKLKRLLDDLRRGGI
>3plus1_GFP11_Key_Nterm_122 (SEQ ID NO: 26723)
SEKEKLLKESEEEVRRLRRTLEELLRKYREVLERLRKELREIEERVRDVVRRLKEVLD
>3plus1_GFP11_Key_Nterm_123 (SEQ ID NO: 26724)
SEKEDAARKLRKLVEELTREYEELVKKLERLIEEIEKVSEESVRKLEKLLAEISEEVR
>3plus1_GFP11_Key_Nterm_124 (SEQ ID NO: 26725)
SKEETLRKEAEDLLRRLEELTRRLEKKARELLERAKKLSRDLAEELKRLLKELREKGV
>3plus1_GFP11_Key_Nterm_125 (SEQ ID NO: 26726)
SEKEDAARKLRKLVEELTREYEELVKKLERLIEEIEKVSEESVRKLEKLLAEISEEVR
>3plus1_GFP11_Key_Nterm_126 (SEQ ID NO: 26727)
SKAEEIAEKLDRLLEENRRALEEITTRLDDLLRRNKDALRKVMEKLKRLLDDLRRGGI
>3plus1_GFP11_Key_Nterm_127 (SEQ ID NO: 26728)
SEKEDAARKLRKLVEELTREYEELVKKLERLIEEIEKVSEESVRKLEKLLAEISEEVR
>3plus1_GFP11_Key_Nterm_128 (SEQ ID NO: 26729)
SKAEEIAEKLDRLLEENRRALEEITTRLDDLLRRNKDALRKVMEKLKRLLDDLRRGGI
>3plus1_GFP11_Key_Nterm_129 (SEQ ID NO: 26730)
SKEETLRKEAEDLLRRLEELTRRLEKKARELLERAKKLSRDLAEELKRLLKELREKGV
>3plus1_GFP11_Key_Nterm_130 (SEQ ID NO: 26731)
SRVEELKKLIEDILRISREVVERIKRVAEDIHRINRRVLDDLRKLIEDILRTVEEILA
>3plus1_GFP11_Key_Nterm_131 (SEQ ID NO: 26732)
SKEETLRKEAEDLLRRLEELTRRLEKKARELLERAKKLSRDLAEELKRLLKELREKGV
>3plus1_GFP11_Key_Nterm_132 (SEQ ID NO: 26733)
SKAEEIAEKLDRLLEENRRALEEITTRLDDLLRRNKDALRKVMEKLKRLLDDLRRGGI
>3plus1_GFP11_Key_Nterm_133 (SEQ ID NO: 26734)
SKEETLRKEAEDLLRRLEELTRRLEKKARELLERAKKLSRDLAEELKRLLKELREKGV
>3plus1_GFP11_Key_Nterm_134 (SEQ ID NO: 26735)
SEKEDAARKLRKLVEELTREYEELVKKLERLIEEIEKVSEESVRKLEKLLAEISEEVR
>3plus1_GFP11_Key_Nterm_135 (SEQ ID NO: 26736)
SKEETLRKEAEDLLRRLEELTRRLEKKARELLERAKKLSRDLAEELKRLLKELREKGV
>3plus1_GFP11_Key_Nterm_136 (SEQ ID NO: 26737)
SERETVKRRLEELLKEVKRTLDKLKEEHDRLLEDVRRVVEELKREHDKLLKEVKDSGV
>3plus1_GFP11_Key_Nterm_137 (SEQ ID NO: 26738)
SEKEDAARKLRKLVEELTREYEELVKKLERLIEEIEKVSEESVRKLEKLLAEISEEVR
>3plus1_GFP11_Key_Nterm_138 (SEQ ID NO: 26739)
SKEETLRKEAEDLLRRLEELTRRLEKKARELLERAKKLSRDLAEELKRLLKELREKGV
>3plus1_GFP11_Key_Nterm_139 (SEQ ID NO: 26740)
KEREEVKEKLDRLLEEVEKTVRELKREHDELLKEVEKLVRDLKKEHDELLKKVKDDGV
>3plus1_GFP11_Key_Nterm_140 (SEQ ID NO: 26741)
SREEVLRELEEVIEDNRRLLEELIEKSKKVLDESLKLIDELLRRLEEVLERVLRLLEE
>2plus1_GFP11_Key_Cterm_1 (SEQ ID NO: 26742)
DEVVKRVRDLLDTVRRRNEKVNEDVKRMNDKLRRDNEDVIRRVEKLLRELEEKRRT
>2plus1_GFP11_Key_Cterm_2 (SEQ ID NO: 26743)
SEDSVERIARELERNLDDLARVLKESEDDLAEILRRLKEVLEESERDLERVEREVRK
>2plus1_GFP11_Key_Cterm_3 (SEQ ID NO: 26744)
SKELLEKAKAVVDEIKRLAEESLKRLEDLSRDHKRRAKELNDEIAKVVDELAKRAT
>2plus1_GFP11_Key_Cterm_4 (SEQ ID NO: 26745)
SKEKIDRIIRELERILEEAKKKHEDVLRRLEDSLRRVAELLKAALDRLREIVDRLRR
>2plus1_GFP11_Key_Cterm_5 (SEQ ID NO: 26746)
DEVVKRVRDLLDTVRRRNEKVNEDVKRMNDKLRRDNEDVIRRVEKLLRELEEKRRT
>2plus1_GFP11_Key_Cterm_6 (SEQ ID NO: 26747)
DIKTLLDRVRKLAEEDAERLDRLRRESEELNERVRRVDKKLLEEIRRKAKKVEDDIR
>2plus1_GFP11_Key_Cterm_7 (SEQ ID NO: 26748)
DAEILLRELEKLSRDNKELLKKIEKEIRDLIKEDKERNIELSERLRKLVEELKKKAT
>2plus1_GFP11_Key_Cterm_8 (SEQ ID NO: 26749)
DEVVKRVRDLLDTVRRRNEKVNEDVKRMNDKLRRDNEDVIRRVEKLLRELEEKRRT
>2plus1_GFP11_Key_Cterm_9 (SEQ ID NO: 26750)
DIKTLLDRVRKLAEEDAERLDRLRRESEELNERVRRVDKKLLEEIRRKAKKVEDDIR
>2plus1_GFP11_Key_Cterm_10 (SEQ ID NO: 26751)
SEELSAEVKKLLDEVRKALARHKDENDKLLKEIEDSLRRHKEENDRLLEKLKESTR
>2plus1_GFP11_Key_Cterm_11 (SEQ ID NO: 26752)
DADDVLARVEELAKRAHDENERLIREVEELVRAHNKRNKELVDEVKRLVEKVIEEER
>2plus1_GFP11_Key_Cterm_12 (SEQ ID NO: 26753)
SKEKIDRIIRELERILEEAKKKHEDVLRRLEDSLRRVAELLKAALDRLREIVDRLRR
>2plus1_GFP11_Key_Cterm_13 (SEQ ID NO: 26754)
DEVVKRVRDLLDTVRRRNEKVNEDVKRMNDKLRRDNEDVIRRVEKLLRELEEKRRT
>2plus1_GFP11_Key_Cterm_14 (SEQ ID NO: 26755)
SEELSAEVKKLLDEVRKALARHKDENDKLLKEIEDSLRRHKEENDRLLEKLKESTR
>2plus1_GFP11_Key_Cterm_15 (SEQ ID NO: 26756)
DAETVLRSAEDIVAKNRKLAEEVLRRVKKIVEENRKIASEVLDDVRKLVEDVLARAS
>2plus1_GFP11_Key_Cterm_16 (SEQ ID NO: 26757)
DADDVLARVEELAKRAHDENERLIREVEELVRAHNKRNKELVDEVKRLVEKVIEEER
>2plus1_GFP11_Key_Cterm_17 (SEQ ID NO: 26758)
SKEKIDRIIRELERILEEAKKKHEDVLRRLEDSLRRVAELLKAALDRLREIVDRLRR
>2plus1_GFP11_Key_Cterm_18 (SEQ ID NO: 26759)
DEEKLKDLIRKLRDILRRAAEAHKKLIDDARESLERAKREHEKLIDRLKKILEELER
>2plus1_GFP11_Key_Cterm_19 (SEQ ID NO: 26760)
DIKTLLDRVRKLAEEDAERLDRLRRESEELNERVRRVDKKLLEEIRRKAKKVEDDTR
>2plus1_GFP11_Key_Cterm_20 (SEQ ID NO: 26761)
DATRVIEEAKRILDEARKLNEETIRRSEELVRRIERVIEEIIKRSEKLLEDVARESK
>2plus1_GFP11_Key_Cterm_21 (SEQ ID NO: 26762)
SEELSAEVKKLLDEVRKALARHKDENDKLLKEIEDSLRRHKEENDRLLEKLKESTR
>2plus1_GFP11_Key_Cterm_22 (SEQ ID NO: 26763)
DAETVLRSAEDIVAKNRKLAEEVLRRVKKIVEENRKIASEVLDDVRKLVEDVLARAS
>2plus1_GFP11_Key_Cterm_23 (SEQ ID NO: 26764)
DADDVLARVEELAKRAHDENERLIREVEELVRAHNKRNKELVDEVKRLVEKVIEEER
>2plus1_GFP11_Key_Cterm_24 (SEQ ID NO: 26765)
SKELLEKAKAVVDEIKRLAEESLKRLEDLSRDHKRRAKELNDEIAKVVDELAKRAT
>2plus1_GFP11_Key_Cterm_25 (SEQ ID NO: 26766)
DEEVLKKLAEIVRRVKEENRKVNEEVEKRLRELEEENKKVIEDLKSTVEELVERLR
>2plus1_GFP11_Key_Cterm_26 (SEQ ID NO: 26767)
DEEVLKKLAEIVRRVKEENRKVNEEVEKRLRELEEENKKVIEDLKSTVEELVERLR
>2plus1_GFP11_Key_Cterm_27 (SEQ ID NO: 26768)
DKLLKEARDLIREIEKRLEELLKRVEKLTEDAKRDLERSNREHKELADRIKETAR
>2plus1_GFP11_Key_Cterm_28 (SEQ ID NO: 26769)
DKDSARELERIVKENAELAERVFREVEKIVRENTKLAEDSVRELKRLVEELKKRAK
>2plus1_GFP11_Key_Cterm_29 (SEQ ID NO: 26770)
SKEKIDRIIRELERILEEAKKKHEDVLRRLEDSLRRVAELLKAALDRLREIVDRLRR
>2plus1_GFP11_Key_Cterm_30 (SEQ ID NO: 26771)
DEEKLKDLIRKLRDILRRAAEAHKKLIDDARESLERAKREHEKLIDRLKKILEELER
>2plus1_GFP11_Key_Cterm_31 (SEQ ID NO: 26772)
DEVVKRVRDLLDTVRRRNEKVNEDVKRMNDKLRRDNEDVIRRVEKLLRELEEKRRT
>2plus1_GFP11_Key_Cterm_32 (SEQ ID NO: 26773)
DEEVLRTLEEIIRRLTKELEDVLREYERELRRLEEENKRVIDKTEEEIRRLADRLRR
>2plus1_GFP11_Key_Cterm_33 (SEQ ID NO: 26774)
DERILRELEERVKELEKEAREILKRSEDETDKLREKAERILEDLERANRRTMDEARR
>2plus1_GFP11_Key_Cterm_34 (SEQ ID NO: 26775)
SEELSAEVKKLLDEVRKALARHKDENDKLLKEIEDSLRRHKEENDRLLEKLKESTR
>2plus1_GFP11_Key_Cterm_35 (SEQ ID NO: 26776)
LPEEVLRELEELLKESEERIKRIEEEIKKIIDKSREDIKRVLEEIERLNAKAADDLRK
>2plus1_GFP11_Key_Cterm_36 (SEQ ID NO: 26777)
DADDVLARVEELAKRAHDENERLIREVEELVRAHNKRNKELVDEVKRLVEKVIEEER
>2plus1_GFP11_Key_Cterm_37 (SEQ ID NO: 26778)
DEEVLKKLAEIVRRVKEENRKVNEEVEKRLRELEEENKKVIEDLKSTVEELVERLR
>2plus1_GFP11_Key_Cterm_38 (SEQ ID NO: 26779)
DKLLKEARDLIREIEKRLEELLKRVEKLTEDAKRDLERSNREHKELADRIKETAR
>2plus1_GFP11_Key_Cterm_39 (SEQ ID NO: 26780)
DEEVLRTLEEIIRRLTKELEDVLREYERELRRLEEENKRVIDKTEEEIRRLADRLRR
>2plus1_GFP11_Key_Cterm_40 (SEQ ID NO: 26781)
DRRIEKVLKEIEEKIREVIKEWERVHREVEELLKRLIDENRKVLDEIRKLLEEKSK
>2plus1_GFP11_Key_Cterm_41 (SEQ ID NO: 26782)
DERILRELEERVKELEKEAREILKRSEDETDKLREKAERILEDLERANRRTMDEARR
>2plus1_GFP11_Key_Cterm_42 (SEQ ID NO: 26783)
SEELSAEVKKLLDEVRKALARHKDENDKLLKEIEDSLRRHKEENDRLLEKLKESTR
>2plus1_GFP11_Key_Cterm_43 (SEQ ID NO: 26784)
DEEVLKKLAEIVRRVKEENRKVNEEVEKRLRELEEENKKVIEDLKSTVEELVERLR
>2plus1_GFP11_Key_Cterm_44 (SEQ ID NO: 26785)
SKEKIDRIIRELERILEEAKKKHEDVLRRLEDSLRRVAELLKAALDRLREIVDRLRR
>2plus1_GFP11_Key_Cterm_45 (SEQ ID NO: 26786)
DRRIEKVLKEIEEKIREVIKEWERVHREVEELLKRLIDENRKVLDEIRKLLEEKSK
>2plus1_GFP11_Key_Cterm_46 (SEQ ID NO: 26787)
TLRELARSIRKLSAENKERLKELLRELKKLSDENKERIKKLLSDAEKIIEDVARRAK
>2plus1_GFP11_Key_Cterm_47 (SEQ ID NO: 26788)
DERILRELEERVKELEKEAREILKRSEDETDKLREKAERILEDLERANRRTMDEARR
>2plus1_GFP11_Key_Cterm_48 (SEQ ID NO: 26789)
EKLKELRDVIAEVAKRIDELDEYTRESIRRAKKEIERLNRETKKVIEEVVKRIEEERK
>2plus1_GFP11_Key_Cterm_49 (SEQ ID NO: 26790)
DERVREELKKLLTRVEEEHRKVLETDKKILKEAHKESKEVNDRDRELLERLEESVR
>2plus1_GFP11_Key_Cterm_50 (SEQ ID NO: 26791)
DADDVLARVEELAKRAHDENERLIREVEELVRAHNKRNKELVDEVKRLVEKVIEEER
>2plus1_GFP11_Key_Cterm_51 (SEQ ID NO: 26792)
TVKRLLDELRELLERLKRTIEELLKRNRDLLADAEEKARRLLEENRKLLKAARDTAT
>2plus1_GFP11_Key_Cterm_52 (SEQ ID NO: 26793)
DEEVLKKLAEIVRRVKEENRKVNEEVEKRLRELEEENKKVIEDLKSTVEELVERLR
>2plus1_GFP11_Key_Cterm_53 (SEQ ID NO: 26794)
SKEKIDRIIRELERILEEAKKKHEDVLRRLEDSLRRVAELLKAALDRLREIVDRLRR
>2plus1_GFP11_Key_Cterm_54 (SEQ ID NO: 26795)
DERILRELEERVKELEKEAREILKRSEDETDKLREKAERILEDLERANRRTMDEARR
>2plus1_GFP11_Key_Cterm_55 (SEQ ID NO: 26796)
DATRVIEEAKRILDEARKLNEETIRRSEELVRRIERVIEEIIKRSEKLLEDVARESK
>2plus1_GFP11_Key_Cterm_56 (SEQ ID NO: 26797)
EAAREIIKRLREVNKRTKEKLDELIKHSEEVLERVKRLIDELRKHSEEVLEDLRRRAK
>2plus1_GFP11_Key_Cterm_57 (SEQ ID NO: 26798)
EKLKELRDVIAEVAKRIDELDEYTRESIRRAKKEIERLNRETKKVIEEVVKRIEEERK
>2plus1_GFP11_Key_Cterm_58 (SEQ ID NO: 26799)
ELLRRIKKLLDEIKKAIEDSSREIKRLLEESERVMKRSSEDIKRTLDDTRRVVEEVRR
>2plus1_GFP11_Key_Cterm_59 (SEQ ID NO: 26800)
SKAIKDVRDIVKKVKDELKEWRDRNKELVDRLSEELKEWLKDVERVLKELTDKDR
>2plus1_GFP11_Key_Cterm_60 (SEQ ID NO: 26801)
DERVREELKKLLTRVEEEHRKVLETDKKILKEAHKESKEVNDRDRELLERLEESVR
>2plus1_GFP11_Key_Cterm_61 (SEQ ID NO: 26802)
DIDKLLKELRDLVEKIKKDLKELLERYEEIVRRIKELLKDLNREAEEVVRRLKEELR
>2plus1_GFP11_Key_Cterm_62 (SEQ ID NO: 26803)
DADDVLARVEELAKRAHDENERLIREVEELVRAHNKRNKELVDEVKRLVEKVIEEER
>2plus1_GFP11_Key_Cterm_63 (SEQ ID NO: 26804)
DEEVLKKLAEIVRRVKEENRKVNEEVEKRLRELEEENKKVIEDLKSTVEELVERLR
>2plus1_GFP11_Key_Cterm_64 (SEQ ID NO: 26805)
EREEELKEVADRVKEKLDRLNRENEKSSEELKRELDKINDENRETSERLKREIDETTR
>2plus1_GFP11_Key_Cterm_65 (SEQ ID NO: 26806)
SKEKIDRIIRELERILEEAKKKHEDVLRRLEDSLRRVAELLKAALDRLREIVDRLRR
>2plus1_GFP11_Key_Cterm_66 (SEQ ID NO: 26807)
SKEKIDRIIRELERILEEAKKKHEDVLRRLEDSLRRVAELLKAALDRLREIVDRLRR
>2plus1_GFP11_Key_Cterm_67 (SEQ ID NO: 26808)
TKDLLDENSKRSNEISREVKKDLERTVRENKKIVDEVAKALEDTVDKNRRIVEEVTT
>2plus1_GFP11_Key_Cterm_68 (SEQ ID NO: 26809)
DEVVKRVRDLLDTVRRRNEKVNEDVKRMNDKLRRDNEDVIRRVEKLLRELEEKRRT
>2plus1_GFP11_Key_Cterm_69 (SEQ ID NO: 26810)
DRRIEKVLKEIEEKIREVIKEWERVHREVEELLKRLIDENRKVLDEIRKLLEEKSK
>2plus1_GFP11_Key_Cterm_70 (SEQ ID NO: 26811)
ELLRRIKKLLDEIKKAIEDSSREIKRLLEESERVMKRSSEDIKRTLDDIRRVVEEVRR
>2plus1_GFP11_Key_Cterm_71 (SEQ ID NO: 26812)
SEELSAEVKKLLDEVRKALARHKDENDKLLKEIEDSLRRHKEENDRLLEKLKESTR
>2plus1_GFP11_Key_Cterm_72 (SEQ ID NO: 26813)
DIDKLLKELRDLVEKIKKDLKELLERYEEIVRRIKELLKDLNREAEEVVRRLKEELR
>2plus1_GFP11_Key_Cterm_73 (SEQ ID NO: 26814)
DADDVLARVEELAKRAHDENERLIREVEELVRAHNKRNKELVDEVKRLVEKVIEEER
>2plus1_GFP11_Key_Cterm_74 (SEQ ID NO: 26815)
DEEVLKKLAEIVRRVKEENRKVNEEVEKRLRELEEENKKVIEDLKSTVEELVERLR
>2plus1_GFP11_Key_Cterm_75 (SEQ ID NO: 26816)
DEEVLKKLAEIVRRVKEENRKVNEEVEKRLRELEEENKKVIEDLKSTVEELVERLR
>2plus1_GFP11_Key_Cterm_76 (SEQ ID NO: 26817)
SKEKIDRIIRELERILEEAKKKHEDVLRRLEDSLRRVAELLKAALDRLREIVDRLRR
>2plus1_GFP11_Key_Cterm_77 (SEQ ID NO: 26818)
SEELREELKKLERKIEKVAKEIHDHDKEVTERLEDLLRRITEHARKSDREIEETAR
>2plus1_GFP11_Key_Cterm_78 (SEQ ID NO: 26819)
DRRIEKVLKEIEEKIREVIKEWERVHREVEELLKRLIDENRKVLDEIRKLLEEKSK
>2plus1_GFP11_Key_Cterm_79 (SEQ ID NO: 26820)
DATRVIEEAKRILDEARKLNEETIRRSEELVRRIERVIEEIIKRSEKLLEDVARESK
>2plus1_GFP11_Key_Cterm_80 (SEQ ID NO: 26821)
EKLKELRDVIAEVAKRIDELDEYTRESIRRAKKEIERLNRETKKVIEEVVKRIEEERK
>2plus1_GFP11_Key_Cterm_81 (SEQ ID NO: 26822)
ELLRRIKKLLDEIKKAIEDSSREIKRLLEESERVMKRSSEDIKRILDDIRRVVEEVRR
>2plus1_GFP11_Key_Cterm_82 (SEQ ID NO: 26823)
DIDKLLKELRDLVEKIKKDLKELLERYEEIVRRIKELLKDLNREAEEVVRRLKEELR
>2plus1_GFP11_Key_Cterm_83 (SEQ ID NO: 26824)
EAKKKLDEVLERAKRTIDRLLETSDRSLEKVEADLRRLNEELDRSLERAERTIRELAK
>2plus1_GFP11_Key_Cterm_84 (SEQ ID NO: 26825)
DEEVLKKLAEIVRRVKEENRKVNEEVEKRLRELEEENKKVIEDLKSTVEELVERLR
>2plus1_GFP11_Key_Cterm_85 (SEQ ID NO: 26826)
DEEVLKKLAEIVRRVKEENRKVNEEVEKRLRELEEENKKVIEDLKSTVEELVERLR
>2plus1_GFP11_Key_Cterm_86 (SEQ ID NO: 26827)
DRRIEKVLKEIEEKIREVIKEWERVHREVEELLKRLIDENRKVLDEIRKLLEEKSK
>2plus1_GFP11_Key_Cterm_87 (SEQ ID NO: 26828)
DERILRELEERVKELEKEAREILKRSEDETDKLREKAERILEDLERANRRTMDEARR
>2plus1_GFP11_Key_Cterm_88 (SEQ ID NO: 26829)
DLKRVEERAREVSRRNEESMRRVKEDADRVSEANKEVLDRVREEVKRLIEEVRETLR
>2plus1_GFP11_Key_Cterm_89 (SEQ ID NO: 26830)
EKLKELRDVIAEVAKRIDELDEYTRESIRRAKKEIERLNRETKKVIEEVVKRIEEERK
>2plus1_GFP11_Key_Cterm_90 (SEQ ID NO: 26831)
ELLRRIKKLLDEIKKAIEDSSREIKRLLEESERVMKRSSEDIKRTLDDTRRVVEEVRR
>2plus1_GFP11_Key_Cterm_91 (SEQ ID NO: 26832)
ELLRRIKKLLDEIKKAIEDSSREIKRLLEESERVMKRSSEDIKRTLDDTRRVVEEVRR
>2plus1_GFP11_Key_Cterm_92 (SEQ ID NO: 26833)
LPEEVLRELEELLKESEERIKRIEEEIKKIIDKSREDIKRVLEEIERLNAKAADDLRK
>2plus1_GFP11_Key_Cterm_93 (SEQ ID NO: 26834)
EAKKKLDEVLERAKRTIDRLLETSDRSLEKVEADLRRLNEELDRSLERAERTIRELAK
>2plus1_GFP11_Key_Cterm_94 (SEQ ID NO: 26835)
DEEVLKKLAEIVRRVKEENRKVNEEVEKRLRELEEENKKVIEDLKSTVEELVERLR
>2plus1_GFP11_Key_Cterm_95 (SEQ ID NO: 26836)
DKLLKEARDLIREIEKRLEELLKRVEKLTEDAKRDLERSNREHKELADRIKETAR
>2plus1_GFP11_Key_Cterm_96 (SEQ ID NO: 26837)
DKLLKEARDLIREIEKRLEELLKRVEKLTEDAKRDLERSNREHKELADRIKETAR
>2plus1_GFP11_Key_Cterm_97 (SEQ ID NO: 26838)
DIVRKIERIVETIEREVRESVKKVEEIARDIRRKVDESVKNVEKLLRDVDKKARDRKK
>2plus1_GFP11_Key_Cterm_98 (SEQ ID NO: 26839)
DEIKRIVDEVRERLKRIVDENAKIVEDARRALEKIVKENEEILRRLKKELRELRK
>2plus1_GFP11_Key_Cterm_99 (SEQ ID NO: 26840)
DRRIEKVLKEIEEKIREVIKEWERVHREVEELLKRLIDENRKVLDEIRKLLEEKSK
>2plus1_GFP11_Key_Cterm_100 (SEQ ID NO: 26841)
DLKRVEERAREVSRRNEESMRRVKEDADRVSEANKEVLDRVREEVKRLIEEVRETLR
>2plus1_GFP11_Key_Cterm_101 (SEQ ID NO: 26842)
DATRVIEEAKRILDEARKLNEETIRRSEELVRRIERVIEEIIKRSEKLLEDVARESK
>2plus1_GFP11_Key_Cterm_102 (SEQ ID NO: 26843)
DAETIERVVRELLEENKEVLRKTEEAVKRSTETNKRLLEASKEVADRLRERIKEAAK
>2plus1_GFP11_Key_Cterm_103 (SEQ ID NO: 26844)
EKLKELRDVIAEVAKRIDELDEYTRESIRRAKKEIERLNRETKKVIEEVVKRIEEERK
>2plus1_GFP11_Key_Cterm_104 (SEQ ID NO: 26845)
ELLRRIKKLLDEIKKAIEDSSREIKRLLEESERVMKRSSEDIKRTLDDIRRVVEEVRR
>2plus1_GFP11_Key_Cterm_105 (SEQ ID NO: 26846)
DEVVERAERISEENKRRVEDVARKSKELVEDVRRHSEEVVRRVEELVKEVEERVR
>2plus1_GFP11_Key_Cterm_106 (SEQ ID NO: 26847)
EAKKKLDEVLERAKRTIDRLLETSDRSLEKVEADLRRLNEELDRSLERAERTIRELAK
>2plus1_GFP11_Key_Cterm_107 (SEQ ID NO: 26848)
TAERARETLKRLLDENRDRSKKVKEEIRRILEDLTRTTERVKREIAKLLKELEDTAR
>2plus1_GFP11_Key_Cterm_108 (SEQ ID NO: 26849)
EAVRRLKEILERLKEEVRRSLEELRKEVERLKKEVEDSLRELKKSLEEWVKSLEEATR
>2plus1_GFP11_Key_Cterm_109 (SEQ ID NO: 26850)
DERILRELEERVKELEKEAREILKRSEDETDKLREKAERILEDLERANRRTMDEARR
>2plus1_GFP11_Key_Cterm_110 (SEQ ID NO: 26851)
DATRVIEEAKRILDEARKLNEETIRRSEELVRRIERVIEEIIKRSEKLLEDVARESK
>2plus1_GFP11_Key_Cterm_111 (SEQ ID NO: 26852)
DKARKVAEVAEKVLRDIDKLDRESKEAFRATNEEIAKLDEDTARVAERVKKAIEDLAK
>2plus1_GFP11_Key_Cterm_112 (SEQ ID NO: 26853)
EKLKELRDVIAEVAKRIDELDEYTRESIRRAKKEIERLNRETKKVIEEVVKRIEEERK
>2plus1_GFP11_Key_Cterm_113 (SEQ ID NO: 26854)
ELLRRIKKLLDEIKKAIEDSSREIKRLLEESERVMKRSSEDIKRTLDDTRRVVEEVRR
>2plus1_GFP11_Key_Cterm_114 (SEQ ID NO: 26855)
DKVERVVREVEKLHEEDRKRLEESTRSVRKLLEELKRELEKSTRSVKALVDELRERVR
>2plus1_GFP11_Key_Cterm_115 (SEQ ID NO: 26856)
DEVVERAERISEENKRRVEDVARKSKELVEDVRRHSEEVVRRVEELVKEVEERVR
>2plus1_GFP11_Key_Cterm_116 (SEQ ID NO: 26857)
EAKKKLDEVLERAKRTIDRLLETSDRSLEKVEADLRRLNEELDRSLERAERTIRELAK
>2plus1_GFP11_Key_Cterm_117 (SEQ ID NO: 26858)
TAERARETLKRLLDENRDRSKKVKEEIRRILEDLTRTTERVKREIAKLLKELEDTAR
>2plus1_GFP11_Key_Cterm_118 (SEQ ID NO: 26859)
DERILRELEERVKELEKEAREILKRSEDETDKLREKAERILEDLERANRRTMDEARR
>2plus1_GFP11_Key_Cterm_119 (SEQ ID NO: 26860)
DERILRELEERVKELEKEAREILKRSEDETDKLREKAERILEDLERANRRTMDEARR
>2plus1_GFP11_Key_Cterm_120 (SEQ ID NO: 26861)
DLKRVEERAREVSRRNEESMRRVKEDADRVSEANKEVLDRVREEVKRLIEEVRETLR
>2plus1_GFP11_Key_Cterm_121 (SEQ ID NO: 26862)
DKARKVAEVAEKVLRDIDKLDRESKEAFRATNEEIAKLDEDTARVAERVKKAIEDLAK
>2plus1_GFP11_Key_Cterm_122 (SEQ ID NO: 26863)
ELLRRIKKLLDEIKKAIEDSSREIKRLLEESERVMKRSSEDIKRILDDTRRVVEEVRR
>2plus1_GFP11_Key_Cterm_123 (SEQ ID NO: 26864)
SKAIKDVRDIVKKVKDELKEWRDRNKELVDRLSEELKEWLKDVERVLKELTDKDR
>2plus1_GFP11_Key_Cterm_124 (SEQ ID NO: 26865)
DKVERVVREVEKLHEEDRKRLEESTRSVRKLLEELKRELEKSTRSVKALVDELRERVR
>2plus1_GFP11_Key_Cterm_125 (SEQ ID NO: 26866)
DIDKLLKELRDLVEKIKKDLKELLERYEEIVRRIKELLKDLNREAEEVVRRLKEELR
>2plus1_GFP11_Key_Cterm_126 (SEQ ID NO: 26867)
EAKKKLDEVLERAKRTIDRLLETSDRSLEKVEADLRRLNEELDRSLERAERTIRELAK
>2plus1_GFP11_Key_Cterm_127 (SEQ ID NO: 26868)
DKLLKEARDLIREIEKRLEELLKRVEKLTEDAKRDLERSNREHKELADRIKETAR
>2plus1_GFP11_Key_Cterm_128 (SEQ ID NO: 26869)
TAERARETLKRLLDENRDRSKKVKEEIRRILEDLTRTTERVKREIAKLLKELEDTAR
>2plus1_GFP11_Key_Cterm_129 (SEQ ID NO: 26870)
DEIKRIVDEVRERLKRIVDENAKIVEDARRALEKIVKENEEILRRLKKELRELRK
>2plus1_GFP11_Key_Cterm_130 (SEQ ID NO: 26871)
DRIEEELKRLIDTLREKNREVEKRARDSNRDLKRTNDEIAKEVRELIKKLREDLK
>2plus1_GFP11_Key_Cterm_131 (SEQ ID NO: 26872)
DERILRELEERVKELEKEAREILKRSEDETDKLREKAERILEDLERANRRTMDEARR
>2plus1_GFP11_Key_Cterm_132 (SEQ ID NO: 26873)
DAETIERVVRELLEENKEVLRKTEEAVKRSTEINKRLLEASKEVADRLRERIKEAAK
>2plus1_GFP11_Key_Cterm_133 (SEQ ID NO: 26874)
DKARKVAEVAEKVLRDIDKLDRESKEAFRATNEEIAKLDEDTARVAERVKKAIEDLAK
>2plus1_GFP11_Key_Cterm_134 (SEQ ID NO: 26875)
DKVERVVREVEKLHEEDRKRLEESTRSVRKLLEELKRELEKSTRSVKALVDELRERVR
>2plus1_GFP11_Key_Cterm_135 (SEQ ID NO: 26876)
DIERILRELEAVLKKLTDESERLNREVERVSRDTKKKSKELNEELKAVLDEVKRKAD
>2plus1_GFP11_Key_Cterm_136 (SEQ ID NO: 26877)
DEVVERAERISEENKRRVEDVARKSKELVEDVRRHSEEVVRRVEELVKEVEERVR
>2plus1_GFP11_Key_Cterm_137 (SEQ ID NO: 26878)
EAKKKLDEVLERAKRTIDRLLETSDRSLEKVEADLRRLNEELDRSLERAERTIRELAK
>2plus1_GFP11_Key_Cterm_138 (SEQ ID NO: 26879)
TAERARETLKRLLDENRDRSKKVKEEIRRILEDLTRTTERVKREIAKLLKELEDTAR
>2plus1_GFP11_Key_Cterm_139 (SEQ ID NO: 26880)
DEIKRIVDEVRERLKRIVDENAKIVEDARRALEKIVKENEEILRRLKKELRELRK
>2plus1_GFP11_Key_Cterm_140 (SEQ ID NO: 26881)
DRRIEKVLKEIEEKIREVIKEWERVHREVEELLKRLIDENRKVLDEIRKLLEEKSK
>2plus1_GFP11_Key_Cterm_141 (SEQ ID NO: 26882)
DLKRVEERAREVSRRNEESMRRVKEDADRVSEANKEVLDRVREEVKRLIEEVRETLR
>2plus1_GFP11_Key_Cterm_142 (SEQ ID NO: 26883)
DAETIERVVRELLEENKEVLRKTEEAVKRSTETNKRLLEASKEVADRLRERIKEAAK
>2plus1_GFP11_Key_Cterm_143 (SEQ ID NO: 26884)
DKARKVAEVAEKVLRDIDKLDRESKEAFRATNEEIAKLDEDTARVAERVKKAIEDLAK
>2plus1_GFP11_Key_Cterm_144 (SEQ ID NO: 26885)
ELLRRIKKLLDEIKKAIEDSSREIKRLLEESERVMKRSSEDIKRTLDDTRRVVEEVRR
>2plus1_GFP11_Key_Cterm_145 (SEQ ID NO: 26886)
DKVERVVREVEKLHEEDRKRLEESTRSVRKLLEELKRELEKSTRSVKALVDELRERVR
>2plus1_GFP11_Key_Cterm_146 (SEQ ID NO: 26887)
EAKKKLDEVLERAKRTIDRLLETSDRSLEKVEADLRRLNEELDRSLERAERTIRELAK
>2plus1_GFP11_Key_Cterm_147 (SEQ ID NO: 26888)
TAERARETLKRLLDENRDRSKKVKEEIRRILEDLTRTTERVKREIAKLLKELEDTAR
>2plus1_GFP11_Key_Cterm_148 (SEQ ID NO: 26889)
DEIKRIVDEVRERLKRIVDENAKIVEDARRALEKIVKENEEILRRLKKELRELRK
>2plus1_GFP11_Key_Cterm_149 (SEQ ID NO: 26890)
DKARKVAEVAEKVLRDIDKLDRESKEAFRATNEEIAKLDEDTARVAERVKKAIEDLAK
>2plus1_GFP11_Key_Cterm_150 (SEQ ID NO: 26891)
DEVTKVKKVADDVLAEIKKLDDETRRVIEDTNKKIADLDKATRDVVRKVLEEVKKLEK
>2plus1_GFP11_Key_Cterm_151 (SEQ ID NO: 26892)
DIDKLLKELRDLVEKIKKDLKELLERYEEIVRRIKELLKDLNREAEEVVRRLKEELR
>2plus1_GFP11_Key_Cterm_152 (SEQ ID NO: 26893)
TAERARETLKRLLDENRDRSKKVKEEIRRILEDLTRTTERVKREIAKLLKELEDTAR
>2plus1_GFP11_Key_Cterm_153 (SEQ ID NO: 26894)
DEIKRIVDEVRERLKRIVDENAKIVEDARRALEKIVKENEEILRRLKKELRELRK
>2plus1_GFP11_Key_Cterm_154 (SEQ ID NO: 26895)
RLVREVEDLVRRLVRRSEKSNEEVKRTVEELVRRMEESNDRVRDLVRRLVEELKRAVD
>2plus1_GFP11_Key_Cterm_155 (SEQ ID NO: 26896)
DKARKVAEVAEKVLRDIDKLDRESKEAFRATNEEIAKLDEDTARVAERVKKAIEDLAK
>2plus1_GFP11_Key_Cterm_156 (SEQ ID NO: 26897)
EAKKKLDEVLERAKRTIDRLLETSDRSLEKVEADLRRLNEELDRSLERAERTIRELAK
>2plus1_GFP11_Key_Cterm_157 (SEQ ID NO: 26898)
RLVREVEDLVRRLVRRSEKSNEEVKRTVEELVRRMEESNDRVRDLVRRLVEELKRAVD
>2plus1_GFP11_Key_Cterm_158 (SEQ ID NO: 26899)
DKARKVAEVAEKVLRDIDKLDRESKEAFRATNEEIAKLDEDTARVAERVKKAIEDLAK
>2plus1_GFP11_Key_Cterm_159 (SEQ ID NO: 26900)
DEVTKVKKVADDVLAEIKKLDDETRRVIEDTNKKIADLDKATRDVVRKVLEEVKKLEK
>2plus1_GFP11_Key_Cterm_160 (SEQ ID NO: 26901)
TAERARETLKRLLDENRDRSKKVKEEIRRILEDLTRTTERVKREIAKLLKELEDTAR
>2plus1_GFP11_Key_Cterm_161 (SEQ ID NO: 26902)
DLKRVEERAREVSRRNEESMRRVKEDADRVSEANKEVLDRVREEVKRLIEEVRETLR
>2plus1_GFP11_Key_Cterm_162 (SEQ ID NO: 26903)
DKARKVAEVAEKVLRDIDKLDRESKEAFRATNEEIAKLDEDTARVAERVKKAIEDLAK
>2plus1_GFP11_Key_Cterm_163 (SEQ ID NO: 26904)
DEVTKVKKVADDVLAEIKKLDDETRRVIEDTNKKIADLDKATRDVVRKVLEEVKKLEK
>2plus1_GFP11_Key_Cterm_164 (SEQ ID NO: 26905)
DKVERVVREVEKLHEEDRKRLEESTRSVRKLLEELKRELEKSTRSVKALVDELRERVR
>2plus1_GFP11_Key_Cterm_165 (SEQ ID NO: 26906)
EAKKKLDEVLERAKRTIDRLLETSDRSLEKVEADLRRLNEELDRSLERAERTIRELAK
>2plus1_GFP11_Key_Cterm_166 (SEQ ID NO: 26907)
TAERARETLKRLLDENRDRSKKVKEEIRRILEDLTRTTERVKREIAKLLKELEDTAR
>2plus1_GFP11_Key_Cterm_167 (SEQ ID NO: 26908)
TAERARETLKRLLDENRDRSKKVKEEIRRILEDLTRTTERVKREIAKLLKELEDTAR
>2plus1_GFP11_Key_Cterm_168 (SEQ ID NO: 26909)
DKARKVAEVAEKVLRDIDKLDRESKEAFRATNEEIAKLDEDTARVAERVKKAIEDLAK
>2plus1_GFP11_Key_Cterm_169 (SEQ ID NO: 26910)
DEVTKVKKVADDVLAEIKKLDDETRRVIEDTNKKIADLDKATRDVVRKVLEEVKKLEK
>2plus1_GFP11_Key_Cterm_170 (SEQ ID NO: 26911)
DKVERVVREVEKLHEEDRKRLEESTRSVRKLLEELKRELEKSTRSVKALVDELRERVR
>2plus1_GFP11_Key_Cterm_171 (SEQ ID NO: 26912)
TAERARETLKRLLDENRDRSKKVKEEIRRILEDLTRTTERVKREIAKLLKELEDTAR
>2plus1_GFP11_Key_Cterm_172 (SEQ ID NO: 26913)
DKARKVAEVAEKVLRDIDKLDRESKEAFRATNEEIAKLDEDTARVAERVKKAIEDLAK
>2plus1_GFP11_Key_Cterm_173 (SEQ ID NO: 26914)
RLVREVEDLVRRLVRRSEKSNEEVKRTVEELVRRMEESNDRVRDLVRRLVEELKRAVD
>2plus1_GFP11_Key_Nterm_174 (SEQ ID NO: 26915)
SESDEVIRDLARLLDELARHVDDSVRRMDEVVKRSTREADELAKRLDELVKEVEKKPG
>2plus1_GFP11_Key_Nterm_175 (SEQ ID NO: 26916)
SRAETVLKEVTDKIKKLADSSDELLRRNKENIDELKKSSEELLRRLTKAIEEIEKGSV
>2plus1_GFP11_Key_Nterm_176 (SEQ ID NO: 26917)
SVDEVLKEIEDALRRLKEEVERVLKENEDELRRLEEEVRRVLKEDEELLESLKRGVGE
>2plus1_GFP11_Key_Nterm_177 (SEQ ID NO: 26918)
SEVDEIIKELERLLAEIARENERIIRESRKLADEVRKRNEDAIRKLEELVARLADAVR
>2plus1_GFP11_Key_Nterm_178 (SEQ ID NO: 26919)
SEVDDVLRRLEELIKTLEDINAKSLEDIKKLIDDLAKILEDALRKHEKLIRELREAKK
>2plus1_GFP11_Key_Nterm_179 (SEQ ID NO: 26920)
SEVDDVLRRLEELIKTLEDINAKSLEDIKKLIDDLAKILEDALRKHEKLIRELREAKK
>2plus1_GFP11_Key_Nterm_180 (SEQ ID NO: 26921)
SRAETVLKEVTDKIKKLADSSDELLRRNKENIDELKKSSEELLRRLTKAIEEIEKGSV
>2plus1_GFP11_Key_Nterm_181 (SEQ ID NO: 26922)
KEVEDAVKELEDLLRANEDKTRSIVEDMRASNKDLEDHSRASEEEVRKLLDDLRRAGV
>2plus1_GFP11_Key_Nterm_182 (SEQ ID NO: 26923)
SEVDDVLRRLEELIKTLEDINAKSLEDIKKLIDDLAKILEDALRKHEKLIRELREAKK
>2plus1_GFP11_Key_Nterm_183 (SEQ ID NO: 26924)
SESDEVIRDLARLLDELARHVDDSVRRMDEVVKRSTREADELAKRLDELVKEVEKKPG
>2plus1_GFP11_Key_Nterm_184 (SEQ ID NO: 26925)
SESDDVIRKLRELLEELRTHVEKSIRDLRKILEDSTRHAKRSIEELERLLEEVRKKPG
>2plus1_GFP11_Key_Nterm_185 (SEQ ID NO: 26926)
SRAETVLKEVTDKIKKLADSSDELLRRNKENIDELKKSSEELLRRLTKAIEEIEKGSV
>2plus1_GFP11_Key_Nterm_186 (SEQ ID NO: 26927)
SEAEKAKETIDRLADRVRKLLEEIKRSLDDSRRKSKETVEENEKTLDRMRKEVDAAKR
>2plus1_GFP11_Key_Nterm_187 (SEQ ID NO: 26928)
SEAEKAKETIDRLADRVRKLLEEIKRSLDDSRRKSKETVEENEKTLDRMRKEVDAAKR
>2plus1_GFP11_Key_Nterm_188 (SEQ ID NO: 26929)
SEVEELIKRLAKVLKELVDKVRKVIEDTKELLERLKRRSEDHIRKLREVLKEAKDQPI
>2plus1_GFP11_Key_Nterm_189 (SEQ ID NO: 26930)
SELEEIEKKVRELTKRHRELVERVRKTVKELIETNRRLLETLTERIKRVLEEVRDLER
>2plus1_GFP11_Key_Nterm_190 (SEQ ID NO: 26931)
SSEERLRAVIEDLKRLAEESRKRHKELIDELAKAVERIERRHKKLLDEIKAVVDDIRR
>2plus1_GFP11_Key_Nterm_191 (SEQ ID NO: 26932)
DEIRKVVKEITDLLKASNDKNRKVVEEIRDLLRKSKKLADELVERLRALVEDLRRRID
>2plus1_GFP11_Key_Nterm_192 (SEQ ID NO: 26933)
STAETVAEEVERVLKHSDDLIKEVEDVNRRVEEEIKRVIRELEEENERLVAEVRKGVK
>2plus1_GFP11_Key_Nterm_193 (SEQ ID NO: 26934)
SEVDEIIKELERLLAEIARENERIIRESRKLADEVRKRNEDAIRKLEELVARLADAVR
>2plus1_GFP11_Key_Nterm_194 (SEQ ID NO: 26935)
SEIDEVLTRLRKISKDLNETSDRVNERARKIIDDIKKESKRVNDEAREIVERLKREID
>2plus1_GFP11_Key_Nterm_195 (SEQ ID NO: 26936)
SEDEDLDRVAEKLAREHKKSVEEIKRVLKSADEESKKLVRDTERVIEEIKREVEEARR
>2plus1_GFP11_Key_Nterm_196 (SEQ ID NO: 26937)
SSVEELLERLRRISEENKRRIEKLLREVEKVLRELKDRHRKLLKRVEEIIRKVKEEIK
>2plus1_GFP11_Key_Nterm_197 (SEQ ID NO: 26938)
SAADEVVERMKELVATVKRENDEVVKELKKLVKELEDDNRRVVEESKKSVEDLARRVG
>2plus1_GFP11_Key_Nterm_198 (SEQ ID NO: 26939)
SESDEVIRDLARLLDELARHVDDSVRRMDEVVKRSTREADELAKRLDELVKEVEKKPG
>2plus1_GFP11_Key_Nterm_199 (SEQ ID NO: 26940)
KEVEDAVKELEDLLRANEDKTRSIVEDMRASNKDLEDHSRASEEEVRKLLDDLRRAGV
>2plus1_GFP11_Key_Nterm_200 (SEQ ID NO: 26941)
DEIRKVVKEITDLLKASNDKNRKVVEEIRDLLRKSKKLADELVERLRALVEDLRRRID
>2plus1_GFP11_Key_Nterm_201 (SEQ ID NO: 26942)
SRVEEIIEDLRRLLEEIRKENEDSIRRSKELLDRVKEINDTIIAELERLLKDIEKEVR
>2plus1_GFP11_Key_Nterm_202 (SEQ ID NO: 26943)
SRVEEIIEDLRRLLEEIRKENEDSIRRSKELLDRVKEINDTIIAELERLLKDIEKEVR
>2plus1_GFP11_Key_Nterm_203 (SEQ ID NO: 26944)
SEVDDVLRRLEELIKTLEDINAKSLEDIKKLIDDLAKILEDALRKHEKLIRELREAKK
>2plus1_GFP11_Key_Nterm_204 (SEQ ID NO: 26945)
SKLEEVEKAVRKVIEDSRRVNEEVNRRSEEVVRELEKVHREVNDASRRVVEKARRVLK
>2plus1_GFP11_Key_Nterm_205 (SEQ ID NO: 26946)
SESDEVIRDLARLLDELARHVDDSVRRMDEVVKRSTREADELAKRLDELVKEVEKKPG
>2plus1_GFP11_Key_Nterm_206 (SEQ ID NO: 26947)
SESDEVIRDLARLLDELARHVDDSVRRMDEVVKRSTREADELAKRLDELVKEVEKKPG
>2plus1_GFP11_Key_Nterm_207 (SEQ ID NO: 26948)
SESDDVIRKLRELLEELRTHVEKSIRDLRKILEDSTRHAKRSIEELERLLEEVRKKPG
>2plus1_GFP11_Key_Nterm_208 (SEQ ID NO: 26949)
DEVRELLERNRRLLEEIKKTVKDLIRANEELLKRIEDDAKRLIDRNEELLDELEKGLS
>2plus1_GFP11_Key_Nterm_209 (SEQ ID NO: 26950)
DEIRKVVKEITDLLKASNDKNRKVVEEIRDLLRKSKKLADELVERLRALVEDLRRRID
>2plus1_GFP11_Key_Nterm_210 (SEQ ID NO: 26951)
SRAETVLKEVTDKIKKLADSSDELLRRNKENIDELKKSSEELLRRLTKAIEEIEKGSV
>2plus1_GFP11_Key_Nterm_211 (SEQ ID NO: 26952)
DEEEDLERAIKKLLDENRELLKRIAEELRRLLEELRRLTEESADRLRRLLKELKDRGV
>2plus1_GFP11_Key_Nterm_212 (SEQ ID NO: 26953)
DEIRKVVKEITDLLKASNDKNRKVVEEIRDLLRKSKKLADELVERLRALVEDLRRRID
>2plus1_GFP11_Key_Nterm_213 (SEQ ID NO: 26954)
SKEDRLREELKKLLARLAEEIERLKRALEESNKDLKRTIDASEKHLRDVNEDVKRGGV
>2plus1_GFP11_Key_Nterm_214 (SEQ ID NO: 26955)
SESDEVIRDLARLLDELARHVDDSVRRMDEVVKRSTREADELAKRLDELVKEVEKKPG
>2plus1_GFP11_Key_Nterm_215 (SEQ ID NO: 26956)
SESDEVIRDLARLLDELARHVDDSVRRMDEVVKRSTREADELAKRLDELVKEVEKKPG
>2plus1_GFP11_Key_Nterm_216 (SEQ ID NO: 26957)
SESDDVIRKLRELLEELRTHVEKSIRDLRKILEDSTRHAKRSIEELERLLEEVRKKPG
>2plus1_GFP11_Key_Nterm_217 (SEQ ID NO: 26958)
SRVEEIIEDLRRLLEEIRKENEDSIRRSKELLDRVKEINDTIIAELERLLKDIEKEVR
>2plus1_GFP11_Key_Nterm_218 (SEQ ID NO: 26959)
SEVDEIIKELERLLAEIARENERIIRESRKLADEVRKRNEDAIRKLEELVARLADAVR
>2plus1_GFP11_Key_Nterm_219 (SEQ ID NO: 26960)
SSVEELLERLRRISEENKRRIEKLLREVEKVLRELKDRHRKLLKRVEEIIRKVKEEIK
>2plus1_GFP11_Key_Nterm_220 (SEQ ID NO: 26961)
SELEEVLRRIEALVRKAHKENEDVLREIERLVRTAHRLNKKVDDDSAKIAEDLKRGGR
>2plus1_GFP11_Key_Nterm_221 (SEQ ID NO: 26962)
SESDEVIRDLARLLDELARHVDDSVRRMDEVVKRSTREADELAKRLDELVKEVEKKPG
>2plus1_GFP11_Key_Nterm_222 (SEQ ID NO: 26963)
SESDDVIRKLRELLEELRTHVEKSIRDLRKILEDSTRHAKRSIEELERLLEEVRKKPG
>2plus1_GFP11_Key_Nterm_223 (SEQ ID NO: 26964)
SRAETVLKEVTDKIKKLADSSDELLRRNKENIDELKKSSEELLRRLTKAIEEIEKGSV
>2plus1_GFP11_Key_Nterm_224 (SEQ ID NO: 26965)
DEVRELLERNRRLLEEIKKTVKDLIRANEELLKRIEDDAKRLIDRNEELLDELEKGLS
>2plus1_GFP11_Key_Nterm_225 (SEQ ID NO: 26966)
STEEVLDEIRKLHKILTEDIKRVLREIEELHRRTIEENKEVLDKIAEDYKRVIDDVRT
>2plus1_GFP11_Key_Nterm_226 (SEQ ID NO: 26967)
SEIEKILKEIEDLARRDEEVSKKIVEDIRRLAKEVEDTSRDIVRKIEELAKRVLDRLR
>2plus1_GFP11_Key_Nterm_227 (SEQ ID NO: 26968)
SEAERLEARARELLRANEELMDDLRAKAEELLKRNDRLVKEIEKKVREVLAAIEELKR
>2plus1_GFP11_Key_Nterm_228 (SEQ ID NO: 26969)
DDLERAREEVADLIRKHEEKTRRILEESRRLNERHRELSARILDEIRKLAERIEELIK
>2plus1_GFP11_Key_Nterm_229 (SEQ ID NO: 26970)
DEEEDLERAIKKLLDENRELLKRIAEELRRLLEELRRLTEESADRLRRLLKELKDRGV
>2plus1_GFP11_Key_Nterm_230 (SEQ ID NO: 26971)
DEIRKVVKEITDLLKASNDKNRKVVEEIRDLLRKSKKLADELVERLRALVEDLRRRID
>2plus1_GFP11_Key_Nterm_231 (SEQ ID NO: 26972)
DEIRKVVKEITDLLKASNDKNRKVVEEIRDLLRKSKKLADELVERLRALVEDLRRRID
>2plus1_GFP11_Key_Nterm_232 (SEQ ID NO: 26973)
STAETVEKKVEEVIRENEKSMRESEEKVDRSTKRIEDVLRRLEETIRKTSDDIAKGVK
>2plus1_GFP11_Key_Nterm_233 (SEQ ID NO: 26974)
SRVEEIIEDLRRLLEEIRKENEDSIRRSKELLDRVKEINDTIIAELERLLKDIEKEVR
>2plus1_GFP11_Key_Nterm_234 (SEQ ID NO: 26975)
SRVEEIIEDLRRLLEEIRKENEDSIRRSKELLDRVKEINDTIIAELERLLKDIEKEVR
>2plus1_GFP11_Key_Nterm_235 (SEQ ID NO: 26976)
REVEEMIKELEELLKDLKEKNERASKRNRELVRRLEEENKRVIEELKKLVKELEDLVR
>2plus1_GFP11_Key_Nterm_236 (SEQ ID NO: 26977)
SEVDDVLRRLEELIKTLEDINAKSLEDIKKLIDDLAKILEDALRKHEKLIRELREAKK
>2plus1_GFP11_Key_Nterm_237 (SEQ ID NO: 26978)
SKLEEVEKAVRKVIEDSRRVNEEVNRRSEEVVRELEKVHREVNDASRRVVEKARRVLK
>2plus1_GFP11_Key_Nterm_238 (SEQ ID NO: 26979)
DEVEDVLRKIEKILDDHRKRIEKNSRDMARIIDEHRRKVEENSREMKKLVDDLKKAVD
>2plus1_GFP11_Key_Nterm_239 (SEQ ID NO: 26980)
SSVEELLERLRRISEENKRRIEKLLREVEKVLRELKDRHRKLLKRVEEIIRKVKEEIK
>2plus1_GFP11_Key_Nterm_240 (SEQ ID NO: 26981)
DEVEKVLEEIKRALDDLRKKVEESKREIKEALKAVEKHTRDSDTANKRTLAEIERGVK
>2plus1_GFP11_Key_Nterm_241 (SEQ ID NO: 26982)
SESDEVIRDLARLLDELARHVDDSVRRMDEVVKRSTREADELAKRLDELVKEVEKKPG
>2plus1_GFP11_Key_Nterm_242 (SEQ ID NO: 26983)
KEVEDAVKELEDLLRANEDKTRSIVEDMRASNKDLEDHSRASEEEVRKLLDDLRRAGV
>2plus1_GFP11_Key_Nterm_243 (SEQ ID NO: 26984)
DEIRKVVKEITDLLKASNDKNRKVVEEIRDLLRKSKKLADELVERLRALVEDLRRRID
>2plus1_GFP11_Key_Nterm_244 (SEQ ID NO: 26985)
STAETVAEEVERVLKHSDDLIKEVEDVNRRVEEEIKRVIRELEEENERLVAEVRKGVK
>2plus1_GFP11_Key_Nterm_245 (SEQ ID NO: 26986)
SRVEEIIEDLRRLLEEIRKENEDSIRRSKELLDRVKEINDTIIAELERLLKDIEKEVR
>2plus1_GFP11_Key_Nterm_246 (SEQ ID NO: 26987)
SEVDEIIKELERLLAEIARENERIIRESRKLADEVRKRNEDAIRKLEELVARLADAVR
>2plus1_GFP11_Key_Nterm_247 (SEQ ID NO: 26988)
SEVDDVLRRLEELIKTLEDINAKSLEDIKKLIDDLAKILEDALRKHEKLIRELREAKK
>2plus1_GFP11_Key_Nterm_248 (SEQ ID NO: 26989)
SKLEEVEKAVRKVIEDSRRVNEEVNRRSEEVVRELEKVHREVNDASRRVVEKARRVLK
>2plus1_GFP11_Key_Nterm_249 (SEQ ID NO: 26990)
SAEEVKEELKRIATKLKEEIKENIRRLEESVEKIAKELAENIKRLEDILRDVKRGLRD
>2plus1_GFP11_Key_Nterm_250 (SEQ ID NO: 26991)
SDVDRVLEEIRKLLEDLKRHSEKVSEENEDLLRANTELNKRVSEDNERLLEELKRLRE
>2plus1_GFP11_Key_Nterm_251 (SEQ ID NO: 26992)
DEVEDVLRKIEKILDDHRKRIEKNSRDMARIIDEHRRKVEENSREMKKLVDDLKKAVD
>2plus1_GFP11_Key_Nterm_252 (SEQ ID NO: 26993)
SSVEELLERLRRISEENKRRIEKLLREVEKVLRELKDRHRKLLKRVEEIIRKVKEEIK
>2plus1_GFP11_Key_Nterm_253 (SEQ ID NO: 26994)
DREREVKKRLDEVRERIERLLRRVEEESRRVAEEIRRLIEEVRRRNKKVTEEIRELLK
>2plus1_GFP11_Key_Nterm_254 (SEQ ID NO: 26995)
SRAETVLKEVIDKIKKLADSSDELLRRNKENIDELKKSSEELLRRLTKAIEEIEKGSV
>2plus1_GFP11_Key_Nterm_255 (SEQ ID NO: 26996)
SEIEKILKEIEDLARRDEEVSKKIVEDIRRLAKEVEDTSRDIVRKIEELAKRVLDRLR
>2plus1_GFP11_Key_Nterm_256 (SEQ ID NO: 26997)
SEAEKAKETIDRLADRVRKLLEEIKRSLDDSRRKSKETVEENEKTLDRMRKEVDAAKR
>2plus1_GFP11_Key_Nterm_257 (SEQ ID NO: 26998)
SEVEELIKRLAKVLKELVDKVRKVIEDTKELLERLKRRSEDHIRKLREVLKEAKDQPI
>2plus1_GFP11_Key_Nterm_258 (SEQ ID NO: 26999)
DEIRKVVKEITDLLKASNDKNRKVVEEIRDLLRKSKKLADELVERLRALVEDLRRRID
>2plus1_GFP11_Key_Nterm_259 (SEQ ID NO: 27000)
DEIRKVVKEITDLLKASNDKNRKVVEEIRDLLRKSKKLADELVERLRALVEDLRRRID
>2plus1_GFP11_Key_Nterm_260 (SEQ ID NO: 27001)
STAETVEKKVEEVIRENEKSMRESEEKVDRSTKRIEDVLRRLEETIRKTSDDIAKGVK
>2plus1_GFP11_Key_Nterm_261 (SEQ ID NO: 27002)
SRVEEIIEDLRRLLEEIRKENEDSIRRSKELLDRVKEINDTIIAELERLLKDIEKEVR
>2plus1_GFP11_Key_Nterm_262 (SEQ ID NO: 27003)
REVEEMIKELEELLKDLKEKNERASKRNRELVRRLEEENKRVIEELKKLVKELEDLVR
>2plus1_GFP11_Key_Nterm_263 (SEQ ID NO: 27004)
DAVEEAEKLIRKVIADSEKLLRDLADLNAKSIRRSEKLVEDDRRANEDVIRKLEELRR
>2plus1_GFP11_Key_Nterm_264 (SEQ ID NO: 27004)
SEVDDVLRRLEELIKTLEDINAKSLEDIKKLIDDLAKILEDALRKHEKLIRELREAKK
>2plus1_GFP11_Key_Nterm_265 (SEQ ID NO: 27006)
SEIERVKKRLEELLAEVEESTRRLEERLKRLLEEAKRSSEEVEKELRRLLEAVRRGLS
>2plus1_GFP11_Key_Nterm_266 (SEQ ID NO: 27007)
SDVDRVLEEIRKLLEDLKRHSEKVSEENEDLLRANTELNKRVSEDNERLLEELKRLRE
>2plus1_GFP11_Key_Nterm_267 (SEQ ID NO: 27008)
DEVEDVLRKIEKILDDHRKRIEKNSRDMARIIDEHRRKVEENSREMKKLVDDLKKAVD
>2plus1_GFP11_Key_Nterm_268 (SEQ ID NO: 27009)
SESDEVIRDLARLLDELARHVDDSVRRMDEVVKRSTREADELAKRLDELVKEVEKKPG
>2plus1_GFP11_Key_Nterm_269 (SEQ ID NO: 27010)
SRAETVLKEVTDKIKKLADSSDELLRRNKENIDELKKSSEELLRRLTKAIEEIEKGSV
>2plus1_GFP11_Key_Nterm_270 (SEQ ID NO: 27011)
DEEEDLERAIKKLLDENRELLKRIAEELRRLLEELRRLTEESADRLRRLLKELKDRGV
>2plus1_GFP11_Key_Nterm_271 (SEQ ID NO: 27012)
DEIRKVVKEITDLLKASNDKNRKVVEEIRDLLRKSKKLADELVERLRALVEDLRRRID
>2plus1_GFP11_Key_Nterm_272 (SEQ ID NO: 27013)
DAVEEAEKLIRKVIADSEKLLRDLADLNAKSIRRSEKLVEDDRRANEDVIRKLEELRR
>2plus1_GFP11_Key_Nterm_273 (SEQ ID NO: 27014)
SEDEDLDRVAEKLAREHKKSVEEIKRVLKSADEESKKLVRDTERVIEEIKREVEEARR
>2plus1_GFP11_Key_Nterm_274 (SEQ ID NO: 27015)
SKEDRLREELKKLLARLAEEIERLKRALEESNKDLKRTIDASEKHLRDVNEDVKRGGV
>3plus1_Key_668_Nterm (SEQ ID NO: 27,322)
DEAKELLDEIRKAVKESEDRLEKLLRDYEKELRRLEKELRDLKRRIEEKLEELRRGSL
>3plus1_Key_668_Cterm (SEQ ID NO: 27,323)
RGADALSRLLEELLRVVDDLIRVLKELIDKSRKVIEELLELLKRINEENLKVLAEIIK
>3plus1_Key_668_Cterm (SEQ ID NO: 27,324)
SEKEKLLKESEEEVRRLRRTLEELLRKYREVLERLRKELREIEERVRDVVRRLKEVLD
>3plus1_Key_668_Cterm (SEQ ID NO: 27,325)
SEKEDAARKLRKLVEELTREYEELVKKLERLIEEIEKVSEESVRKLEKLLAEISEEVR
>3plus1_Key_668_Cterm (SEQ ID NO: 27,326)
EALRKLVELLVEVLRRLIRVNRELVKLLREVLERLLRILRESVKKLKRLIEKVIKDAT
>3plus1_Key_669_Nterm (SEQ ID NO: 27,327)
RAVKKLDEIVKEVAKKLEDVVRANEELWRALVELNKESVRRLREIVERVARDLEETAR
>3plus1_Key_670_Nterm (SEQ ID NO: 27,328)
SDERRIAERIRELLRESKKLVRDVVEEAKRLLKENRDSTRKIIEDIRRLLRKIEDSTR
>3plus1_Key_670_Cterm (SEQ ID NO: 27,329)
SKEETLRKEAEDLLRRLEELTRRLEKKARELLERAKKLSRDLAEELKRLLKELREKGV
>3plus1_Key_670_Cterm (SEQ ID NO: 27,330)
AAKRLVEELLKAVTDLSRKNKRILEELLKAIETLSDENKKILTEILDALRRLVEKIEK
>3plus1_Key_670_Cterm (SEQ ID NO: 27,331)
KEREEVKEKLDRLLEEVEKTVRELKREHDELLKEVEKLVRDLKKEHDELLKKVKDDGV
>3plus1_Key_670_Nterm (SEQ ID NO: 27,332)
DRLDKVEELVKKLLEDTKRTVDRVRELVRKILKKSRETLEELERLIEKILRELEKDAR
>3plus1_Key_670_Cterm (SEQ ID NO: 27,333)
SERETVKRRLEELLKEVKRTLDKLKEEHDRLLEDVRRVVEELKREHDKLLKEVKDSGV
>3plus1_Key_670_Nterm (SEQ ID NO: 27,334)
SEDEIIKKIIEDLRRVLKEVEEIHKEVEERLDKVLKEAEEMHKEVLKELDRVLDEVKR
>3plus1_Key_670_Nterm (SEQ ID NO: 27,335)
SREEVLRELEEVIEDNRRLLEELIEKSKKVLDESLKLIDELLRRLEEVLERVLRLLEE
>3plus1_Key_670_Nterm (SEQ ID NO: 27,336)
ISEDDLKRVVDEVEKKLRELKRRYAEALERIKEKIKELKDRYERAVREVVAELRKTTK
>3plus1_Key_670_Nterm (SEQ ID NO: 27,337)
SKAEEIAEKLDRLLEENRRALEEITTRLDDLLRRNKDALRKVMEKLKRLLDDLRRGGI
>3plus1_Key_671_Cterm (SEQ ID NO: 27,338)
VDSERVKEILERILRVVEEAVRLNEESLRRILDVVRKAVKLDRESLKKILDVVEEAVR
>3plus1_Key_671_Cterm (SEQ ID NO: 27,339)
VKDDEVEREIRRVKEDLDRILEEYRRLLEEIKRKLEEILRRVEELHRRLRRKLEEIDR
>3plus1_Key_671_Cterm (SEQ ID NO: 27,340)
SRVEELKKLIEDILRISREVVERIKRVAEDIHRINRRVLDDLRKLIEDILRTVEEILA
>3plus1_Key_671_Cterm (SEQ ID NO: 27,341)
RGADALSRLLEELLRVVDDLIRVLKELIDKSRKVIEELLELLKRINEENLKVLAEIIK
>3plus1_Key_672_Cterm (SEQ ID NO: 27,342)
RGADALSRLLEELLRVVDDLIRVLKELIDKSRKVIEELLELLKRINEENLKVLAEIIK
>3plus1_Key_67>3_Nterm (SEQ ID NO: 27,343)
EALRKLVELLVEVLRRLIRVNRELVKLLREVLERLLRILRESVKKLKRLIEKVIKDAT
>3plus1_Key_67>3_Cterm (SEQ ID NO: 27,344)
SEKEDAARKLRKLVEELTREYEELVKKLERLIEEIEKVSEESVRKLEKLLAEISEEVR
>3plus1_Key_67>3_Nterm (SEQ ID NO: 27,345)
SEKEDAARKLRKLVEELTREYEELVKKLERLIEEIEKVSEESVRKLEKLLAEISEEVR
>3plus1_Key_674_Nterm (SEQ ID NO: 27,346)
SEKEDAARKLRKLVEELTREYEELVKKLERLIEEIEKVSEESVRKLEKLLAEISEEVR
>3plus1_Key_674_Cterm (SEQ ID NO: 27,347)
RAVKKLDEIVKEVAKKLEDVVRANEELWRALVELNKESVRRLREIVERVARDLEETAR
>3plus1_Key_675_Nterm (SEQ ID NO: 27,348)
RAVKKLDEIVKEVAKKLEDVVRANEELWRALVELNKESVRRLREIVERVARDLEETAR
>3plus1_Key_676_Nterm (SEQ ID NO: 27,349)
RAVKKLDEIVKEVAKKLEDVVRANEELWRALVELNKESVRRLREIVERVARDLEETAR
>3plus1_Key_677_Nterm (SEQ ID NO: 27,350)
SDERRIAERIRELLRESKKLVRDVVEEAKRLLKENRDSTRKIIEDIRRLLRKIEDSTR
>3plus1_Key_677_Cterm (SEQ ID NO: 27,351)
SKEETLRKEAEDLLRRLEELTRRLEKKARELLERAKKLSRDLAEELKRLLKELREKGV
>3plus1_Key_678_Nterm (SEQ ID NO: 27,352)
SKEETLRKEAEDLLRRLEELTRRLEKKARELLERAKKLSRDLAEELKRLLKELREKGV
>3plus1_Key_678_Cterm (SEQ ID NO: 27,353)
SKEETLRKEAEDLLRRLEELTRRLEKKARELLERAKKLSRDLAEELKRLLKELREKGV
>3plus1_Key_678_Cterm (SEQ ID NO: 27,354)
ISEDDLKRVVDEVEKKLRELKRRYAEALERIKEKIKELKDRYERAVREVVAELRKTTK
>3plus1_Key_678_Nterm (SEQ ID NO: 27,355)
SKAEEIAEKLDRLLEENRRALEEITTRLDDLLRRNKDALRKVMEKLKRLLDDLRRGGI
>3plus1_Key_678_Nterm (SEQ ID NO: 27,356)
VDSERVKEILERILRVVEEAVRLNEESLRRILDVVRKAVKLDRESLKKILDVVEEAVR
>3plus1_Key_679_Cterm (SEQ ID NO: 27,357)
SKAEEIAEKLDRLLEENRRALEEITTRLDDLLRRNKDALRKVMEKLKRLLDDLRRGGI
>3plus1_Key_679_Nterm (SEQ ID NO: 27,358)
SKAEEIAEKLDRLLEENRRALEEITTRLDDLLRRNKDALRKVMEKLKRLLDDLRRGGI

TABLE 6

| Row | | |
|---|---|---|
| number | Cage (column 1) | Key (column 2) |
| 1 | SB76L (SEQ ID NO: 1), | SB76_C-helix (SEQ ID NO: 27016), |
| | SB76L_17 (SEQ ID NO: 2), | SB76_C-helix-biotin (SEQ ID NO: 27017), |
| | SB76L_18 (SEQ ID NO: 3), | p5_MBP (SEQ ID NO: 27018), |
| | LOCKR_extend5 (SEQ ID NO: 4), | p9_MBP (SEQ ID NO: 27019), |
| | LOCKR_extend9 (SEQ ID NO: 5), | p18_MBP (SEQ ID NO: 27020), |
| | LOCKR_extend18 (SEQ ID NO: 6), | p76-long (SEQ ID NO: 27027), |
| | miniLOCKRa_1 (SEQ ID NO: 12), | p76-short (SEQ ID NO: 27028), |
| | miniLOCKRa_2 (SEQ ID NO: 13), | k76-long (SEQ ID NO: 27029), |
| | aBcl2LOCKR (SEQ ID NO: 18), | k76-short (SEQ ID NO: 27030), |
| | pBimLOCKR (SEQ ID NO: 19), | p76_GLISE (SEQ ID NO: 27031), |
| | BimLOCKR_extend5 (SEQ ID NO: 20), | p76_GSSEKIS (SEQ ID NO: 27032), |
| | BimLOCKR_extend9 (SEQ ID NO: 21), | p76_R26G (SEQ ID NO: 27033), |
| | BimLOCKR_extend18 (SEQ ID NO: 22), | p76-short_E19G (SEQ ID NO: 27034), |
| | strepLOCKRa (all variants; SEQ ID NOs: 26-34), | p76-short_GLISE_E01_EGFR (SEQ ID NO: 27035), |
| | SB13_LOCKR (SEQ ID NO: 35), | p76-short_AE_EGFR (SEQ ID NO: 27036), |
| | SB13_LOCKR_extend18 (SEQ ID NO: 37), | p76-short_AAE_EGFR (SEQ ID NO: 27037), |
| | ZCX12_LOCKR (SEQ ID NO: 36), | p76-short_EE_EGFR (SEQ ID NO: 27038) |
| | ZCX12_LOCKR_extend18 (SEQ ID NO: 38), | |
| | fretLOCKRa (SEQ ID NO: 39), | |
| | 1fix-latch_Mad1SID_t0_1 (SEQ ID NO: 61), | |
| | 1fix-latch_Mad1SID_T0_2 (SEQ ID NO: 65), | |
| | 1fix-long-Bim-t0 (SEQ ID NO: 54), | |
| | 1fix-long-GFP-t0 (SEQ ID NO: 55), | |
| | 1fix-short-BIM-t0 (SEQ ID NO: 56), | |
| | 1fix-short-GFP-t0 (SEQ ID NO: 57), | |
| | 1fix-short-noBim-t0 (SEQ ID NO: 16), | |
| | 1fix-short-noBim(AYYA)-t0 (SEQ ID NO: 17), | |
| | 1fix-short-Bim-t0-relooped (SEQ ID NO: 67), | |
| | 1fix-short-spytag-t0_2 (SEQ ID NO: 68), | |
| | 1fix-short-spytag-t0_8 (SEQ ID NO: 69), | |
| | 1fix-short-TEV-t0_1 (SEQ ID NO: 70), | |
| | 1fix-short-TEV-t0_6 (SEQ ID NO: 71), | |
| | 1fix-short-nanoBit-t0_1 (SEQ ID NO: 72), | |
| | 1fix-short-nanoBit-t0_3 (SEQ ID NO: 73), | |
| | 1fix-short-RHIM-t0_8 (SEQ ID NO: 74), | |
| | 1fix-short-RHIM-t0_19 (SEQ ID NO: 75), | |
| | 1fix-short-RHIM-t0_22 (SEQ ID NO: 76), | |
| | 1fix-short-gcn4-t0_4 (SEQ ID NO: 77), | |
| | 1fix-short-ccDi-t0_6 (SEQ ID NO: 78), | |
| | 1fix-short-cc-a-t0_6 (SEQ ID NO: 79), | |
| | 1fix-short-cc-b-t0_6 (SEQ ID NO: 80) | |
| 2 | LOCKRb (SEQ ID NO: 7), | key_b (SEQ ID NO: 27022) |
| | BimLOCKRb (SEQ ID NO: 23), | |
| | fretLOCKRb (SEQ ID NO: 40) | |
| 3 | LOCKRc (SEQ ID NO: 8), | key_c (SEQ ID NO: 27023) |
| | miniLOCKRc_1 (SEQ ID NO: 14), | |
| | miniLOCKRc_2 (SEQ ID NO: 15), | |
| | BimLOCKRc (SEQ ID NO: 24), | |
| | fretLOCKRc (SEQ ID NO: 41) | |
| 4 | LOCKRd (SEQ ID NO: 9), | key_d (SEQ ID NO: 27024) |
| | BimLOCKRd (SEQ ID NO: 25), | |
| | fretLOCKRd (SEQ ID NO: 42) | |
| 5 | LOCKRe (SEQ ID NO: 10) | key_e (SEQ ID NO: 27025) |
| 6 | LOCKRf (SEQ ID NO: 11) | key_f (SEQ ID NO: 27026) |
| 7 | 1fix_VMAc_C_BIMlatcht9 (SEQ ID 51) | sfGFP_VMAn_p18 (SEQ ID NO: 27041) |
| | sfGFP_VMAn_1fix_BIM_t0_latch (SEQ ID NO: 52) | p18_VMAc_mCherry (SEQ ID NO: 27042) |
| | sfGFP_VMAn_1fix_BIM_t0_latch (SEQ ID NO: 53) | |
| 8 | Spycatcher-1fix-long-GFP-t0 (SEQ ID NO: 58), | p76-spytag (SEQ ID NO: 27039), |
| | Spycatcher-1fix-short-GFP-t0 (SEQ ID NO: 59) | p76-short-spytag (SEQ ID NO: 27040) |
| 9 | STREPII-2plus1_LOCK_1 (SEQ ID NO: 81) | 2plus1_Key_1 (SEQ ID NO: 27043) |
| 10 | STREPII-2plus1_LOCK_2 (SEQ ID NO: 82) | 2plus1_Key_2 (SEQ ID NO: 27044) |
| 11 | STREPII-2plus1_LOCK_3, (SEQ ID NO: 83) | 2plus1_Key_3 (SEQ ID NO: 27045) |
| | STREPII-2plus1_LOCK_3-relooped (SEQ ID NO: 91) | |
| 12 | STREPII-2plus1_LOCK_4C (SEQ ID NO: 84) | 2plus1_Key_4C (SEQ ID NO: 27046) |
| 13 | STREPII-3plus1_LOCK_1 (SEQ ID NO: 86) | 3plus1_Key_1 (SEQ ID NO: 27047) |
| 14 | STREPII-3plus1_LOCK_2 (SEQ ID NO: 87) | 3plus1_Key_2 (SEQ ID NO: 27048) |
| 15 | STREPII-3plus1_LOCK_3 (SEQ ID NO: 88), | 3plus1_Key_3 SEQ ID NO: 27049) |
| | STREPII-3plus1_LOCK_3-relooped (SEQ ID NO: 90) | |
| 16 | STREPII-3plus1_LOCK_4 (SEQ ID NO: 89) | 3plus1_Key_4 (SEQ ID NO: 27050) |

TABLE 7

| Cage Name | Cage Sequence | Key Name | Key Sequence |
|---|---|---|---|
| 2plus1_Cage_Cterm_2406 | SEVDEVVKEVEDLVRRNEELVEEVVRRVEKVVTDDRRLVEEVVREI | 2plus1_Key_Cterm_2406 | EKVLRKLEKVIREVRERSTRALRKVE |
| | RKIVKDVEDLARKLDKEELKRVLDEMRERIERLLEKLRRHSKKLDD | | EVIRRVREESERALRDLERVVKEVEK |
| | ELKRLLEELREHSRRVEKRLEDLLKELRERGVDEKVLRKLEKVIRE | | RMREAAR (SEQ ID NO: 27127) |
| | VRERSTRALRKVEEVIRRVREESERALRDLERVVKEVEKRMREAAR | | |
| | (SEQ ID NO: 27126) | | |
| 2plus1_Cage_Cterm_5398 | SVEELLRKLEEVLRKIREENERSLKELRDRAREIVKRNRETNRELE | 2plus1_Key_Cterm_5398 | EDIVRKIERIVETIEREVRESVKKVE |
| | EVIKELEKRLSGADKEKVEELVRRIRRIVERVVEEDRRTVEEIEKI | | EIARDIRRKVDESVKNVEKLLRDVDK |
| | AREVVKRDRDSADRVRRTVEDVLRKATGSEDIVRKIERIVETIERE | | KARDRKK (SEQ ID NO: 27129) |
| | VRESVKKVEEIARDIRRKVDESVKNVEKLLRDVDKKARDRKK (SEQ ID | | |
| | NO: 27128) | | |
| 2plus1_Cage_Cterm_5405 | SESDDVIRKLRELLEELRTHVEKSIRDLRKILEDSTRHAKRSIEEL | 2plus1_Key_Cterm_5405 | EEKLKDLIRKLRDILRRAAEAHKKLI |
| | ERLLEEVRKKPGDEEVRKTVEEISRRVAENVKRLEDLYRRMEEEVK | | DDARESLERAKREHEKLIDRLKKILE |
| | KNLDRLRKRVEDIIREVEEARKKGVDEEKLKDLIRKLRDILRRAAE | | ELER (SEQ ID NO: 27131) |
| | AHKKLIDDARESLERAKREHEKLIDRLKKILEELER (SEQ ID | | |
| | NO: 27130) | | |
| 2plus1_Cage_Cterm_5406 | DREREVKKRLDEVRERIERLLRRVEEESRRVAEEIRRLIEEVRRRN | 2plus1_Key_Cterm_5406 | EELREELKKLERKIEKVAKEIHDHDK |
| | KKVTEEIRELLKGLKDKEEVRRVLERLRKLNAESDELLERILERLR | | EVTERLEDLLRRITEHARKSDREIEE |
| | RLVEATNRLVKAIIEELRRLVEKIVREVPDSEELREELKKLERKIE | | TAR (SEQ ID NO: 27133) |
| | KVAKEIHDHDKEVTERLEDLLRRITEHARKSDREIEETAR (SEQ ID | | |
| | NO: 27132) | | |
| 2plus1_Cage_Cterm_5409 | SEAEELLKRLEDRAEEILRRLEEILRTSRKLAEDVLRELEKLLRES | 2plus1_Key_Cterm_5409 | KEVVDEIKRIVDEVRERLKRIVDENA |
| | ERRIREVLEELRGIKDKKELEDVIREVEKELDESLERSRELLKDVL | | KIVEDARRALEKIVKENEEILRRLKK |
| | KKLDDNLKESERLVEDIDRELAKILEDLKKAGVPKEVVDEIKRIVD | | ELRELRK (SEQ ID NO: 27135) |
| | EVRERLKRIVDENAKIVEDARRALEKIVKENEEILRRLKKELRELR | | |
| | K (SEQ ID NO: 27134) | | |
| 2plus1_Cage_Cterm_5410 | SRAETVLKEVTDKIKKLADSSDELLRRNKENIDELKKSSEELLRRL | 2plus1_Key_Cterm_5410 | DEVVKRVRDLLDTVRRRNEKVNEDVK |
| | TKAIEEIEKGSVDEETLEELIRRVEAELEAHHRELEKNSREDEKRN | | RMNDKLRRDNEDVIRRVEKLLRELEE |
| | RDHHAKLEEEMRRVEERLEREGIDDEVVKRVRDLLDTVRRRNEKVN | | KRRT (SEQ ID NO: 27137) |
| | EDVKRMNDKLRRDNEDVIRRVEKLLRELEEKRRT (SEQ ID | | |
| | NO: 27136) | | |
| 2plus1_Cage_Cterm_5413 | STEEVLDEIRKLHKTLTEDIKRVLREIEELHRRTIEENKEVLDKIA | 2plus1_Key_Cterm_5413 | AEELLRESKEAIKEVKRVLEELRKES |
| | EDYKRVIDDVRTKDTPNVEKLLKDLEKSAKENIEHNERTLREDDRV | | KRVVDETRKLSEENLEHSERVLRKVE |
| | LKEIRRRATELLKANEEMLRRIEEVARKGGVDAEELLRESKEAIKE | | EDLR (SEQ ID NO: 27139) |
| | VKRVLEELRKESKRVVDETRKLSEENLEHSERVLRKVEEDLR (SEQ ID | | |
| | NO: 27138) | | |
| 2plus1_Cage_5414_GFP11_Cterm | SEIEKILKEIEDLARRDEEVSKKIVEDIRRLAKEVEDTSRDIVRKI | 2plus1_Key_Cterm_5414 | EDSERLVREVEDLVRRLVRRSEKSNE |
| | EELAKRVLDRLRKDGSKEELEKEVREVVKTLEELVKDNHRLIRRAV | | EVKRTVEELVRRMEESNDRVRDLVRR |
| | EEMKRLVEENHRHSREVVKELEDLVRELRKGSGSEDSERDHMVLHE | | LVEELKRAVD (SEQ ID NO: 27141) |
| | YVNAAGITSEKSNEEVKRTVEELVRRMEESNDRVRDLVRRLVEELK | | |
| | RAVD (SEQ ID NO: 27140) | | |
| 2plus1_Cage_5414_GFP11_Cterm | SEIEKILKEIEDLARRDEEVSKKIVEDIRRLAKEVEDTSRDIVRKI | 2plus1_Key_Cterm_5414 | EDSERLVREVEDLVRRLVRRSEKSNE |
| | EELAKRVLDRLRKDGSKEELEKEVREVVKTLEELVKDNHRLIRRAV | | EVKRTVEELVRRMEESNDRVRDLVRR |
| | EEMKRLVEENHRHSREVVKELEDLVRELRKGSGSEDSERRDHMVLH | | LVEELKRAVD (SEQ ID NO: 27143) |
| | EYVNAAGITEKSNEEVKRTVEELVRRMEESNDRVRDLVRRLVEELK | | |
| | RAVD (SEQ ID NO: 27142) | | |
| 2plus1_Cage_5414_GFP11_Cterm | SEIEKILKEIEDLARRDEEVSKKIVEDIRRLAKEVEDTSRDIVRKI | 2plus1_Key_Cterm_5414 | EDSERLVREVEDLVRRLVRRSEKSNE |
| | EELAKRVLDRLRKDGSKEELEKEVREVVKTLEELVKDNHRLIRRAV | | EVKRTVEELVRRMEESNDRVRDLVRR |
| | EEMKRLVEENHRHSREVVKELEDLVRELRKGSGSEDSERLVREVRD | | LVEELKRAVD (SEQ ID NO: 27145) |
| | HMVLHEYVNAAGITEVKRTVEELVRRMEESNDRVRDLVRRLVEELK | | |
| | RAVD (SEQ ID NO: 27144) | | |
| 2plus1_Cage_Cterm_5421 | SVDEVLKEIEDALRRLKEEVERVLKENEDELRRLEEEVRRVLKEDE | 2plus1_Key_Cterm_5421 | EKAIRDVAKEIRDRLKELEEEIEEVT |
| | ELLESLKRGVGESDEVDRVVDEIAKLSAEILEKVKKVVKEIRDSLE | | RRNLKLLADVEEEIRRVHEKTRRLLE |
| | TVKRRVDDVVRRLKELLDEIKRGSDEKAIRDVAKEIRDRLKELEEE | | TVLRRAT (SEQ ID NO: 27147) |
| | IEEVTRRNLKLLADVEEEIRRVHEKTRRLLETVLRRAT (SEQ ID | | |
| | NO: 27146) | | |
| 2plus1_Cage_Cterm_5432 | DEIRKVVKEITDLLKASNDKNRKVVEEIRDLLRKSKKLADELVERL | 2plus1_Key_Cterm_5432 | SEDLKRVEERAREVSRRNEESMRRVK |
| | RALVEDLRRRIDKSGDKETAEDIVRRIIEELKRILKEIEDLARRIN | | EDADRVSEANKEVLDRVREEVKRLIE |
| | REIERLVEEVERDNRDVNRAIEELLKDIARRGGSEDLKRVEERARE | | EVRETLR (SEQ ID NO: 27149) |
| | VSRRNEESMRRVKEDADRVSEANKEVLDRVREEVKRLIEEVRETLR | | |
| | (SEQ ID NO: 27148) | | |
| 2plus1_Cage_Cterm_5435 | STAETVAEEVERVLKHSDDLIKEVEDVNRRVEEEIKRVIRELEEEN | 2plus1_Key_Cterm_5435 | EEAAREIIKRLREVNKRTKEKLDELI |
| | ERLVAEVRKGVKGEILAEIEKRLADNSEKVREVAERAKKLLEENTA | | KHSEEVLERVKRLIDELRKHSEEVLE |
| | RVKDILRESRKLVKDLLDEVRGTGSEEAAREIIKRLREVNKRIKEK | | DLRRRAK (SEQ ID NO: 27151) |
| | LDELIKHSEEVLERVKRLIDELRKHSEEVLEDLRRRAK (SEQ ID | | |
| | NO: 27150) | | |
| 2plus1_Cage_Cterm_5437 | DEVREVAERLRRLVDESRKRNEEVIKESEALVDRVRKTNEEVMKRL | 2plus1_Key_Cterm_5437 | AETIERVVRELLEENKEVLRKTEEAV |
| | RELIDKLEKDIRRSGDKETVEKIIREVLSAIDELLKRVERTNAEIS | | KRSTETNKRLLEASKEVADRLRERIK |
| | KENARLLDEVRKTNEEISRRLAKLLEDIRRGSGDAETIERVVRELL | | EAAK (SEQ ID NO: 27153) |
| | EENKEVLRKTEEAVKRSTETNKRLLEASKEVADRLRERIKEAAK | | |
| | (SEQ ID NO: 27152) | | |
| 2plus1_Cage_Cterm_5439 | SRVEEIIEDLRRLLEEIRKENEDSIRRSKELLDRVKEINDTIIAEL | 2plus1_Key_Cterm_5439 | EDKARKVAEVAEKVLRDIDKLDRESK |
| | ERLLKDIEKEVREKGSESEEVKKALRAVLEELEKLLRRVAEINEEV | | EAFRATNEEIAKLDEDTARVAERVKK |
| | LRRNSKLVEEDERRNREVLKELARLVEELIREIGDEDKARKVAEVA | | AIEDLAK (SEQ ID NO: 27155) |
| | EKVLRDIDKLDRESKEAFRATNEEIAKLDEDTARVAERVKKAIEDL | | |
| | AK (SEQ ID NO: 27154) | | |
| 2plus1_Cage_Cterm_5447 | SEADDVLKKLAETVKRIIERLKKLTDDSRRLVEEVHRRNDKLSKES | 2plus1_Key_Cterm_5447 | EELSAEVKKLLDEVRKALARHKDEND |
| | AEAVRKAEERGIDEKDVRKLLEDLKKKSEEVAERNKRILDTLREIS | | KLLKEIEDSLRRHKEENDRLLEKLKE |
| | KRAEDEVRKVLKELEKTLKELEDRRPDSEELSAEVKKLLDEVRKAL | | STR (SEQ ID NO: 27157) |
| | ARHKDENDKLLKEIEDSLRRHKEENDRLLEKLKESTR (SEQ ID | | |
| | NO: 27156) | | |
| 2plus1_Cage_Cterm_5456 | SAEELLREVAELVKRVDEDLRRLLEEVRASNEEVIRRLEEILKRIE | 2plus1_Key_Cterm_5465 | EETVKRLLDELRELLERLKRTIEELL |
| | EENRKVVEELRRGGVSEDLVRESKRLVDESRRVIEKLVKESADSVE | | KRNRDLLADAEEKARRLLEENRKLLK |
| | RTRETVDRLREELKRLVEEIAKMVKGGSSEETVKRLLDELRELLER | | AARDTAT (SEQ ID NO: 27159) |
| | LKRTIEELLKRNRDLLADAEEKARRLLEENRKLLKAARDTAT (SEQ ID | | |
| | NO: 27158) | | |
| 2plus1_Cage_Cterm_5470 | SKEDRLREELKKLLARLAEEIERLKRALEESNKDLKRTIDASEKHL | 2plus1_Key_Cterm_5470 | EEELDKLLKEARDLIREIEKRLEELL |
| | RDVNEDVKRGGVSEELLRELERSTAENKERAKELLKRHEDLVRKVE | | KRVEKLTEDAKRDLERSNREHKELAD |
| | KELADLLRRLEEIVARVDEALKRGISEEELDKLLKEARDLIREIEK | | RIKETAR (SEQ ID NO: 27161) |
| | RLEELLKRVEKLTEDAKRDLERSNREHKELADRIKETAR (SEQ ID | | |
| | NO: 27160) | | |
| 2plus1_Cage_Nterm_2406 | SEVDEVVKEVEDLVRRNEELVEEVVRRVEKVVTDDRRLVEEVVREI | 2plus1_Key_Nterm_2406 | SEVDEVVKEVEDLVRRNEELVEEVVR |
| | RKIVKDVEDLARKLDKEELKRVLDEMRERIERLLEKLRRHSKKLDD | | RVEKVVTDDRRLVEEVVREIRKIVKD |
| | ELKRLLEELREHSRRVEKRLEDLLKELRERGVDEKVLRKLEKVIRE | | VEDLARK (SEQ ID NO: 27163) |
| | VRERSTRALRKVEEVIRRVREESERALRDLERVVKEVEKRMREAAR | | |
| | (SEQ ID NO: 27162) | | |
| 2plus1_Cage_Nterm_5406 | DREREVKKRLDEVRERIERLLRRVEEESRRVAEEIRRLIEEVRRRN | 2plus1_Key_Nterm_5406 | DREREVKKRLDEVRERIERLLRRVEE |
| | KKVTEEIRELLKGLKDKEEVRRVLERLRKLNAESDELLERILERLR | | ESRRVAEEIRRLIEEVRRRNKKVTEE |
| | RLVEATNRLVKAIIEELRRLVEKIVREVPDSEELREELKKLERKIE | | IRELLKGL (SEQ ID NO: 27165) |
| | KVAKEIHDHDKEVTERLEDLLRRITEHARKSDREIEETAR (SEQ ID | | |
| | NO: 27164) | | |
| 2plus1_Cage_Nterm_5409 | SEAEELLKRLEDRAEEILRRLEEILRTSRKLAEDVLRELEKLLRES | 2plus1_Key_Nterm_5409 | SEAEELLKRLEDRAEEILRRLEEILR |
| | ERRIREVLEELRGIKDKKELEDVIREVEKELDESLERSRELLKDVL | | TSRKLAEDVLRELEKLLRESERRIRE |
| | KKLDDNLKESERLVEDIDRELAKILEDLKKAGVPKEVVDEIKRIVD | | VLEELRGI (SEQ ID NO: 27167) |
| | EVRERLKRIVDENAKIVEDARRALEKIVKENEEILRRLKKELRELR | | |
| | K (SEQ ID NO: 27166) | | |
| 2plus1_Cage_Nterm_5410 | SRAETVLKEVTDKIKKLADSSDELLRRNKENIDELKKSSEELLRRL | 2plus1_Key_Nterm_5410 | SRAETVLKEVTDKIKKLADSSDELLR |
| | TKAIEEIEKGSVDEETLEELIRRVEAELEAHHRELEKNSREDEKRN | | RNKENIDELKKSSEELLRRLTKAIEE |
| | RDHHAKLEEEMRRVEERLEREGIDDEVVKRVRDLLDTVRRRNEKVN | | IEKGS (SEQ ID NO: 27169) |
| | EDVKRMNDKLRRDNEDVIRRVEKLLRELEEKRRT (SEQ ID | | |
| | NO: 27168) | | |
| 2plus1_Cage_Nterm_5413 | STEEVLDEIRKLHKTLTEDIKRVLREIEELHRRTIEENKEVLDKIA | 2plus1_Key_Nterm_5413 | STEEVLDEIRKLHKTLTEDIKRVLRE |
| | EDYKRVIDDVRTKDTPNVEKLLKDLEKSAKENIEHNERTLREDDRV | | IEELHRRTIEENKEVLDKIAEDYKRV |
| | LKEIRRRATELLKANEEMLRRIEEVARKGGVDAEELLRESKEAIKE | | IDDVRTKD (SEQ ID NO: 27171) |
| | VKRVLEELRKESKRVVDETRKLSEENLEHSERVLRKVEEDLR (SEQ ID | | |
| | NO: 27170) | | |
| 2plus1_Cage_5414_GFP11_Nterm | SEIEKILKEIEDLARRDEEVSKKIVEDIRRLAKEVEDTSRRDHMVL | 2plus1_Key_Nterm_5414 | SEIEKILKEIEDLARRDEEVSKKIVE |
| | HEYVNAAGITLRKDGSKEELEKEVREVVKTLEELVKDNHRLIRRAV | | DIRRLAKEVEDTSRDIVRKIEELAKR |
| | EEMKRLVEENHRHSREVVKELEDLVRELRKGSGSEDSERLVREVED | | VLDRLRKD (SEQ ID NO: 27173) |
| | LVRRLVRRSEKSNEEVKRTVEELVRRMEESNDRVRDLVRRLVEELK | | |
| | RAVD (SEQ ID NO: 27172) | | |
| 2plus1_Cage_5414_GFP11_Nterm | SEIEKILKEIEDLARRDEEVSKKIVEDIRRLAKEVEDTSRDIRDHM | 2plus1_Key_Nterm_5414 | SEIEKILKEIEDLARRDEEVSKKIVE |
| | VLHEYVNAAGITKDGSKEELEKEVREVVKTLEELVKDNHRLIRRAV | | DIRRLAKEVEDTSRDIVRKIEELAKR |
| | EEMKRLVEENHRHSREVVKELEDLVRELRKGSGSEDSERLVREVED | | VLDRLRKD (SEQ ID NO: 27175) |
| | LVRRLVRRSEKSNEEVKRTVEELVRRMEESNDRVRDLVRRLVEELK | | |
| | RAVD (SEQ ID NO: 27174) | | |
| 2plus1_Cage_Nterm_5439 | SRVEEIIEDLRRLLEEIRKENEDSIRRSKELLDRVKEINDTIIAEL | 2plus1_Key_Nterm_5439 | SRVEEIIEDLRRLLEEIRKENEDSIR |
| | ERLLKDIEKEVREKGSESEEVKKALRAVLEELEKLLRRVAEINEEV | | RSKELLDRVKEINDTIIAELERLLKD |
| | LRRNSKLVEEDERRNREVLKELARLVEELIREIGDEDKARKVAEVA | | IEKEVREKG (SEQ ID NO: 27177) |
| | EKVLRDIDKLDRESKEAFRATNEEIAKLDEDTARVAERVKKAIEDL | | |
| | AK (SEQ ID NO: 27176) | | |
| 3plus_Cage_529_GFP11_Cterm | SEAEDLEELIKELAELLKDVIRKLEKINRRLVKILEDIIRRLKEIS | 3plus_Key_Cterm_529 | KDEAERRRRELKDKLDRLREEHEEVK |
| | KEAEEELRKGTVEDKDILRDLERRLREILEESDRLLEELKRRLEEI | | RRLEEELTRLRETHKKIEKELREALK |
| | LRKSKELLRRLEEVLREILKRAEEVKRSNLPKEELIKEIVKLLEEL | | RVRDRST (SEQ ID NO: 27179) |
| | LRVIEKILEDNIRLLEELVEVIKEILEKHLRLLEELVRVIERILRE | | |
| | VGKDKDEAERRDHMVLHEYVNAAGITEEVKRRLEEELTRLRETHKK | | |
| | IEKELREALKRVRDRST (SEQ ID NO: 27178) | | |
| 3plus1_Cage_Cterm_263 | SLVDELRKSLERNVRVSEEVARRLKEALKRWVDVVRKVVEDLIRLN | 3plus1_Key_Cterm_263 | DEELKRVLEKAADLHRRLKDRHRKLL |
| | EDVVRVVEKVTVDESAIERVRRIIEELNRKLDAVLKKNEDLVRRLT | | EDLERIIRELKKKLDEVVEENKRSVD |
| | ELLDKLLEENRRLVEELDEDLKRRGGTEEVIDTILELIERSIERLK | | ELKR (SEQ ID NO: 27181) |
| | RLLDELLRIVREALKDNKRVADENLKKLKEILDELRKDGVEDEELK | | |
| | RVLEKAADLHRRLKDRHRKLLEDLERIIRELKKKLDEVVEENKRSV | | |
| | DELKR (SEQ ID NO: 27180) | | |
| 3plus1_Cage_Cterm_494 | SKEDKARELEKRLRDNLKKLEEVVRELAEVLKRNLEKLRRLAEELL | 3plus1_Key_Cterm_494 | EDLVRDIRRELKELEERARKILRDDE |
| | RALKRLLDKLRAGGLPKDELEDLRREVEDVLRRLEDLLRKLKKAND | | RDLRALEKRIRDIIREDREELERLKE |
| | ESLIRLEELLRRAEEENRRVLTTLRELLRGNGDDRDLARLVARLVE | | RARK (SEQ ID NO: 27183) |
| | ANNRALEELLRLVAKNVEDNNRVLEELLRLVKELAKRLLGRIRDED | | |
| | LVRDIRRELKELEERARKILRDDERDLRALEKRIRDIIREDREELE | | |
| | RLKERARK (SEQ ID NO: 27182) | | |
| 3plus1_Cage_Cterm_500 | SEKEELKRLLDKLLKELKRLSDELKATIDKILKILKEVSEEVKRTA | 3plus1_Key_Cterm_500 | EDELRKVEEDLKRLEDKLKKLLEDYE |
| | DELLDAIRRGGVDEEVLREIKREIEEIEKKLRKVNKEIEDEIREIK | | KKVRELEETLDDLLRKYEETLRRLEK |
| | KKLDEVDDKITKEVEKIKEALDKGGVDAKEVIKALKEILKEHADVF | | ELEEAER (SEQ ID NO: 27185) |
| | EDVLRRLKEIIKRHRDVVKEVLEELRKILEKVAEVLKRQGRSEDEL | | |
| | RKVEEDLKRLEDKLKKLLEDYEKKVRELEETLDDLLRKYEETLRRL | | |
| | EKELEEAER (SEQ ID NO: 27184) | | |
| 3plus1_Cage_506_GFP11_Cterm | SAEETLRRITEESKRVLEEITKKYEDLERESREVLRKLREDLDRIK | 3plus1_Key_Cterm_506 | RERLARLLKALADKLIRVLEEILKIN |
| | RELEDVLTKGGDDKDEVLTVLEKVLDELLKLHRENLRVIKELLRKV | | EELNRKIIKFARENLERNRRVNKKVI |
| | LEVDRENLEVLKRLLEKARDLLRRSGRDEKDLKKLEDELRKVKEEL | | EVLREAAR (SEQ ID NO: 27187) |
| | EKKHEASKRDIEDLERELDKVTEEVERLLRKLKEELRRAAEGSDER | | |
| | DHMVLHEYVNAAGITTRVLEEILKINEELNRKIIKFARENLERNRR | | |
| | VNKKVIEVLREAAR (SEQ ID NO: 27186) | | |
| 3plus1_Cage_508_GFP11_Cterm | STEEILKRVKEILDELAKELRDIIRESLRTIEELLDELRRILEESE | 3plus1_Key_Cterm_508 | DEVKRRLEEKERRIRTRYEELRRRLR |
| | RTLEELVKTIKDGVKDSEELLRRLKRLLEDLRRAHEELLKRLKEAV | | KRVKDYEDKLREIEKKVRRDAERIEE |
| | DEHRDRLRKILEELERVLKELRKRIEELRRSGDRISEKDVLRKLEE | | ELERAKK (SEQ ID NO: 27189) |
| | VLRKALEILERLLKKIRESARELLKIIEEVLDANIKVMEEALKTIK | | |
| | ELLKDGRDHMVLHEYVNAAGITTRYEELRRRLRKRVKDYEDKLREI | | |
| | EKKVRRDAERIEEELERAKK (SEQ ID NO: 27188) | | |
| 3plus1_Cage_Cterm_510 | SEKEELLKLIKRVIELLKRVLEEHLRLVEDVIRRLKELLDSNEKIV | 3plus1_Key_Cterm_510 | EDLLRKAKKVITEVREKLKRNLEDVR |
| | REVIEDLKRLLDEVRGDKEELDRIKEKLEEVLERYKRRLEEIKRDL | | RVIEDVKRKSARILEEARRLIEEVER |
| | ERMLEDYKRELKRIEEDLRRVLEEVERIATRGEGPAEALIDKLRKI | | ELEKIRK (SEQ ID NO: 27191) |
| | LERALRELDKLSKKLDELLKKVLEELEKSNREIDKLLKDVLRRVEE | | |
| | GGASEDLLRKAKKVITEVREKLKRNLEDVRRVIEDVKRKSARILEE | | |
| | ARRLIEEVERELEKIRK (SEQ ID NO: 27190) | | |
| 3plus1_Cage_528_GFP11_Cterm | SEAEDLEELIKELAELLKDVIRKLEKINRRLVKILEDIIRRLKEIS | 3plus1_Key_Cterm_528 | KDEAERRRRELKDKLDRLREEHEEVK |
| | KEAEEELRKGTVEDKDILRDLERRLREILEESDRLLEELKRRLEEI | | RRLEEELTRLRETHKKIEKELREALK |
| | LRKSKELLRRLEEVLREILKRAEEVKRSNLPKEELIKEIVKLLEEL | | RVRDRST (SEQ ID NO: 27193) |
| | LRVIEKILEDNIRLLEELVEVIKEILEKHLRLLEELVRVIERILRE | | |
| | VGRDHMVLHEYVNAAGITLDRLREEHEEVKRRLEEELTRLRETHKK | | |
| | IEKELREALKRVRDRST (SEQ ID NO: 27192) | | |
| 3plus1_Cage_528_GFP11_Cterm | SEAEDLEELIKELAELLKDVIRKLEKINRRLVKILEDIIRRLKEIS | 3plus1_Key_Cterm_528 | KDEAERRRRELKDKLDRLREEHEEVK |
| | KEAEEELRKGTVEDKDILRDLERRLREILEESDRLLEELKRRLEEI | | RRLEEELTRLRETHKKIEKELREALK |
| | LRKSKELLRRLEEVLREILKRAEEVKRSNLPKEELIKEIVKLLEEL | | RVRDRST (SEQ ID NO: 27195) |
| | LRVIEKILEDNIRLLEELVEVIKEILEKHLRLLEELVRVIERILRE | | |
| | VGKDKRDHMVLHEYVNAAGITLREEHEEVKRRLEEELTRLRETHKK | | |
| | IEKELREALKRVRDRST (SEQ ID NO: 27194) | | |
| 3plus1_Cage_528_GFP11_Cterm | SEAEDLEELIKELAELLKDVIRKLEKINRRLVKILEDIIRRLKEIS | 3plus1_Key_Cterm_528 | KDEAERRRRELKDKLDRLREEHEEVK |
| | KEAEEELRKGTVEDKDILRDLERRLREILEESDRLLEELKRRLEEI | | RRLEEELTRLRETHKKIEKELREALK |
| | LRKSKELLRRLEEVLREILKRAEEVKRSNLPKEELIKEIVKLLEEL | | RVRDRST (SEQ ID NO: 27197) |
| | LRVIEKILEDNIRLLEELVEVIKEILEKHLRLLEELVRVIERILRE | | |
| | VGKDKDEAERDHMVLHEYVNAAGITHEEVKRRLEEELTRLRETHKK | | |
| | IEKELREALKRVRDRST (SEQ ID NO: 27196) | | |
| 3plus1_Cage_529_GFP11_Cterm | SEAEDLEELIKELAELLKDVIRKLEKINRRLVKILEDIIRRLKEIS | 3plus1_Key_Cterm_529 | KDEAERRRRELKDKLDRLREEHEEVK |
| | KEAEEELRKGTVEDKDILRDLERRLREILEESDRLLEELKRRLEEI | | RRLEEELTRLRETHKKIEKELREALK |
| | LRKSKELLRRLEEVLREILKRAEEVKRSNLPKEELIKEIVKLLEEL | | RVRDRST (SEQ ID NO: 27199) |
| | LRVIEKILEDNIRLLEELVEVIKEILEKHLRLLEELVRVIERILRE | | |
| | VGKRDHMVLHEYVNAAGITDRLREEHEEVKRRLEEELTRLRETHKK | | |
| | IEKELREALKRVRDRST (SEQ ID NO: 27198) | | |
| 3plus1_Cage_529_GFP11_Cterm | SEAEDLEELIKELAELLKDVIRKLEKINRRLVKILEDIIRRLKEIS | 3plus1_Key_Cterm_529 | KDEAERRRRELKDKLDRLREEHEEVK |
| | KEAEEELRKGTVEDKDILRDLERRLREILEESDRLLEELKRRLEEI | | RRLEEELTRLRETHKKIEKELREALK |
| | LRKSKELLRRLEEVLREILKRAEEVKRSNLPKEELIKEIVKLLEEL | | RVRDRST (SEQ ID NO: 27201) |
| | LRVIEKILEDNIRLLEELVEVIKEILEKHLRLLEELVRVIERILRE | | |
| | VGKDRDHMVLHEYVNAAGITRLREEHEEVKRRLEEELTRLRETHKK | | |
| | IEKELREALKRVRDRST (SEQ ID NO: 27200) | | |
| 3plus1_Cage_529_GFP11_Cterm | SEAEDLEELIKELAELLKDVIRKLEKINRRLVKILEDIIRRLKEIS | 3plus1_Key_Cterm_529 | KDEAERRRRELKDKLDRLREEHEEVK |
| | KEAEEELRKGTVEDKDILRDLERRLREILEESDRLLEELKRRLEEI | | RRLEEELTRLRETHKKIEKELREALK |
| | LRKSKELLRRLEEVLREILKRAEEVKRSNLPKEELIKEIVKLLEEL | | RVRDRST (SEQ ID NO: 27203) |
| | LRVIEKILEDNIRLLEELVEVIKEILEKHLRLLEELVRVIERILRE | | |
| | VGKDKDRDHMVLHEYVNAAGITREEHEEVKRRLEEELTRLRETHKK | | |
| | IEKELREALKRVRDRST (SEQ ID NO: 27202) | | |
| 3plus1_Cage_Cterm_530 | SDKEEIKRRVEKTARDLETEHDKIKKRLEDTVRDIKRELDELLEKY | 3plus1_Key_Cterm_530 | EKRLIEEVVRLLRENLDVVRRILEAL |
| | ERVLRKIEKTLREGDGDAIDLVDKLLETVKRLNEEVLRIVKKIADE | | AKLIKELLEALEEVLRRNKELIRELL |
| | LRRLVEEVLRVLRESNETLARLLEEGNVGDEVRERLEEVLRRTEEI | | RVLDEALK (SEQ ID NO: 27205) |
| | IKTLEEIVKRLLDELRRLVEELRRISKKLKDELDKIKEKGKDEKRL | | |
| | IEEVVRLLRENLDVVRRILEALAKLIKELLEALEEVLRRNKELIRE | | |
| | LLRVLDEALK (SEQ ID NO: 27204) | | |
| 3plus1_Cage_534_GFP11_Cterm | DEDRIIEEIARLLEELLRELLELIKKLIETNRRLNEEHERAVRELA | 3plus1_Key_Cterm_534 | EKIAEEIERELEELRRMIKRLHEDLE |
| | RLLEELLDRLVKKGISDEKLKRIRERLKRALDDLERLHREINKRLE | | RKLKESEDELREIEARLEEKIRRLEE |
| | DLVRELEKLVREILKELKDALEELRRASARAGGEEVLRRLEEIVKK | | KLERKRR (SEQ ID NO: 27207) |
| | LLDLVRRILERLKEIHKDNVRLLRELNERLTRIVEDLVRLIREILR | | |
| | EAGVDERDHMVLHEYVNAAGITIKRLHEDLERKLKESEDELREIEA | | |
| | RLEEKIRRLEEKLERKRR (SEQ ID NO: 27206) | | |
| 3plus1_Cage_534_GFP11_Cterm | DEDRIIEEIARLLEELLRELLELIKKLIETNRRLNEEHERAVRELA | 3plus1_Key_Cterm_534 | EKIAEEIERELEELRRMIKRLHEDLE |
| | RLLEELLDRLVKKGISDEKLKRIRERLKRALDDLERLHREINKRLE | | RKLKESEDELREIEARLEEKIRRLEE |
| | DLVRELEKLVREILKELKDALEELRRASARAGGEEVLRRLEEIVKK | | KLERKRR (SEQ ID NO: 27209) |
| | LLDLVRRILERLKEIHKDNVRLLRELNERLTRIVEDLVRLIREILR | | |
| | EAGVDEKIRDHMVLHEYVNAAGITRLHEDLERKLKESEDELREIEA | | |
| | RLEEKIRRLEEKLERKRR (SEQ ID NO: 27208) | | |
| 3plus1_Cage_534_GFP11_Cterm | DEDRIIEEIARLLEELLRELLELIKKLIETNRRLNEEHERAVRELA | 3plus1_Key_Cterm_534 | EKIAEEIERELEELRRMIKRLHEDLE |
| | RLLEELLDRLVKKGISDEKLKRIRERLKRALDDLERLHREINKRLE | | RKLKESEDELREIEARLEEKIRRLEE |
| | DLVRELEKLVREILKELKDALEELRRASARAGGEEVLRRLEEIVKK | | KLERKRR (SEQ ID NO: 27211) |
| | LLDLVRRILERLKEIHKDNVRLLRELNERLTRIVEDLVRLIREILR | | |
| | EAGVDEKIAEEIERDHMVLHEYVNAAGITLERKLKESEDELREIEA | | |
| | RLEEKIRRLEEKLERKRR (SEQ ID NO: 27210) | | |
| 3plus1_Cage_Cterm_539 | SEKEKLLKESEEEVRRLRRTLEELLRKYREVLERLRKELREIEERV | 3plus1_Key_Cterm_539 | ERLVKTLIEDVEAVIKRILELITRVA |
| | RDVVRRLKEVLDRKGLDIDTIIKEVEDLLKTVLDRLRELLDKIRRL | | EDNERVLERIIRELTDNLERHLKIVR |
| | TKEAIEVVREIIERIVRHAERVKDELRKEGGDKEKLDRVDRLIKEN | | EIVK (SEQ ID NO: 27213) |
| | TRHLKEILDRIEDLVRRSEKKLRDIIREVRRLIEELRKKAEEIKKG | | |
| | PDERLVKILIEDVEAVIKRILELITRVAEDNERVLERIIRELTDNL | | |
| | ERHLKIVREIVK (SEQ ID NO: 27212) | | |
| 3plus1_Cage_Cterm_548 | DKAEVLREALKLLKDLLEELIKIHEESLKRILDLIDTLVKVHEDAL | 3plus1_Key_Cterm_548 | EEIDRELKRVVEELRRLHEEIKERLD |
| | RALKELLERSGLDERELRKVERMATESLRTIAKLKEELRDLARRSL | | DVARRSEEELRRIIKKLKEVVKEIRK |
| | EKLREDLKRVDDILRKVEEKVRRTGPSEELIEELIRTIEKLLKEIV | | KLK (SEQ ID NO: 27215) |
| | RINEEVLKAVRELLKTLLKLSEDVVRRIEEILRKGGVPEEIDRELK | | |
| | RVVEELRRLHEEIKERLDDVARRSEEELRRIIKKLKEVVKEIRKKL | | |
| | K (SEQ ID NO: 27214) | | |
| 3plus1_Cage_Cterm_556 | SERELIERWLELHKEILRLIRELVERLLKLHREILDTIKKLIRELL | 3plus1_Key_Cterm_556 | DDERRTLTELLKRMEDILEKVERTLK |
| | ELLEDIARKLGLDKEAKDELREIAKRVEDKLEKLERESRKVEEDLK | | KLLDDSARMAEEVKKTLKELLERSEK |
| | RKLKELTDESDIVEKRVRDVVRRGTQSREEIAEELLRLDRKLLKAV | | VAEDVRK (SEQ ID NO: 27217) |
| | EELLKEILDLNKKLLDDVRAILEETRRVLEKLLDRVRRGERTDDER | | |
| | RTLTELLKRMEDILEKVERTLKKLLDDSARMAEEVKKTLKELLERS | | |
| | EKVAEDVRK (SEQ ID NO: 27216) | | |
| 3plus1_Cage_Cterm_560 | SKKELLEEVVRRAIELLKRHLEKLKRILEEIVRLLEEHLEKVERVL | 3plus1_Key_Cterm_560 | EDKLKEIEDELRRLLEELRRLDKAIK |
| | EAILSLLDDLLRRGGDERAIRTLEDVKRRLREILERLADENAKAIK | | DRLRELKKDLDEANRRIKETLKKLLR |
| | RLADLLDKLEKRNKEAIERLEEILEELKRVRRDEELLRVLETLLKI | | EVEK (SEQ ID NO: 27219) |
| | IEDILRENTKVLEDLLRLVEEILEANLRVVEELLRLAREILTEIVG | | |
| | DEDKLKEIEDELRRLLEELRRLDKAIKDRLRELKKDLDEANRRIKE | | |
| | TLKKLLREVEK (SEQ ID NO: 27218) | | |
| 3plus1_Cage_568_GFP11_Cterm | KEIEETLKELEDLNREMVETNRRVLEETRRLNKETVDRVKATLDEL | 3plus1_Key_Cterm_568 | KAVEELEKALEEIKRRLKEVIDRYED |
| | AKMLKKLVDDVRKGPTSEELKRLLAELEELLARVVRRVEELLKKST | | ELRKLRKEYKEKIDKYERKLEEIERR |
| | DLLERAVKDSADALRRSHEVLKEVASRVKRAKDEGLPREEVLRLLR | | ERT (SEQ ID NO: 27221) |
| | ELLERHAKVLKDIVRVSEKLLREHLKVLREIVEVLEELLERILKVI | | |
| | LDTTRDHMVLHEYVNAAGITKRRLKEVIDRYEDELRKLRKEYKEKI | | |
| | DKYERKLEEIERRERT (SEQ ID NO: 27220) | | |
| 3plus1_Cage_568_GFP11_Cterm | KEIEETLKELEDLNREMVETNRRVLEETRRLNKETVDRVKATLDEL | 3plus1_Key_Cterm_568 | KAVEELEKALEEIKRRLKEVIDRYED |
| | AKMLKKLVDDVRKGPTSEELKRLLAELEELLARVVRRVEELLKKST | | ELRKLRKEYKEKIDKYERKLEEIERR |
| | DLLERAVKDSADALRRSHEVLKEVASRVKRAKDEGLPREEVLRLLR | | ERT (SEQ ID NO: 27223) |
| | ELLERHAKVLKDIVRVSEKLLREHLKVLREIVEVLEELLERILKVI | | |
| | LDTTGGDRDHMVLHEYVNAAGITLKEVIDRYEDELRKLRKEYKEKI | | |
| | DKYERKLEEIERRERT (SEQ ID NO: 27222) | | |
| 3plus1_Cage_Cterm_572 | DEDELIRKLLEDLKDIVRKILELIERDLRDIERVVRRIVKVIRDDL | 3plus1_Key_Cterm_572 | SELADRIRKLIEDLERHTAKVLEDVK |
| | KKIKEVVDDIARGVPRTEELERVIKRIEELLRTSEEELDRILKEIE | | RAITELRKNSKDILEEVRKLIDELRK |
| | ELLRESRRRLEEVVSAVEELLRRVEEIVDKGRESKEDVIKLLREVV | | RIKEVED (SEQ ID NO: 27225) |
| | DDILRLVEEVVRTNLEIIKRILELIERVIRLNLSIIRDILRLLEGT | | |
| | VDSELADRIRKLIEDLERHTAKVLEDVKRAITELRKNSKDILEEVR | | |
| | KLIDELRKRIKEVED (SEQ ID NO: 27224) | | |
| 3plus1_Cage_Cterm_581 | SALETVKKLLEDSSEKIERIVEEDERVAKESSDRIRRLVEEDKRVA | 3plus1_Key_Cterm_581 | AEAVIKVIEKLIRANKRVWDALLKIN |
| | DEILDLIEKIGDTDTLLKLVEEWSRTSKKLLDDVLKLHKDWSDDSR | | EDLVRVNKTVWKELLRVNEKLARDLE |
| | RLLEEILRVHEELIRRVKEILDREGKPEEVVRELEKVLKESLDTLE | | RVVK (SEQ ID NO: 27227) |
| | EIIRRLDEANAATVKRVADVIRELEDINRKVLEEIKRGSDDAEAVI | | |
| | KVIEKLIRANKRVWDALLKINEDLVRVNKTVWKELLRVNEKLARDL | | |
| | ERVVK (SEQ ID NO: 27226) | | |
| 3plus1_Cage_Cterm_585 | SKEEKLKDDVRAVLEDLDRVLKELEKLSEDNLRELKRVLDRITDLH | 3plus1_Key_Cterm_585 | SKAAEDILRVLEKLVKVSREAIKLIL |
| | RRILDELRKGIGSEELLRRVEKVLKDNLDLLRKLVEEHKESSERDL | | ELSEHHVRVSTRIARLLLDVARKLAE |
| | KRVEDLVREIKEVLRKLLELEDRGTDIRKIEEEIERLLRKIRKAVE | | VIKEAER (SEQ ID NO: 27229) |
| | ESKDLNRRNSERIEEVARRSEELARRLLKEIRERGDSKAAEDILRV | | |
| | LEKLVKVSREAIKLILELSEHHVRVSTRIARLLLDVARKLAEVIKE | | |
| | AER (SEQ ID NO: 27228) | | |
| 3plus1_Cage_Cterm_587 | SEIEDVIRRLRKILEDLERVSEKLLREIKKILDEARRLNEEVIKEI | 3plus1_Key_Cterm_587 | IEDLVREVERLIKRIEDSLRELEKTV |
| | KRVLEDAVRVFRDGSGSKEELAKLVEELIRELAKLAKEVDEIHKRI | | RELLKRIKEASDKVREDVDRLIKELK |
| | VERLKALVEDAERIHRKIVETLEEIVRGVPSEELKRVVEAIVEVIK | | EAAD (SEQ ID NO: 27231) |
| | EHLKVLADVIRRIIKAIEENAETIKRVLEDIVRVLELVLRGEGSIE | | |
| | DLVREVERLIKRIEDSLRELEKTVRELLKRIKEASDKVREDVDRLI | | |
| | KELKEAAD (SEQ ID NO: 27230) | | |
| 3plus1_Cage_Cterm_605 | SREELLDRILEAIAKILEDLKRLIDENLARLEEVVRELERIIDRNL | 3plus1_Key_Cterm_605 | DEIIRKLDELLKEVEKVHKEVKDRIR |
| | KLIREILDELKKGSGSEEILEKIKKVDKELEDLIRRLLKKLEDLIR | | KLLEDHKRSLDEVKKKLERLLERAKE |
| | ETERRLREILKRIRDLLKEVKDRDKDLERLLEVLEEVLRVIAELAK | | VVEREKK (SEQ ID NO: 27233) |
| | ELLDSLRKVLKVVEEVLRLLNEVNKEVLDVIRELAKDGGSDEIIRK | | |
| | LDELLKEVEKVHKEVKDRIRKLLEDHKRSLDEVKKKLERLLERAKE | | |
| | VVEREKK (SEQ ID NO: 27232) | | |
| 3plus1_Cage_Cterm_607 | SEREELLERIKEILKRVKDKLDEDLKRLKEILEKLKEKADRDLEEL | 3plus1_Key_Cterm_607 | SETAVRAIIRVLEKHLEAVRRVLEEL |
| | RRRIEEVREKLERTGRTDELVKEVLDTVRRNLENLKRLVEDILRKL | | LKVLAEHLETVRELIERLKRVLEEAI |
| | EENVKNLTDLVREILKLITELIKRLEDGGLPKEVLDALRRVLEKLE | | EVVERVAR (SEQ ID NO: 27235) |
| | ELLREILERLKRSLEAVKRKIEELLKELERSLDELRRALERIRKEI | | |
| | GDSETAVRAIIRVLEKHLEAVRRVLEELLKVLAEHLETVRELIERL | | |
| | KRVLEEAIEVVERVAR (SEQ ID NO: 27234) | | |
| 3plus1_Cage_Cterm_610 | SLEEITKRLLELVEENLARHEEILRELLELAKRLAKEDRDILEEVL | 3plus1_Key_Cterm_610 | ERTLREVVRKVLEEAKRLLDELEEVH |
| | KLIEELLKLLEDNGSSEEDLKRLLKEVIEELRAVVKRVKDKWDEVV | | KRVKKELEDIIEENRRVVKRVRDELR |
| | KRIEDLVKKLKELHDDILRKLRELVRKIVTDISESGGEAEKVKRVV | | EIKRELDE (SEQ ID NO: 27237) |
| | EKILELVERLAKVVKESVEKLLEILRELAEVSKRVAEALLRLLEEL | | |
| | VRVIRIKDERTLREVVRKVLEEAKRLLDELEEVHKRVKKELEDIIE | | |
| | ENRRVVKRVRDELREIKRELDE (SEQ ID NO: 27236) | | |
| 3plus1_Cage_611_GFP11_Cterm | SLEEITKRLLELVEENLARHEEILRELLELAKRLAKEDRDILEEVL | 3plus1_Key_Cterm_611 | ERTLREVVRKVLEEAKRLLDELEEVH |
| | KLIEELLKLLEDNGSSEEDLKRLLKEVIEELRAVVKRVKDKWDEVV | | KRVKKELEDIIEENRRVVKRVRDELR |
| | KRIEDLVKKLKELHDDTLRKLRELVRKIVTDISESGGEAEKVKRVV | | EIKRELDE (SEQ ID NO: 27239) |
| | EKILELVERLAKVVKESVEKLLEILRELAEVSKRVAEALLRLLEEL | | |
| | VRVIRIKDERDHMVLHEYVNAAGITLLDELEEVHKRVKKELEDIIE | | |
| | ENRRVVKRVRDELREIKRELDE (SEQ ID NO: 27238) | | |
| 3plus1_Cage_Cterm_632 | SEKELVDDIRRILEEILRLLRSLLEEVIRLLEENEKLVRRHLKTVI | 3plus1_Key_Cterm_632 | DSLVREVEELIKRLEKHIDDLLKTSR |
| | DILRRVAKLLDENGIRTDEADRVLERLEKAHRELLEDYKRALEKIK | | DLVKRVLDLVDEVVKRVEDLVERVKE |
| | ETLERVLREAEEVVKKIDDALRKLGGSKEVLKRLLEELLRLVEKIA | | KIDT (SEQ ID NO: 27241) |
| | EEIKRLLSELVRVTEELVRTNKELLEEAVRVIRKEVGDDSLVREVE | | |
| | ELIKRLEKHIDDLLKTSRDLVKRVLDLVDEVVKRVEDLVERVKEKI | | |
| | DT (SEQ ID NO: 27240) | | |
| 3plus1_Cage_Cterm_641 | DEVEELLKRVRELLKASEELVRKILEDVKRLLERSIEEVEDLLRKV | 3plus1_Key_Cterm_641 | EDLIRDVDRVLKEIMDLLRDLVRRTK |
| | EELLKRLLDLVERGGSLDEILRELVRLLKEIVRKVLELNRKLVEDV | | ELLEELLRLLEELVRAHKELVRSILD |
| | IRIAKRLLELNAESVKEWDRILKLLRDGTGDKEELLKDAEEALEKY | | EIRK (SEQ ID NO: 27243) |
| | ERAVREILKELEEIIKEYVRRVEELLKELKDAVDKAKDEVRKGSGD | | |
| | EDLIRDVDRVLKEIMDLLRDLVRRTKELLEELLRLLEELVRAHKEL | | |
| | VRSILDEIRK (SEQ ID NO: 27242) | | |
| 3plus1_Cage_646_GFP11_Cterm | DAEEVVKRLADVLRENDETIRKVVEDLVRIAEENDRLWKKLVEDIA | 3plus1_Key_Cterm_646 | EDVKRALEELVSRLRKLLEDVKKASE |
| | EILRRIVELLRRGGVPEELLDRLAKVVKSIVEKAEKILERLNRVSK | | DIVREVERIVRELAKRSDEILKKLED |
| | AIAEKLKTIVDELNEVSKEIVKRAEDILRKGKDKETVLRALRTLVK | | IVEKLRE (SEQ ID NO: 27245) |
| | EYADLSKEVLERVERIVREYVKLSDEVVKSLAEIVEELIRIIEDLL | | |
| | RKGNRDHMVLHEYVNAAGITRKLLEDVKKASEDIVREVERIVRELA | | |
| | KRSDEILKKLEDIVEKLRE (SEQ ID NO: 27244) | | |
| 3plus1_Cage_646_GFP11_Cterm | DAEEVVKRLADVLRENDETIRKVVEDLVRIAEENDRLWKKLVEDIA | 3plus1_Key_Cterm_646 | EDVKRALEELVSRLRKLLEDVKKASE |
| | EILRRIVELLRRGGVPEELLDRLAKVVKSIVEKAEKILERLNRVSK | | DIVREVERIVRELAKRSDEILKKLED |
| | AIAEKLKTIVDELNEVSKEIVKRAEDILRKGKDKETVLRALRTLVK | | IVEKLRE (SEQ ID NO: 27247) |
| | EYADLSKEVLERVERIVREYVKLSDEVVKSLAEIVEELIRIIEDLL | | |
| | RKGNLDEDRDHMVLHEYVNAAGITEDVKKASEDIVREVERIVRELA | | |
| | KRSDEILKKLEDIVEKLRE (SEQ ID NO: 27246) | | |
| 3plus1_Cage_646_GFP11_Cterm | DAEEVVKRLADVLRENDETIRKVVEDLVRIAEENDRLWKKLVEDIA | 3plus1_Key_Cterm_646 | EDVKRALEELVSRLRKLLEDVKKASE |
| | EILRRIVELLRRGGVPEELLDRLAKVVKSIVEKAEKILERLNRVSK | | DIVREVERIVRELAKRSDEILKKLED |
| | AIAEKLKTIVDELNEVSKEIVKRAEDILRKGKDKETVLRALRTLVK | | IVEKLRE (SEQ ID NO: 27249) |
| | EYADLSKEVLERVERIVREYVKLSDEVVKSLAEIVEELIRIIEDLL | | |
| | RKGNLDEDVRDHMVLHEYVNAAGITDVKKASEDIVREVERIVRELA | | |
| | KRSDEILKKLEDIVEKLRE (SEQ ID NO: 27248) | | |
| 3plus1_Cage_647_GFP11_Cterm | DAEEVVKRLADVLRENDETIRKVVEDLVRIAEENDRLWKKLVEDIA | 3plus1_Key_Cterm_647 | EDVKRALEELVSRLRKLLEDVKKASE |
| | EILRRIVELLRRGGVPEELLDRLAKVVKSIVEKAEKILERLNRVSK | | DIVREVERIVRELAKRSDEILKKLED |
| | AIAEKLKTIVDELNEVSKEIVKRAEDILRKGKDKETVLRALRTLVK | | IVEKLRE (SEQ ID NO: 27251) |
| | EYADLSKEVLERVERIVREYVKLSDEVVKSLAEIVEELIRIIEDLL | | |
| | RKGNLRDHMVLHEYVNAAGITKLLEDVKKASEDIVREVERIVRELA | | |
| | KRSDEILKKLEDIVEKLRE (SEQ ID NO: 27250) | | |
| 3plus1_Cage_647_GFP11_Cterm | DAEEVVKRLADVLRENDETIRKVVEDLVRIAEENDRLWKKLVEDIA | 3plus1_Key_Cterm_647 | EDVKRALEELVSRLRKLLEDVKKASE |
| | EILRRIVELLRRGGVPEELLDRLAKVVKSIVEKAEKILERLNRVSK | | DIVREVERIVRELAKRSDEILKKLED |
| | AIAEKLKTIVDELNEVSKEIVKRAEDILRKGKDKETVLRALRTLVK | | IVEKLRE (SEQ ID NO: 27253) |
| | EYADLSKEVLERVERIVREYVKLSDEVVKSLAEIVEELIRIIEDLL | | |
| | RKGNLDEDVKRALERDHMVLHEYVNAAGITSEDIVREVERIVRELA | | |
| | KRSDEILKKLEDIVEKLRE (SEQ ID NO: 27252) | | |
| 3plus1_Cage_Cterm_647 | DAEEVVKRLADVLRENDETIRKVVEDLVRIAEENDRLWKKLVEDIA | 3plus1_Key_Cterm_647 | EDVKRALEELVSRLRKLLEDVKKASE |
| | EILRRIVELLRRGGVPEELLDRLAKVVKSIVEKAEKILERLNRVSK | | DIVREVERIVRELAKRSDEILKKLED |
| | AIAEKLKTIVDELNEVSKEIVKRAEDILRKGKDKETVLRALRTLVK | | IVEKLRE (SEQ ID NO: 27255) |
| | EYADLSKEVLERVERIVREYVKLSDEVVKSLAEIVEELIRIIEDLL | | |
| | RKGNLDEDVKRALEELVSRLRKLLEDVKKASEDIVREVERIVRELA | | |
| | KRSDEILKKLEDIVEKLRE (SEQ ID NO: 27254) | | |
| 3plus1_Cage_Cterm_653 | DEEETLRRLLERKVELAKEYLDVSKEVIDRTTKLLDEYLKTSKRIV | 3plus1_Key_Cterm_653 | SREALEEARRRLEELLRELNEITKDL |
| | DATVELLERGDLGPDELIKRLAEELERSLRELEEEIKRLKRELEES | | EAKLEKLLRDLNELTKALEEELKRLL |
| | LKKLKEIIDRLAEEAEKLLAVLKRGEGSEEEALRALASLVRELIEV | | DELKKRTD (SEQ ID NO: 27257) |
| | LRENDERLRDVLRRLIEALRKNNEILERVLRKLVRAAEERGRDESS | | |
| | REALEEARRRLEELLRELNEITKDLEAKLEKLLRDLNELTKALEEE | | |
| | LKRLLDELKKRTD (SEQ ID NO: 27256) | | |
| 3plus1_Cage_Cterm_658 | DEERIIKTLEDINAKLVEDIKRILDKVAELNERLADAIRKILEETK | 3plus1_Key_Cterm_658 | KDTLRTVEKLVEDVKRRLDKLLEDYK |
| | RILEATTRKVRKDGEISEELLRRLEEKLRKLLEDLERVLAEHEDES | | RLIEEVKKELDKLLKEYEDALREIKK |
| | RRILEEVERLLKRHADASKELLDRARSVARGVKSDKELVDRLKKLI | | RIDE (SEQ ID NO: 27259) |
| | DDSLESVRELIERLKELLDRLVKSVEDLIRTIKELLDRLVEVLREG | | |
| | VSDKDTLRTVEKLVEDVKRRLDKLLEDYKRLIEEVKKELDKLLKEY | | |
| | EDALREIKKRIDE (SEQ ID NO: 27258) | | |
| 3plus1_Cage_Cterm_660 | TEEEVVEDVKRVLDESHDDLRRLIETLTRVLRESLKRIKEALEELE | 3plus1_Key_Cterm_660 | REEVKRVLEEARDELRRLLEEYKAII |
| | RVLKKLLDLLEGGRDAREVLDEIRKVLERLREVIEELLRINKEVLR | | EELERELERLLREHREVIRRIKEEID |
| | ELERVIRELLKKNEDLARRVRSGVKSRLLEVLERLARESLELNRAI | | KSSK (SEQ ID NO: 27261) |
| | LEELRKLVEKSLRAVEKILKRLEEIVRKLLKLVEDGGPREEVKRVL | | |
| | EEARDELRRLLEEYKAIIEELERELERLLREHREVIRRIKEEIDKS | | |
| | SK (SEQ ID NO: 27260) | | |
| 3plus1_Cage_Nterm_263 | SLVDELRKSLERNVRVSEEVARRLKEALKRWVDVVRKVVEDLIRLN | 3plus1_Key_Nterm_263 | SLVDELRKSLERNVRVSEEVARRLKE |
| | EDVVRVVEKVTVDESAIERVRRIIEELNRKLDAVLKKNEDLVRRLT | | ALKRWVDVVRKVVEDLIRLNEDVVRV |
| | ELLDKLLEENRRLVEELDEDLKRRGGTEEVIDTILELIERSIERLK | | VEKV (SEQ ID NO: 27263) |
| | RLLDELLRIVREALKDNKRVADENLKKLKEILDELRKDGVEDEELK | | |
| | RVLEKAADLHRRLKDRHRKLLEDLERIIRELKKKLDEVVEENKRSV | | |
| | DELKR (SEQ ID NO: 27262) | | |
| 3plus1_Cage_Nterm_500 | SEKEELKRLLDKLLKELKRLSDELKATIDKILKILKEVSEEVKRTA | 3plus1_Key_Nterm_500 | SEKEELKRLLDKLLKELKRLSDELKA |
| | DELLDAIRRGGVDEEVLREIKREIEEIEKKLRKVNKEIEDEIREIK | | TIDKILKILKEVSEEVKRTADELLDA |
| | KKLDEVDDKITKEVEKIKEALDKGGVDAKEVIKALKEILKEHADVF | | IRRG (SEQ ID NO: 27265) |
| | EDVLRRLKEIIKRHRDVVKEVLEELRKILEKVAEVLKRQGRSEDEL | | |
| | RKVEEDLKRLEDKLKKLLEDYEKKVRELEETLDDLLRKYEETLRRL | | |
| | EKELEEAER (SEQ ID NO: 27264) | | |
| 3plus1_Cage_Nterm_510 | SEKEELLKLIKRVIELLKRVLEEHLRLVEDVIRRLKELLDSNEKIV | 3plus1_Key_Nterm_510 | SEKEELLKLIKRVIELLKRVLEEHLR |
| | REVIEDLKRLLDEVRGDKEELDRIKEKLEEVLERYKRRLEEIKRDL | | LVEDVIRRLKELLDSNEKIVREVIED |
| | ERMLEDYKRELKRIEEDLRRVLEEVERIATRGEGPAEALIDKLRKI | | LKRLLDEV (SEQ ID NO: 27267) |
| | LERALRELDKLSKKLDELLKKVLEELEKSNREIDKLLKDVLRRVEE | | |
| | GGASEDLLRKAKKVITEVREKLKRNLEDVRRVIEDVKRKSARILEE | | |
| | ARRLIEEVERELEKIRK (SEQ ID NO: 27266) | | |
| 3plus1_Cage_529_GFP11_Nterm | SEAEDLEELIKELAELLKDVIRKLEKINRRLVKILRDHMVLHEYVN | 3plus1_Key_Nterm_529 | SEAEDLEELIKELAELLKDVIRKLEK |
| | AAGITELRKGTVEDKDILRDLERRLREILEESDRLLEELKRRLEEI | | INRRLVKILEDIIRRLKEISKEAEEE |
| | LRKSKELLRRLEEVLREILKRAEEVKRSNLPKEELIKEIVKLLEEL | | LRKG (SEQ ID NO: 27269) |
| | LRVIEKILEDNIRLLEELVEVIKEILEKHLRLLEELVRVIERILRE | | |
| | VGKDKDEAERRRRELKDKLDRLREEHEEVKRRLEEELTRLRETHKK | | |
| | IEKELREALKRVRDRST (SEQ ID NO: 27268) | | |
| 3plus1_Cage_568_GFP11_Nterm | KEIEETLKELEDLNREMVETNRRVLEETRRLNKETVDRVKATRDHM | 3plus1_Key_Nterm_568 | KEIEETLKELEDLNREMVETNRRVLE |
| | VLHEYVNAAGITKGPTSEELKRLLAELEELLARVVRRVEELLKKST | | ETRRLNKETVDRVKATLDELAKMLKK |
| | DLLERAVKDSADALRRSHEVLKEVASRVKRAKDEGLPREEVLRLLR | | LVDDVRKG (SEQ ID NO: 27271) |
| | ELLERHAKVLKDIVRVSEKLLREHLKVLREIVEVLEELLERILKVI | | |
| | LDTTGGDKAVEELEKALEEIKRRLKEVIDRYEDELRKLRKEYKEKI | | |
| | DKYERKLEEIERRERT (SEQ ID NO: 27270) | | |
| 3plus1_Cage_Nterm_581 | SALETVKKLLEDSSEKIERIVEEDERVAKESSDRIRRLVEEDKRVA | 3plus1_Key_Nterm_581 | SALETVKKLLEDSSEKIERIVEEDER |
| | DEILDLIEKIGDTDTLLKLVEEWSRTSKKLLDDVLKLHKDWSDDSR | | VAKESSDRIRRLVEEDKRVADEILDL |
| | RLLEEILRVHEELIRRVKEILDREGKPEEVVRELEKVLKESLDTLE | | IEKI (SEQ ID NO: 27273) |
| | EIIRRLDEANAATVKRVADVIRELEDINRKVLEEIKRGSDDAEAVI | | |
| | KVIEKLIRANKRVWDALLKINEDLVRVNKTVWKELLRVNEKLARDL | | |
| | ERVVK (SEQ ID NO: 27272) | | |
| 3plus1_Cage_610_GFP11_Nterm | SLEEITKRLLELVEENLARHEEILRELLELAKRLAKRDHMVLHEYV | 3plus1_Key_Nterm_610 | SLEEITKRLLELVEENLARHEEILRE |
| | NAAGITLKLLEDNGSSEEDLKRLLKEVIEELRAVVKRVKDKWDEVV | | LLELAKRLAKEDRDILEEVLKLIEEL |
| | KRIEDLVKKLKELHDDTLRKLRELVRKIVTDISESGGEAEKVKRVV | | LKLLEDN (SEQ ID NO: 27275) |
| | EKILELVERLAKVVKESVEKLLEILRELAEVSKRVAEALLRLLEEL | | |
| | VRVIRIKDERTLREVVRKVLEEAKRLLDELEEVHKRVKKELEDIIE | | |
| | ENRRVVKRVRDELREIKRELDE (SEQ ID NO: 27274) | | |
| 3plus1_Cage_647_GFP11_Nterm | DAEEVVKRLADVLRENDETIRKVVEDLVRIAEENDRLWRDHMVLHE | 3plus1_Key_Nterm_647 | DAEEVVKRLADVLRENDETIRKVVED |
| | YVNAAGITLLRRGGVPEELLDRLAKVVKSIVEKAEKILERLNRVSK | | LVRIAEENDRLWKKLVEDIAEILRRI |
| | AIAEKLKTIVDELNEVSKEIVKRAEDILRKGKDKETVLRALRTLVK | | VELLRRG (SEQ ID NO: 27277) |
| | EYADLSKEVLERVERIVREYVKLSDEVVKSLAEIVEELIRIIEDLL | | |
| | RKGNLDEDVKRALEELVSRLRKLLEDVKKASEDIVREVERIVRELA | | |
| | KRSDEILKKLEDIVEKLRE (SEQ ID NO: 27276) | | |

TABLE 8

| Row | | |
|---|---|---|
| number | Cage (column 1) | Key (column 2) |
| 1 | LOCKR_extend18 (SEQ ID NO: 6), | p18_MBP |
| | | (SEQ ID NO: 27020), |
| | BimLOCKR_extend18 | p76-long |
| | (SEQ ID NO: 22),, | (SEQ ID NO: 27027), |
| | 1fix-long-Bim-t0 (SEQ ID NO: 54), | p76-short |
| | | (SEQ ID NO: 27028), |
| | 1fix-long-GFP-t0 (SEQ ID NO: 55), | |
| | 1fix-short-BIM-t0 (SEQ ID NO: 56), | |
| | 1fix-short-GFP-t0 (SEQ ID NO: 57), | |
| 2 | LOCKRb (SEQ ID NO: 7), | key_b |
| | | (SEQ ID NO: 27022) |
| 3 | LOCKRc (SEQ ID NO: 8), | key_c |
| | | (SEQ ID NO: 27023) |

TABLE 9

| Cage Name | Cage Sequence | Key Name | Key Sequence |
|---|---|---|---|
| 2plus1_Cage_ | SEVDEVVKEVEDLVRRNEELVEEVVRRVEKVVTDDRRLVEEVVREI | 2plus1_Key_ | EKVLRKLEKVIREVRERSTRALRKVE |
| Cterm_2406 | RKIVKDVEDLARKLDKEELKRVLDEMRERIERLLEKLRRHSKKLDD | Cterm_2406 | EVIRRVREESERALRDLERVVKEVEK |
| | ELKRLLEELREHSRRVEKRLEDLLKELRERGVDEKVLRKLEKVIRE | | RMREAAR (SEQ ID NO: 27127) |
| | VRERSTRALRKVEEVIRRVREESERALRDLERVVKEVEKRMREAAR | | |
| | (SEQ ID NO: 27126) | | |
| 2plus1_Cage_ | SVEELLRKLEEVLRKIREENERSLKELRDRAREIVKRNRETNRELE | 2plus1_Key_ | EDIVRKIERIVETIEREVRESVKKVE |
| Cterm_5398 | EVIKELEKRLSGADKEKVEELVRRIRRIVERVVEEDRRTVEEIEKI | Cterm_5398 | EIARDIRRKVDESVKNVEKLLRDVDK |
| | AREVVKRDRDSADRVRRIVEDVLRKATGSEDIVRKIERIVETIERE | | KARDRKK (SEQ ID NO: 27129) |
| | VRESVKKVEEIARDIRRKVDESVKNVEKLLRDVDKKARDRKK (SEQ | | |
| | ID NO: 27128) | | |
| 2plus1_Cage_ | SESDDVIRKLRELLEELRTHVEKSIRDLRKILEDSTRHAKRSIEEL | 2plus1_Key_ | EEKLKDLIRKLRDILRRAAEAHKKLI |
| Cterm_5405 | ERLLEEVRKKPGDEEVRKTVEEISRRVAENVKRLEDLYRRMEEEVK | Cterm_5405 | DDARESLERAKREHEKLIDRLKKILE |
| | KNLDRLRKRVEDIIREVEEARKKGVDEEKLKDLIRKLRDILRRAAE | | ELER (SEQ ID NO: 27131) |
| | AHKKLIDDARESLERAKREHEKLIDRLKKILEELER (SEQ ID | | |
| | NO: 27130) | | |
| 2plus1_Cage_ | DREREVKKRLDEVRERIERLLRRVEEESRRVAEEIRRLIEEVRRRN | 2plus1_Key_ | EELREELKKLERKIEKVAKEIHDHDK |
| Cterm_5406 | KKVTEEIRELLKGLKDKEEVRRVLERLRKLNAESDELLERILERLR | Cterm_5406 | EVTERLEDLLRRITEHARKSDREIEE |
| | RLVEATNRLVKAIIEELRRLVEKIVREVPDSEELREELKKLERKIE | | TAR (SEQ ID NO: 27133) |
| | KVAKEIHDHDKEVTERLEDLLRRITEHARKSDREIEETAR (SEQ | | |
| | ID NO: 27132) | | |
| 2plus1_Cage_ | SEAEELLKRLEDRAEEILRRLEEILRTSRKLAEDVLRELEKLLRES | 2plus1_Key_ | KEVVDEIKRIVDEVRERLKRIVDENA |
| Cterm_5409 | ERRIREVLEELRGIKDKKELEDVIREVEKELDESLERSRELLKDVL | Cterm_5409 | KIVEDARRALEKIVKENEEILRRLKK |
| | KKLDDNLKESERLVEDIDRELAKILEDLKKAGVPKEVVDEIKRIVD | | ELRELRK (SEQ ID NO: 27135) |
| | EVRERLKRIVDENAKIVEDARRALEKIVKENEEILRRLKKELRELR | | |
| | K (SEQ ID NO: 27134) | | |
| 2plus1_Cage_ | SEIEKILKEIEDLARRDEEVSKKIVEDIRRLAKEVEDTSRDIVRKI | 2plus1_Key_ | EDSERLVREVEDLVRRLVRRSEKSNE |
| 5414_GFP11_ | EELAKRVLDRLRKDGSKEELEKEVREVVKTLEELVKDNHRLIRRAV | Cterm_5414 | EVKRTVEELVRRMEESNDRVRDLVRR |
| Cterm_ | EEMKRLVEENHRHSREVVKELEDLVRELRKGSGSEDSERDHMVLHE | | LVEELKRAVD (SEQ ID |
| | YVNAAGITSEKSNEEVKRTVEELVRRMEESNDRVRDLVRRLVEELK | | NO: 27141) |
| | RAVD (SEQ ID NO: 27140) | | |
| 2plus1_Cage_ | SVDEVLKEIEDALRRLKEEVERVLKENEDELRRLEEEVRRVLKEDE | 2plus1_Key_ | EKAIRDVAKEIRDRLKELEEEIEEVT |
| Cterm_5421 | ELLESLKRGVGESDEVDRVVDEIAKLSAEILEKVKKVVKEIRDSLE | Cterm_5421 | RRNLKLLADVEEEIRRVHEKTRRLLE |
| | TVKRRVDDVVRRLKELLDEIKRGSDEKAIRDVAKEIRDRLKELEEE | | TVLRRAT (SEQ ID NO: 27147) |
| | IEEVIRRNLKLLADVEEEIRRVHEKTRRLLETVLRRAT (SEQ ID | | |
| | NO: 27146) | | |
| 2plus1_Cage_ | DEIRKVVKEITDLLKASNDKNRKVVEEIRDLLRKSKKLADELVERL | 2plus1_Key_ | SEDLKRVEERAREVSRRNEESMRRVK |
| Cterm_5432 | RALVEDLRRRIDKSGDKETAEDIVRRIIEELKRILKEIEDLARRIN | Cterm_5432 | EDADRVSEANKEVLDRVREEVKRLIE |
| | REIERLVEEVERDNRDVNRAIEELLKDIARRGGSEDLKRVEERARE | | EVRETLR (SEQ ID NO: 27149) |
| | VSRRNEESMRRVKEDADRVSEANKEVLDRVREEVKRLIEEVRETLR | | |
| | (SEQ ID NO: 27148) | | |
| 2plus1_Cage_ | STAETVAEEVERVLKHSDDLIKEVEDVNRRVEEEIKRVIRELEEEN | 2plus1_Key_ | EEAAREIIKRLREVNKRTKEKLDELI |
| Cterm_5435 | ERLVAEVRKGVKGEILAEIEKRLADNSEKVREVAERAKKLLEENTA | Cterm_5435 | KHSEEVLERVKRLIDELRKHSEEVLE |
| | RVKDILRESRKLVKDLLDEVRGIGSEEAAREIIKRLREVNKRIKEK | | DLRRRAK (SEQ ID NO: 27151) |
| | LDELIKHSEEVLERVKRLIDELRKHSEEVLEDLRRRAK (SEQ ID | | |
| | NO: 27150) | | |
| 2plus1_Cage_ | SRVEEIIEDLRRLLEEIRKENEDSIRRSKELLDRVKEINDTIIAEL | 2plus1_Key_ | EDKARKVAEVAEKVLRDIDKLDRESK |
| Cterm_5439 | ERLLKDIEKEVREKGSESEEVKKALRAVLEELEKLLRRVAEINEEV | Cterm_5439 | EAFRATNEEIAKLDEDTARVAERVKK |
| | LRRNSKLVEEDERRNREVLKELARLVEELIREIGDEDKARKVAEVA | | AIEDLAK (SEQ ID NO: 27155) |
| | EKVLRDIDKLDRESKEAFRATNEEIAKLDEDTARVAERVKKAIEDL | | |
| | AK (SEQ ID NO: 27154) | | |
| 2plus1_Cage_ | SEADDVLKKLAETVKRIIERLKKLTDDSRRLVEEVHRRNDKLSKES | 2plus1_Key_ | EELSAEVKKLLDEVRKALARHKDEND |
| Cterm_5447 | AEAVRKAEERGIDEKDVRKLLEDLKKKSEEVAERNKRILDTLREIS | Cterm_5447 | KLLKEIEDSLRRHKEENDRLLEKLKE |
| | KRAEDEVRKVLKELEKTLKELEDRRPDSEELSAEVKKLLDEVRKAL | | STR (SEQ ID NO: 27157) |
| | ARHKDENDKLLKEIEDSLRRHKEENDRLLEKLKESTR (SEQ ID | | |
| | NO: 27156) | | |
| 2plus1_Cage_ | SAEELLREVAELVKRVDEDLRRLLEEVRASNEEVIRRLEEILKRIE | 2plus1_Key_ | EETVKRLLDELRELLERLKRTIEELL |
| Cterm_5456 | EENRKVVEELRRGGVSEDLVRESKRLVDESRRVIEKLVKESADSVE | Cterm_5465 | KRNRDLLADAEEKARRLLEENRKLLK |
| | RTRETVDRLREELKRLVEEIAKMVKGGSSEETVKRLLDELRELLER | | AARDTAT (SEQ ID NO: 27159) |
| | LKRTIEELLKRNRDLLADAEEKARRLLEENRKLLKAARDTAT (SEQ | | |
| | ID NO: 27158) | | |
| 2plus1_Cage_ | SEVDEVVKEVEDLVRRNEELVEEVVRRVEKVVTDDRRLVEEVVREI | 2plus1_Key_ | SEVDEVVKEVEDLVRRNEELVEEVVR |
| Nterm_2406 | RKIVKDVEDLARKLDKEELKRVLDEMRERIERLLEKLRRHSKKLDD | Nterm_2406 | RVEKVVTDDRRLVEEVVREIRKIVKD |
| | ELKRLLEELREHSRRVEKRLEDLLKELRERGVDEKVLRKLEKVIRE | | VEDLARK (SEQ ID NO: 27163) |
| | VRERSTRALRKVEEVIRRVREESERALRDLERVVKEVEKRMREAAR | | |
| | (SEQ ID NO: 27162) | | |
| 2plus1_Cage_ | DREREVKKRLDEVRERIERLLRRVEEESRRVAEEIRRLIEEVRRRN | 2plus1_Key_ | DREREVKKRLDEVRERIERLLRRVEE |
| Nterm_5406 | KKVTEEIRELLKGLKDKEEVRRVLERLRKLNAESDELLERILERLR | Nterm_5406 | ESRRVAEEIRRLIEEVRRRNKKVTEE |
| | RLVEATNRLVKAIIEELRRLVEKIVREVPDSEELREELKKLERKIE | | IRELLKGL (SEQ ID NO: 27165) |
| | KVAKEIHDHDKEVTERLEDLLRRITEHARKSDREIEETAR(SEQ | | |
| | ID NO: 27164) | | |
| 2plus1_Cage_ | SEAEELLKRLEDRAEEILRRLEEILRTSRKLAEDVLRELEKLLRES | 2plus1_Key_ | SEAEELLKRLEDRAEEILRRLEEILR |
| Nterm_5409 | ERRIREVLEELRGIKDKKELEDVIREVEKELDESLERSRELLKDVL | Nterm_5409 | TSRKLAEDVLRELEKLLRESERRIRE |
| | KKLDDNLKESERLVEDIDRELAKILEDLKKAGVPKEVVDEIKRIVD | | VLEELRGI (SEQ ID NO: 27167) |
| | EVRERLKRIVDENAKIVEDARRALEKIVKENEEILRRLKKELRELR | | |
| | K (SEQ ID NO: 27166) | | |
| 3plus_Cage_ | SEAEDLEELIKELAELLKDVIRKLEKINRRLVKILEDIIRRLKEIS | 3plus_Key_ | KDEAERRRRELKDKLDRLREEHEEVK |
| 529_GFP11_ | KEAEEELRKGTVEDKDILRDLERRLREILEESDRLLEELKRRLEEI | Cterm_529 | RRLEEELTRLRETHKKIEKELREALK |
| Cterm | LRKSKELLRRLEEVLREILKRAEEVKRSNLPKEELIKEIVKLLEEL | | RVRDRST (SEQ ID NO: 27179) |
| | LRVIEKILEDNIRLLEELVEVIKEILEKHLRLLEELVRVIERILRE | | |
| | VGKDKDEAERRDHMVLHEYVNAAGITEEVKRRLEEELTRLRETHKK | | |
| | IEKELREALKRVRDRST (SEQ ID NO: 27178) | | |
| 3plus1_Cage_ | SEKEELKRLLDKLLKELKRLSDELKATIDKILKILKEVSEEVKRTA | 3plus1_Key_ | EDELRKVEEDLKRLEDKLKKLLEDYE |
| Cterm_500 | DELLDAIRRGGVDEEVLREIKREIEEIEKKLRKVNKEIEDEIREIK | Cterm_500 | KKVRELEETLDDLLRKYEETLRRLEK |
| | KKLDEVDDKITKEVEKIKEALDKGGVDAKEVIKALKEILKEHADVF | | ELEEAER (SEQ ID NO: 27185) |
| | EDVLRRLKEIIKRHRDVVKEVLEELRKILEKVAEVLKRQGRSEDEL | | |
| | RKVEEDLKRLEDKLKKLLEDYEKKVRELEETLDDLLRKYEETLRRL | | |
| | EKELEEAER (SEQ ID NO: 27184) | | |
| 3plus1_Cage_ | SEKEELLKLIKRVIELLKRVLEEHLRLVEDVIRRLKELLDSNEKIV | 3plus1_Key_ | EDLLRKAKKVITEVREKLKRNLEDVR |
| Cterm_510 | REVIEDLKRLLDEVRGDKEELDRIKEKLEEVLERYKRRLEEIKRDL | Cterm_510 | RVIEDVKRKSARILEEARRLIEEVER |
| | ERMLEDYKRELKRIEEDLRRVLEEVERIATRGEGPAEALIDKLRKI | | ELEKIRK (SEQ ID NO: 27191) |
| | LERALRELDKLSKKLDELLKKVLEELEKSNREIDKLLKDVLRRVEE | | |
| | GGASEDLLRKAKKVITEVREKLKRNLEDVRRVIEDVKRKSARILEE | | |
| | ARRLIEEVERELEKIRK (SEQ ID NO: 27190) | | |
| 3plus1_Cage_ | SEAEDLEELIKELAELLKDVIRKLEKINRRLVKILEDIIRRLKEIS | 3plus1_Key_ | KDEAERRRRELKDKLDRLREEHEEVK |
| 528_GFP11_ | KEAEEELRKGTVEDKDILRDLERRLREILEESDRLLEELKRRLEEI | Cterm_528 | RRLEEELTRLRETHKKIEKELREALK |
| Cterm | LRKSKELLRRLEEVLREILKRAEEVKRSNLPKEELIKEIVKLLEEL | | RVRDRST (SEQ ID NO: 27193) |
| | LRVIEKILEDNIRLLEELVEVIKEILEKHLRLLEELVRVIERILRE | | |
| | VGRDHMVLHEYVNAAGITLDRLREEHEEVKRRLEEELTRLRETHKK | | |
| | IEKELREALKRVRDRST (SEQ ID NO: 27192) | | |
| 3plus1_Cage_ | SEAEDLEELIKELAELLKDVIRKLEKINRRLVKILEDIIRRLKEIS | 3plus1_Key_ | KDEAERRRRELKDKLDRLREEHEEVK |
| 528_GFP11_ | KEAEEELRKGTVEDKDILRDLERRLREILEESDRLLEELKRRLEEI | Cterm_528 | RRLEEELTRLRETHKKIEKELREALK |
| Cterm_ | LRKSKELLRRLEEVLREILKRAEEVKRSNLPKEELIKEIVKLLEEL | | RVRDRST (SEQ ID NO: 27195) |
| | LRVIEKILEDNIRLLEELVEVIKEILEKHLRLLEELVRVIERILRE | | |
| | VGKDKRDHMVLHEYVNAAGITLREEHEEVKRRLEEELTRLRETHKK | | |
| | IEKELREALKRVRDRST (SEQ ID NO: 27194) | | |
| 3plus1_Cage_ | SEAEDLEELIKELAELLKDVIRKLEKINRRLVKILEDIIRRLKEIS | 3plus1_Key_ | KDEAERRRRELKDKLDRLREEHEEVK |
| 528_GFP11_ | KEAEEELRKGTVEDKDILRDLERRLREILEESDRLLEELKRRLEEI | Cterm_528 | RRLEEELTRLRETHKKIEKELREALK |
| Cterm | LRKSKELLRRLEEVLREILKRAEEVKRSNLPKEELIKEIVKLLEEL | | RVRDRST (SEQ ID NO: 27197) |
| | LRVIEKILEDNIRLLEELVEVIKEILEKHLRLLEELVRVIERILRE | | |
| | VGKDKDEAERDHMVLHEYVNAAGITHEEVKRRLEEELTRLRETHKK | | |
| | IEKELREALKRVRDRST (SEQ ID NO: 27196) | | |
| 3plus1_Cage_ | SEAEDLEELIKELAELLKDVIRKLEKINRRLVKILEDIIRRLKEIS | 3plus1_Key_ | KDEAERRRRELKDKLDRLREEHEEVK |
| 529_GFP11_ | KEAEEELRKGTVEDKDILRDLERRLREILEESDRLLEELKRRLEEI | Cterm_529 | RRLEEELTRLRETHKKIEKELREALK |
| Cterm | LRKSKELLRRLEEVLREILKRAEEVKRSNLPKEELIKEIVKLLEEL | | RVRDRST (SEQ ID NO: 27199) |
| | LRVIEKILEDNIRLLEELVEVIKEILEKHLRLLEELVRVIERILRE | | |
| | VGKRDHMVLHEYVNAAGITDRLREEHEEVKRRLEEELTRLRETHKK | | |
| | IEKELREALKRVRDRST (SEQ ID NO: 27198) | | |
| 3plus1_Cage_ | SEAEDLEELIKELAELLKDVIRKLEKINRRLVKILEDIIRRLKEIS | 3plus1_Key_ | KDEAERRRRELKDKLDRLREEHEEVK |
| 529_GFP11_ | KEAEEELRKGTVEDKDILRDLERRLREILEESDRLLEELKRRLEEI | Cterm_529 | RRLEEELTRLRETHKKIEKELREALK |
| Cterm | LRKSKELLRRLEEVLREILKRAEEVKRSNLPKEELIKEIVKLLEEL | | RVRDRST (SEQ ID NO: 27201) |
| | LRVIEKILEDNIRLLEELVEVIKEILEKHLRLLEELVRVIERILRE | | |
| | VGKDRDHMVLHEYVNAAGITRLREEHEEVKRRLEEELTRLRETHKK | | |
| | IEKELREALKRVRDRST (SEQ ID NO: 27200) | | |
| 3plus1_Cage_ | SEAEDLEELIKELAELLKDVIRKLEKINRRLVKILEDIIRRLKEIS | 3plus1_Key_ | KDEAERRRRELKDKLDRLREEHEEVK |
| 529_GFP11_ | KEAEEELRKGTVEDKDILRDLERRLREILEESDRLLEELKRRLEEI | Cterm_529 | RRLEEELTRLRETHKKIEKELREALK |
| Cterm | LRKSKELLRRLEEVLREILKRAEEVKRSNLPKEELIKEIVKLLEEL | | RVRDRST (SEQ ID NO: 27203) |
| | LRVIEKILEDNIRLLEELVEVIKEILEKHLRLLEELVRVIERILRE | | |
| | VGKDKDRDHMVLHEYVNAAGITREEHEEVKRRLEEELTRLRETHKK | | |
| | IEKELREALKRVRDRST (SEQ ID NO: 27202) | | |
| 3plus1_Cage_ | DEDRIIEEIARLLEELLRELLELIKKLIETNRRLNEEHERAVRELA | 3plus1_Key_ | EKIAEEIERELEELRRMIKRLHEDLE |
| 534_GFP11_ | RLLEELLDRLVKKGISDEKLKRIRERLKRALDDLERLHREINKRLE | Cterm_534 | RKLKESEDELREIEARLEEKIRRLEE |
| Cterm | DLVRELEKLVREILKELKDALEELRRASARAGGEEVLRRLEEIVKK | | KLERKRR (SEQ ID NO: 27207) |
| | LLDLVRRILERLKEIHKDNVRLLRELNERLTRIVEDLVRLIREILR | | |
| | EAGVDERDHMVLHEYVNAAGITIKRLHEDLERKLKESEDELREIEA | | |
| | RLEEKIRRLEEKLERKRR (SEQ ID NO: 27206) | | |
| 3plus1_Cage_ | DEDRIIEEIARLLEELLRELLELIKKLIETNRRLNEEHERAVRELA | 3plus1_Key_ | EKIAEEIERELEELRRMIKRLHEDLE |
| 534_GFP11_ | RLLEELLDRLVKKGISDEKLKRIRERLKRALDDLERLHREINKRLE | Cterm_534 | RKLKESEDELREIEARLEEKIRRLEE |
| Cterm | DLVRELEKLVREILKELKDALEELRRASARAGGEEVLRRLEEIVKK | | KLERKRR (SEQ ID NO: 27209) |
| | LLDLVRRILERLKEIHKDNVRLLRELNERLTRIVEDLVRLIREILR | | |
| | EAGVDEKIRDHMVLHEYVNAAGITRLHEDLERKLKESEDELREIEA | | |
| | RLEEKIRRLEEKLERKRR (SEQ ID NO: 27208) | | |
| 3plus1_Cage_ | DEDRIIEEIARLLEELLRELLELIKKLIETNRRLNEEHERAVRELA | 3plus1_Key_ | EKIAEEIERELEELRRMIKRLHEDLE |
| 534_GFP11_ | RLLEELLDRLVKKGISDEKLKRIRERLKRALDDLERLHREINKRLE | Cterm_534 | RKLKESEDELREIEARLEEKIRRLEE |
| Cterm | DLVRELEKLVREILKELKDALEELRRASARAGGEEVLRRLEEIVKK | | KLERKRR (SEQ ID NO: 27211) |
| | LLDLVRRILERLKEIHKDNVRLLRELNERLTRIVEDLVRLIREILR | | |
| | EAGVDEKIAEEIERDHMVLHEYVNAAGITLERKLKESEDELREIEA | | |
| | RLEEKIRRLEEKLERKRR (SEQ ID NO: 27210) | | |
| 3plus1_Cage_ | SEKEKLLKESEEEVRRLRRTLEELLRKYREVLERLRKELREIEERV | 3plus1_Key_ | ERLVKTLIEDVEAVIKRILELITRVA |
| Cterm_539 | RDVVRRLKEVLDRKGLDIDTIIKEVEDLLKTVLDRLRELLDKIRRL | Cterm_539 | EDNERVLERIIRELTDNLERHLKIVR |
| | TKEAIEVVREIIERIVRHAERVKDELRKEGGDKEKLDRVDRLIKEN | | EIVK (SEQ ID NO: 27213) |
| | TRHLKEILDRIEDLVRRSEKKLRDIIREVRRLIEELRKKAEEIKKG | | |
| | PDERLVKILIEDVEAVIKRILELITRVAEDNERVLERIIRELTDNL | | |
| | ERHLKIVREIVK (SEQ ID NO: 27212) | | |
| 3plus1_Cage_ | DKAEVLREALKLLKDLLEELIKIHEESLKRILDLIDTLVKVHEDAL | 3plus1_Key_ | EEIDRELKRVVEELRRLHEEIKERLD |
| Cterm_548 | RALKELLERSGLDERELRKVERMATESLRTIAKLKEELRDLARRSL | Cterm_548 | DVARRSEEELRRIIKKLKEVVKEIRK |
| | EKLREDLKRVDDILRKVEEKVRRTGPSEELIEELIRTIEKLLKEIV | | KLK (SEQ ID NO: 27215) |
| | RINEEVLKAVRELLKTLLKLSEDVVRRIEEILRKGGVPEEIDRELK | | |
| | RVVEELRRLHEEIKERLDDVARRSEEELRRIIKKLKEVVKEIRKKL | | |
| | K (SEQ ID NO: 27214) | | |
| 3plus1_Cage_ | SERELIERWLELHKEILRLIRELVERLLKLHREILDTIKKLIRELL | 3plus1_Key_ | DDERRILTELLKRMEDILEKVERTLK |
| Cterm_556 | ELLEDIARKLGLDKEAKDELREIAKRVEDKLEKLERESRKVEEDLK | Cterm_556 | KLLDDSARMAEEVKKTLKELLERSEK |
| | RKLKELTDESDIVEKRVRDVVRRGIQSREEIAEELLRLDRKLLKAV | | VAEDVRK (SEQ ID NO: 27217) |
| | EELLKEILDLNKKLLDDVRAILEETRRVLEKLLDRVRRGERTDDER | | |
| | RILTELLKRMEDILEKVERTLKKLLDDSARMAEEVKKILKELLERS | | |
| | EKVAEDVRK (SEQ ID NO: 27216) | | |
| 3plus1_Cage_ | SKKELLEEVVRRAIELLKRHLEKLKRILEEIVRLLEEHLEKVERVL | 3plus1_Key_ | EDKLKEIEDELRRLLEELRRLDKAIK |
| Cterm_560 | EAILSLLDDLLRRGGDERAIRTLEDVKRRLREILERLADENAKAIK | Cterm_560 | DRLRELKKDLDEANRRIKETLKKLLR |
| | RLADLLDKLEKRNKEAIERLEEILEELKRVRRDEELLRVLETLLKI | | EVEK (SEQ ID NO: 27219) |
| | IEDILRENTKVLEDLLRLVEEILEANLRVVEELLRLAREILTEIVG | | |
| | DEDKLKEIEDELRRLLEELRRLDKAIKDRLRELKKDLDEANRRIKE | | |
| | TLKKLLREVEK (SEQ ID NO: 27218) | | |
| 3plus1_Cage_ | KEIEETLKELEDLNREMVETNRRVLEETRRLNKETVDRVKATLDEL | 3plus1_Key_ | KAVEELEKALEEIKRRLKEVIDRYED |
| 568_GFP11_ | AKMLKKLVDDVRKGPTSEELKRLLAELEELLARVVRRVEELLKKST | Cterm_568 | ELRKLRKEYKEKIDKYERKLEEIERR |
| Cterm | DLLERAVKDSADALRRSHEVLKEVASRVKRAKDEGLPREEVLRLLR | | ERT (SEQ ID NO: 27221) |
| | ELLERHAKVLKDIVRVSEKLLREHLKVLREIVEVLEELLERILKVI | | |
| | LDTTRDHMVLHEYVNAAGITKRRLKEVIDRYEDELRKLRKEYKEKI | | |
| | DKYERKLEEIERRERT (SEQ ID NO: 27220) | | |
| 3plus1_Cage_ | KEIEETLKELEDLNREMVETNRRVLEETRRLNKETVDRVKATLDEL | 3plus1_Key_ | KAVEELEKALEEIKRRLKEVIDRYED |
| 568_GFP11 | AKMLKKLVDDVRKGPTSEELKRLLAELEELLARVVRRVEELLKKST | Cterm_568 | ELRKLRKEYKEKIDKYERKLEEIERR |
| Cterm | DLLERAVKDSADALRRSHEVLKEVASRVKRAKDEGLPREEVLRLLR | | ERT (SEQ ID NO: 27223) |
| | ELLERHAKVLKDIVRVSEKLLREHLKVLREIVEVLEELLERILKVI | | |
| | LDTTGGDRDHMVLHEYVNAAGITLKEVIDRYEDELRKLRKEYKEKI | | |
| | DKYERKLEEIERRERT (SEQ ID NO: 27222) | | |
| 3plus1_Cage_ | SALETVKKLLEDSSEKIERIVEEDERVAKESSDRIRRLVEEDKRVA | 3plus1_Key_ | AEAVIKVIEKLIRANKRVWDALLKIN |
| Cterm_581 | DEILDLIEKIGDIDILLKLVEEWSRTSKKLLDDVLKLHKDWSDDSR | Cterm_581 | EDLVRVNKTVWKELLRVNEKLARDLE |
| | RLLEEILRVHEELIRRVKEILDREGKPEEVVRELEKVLKESLDTLE | | RVVK (SEQ ID NO: 27227) |
| | EIIRRLDEANAATVKRVADVIRELEDINRKVLEEIKRGSDDAEAVI | | |
| | KVIEKLIRANKRVWDALLKINEDLVRVNKTVWKELLRVNEKLARDL | | |
| | ERVVK (SEQ ID NO: 27226) | | |
| 3plus1_Cage_ | SKEEKLKDDVRAVLEDLDRVLKELEKLSEDNLRELKRVLDRITDLH | 3plus1_Key_ | SKAAEDILRVLEKLVKVSREAIKLIL |
| Cterm_585 | RRILDELRKGIGSEELLRRVEKVLKDNLDLLRKLVEEHKESSERDL | Cterm_585 | ELSEHHVRVSTRIARLLLDVARKLAE |
| | KRVEDLVREIKEVLRKLLELEDRGTDIRKIEEEIERLLRKIRKAVE | | VIKEAER (SEQ ID NO: 27229) |
| | ESKDLNRRNSERIEEVARRSEELARRLLKEIRERGDSKAAEDILRV | | |
| | LEKLVKVSREAIKLILELSEHHVRVSTRIARLLLDVARKLAEVIKE | | |
| | AER (SEQ ID NO: 27228) | | |
| 3plus1_Cage_ | SEIEDVIRRLRKILEDLERVSEKLLREIKKILDEARRLNEEVIKEI | 3plus1_Key_ | IEDLVREVERLIKRIEDSLRELEKTV |
| Cterm_587 | KRVLEDAVRVFRDGSGSKEELAKLVEELIRELAKLAKEVDEIHKRI | Cterm_587 | RELLKRIKEASDKVREDVDRLIKELK |
| | VERLKALVEDAERIHRKIVETLEEIVRGVPSEELKRVVEAIVEVIK | | EAAD (SEQ ID NO: 27231) |
| | EHLKVLADVIRRIIKAIEENAETIKRVLEDIVRVLELVLRGEGSIE | | |
| | DLVREVERLIKRIEDSLRELEKTVRELLKRIKEASDKVREDVDRLI | | |
| | KELKEAAD (SEQ ID NO: 27230) | | |
| 3plus1_Cage_ | SREELLDRILEAIAKILEDLKRLIDENLARLEEVVRELERIIDRNL | 3plus1_Key_ | DEIIRKLDELLKEVEKVHKEVKDRIR |
| Cterm_605 | KLIREILDELKKGSGSEEILEKIKKVDKELEDLIRRLLKKLEDLIR | Cterm_605 | KLLEDHKRSLDEVKKKLERLLERAKE |
| | ETERRLREILKRIRDLLKEVKDRDKDLERLLEVLEEVLRVIAELAK | | VVEREKK (SEQ ID NO: 27233) |
| | ELLDSLRKVLKVVEEVLRLLNEVNKEVLDVIRELAKDGGSDEIIRK | | |
| | LDELLKEVEKVHKEVKDRIRKLLEDHKRSLDEVKKKLERLLERAKE | | |
| | VVEREKK (SEQ ID NO: 27232) | | |
| 3plus1_Cage_ | SEREELLERIKEILKRVKDKLDEDLKRLKEILEKLKEKADRDLEEL | 3plus1_Key_ | SETAVRAIIRVLEKHLEAVRRVLEEL |
| Cterm_607 | RRRIEEVREKLERTGRIDELVKEVLDTVRRNLENLKRLVEDILRKL | Cterm_607 | LKVLAEHLETVRELIERLKRVLEEAI |
| | EENVKNLTDLVREILKLITELIKRLEDGGLPKEVLDALRRVLEKLE | | EVVERVAR (SEQ ID NO: 27235) |
| | ELLREILERLKRSLEAVKRKIEELLKELERSLDELRRALERIRKEI | | |
| | GDSETAVRAIIRVLEKHLEAVRRVLEELLKVLAEHLETVRELIERL | | |
| | KRVLEEAIEVVERVAR (SEQ ID NO: 27234) | | |
| 3plus1_Cage_ | SLEEITKRLLELVEENLARHEEILRELLELAKRLAKEDRDILEEVL | 3plus1_Key_ | ERTLREVVRKVLEEAKRLLDELEEVH |
| 611_GFP11_ | KLIEELLKLLEDNGSSEEDLKRLLKEVIEELRAVVKRVKDKWDEVV | Cterm_611 | KRVKKELEDIIEENRRVVKRVRDELR |
| Cterm | KRIEDLVKKLKELHDDILRKLRELVRKIVIDISESGGEAEKVKRVV | | EIKRELDE (SEQ ID NO: 27239) |
| | EKILELVERLAKVVKESVEKLLEILRELAEVSKRVAEALLRLLEEL | | |
| | VRVIRIKDERDHMVLHEYVNAAGITLLDELEEVHKRVKKELEDIIE | | |
| | ENRRVVKRVRDELREIKRELDE (SEQ ID NO: 27238) | | |
| 3plus1_Cage_ | SEKELVDDIRRILEEILRLLRSLLEEVIRLLEENEKLVRRHLKTVI | 3plus1_Key_ | DSLVREVEELIKRLEKHIDDLLKTSR |
| Cterm_632 | DILRRVAKLLDENGIRTDEADRVLERLEKAHRELLEDYKRALEKIK | Cterm_632 | DLVKRVLDLVDEVVKRVEDLVERVKE |
| | ETLERVLREAEEVVKKIDDALRKLGGSKEVLKRLLEELLRLVEKIA | | KIDT (SEQ ID NO: 27241) |
| | EEIKRLLSELVRVIEELVRINKELLEEAVRVIRKEVGDDSLVREVE | | |
| | ELIKRLEKHIDDLLKTSRDLVKRVLDLVDEVVKRVEDLVERVKEKI | | |
| | DT (SEQ ID NO: 27240) | | |
| 3plus1_Cage_ | DAEEVVKRLADVLRENDETIRKVVEDLVRIAEENDRLWKKLVEDIA | 3plus1_Key_ | EDVKRALEELVSRLRKLLEDVKKASE |
| 646_GFP11_ | EILRRIVELLRRGGVPEELLDRLAKVVKSIVEKAEKILERLNRVSK | Cterm_646 | DIVREVERIVRELAKRSDEILKKLED |
| Cterm | AIAEKLKTIVDELNEVSKEIVKRAEDILRKGKDKETVLRALRTLVK | | IVEKLRE (SEQ ID NO: 27245) |
| | EYADLSKEVLERVERIVREYVKLSDEVVKSLAEIVEELIRIIEDLL | | |
| | RKGNRDHMVLHEYVNAAGITRKLLEDVKKASEDIVREVERIVRELA | | |
| | KRSDEILKKLEDIVEKLRE (SEQ ID NO: 27244) | | |
| 3plus1_Cage_ | DAEEVVKRLADVLRENDETIRKVVEDLVRIAEENDRLWKKLVEDIA | 3plus1_Key_ | EDVKRALEELVSRLRKLLEDVKKASE |
| 646_GFP11_ | EILRRIVELLRRGGVPEELLDRLAKVVKSIVEKAEKILERLNRVSK | Cterm_646 | DIVREVERIVRELAKRSDEILKKLED |
| Cterm | AIAEKLKTIVDELNEVSKEIVKRAEDILRKGKDKETVLRALRTLVK | | IVEKLRE (SEQ ID NO: 27247) |
| | EYADLSKEVLERVERIVREYVKLSDEVVKSLAEIVEELIRIIEDLL | | |
| | RKGNLDEDRDHMVLHEYVNAAGITEDVKKASEDIVREVERIVRELA | | |
| | KRSDEILKKLEDIVEKLRE (SEQ ID NO: 27246) | | |
| 3plus1_Cage_ | DAEEVVKRLADVLRENDETIRKVVEDLVRIAEENDRLWKKLVEDIA | 3plus1_Key_ | EDVKRALEELVSRLRKLLEDVKKASE |
| 646_GFP11_ | EILRRIVELLRRGGVPEELLDRLAKVVKSIVEKAEKILERLNRVSK | Cterm_646 | DIVREVERIVRELAKRSDEILKKLED |
| Cterm | AIAEKLKTIVDELNEVSKEIVKRAEDILRKGKDKETVLRALRTLVK | | IVEKLRE (SEQ ID NO: 27249) |
| | EYADLSKEVLERVERIVREYVKLSDEVVKSLAEIVEELIRIIEDLL | | |
| | RKGNLDEDVRDHMVLHEYVNAAGITDVKKASEDIVREVERIVRELA | | |
| | KRSDEILKKLEDIVEKLRE (SEQ ID NO: 27248) | | |
| 3plus1_Cage_ | DAEEVVKRLADVLRENDETIRKVVEDLVRIAEENDRLWKKLVEDIA | 3plus1_Key_ | EDVKRALEELVSRLRKLLEDVKKASE |
| 647_GFP11_ | EILRRIVELLRRGGVPEELLDRLAKVVKSIVEKAEKILERLNRVSK | Cterm_647 | DIVREVERIVRELAKRSDEILKKLED |
| Cterm | AIAEKLKTIVDELNEVSKEIVKRAEDILRKGKDKETVLRALRTLVK | | IVEKLRE (SEQ ID NO: 27251) |
| | EYADLSKEVLERVERIVREYVKLSDEVVKSLAEIVEELIRIIEDLL | | |
| | RKGNLRDHMVLHEYVNAAGITKLLEDVKKASEDIVREVERIVRELA | | |
| | KRSDEILKKLEDIVEKLRE (SEQ ID NO: 27250) | | |
| 3plus1_Cage_ | DAEEVVKRLADVLRENDETIRKVVEDLVRIAEENDRLWKKLVEDIA | 3plus1_Key_ | EDVKRALEELVSRLRKLLEDVKKASE |
| 647_GFP11_ | EILRRIVELLRRGGVPEELLDRLAKVVKSIVEKAEKILERLNRVSK | Cterm_647 | DIVREVERIVRELAKRSDEILKKLED |
| Cterm | AIAEKLKTIVDELNEVSKEIVKRAEDILRKGKDKETVLRALRTLVK | | IVEKLRE (SEQ ID NO: 27253) |
| | EYADLSKEVLERVERIVREYVKLSDEVVKSLAEIVEELIRIIEDLL | | |
| | RKGNLDEDVKRALERDHMVLHEYVNAAGITS EDIVREVERIVRELA | | |
| | KRSDEILKKLEDIVEKLRE (SEQ ID NO:27252) | | |
| 3plus1_Cage_ | DAEEVVKRLADVLRENDETIRKVVEDLVRIAEENDRLWKKLVEDIA | 3plus1_Key_ | EDVKRALEELVSRLRKLLEDVKKASE |
| Cterm_647 | EILRRIVELLRRGGVPEELLDRLAKVVKSIVEKAEKILERLNRVSK | Cterm_647 | DIVREVERIVRELAKRSDEILKKLED |
| | AIAEKLKTIVDELNEVSKEIVKRAEDILRKGKDKETVLRALRTLVK | | IVEKLRE (SEQ ID NO: 27255) |
| | EYADLSKEVLERVERIVREYVKLSDEVVKSLAEIVEELIRIIEDLL | | |
| | RKGNLDEDVKRALEELVSRLRKLLEDVKKASEDIVREVERIVRELA | | |
| | KRSDEILKKLEDIVEKLRE (SEQ ID NO: 27254) | | |
| 3plus1_Cage_ | DEEETLRRLLERKVELAKEYLDVSKEVIDRITKLLDEYLKISKRIV | 3plus1_Key_ | SREALEEARRRLEELLRELNEITKDL |
| Cterm_653 | DATVELLERGDLGPDELIKRLAEELERSLRELEEEIKRLKRELEES | Cterm_653 | EAKLEKLLRDLNELTKALEEELKRLL |
| | LKKLKEIIDRLAEEAEKLLAVLKRGEGSEEEALRALASLVRELIEV | | DELKKRTD (SEQ ID NO: 27257) |
| | LRENDERLRDVLRRLIEALRKNNEILERVLRKLVRAAEERGRDESS | | |
| | REALEEARRRLEELLRELNEITKDLEAKLEKLLRDLNELTKALEEE | | |
| | LKRLLDELKKRTD (SEQ ID NO: 27256) | | |
| 3plus1_Cage_ | DEERIIKTLEDINAKLVEDIKRILDKVAELNERLADAIRKILEETK | 3plus1_Key_ | KDTLRTVEKLVEDVKRRLDKLLEDYK |
| Cterm_658 | RILEATTRKVRKDGEISEELLRRLEEKLRKLLEDLERVLAEHEDES | Cterm_658 | RLIEEVKKELDKLLKEYEDALREIKK |
| | RRILEEVERLLKRHADASKELLDRARSVARGVKSDKELVDRLKKLI | | RIDE (SEQ ID NO: 27259) |
| | DDSLESVRELIERLKELLDRLVKSVEDLIRTIKELLDRLVEVLREG | | |
| | VSDKDILRIVEKLVEDVKRRLDKLLEDYKRLIEEVKKELDKLLKEY | | |
| | EDALREIKKRIDE (SEQ ID NO: 27258) | | |
| 3plus1_Cage_ | SLVDELRKSLERNVRVSEEVARRLKEALKRWVDVVRKVVEDLIRLN | 3plus1_Key_ | SLVDELRKSLERNVRVSEEVARRLKE |
| Nterm_263 | EDVVRVVEKVIVDESAIERVRRIIEELNRKLDAVLKKNEDLVRRLT | Nterm_263 | ALKRWVDVVRKVVEDLIRLNEDVVRV |
| | ELLDKLLEENRRLVEELDEDLKRRGGTEEVIDTILELIERSIERLK | | VEKV (SEQ ID NO: 27263) |
| | RLLDELLRIVREALKDNKRVADENLKKLKEILDELRKDGVEDEELK | | |
| | RVLEKAADLHRRLKDRHRKLLEDLERIIRELKKKLDEVVEENKRSV | | |
| | DELKR (SEQ ID NO: 27262) | | |
| 3plus1_Cage_ | DAEEVVKRLADVLRENDETIRKVVEDLVRIAEENDRLWRDHMVLHE | 3plus1_Key_ | DAEEVVKRLADVLRENDETIRKVVED |
| 647_GFP11_ | YVNAAGITLLRRGGVPEELLDRLAKVVKSIVEKAEKILERLNRVSK | Nterm_647 | LVRIAEENDRLWKKLVEDIAEILRRI |
| Nterm | AIAEKLKTIVDELNEVSKEIVKRAEDILRKGKDKETVLRALRTLVK | | VELLRRG (SEQ ID NO: 27277) |
| | EYADLSKEVLERVERIVREYVKLSDEVVKSLAEIVEELIRIIEDLL | | |
| | RKGNLDEDVKRALEELVSRLRKLLEDVKKASEDIVREVERIVRELA | | |
| | KRSDEILKKLEDIVEKLRE (SEQ ID NO: 27276) | | |

TABLE 10
Exemplary binding domains

>Her2_DARPin (SEQ ID NO: 27399)
DLGKKLLEAARAGQDDEVRILMANGADVNAKDEYGLTPLYLATAHGHLEIVEVLLKNGADVNAVDAIGFTPLHLA
AFIGHLEIAEVLLKHGADVNAQDKFGKTAFDISIGNGNEDLAEILQKLN
>EGER_DARPin (SEQ ID NO: 27400)
DLGKKLLEAARAGQDDEVRILMANGADVNADDTWGWTPLHLAAYQGHLEIVEVLLKNGADVNAYDYIGWTPLHLA
ADGHLEIVEVLLKNGADVNASDYIGDTPLHLAAHNGHLEIVEVLLKHGADVNAQDKEGKTAFDISIDNGNEDLAE
ILQKLN
>EpCAM_DARPin DARPin (SEQ ID NO: 27401)
DLGKKLLEAARAGQDDEVRILVANGADVNAYEGTTPLHLAAAHGRLEIVEVLLKNGADVNAQDVWGITPLHLAAY
NGHLEIVEVLLKYGADVNAHDTRGWTPLHLAAINGHLEIVEVLLKNVADVNAQDRSGKTPFDLAIDNGNEDIAEV
LQKAAKLN
>Anti-Her2 scFv (SEQ ID NO: 27402)
DIQMTQSPSSLSASVGDRVTITCRASQDVNTAVAWYQQKPGKAPKLLIYSASFLYSGVPSRFSGSRSGTDFTLTI
SSLQPEDFATYYCQQHYTTPPTFGQGTKVEIKGSTSGSGKPGSGEGSGEVQLVESGGGLVQPGGSLRLSCAASGF
NIKDTYTHWVRQAPGKGLEWVARTYPTNGYTRYADSVKGRETISADTSKNTAYLQMNSLRAEDTAVYYCSRWGGD
GFYAMDYWGQGTLVTVSS
>Anti-EGFR scFv (SEQ ID NO: 27403)
QVQLKQSGPGLVQPSQSLSITCTVSGFSLTNYGVHWVRQSPGKGLEWLGVIWSGGNTDYNTPFTSRLSINKDNSK
SQVFFKMNSLQSNDTAIYYCARALTYYDYEFAYWGQGTLVTVSAGGGGSGGGGSGGGGSDILLTQSPVILSVSPG
ERVSFSCRASQSIGTNIHWYQQRTNGSPRLLIKYASESISGIPSRFSGSGSGTDFTLSINSVESEDIADYYCQQN
NNWPTTFGAGTKLELKRT

TABLE 11
Exemplary cage polypeptides with binding domains
Co-LOCKR Cage proteins (These proteins may alternatively be used as Decoys
for effector proteins that do not interact with Bim)
(parentheses are optional sequences)

>Her2_Cage Original Cage targeted to Her2 by DARPin SEQ ID NO: 27404
(MGSHHHHHHGSGSENLYFQGSGGS)DLGKKLLEAARAGQDDEVRILMANGADVNAKDEYGLIPLYLATAHGHLE
IVEVLLKNGADVNAVDAIGFTPLHLAAFIGHLEIAEVLLKHGADVNAQDKEGKTAFDISIGNGNEDLAEILQKLN
(SGSGSGKPGQASGS)ELARKLLEASTKLQRLNIRLAEALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRA
VELLVKLTDPATIREALEHAKRRSKEIIDEAERAIRAAKRESERIIEEARRLIEKGSGSGSELARELLRAHAQLQ
RLNLELLRELLRALAQLQELNLDLLRLASELTDEIWIAQELRRIGDEFNAYYADAERLIREAAAASEKISREAER
LIR
>EGER_Cage Original Cage targeted to EGFR by DARPin SEQ ID NO: 27405
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKEGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELTDEIWIAQELRR
IGDEFNAYYADAERLIREAAAASEKISREAERLIR
>EpCAM_Cage Original Cage targeted to EpCAM by DARPin SEQ ID NO: 27406
(MGSHHHHHHGSGSENLYFQGSGGS)DLGKKLLEAARAGQDDEVRILVANGADVNAYEGTTPLHLAAAHGRLEIV
EVLLKNGADVNAQDVWGITPLHLAAYNGHLEIVEVLLKYGADVNAHDTRGWTPLHLAAINGHLEIVEVLLKNVAD
VNAQDRSGKTPFDLAIDNGNEDIAEVLQKAAKLN(SGSGSGKPGQASGS)ELARKLLEASTKLQRLNIRLAEALL
EAIARLQELNLELVYLAVELTDPKRIRDEIKEVKDKSKEIIRRAEKEIDDAAKESEKILEEAREAISGSGSELAK
LLLKAIAETQDLNLRAAKAFLEAAAKLQELNIRAVELLVKLTDPATIREALEHAKRRSKEIIDEAERAIRAAKRE
SERIIEEARRLIEKGSGSGSELARELLRAHAQLQRLNLELLRELLRALAQLQELNLDLLRLASELTDEIWIAQEL
RRIGDEFNAYYADAERLIREAAAASEKISREAERLIR
>Her2_Cage_I287A Tuned Cage targeted to Her2 by DARPin (I287A) SEQ ID
NO: 27407
(MGSHHHHHHGSGSENLYFQGSGGS)DLGKKLLEAARAGQDDEVRILMANGADVNAKDEYGLIPLYLATAHGHLE
IVEVLLKNGADVNAVDAIGFTPLHLAAFIGHLEIAEVLLKHGADVNAQDKFGKTAFDISIGNGNEDLAEILQKLN
(SGSGSGKPGQASGS)ELARKLLEASTKLQRLNIRLAEALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRA
VELLVKLTDPATIREALEHAKRRSKEIIDEAERAIRAAKRESERIIEEARRLIEKGSGSGSELARELLRAHAQLQ
RLNLELLRELLRALAQLQELNLDLLRLASELTDEIWIAQELRRIGDEFNAYYADAERLIREAAAASEKISREAER
LAR
>Her2_Cage_I287S Tuned Cage targeted to Her2 by DARPin (I287S) SEQ ID
NO: 27408
(MGSHHHHHHGSGSENLYFQGSGGS)DLGKKLLEAARAGQDDEVRILMANGADVNAKDEYGLIPLYLATAHGHLE
IVEVLLKNGADVNAVDAIGFTPLHLAAFIGHLEIAEVLLKHGADVNAQDKFGKTAFDISIGNGNEDLAEILQKLN
(SGSGSGKPGQASGS)ELARKLLEASTKLQRLNIRLAEALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRA
VELLVKLTDPATIREALEHAKRRSKEIIDEAERAIRAAKRESERIIEEARRLIEKGSGSGSELARELLRAHAQLQ
RLNLELLRELLRALAQLQELNLDLLRLASELTDEIWIAQELRRIGDEFNAYYADAERLIREAAAASEKISREAER
LSR
>Her2_Cage_I269S Tuned Cage targeted to Her2 by DARPin (I269S) SEQ ID
NO: 27409
(MGSHHHHHHGSGSENLYFQGSGGS)DLGKKLLEAARAGQDDEVRILMANGADVNAKDEYGLIPLYLATAHGHLE
IVEVLLKNGADVNAVDAIGFTPLHLAAFIGHLEIAEVLLKHGADVNAQDKFGKTAFDISIGNGNEDLAEILQKLN
(SGSGSGKPGQASGS)ELARKLLEASTKLQRLNIRLAEALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRA
VELLVKLTDPATIREALEHAKRRSKEIIDEAERAIRAAKRESERIIEEARRLIEKGSGSGSELARELLRAHAQLQ
RLNLELLRELLRALAQLQELNLDLLRLASELTDEIWIAQELRRIGDEFNAYYADAERLSREAAAASEKISREAER
LIR
>Her2_Cage_I269S_I287A Tuned Cage targeted to Her2 by DARPin (I269S,
I287A) SEQ ID NO: 27410
(MGSHHHHHHGSGSENLYFQGSGGS)DLGKKLLEAARAGQDDEVRILMANGADVNAKDEYGLIPLYLATAHGHLE
IVEVLLKNGADVNAVDAIGFTPLHLAAFIGHLEIAEVLLKHGADVNAQDKFGKTAFDISIGNGNEDLAEILQKLN
(SGSGSGKPGQASGS)ELARKLLEASTKLQRLNIRLAEALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRA
VELLVKLTDPATIREALEHAKRRSKEIIDEAERAIRAAKRESERIIEEARRLIEKGSGSGSELARELLRAHAQLQ
RLNLELLRELLRALAQLQELNLDLLRLASELTDEIWIAQELRRIGDEFNAYYADAERLSREAAAASEKISREAER
LAR
>Her2_Cage_I269S_I287S Tuned Cage targeted to Her2 by DARPin (I269S,
I287A) SEQ ID NO: 27411
(MGSHHHHHHGSGSENLYFQGSGGS)DLGKKLLEAARAGQDDEVRILMANGADVNAKDEYGLIPLYLATAHGHLE
IVEVLLKNGADVNAVDAIGFTPLHLAAFIGHLEIAEVLLKHGADVNAQDKFGKTAFDISIGNGNEDLAEILQKLN
(SGSGSGKPGQASGS)ELARKLLEASTKLQRLNIRLAEALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRA
VELLVKLTDPATIREALEHAKRRSKEIIDEAERAIRAAKRESERIIEEARRLIEKGSGSGSELARELLRAHAQLQ
RLNLELLRELLRALAQLQELNLDLLRLASELTDEIWIAQELRRIGDEFNAYYADAERLSREAAAASEKISREAER
LSR
>Her2_Cage_L209A Tuned Cage targeted to Her2 by DARPin (L209A) SEQ ID
NO: 27412
(MGSHHHHHHGSGSENLYFQGSGGS)DLGKKLLEAARAGQDDEVRILMANGADVNAKDEYGLIPLYLATAHGHLE
IVEVLLKNGADVNAVDAIGFTPLHLAAFIGHLEIAEVLLKHGADVNAQDKFGKTAFDISIGNGNEDLAEILQKLN
(SGSGSGKPGQASGS)ELARKLLEASTKLQRLNIRLAEALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRA
VELLVKLTDPATIREALEHAKRRSKEIIDEAERAIRAAKRESERIIEEARRLIEKGSGSGSELARELLRAHAQAQ
RLNLELLRELLRALAQLQELNLDLLRLASELTDEIWIAQELRRIGDEFNAYYADAERLIREAAAASEKISREAER
LIR
>herceptin_Cage_I269S Tuned Cage targeted to Her2 by herceptin scEv SEQ
ID NO: 27413
(METDILLLWVLLLWVPGSTGDYKDEHHHHHHGGSENLYFQGSG)DIQMIQSPSSLSASVGDRVTITCRASQDVN
TAVAWYQQKPGKAPKLLIYSASFLYSGVPSRFSGSRSGTDFILTISSLQPEDFATYYCQQHYTTPPTEGQGTKVE
IKGSTSGSGKPGSGEGSGEVQLVESGGGLVQPGGSLRLSCAASGFNIKDIYIHWVRQAPGKGLEWVARIYPINGY
TRYADSVKGRFTISADTSKNTAYLQMNSLRAEDTAVYYCSRWGGDGFYAMDYWGQGTLVIVS(SGSGSGKPGQAS
GS)ELARKLLEASTKLQRLNIRLAEALLEAIARLQELNLELVYLAVELTDPKRIRDEIKEVKDKSKEIIRRAEKE
IDDAAKESEKILEEAREAISGSGSELAKLLLKAIAETQDLNLRAAKAFLEAAAKLQELNIRAVELLVKLTDPATI
REALEHAKRRSKEIIDEAERAIRAAKRESERIIEEARRLIEKGSGSGSELARELLRAHAQLQRLNLELLRELLRA
LAQLQELNLDLLRLASELTDEIWIAQELRRIGDEFNAYYADAERLSREAAAASEKISREAERLIR
>Her2_Cage_L209A_L216A Tuned Cage targeted to Her2 by DARPin (L209A) SEQ
ID NO: 27414
(MGSHHHHHHGSGSENLYFQGSGGS)DLGKKLLEAARAGQDDEVRILMANGADVNAKDEYGLIPLYLATAHGHLE
IVEVLLKNGADVNAVDAIGFTPLHLAAFIGHLEIAEVLLKHGADVNAQDKFGKTAFDISIGNGNEDLAEILQKLN
(SGSGSGKPGQASGS)ELARKLLEASTKLQRLNIRLAEALLEAIARLQELNLELVYLAVELTDPKRIRDEIKEVK
DKSKEIIRRAEKEIDDAAKESEKILEEAREAISGSGSELAKLLLKAIAETQDLNLRAAKAFLEAAAKLQELNIRA
VELLVKLTDPATIREALEHAKRRSKEIIDEAERAIRAAKRESERIIEEARRLIEKGSGSGSELARELLRAHAQAQ
RLNLEALRELLRALAQLQELNLDLLRLASELTDEIWIAQELRRIGDEFNAYYADAERLIREAAAASEKISREAER
LIR
>EGER_Cage_I269S Tuned Cage targeted to EGFR by DARPin (I269S) SEQ ID
NO: 27415
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELTDEIWIAQELRR
IGDEFNAYYADAERLSREAAAASEKISREAERLIR
>EGER_Cage_I287A Tuned Cage targeted to EGFR by DARPin (I287A) SEQ ID
NO: 27416
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELTDEIWIAQELRR
IGDEFNAYYADAERLIREAAAASEKISREAERLAR
>EGER_Cage_L209A Tuned Cage targeted to EGFR by DARPin (L209A) SEQ ID
NO: 27417
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQAQRLNLELLRELLRALAQLQELNLDLLRLASELTDEIWIAQELRR
IGDEFNAYYADAERLIREAAAASEKISREAERLIR
>EGER_Cage_I269S long linker Tuned Cage targeted to EGFR by DARPin via
long linker (I269S) SEQ ID NO: 27418
(MGSHHHHHHGSGSENLYFQGSGGS)DLGKKLLEAARAGQDDEVRILVANGADVNAYFGTTPLHLAAAHGRLEIV
EVLLKNGADVNAQDVWGITPLHLAAYNGHLEIVEVLLKYGADVNAHDIRGWIPLHLAAINGHLEIVEVLLKNVAD
VNAQDRSGKTPFDLAIDNGNEDIAEVLQKAAKLN(SGSGSGSGSGSGSGSGSGSGSGSGSGSGSGSGSGSGSGSG
SGSGSGSGSGSGSGSGSGKPGQASGS)ELARKLLEASTKLQRLNIRLAEALLEAIARLQELNLELVYLAVELTDP
KRIRDEIKEVKDKSKEIIRRAEKEIDDAAKESEKILEEAREAISGSGSELAKLLLKAIAETQDLNLRAAKAFLEA
AAKLQELNIRAVELLVKLTDPATIREALEHAKRRSKEIIDEAERAIRAAKRESERIIEEARRLIEKGSGSGSELA
RELLRAHAQLQRLNLELLRELLRALAQLQELNLDLLRLASELTDEIWIAQELRRIGDEFNAYYADAERLSREAAA
ASEKISREAERLIR
>EpCAM_Cage_I269S Tuned Cage targeted to EpCAM by DARPin (I269S) SEQ ID
NO: 27419
(MGSHHHHHHGSGSENLYFQGSGGS)DLGKKLLEAARAGQDDEVRILVANGADVNAYFGTTPLHLAAAHGRLEIV
EVLLKNGADVNAQDVWGITPLHLAAYNGHLEIVEVLLKYGADVNAHDIRGWIPLHLAAINGHLEIVEVLLKNVAD
VNAQDRSGKTPFDLAIDNGNEDIAEVLQKAAKLN(SGSGSGKPGQASGS)ELARKLLEASTKLQRLNIRLAEALL
EAIARLQELNLELVYLAVELTDPKRIRDEIKEVKDKSKEIIRRAEKEIDDAAKESEKILEEAREAISGSGSELAK
LLLKAIAETQDLNLRAAKAFLEAAAKLQELNIRAVELLVKLTDPATIREALEHAKRRSKEIIDEAERAIRAAKRE
SERIIEEARRLIEKGSGSGSELARELLRAHAQLQRLNLELLRELLRALAQLQELNLDLLRLASELTDEIWIAQEL
RRIGDEFNAYYADAERLSREAAAASEKISREAERLIR
>EpCAM_Cage_I287A Tuned Cage targeted to EpCAM by DARPin (I287A) SEQ ID
NO: 27420
(MGSHHHHHHGSGSENLYFQGSGGS)DLGKKLLEAARAGQDDEVRILVANGADVNAYFGTTPLHLAAAHGRLEIV
EVLLKNGADVNAQDVWGITPLHLAAYNGHLEIVEVLLKYGADVNAHDIRGWIPLHLAAINGHLEIVEVLLKNVAD
VNAQDRSGKTPFDLAIDNGNEDIAEVLQKAAKLN(SGSGSGKPGQASGS)ELARKLLEASTKLQRLNIRLAEALL
EAIARLQELNLELVYLAVELTDPKRIRDEIKEVKDKSKEIIRRAEKEIDDAAKESEKILEEAREAISGSGSELAK
LLLKAIAETQDLNLRAAKAFLEAAAKLQELNIRAVELLVKLTDPATIREALEHAKRRSKEIIDEAERAIRAAKRE
SERIIEEARRLIEKGSGSGSELARELLRAHAQLQRLNLELLRELLRALAQLQELNLDLLRLASELTDEIWIAQEL
RRIGDEFNAYYADAERLIREAAAASEKISREAERLAR
>EpCAM_Cage_L209A Tuned Cage targeted to EpCAM by DARPin (L209A) SEQ ID
NO: 27421
(MGSHHHHHHGSGSENLYFQGSGGS)DLGKKLLEAARAGQDDEVRILVANGADVNAYFGTTPLHLAAAHGRLEIV
EVLLKNGADVNAQDVWGITPLHLAAYNGHLEIVEVLLKYGADVNAHDIRGWIPLHLAAINGHLEIVEVLLKNVAD
VNAQDRSGKTPFDLAIDNGNEDIAEVLQKAAKLN(SGSGSGKPGQASGS)ELARKLLEASTKLQRLNIRLAEALL
EAIARLQELNLELVYLAVELTDPKRIRDEIKEVKDKSKEIIRRAEKEIDDAAKESEKILEEAREAISGSGSELAK
LLLKAIAETQDLNLRAAKAFLEAAAKLQELNIRAVELLVKLTDPATIREALEHAKRRSKEIIDEAERAIRAAKRE
SERIIEEARRLIEKGSGSGSELARELLRAHAQAQRLNLELLRELLRALAQLQELNLDLLRLASELTDEIWIAQEL
RRIGDEFNAYYADAERLIREAAAASEKISREAERLIR
Co-LOCKR Decoy proteins (for ′NOT′ gates) (proteins containing
RDHMVLHEYVNAAGITF (SEQ ID No: 27447) may alternatively be used as Cage
proteins in cases when the effector protein interacts with GFP11)
>EGER_Decoyl Original decoy targeted to EGFR by DARPin (for NOT logic)
SEQ ID NO: 27422
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELRDHMVLHEYVNA
AGITFNAYYADAERLIREAAAASEKISREAG
>EGER_Decoy_G24 Tuned Decoy targeted to EGFR by DARPin SEQ ID NO: 27423
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELGRDHMVLHEYVN
AAGITFNAYYADAERLIREAAAASEKISREAG
>EGER_Decoy_G25 Tuned Decoy targeted to EGFR by DARPin SEQ ID NO: 27424
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELRDHMVLHEYVNA
AGITFNAYYADAERLSREAAAASEKISREAG
>EGER_Decoy_G26 Tuned Decoy targeted to EGFR by DARPin SEQ ID NO: 27425
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELRDHMVLHEYVNA
AGITFNAYYADAERLIREAAAASEKISREAER
>EGER_Decoy_G29 Tuned Decoy targeted to EGFR by DARPin SEQ ID NO: 27426
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELTDPDEARKAIAR
VKRESNAYYADAERLSREAAAASEKISREAERLIR
>EGER_Decoy_G31 Tuned Decoy targeted to EGFR by DARPin SEQ ID NO: 27427
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQAQRLNLELLRELLRALAQLQELNLDLLRLASELGTDPDEARKAIA
RVKRESNAYYADAERLSREAAAASEKISREAERLIR
>EGER_Decoy_G33 Tuned Decoy targeted to EGFR by DARPin SEQ ID NO: 27428
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELGTDPDEARKAIA
RVKRESNAYYADAERLSREAAAASEKISREAG
>EGER_Decoy_G34 Tuned Decoy targeted to EGFR by DARPin SEQ ID NO: 27429
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELGTDPDEARKAIA
RVKRESNAYYADAERLSREAAAASEKISREAER
>EGER_Decoy_G35 Tuned Decoy targeted to EGFR by DARPin SEQ ID NO: 27430
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELGTDPDEARKAIA
RVKRESNAYYADAERLSREAAAASEKISREAERLI
>EGER_Decoy_G7(1A7) Tuned Decoy targeted to EGFR by DARPin SEQ ID
NO: 27431
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELGTDPDEARKAIA
RVKRESNAYYADAERLSREAAAASEKISREAERLIR
>EGER_Decoy_Box1C1 Tuned Decoy targeted to EGFR by DARPin SEQ ID
NO: 27432
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELGTDPDEARKAIA
RVKRESNAYYADAERLIREAAAASEKISREAERLIR
>EGER_Decoy3 Tuned Decoy targeted to EGFR by DARPin SEQ ID
NO: 27433
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELGTDPDEARKAIA
RVKRESNAYYADAERLSREAAAASEKISREAERLAR
>EGER_Decoy5 Tuned Decoy targeted to EGFR by DARPin SEQ ID NO: 27434
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELGTDPDEARKAIA
RVKRESNAYYADAERLIREAAAASEKISREAERLAR
>EGER_Decoy7 Tuned Decoy targeted to EGFR by DARPin SEQ ID
NO: 27435
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELTDPDEARKAIAR
VKRESNAYYADAERLSREAAAASEKISREAERLAR
>EGER_Decoy8 Tuned Decoy targeted to EGFR by DARPin SEQ ID NO: 27436
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELTDPDEARKAIAR
VKRESNAYYADAERLSREAAAASEKISREAERLIR
>EGER_Decoy9 Tuned Decoy targeted to EGFR by DARPin SEQ ID
NO: 27437
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELTDPDEARKAIAR
VKRESNAYYADAERLIREAAAASEKISREAERLIR
>EGER_Decoy10 Tuned Decoy targeted to EGFR by DARPin SEQ ID
NO: 27438
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELGSEDLYFQGSTD
PDEARKAIARVKRESNAYYADAERLSREAAAASEKISREAERLAR
>EGER_Decoy11 Tuned Decoy targeted to EGFR by DARPin SEQ ID
NO: 27439
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELGSEDLYFQGSTD
PDEARKAIARVKRESNAYYADAERLSREAAAASEKISREAERLIR
>EGER_Decoy12 Tuned Decoy targeted to EGFR by DARPin SEQ ID
NO: 27440
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELGSEDLYFQGSTD
PDEARKAIARVKRESNAYYADAERLIREAAAASEKISREAERLAR
>EGER_Decoy13 Tuned Decoy targeted to EGFR by DARPin SEQ ID
NO: 27441
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELGSEDLYFQGSTD
PDEARKAIARVKRESNAYYADAERLIREAAAASEKISREAERLIR
>EGER_Decoy14 Tuned Decoy targeted to EGFR by DARPin SEQ ID
NO: 27442
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELTDPDEARKAIAR
VKRESNAYYADAERLIREAAAASEKISREAERLIR
>EGER_Decoy_G27 Tuned Decoy targeted to EGFR by DARPin SEQ ID
NO: 27443
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELRDHMVLHEYVNA
AGITFNAYYADAERLIREAAAASEKISREAERLI
>EGER_Decoy_G28 Tuned Decoy targeted to EGFR by DARPin SEQ ID
NO: 27444
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELRDHMVLHEYVNA
AGITFNAYYADAERLIREAAAASEKISREAERLIR
>EGER_Decoy_G30 Tuned Decoy targeted to EGFR by DARPin SEQ ID
NO: 27445
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLELLRELLRALAQLQELNLDLLRLASELGTDPDEARKAIA
RVKRESNAYYADAERLSREAAAASEKISREAERLSR
>EGER_Decoy_G32 Tuned Decoy targeted to EGFR by DARPin SEQ ID
NO: 27446
(MGSHHHHHHGSGSENLYFQGSGGS)DLGKKLLEAARAGQDDEVRILMANGADVNADDIWGWIPLHLAAYQGHLE
IVEVLLKNGADVNAYDYIGWIPLHLAADGHLEIVEVLLKNGADVNASDYIGDTPLHLAAHNGHLEIVEVLLKHGA
DVNAQDKFGKTAFDISIDNGNEDLAEILQKLN(SGSGSGKPGQASGS)ELARKLLEASTKLQRLNIRLAEALLEA
IARLQELNLELVYLAVELTDPKRIRDEIKEVKDKSKEIIRRAEKEIDDAAKESEKILEEAREAISGSGSELAKLL
LKAIAETQDLNLRAAKAFLEAAAKLQELNIRAVELLVKLTDPATIREALEHAKRRSKEIIDEAERAIRAAKRESE
RIIEEARRLIEKGSGSGSELARELLRAHAQLQRLNLEALRELLRALAQLQELNLDLLRLASELGTDPDEARKAIA
RVKRESNAYYADAERLSREAAAASEKISREAERLIR

TABLE 12
Co-LOCKR Key proteins

>HA_Key_Her2 HA-tagged original Key targeted to Her2 by DARPin SEQ ID
NO: 27448
(MGSHHHHHHGSGSENLYFQGSYPYDVPDYAGGS)DEARKAIARVKRESKRIVEDAERLIREAAAASEKISREAE
RLIR(GGGSGSGSGSGKPGQASGS)DLGKKLLEAARAGQDDEVRILMANGADVNAKDEYGLIPLYLATAHGHLEI
VEVLLKNGADVNAVDAIGFTPLHLAAFIGHLEIAEVLLKHGADVNAQDKEGKTAFDISIGNGNEDLAEILQKLN
>Key_EGFR Original Key targeted to EGFR by DARPin SEQ ID NO: 27449
(MGSHHHHHHGSGSENLYFQGSGGS)DEARKAIARVKRESKRIVEDAERLIREAAAASEKISREAERLIR(GGGS
GSGSGSGKPGQASGS)DLGKKLLEAARAGQDDEVRILMANGADVNADDIWGWIPLHLAAYQGHLEIVEVLLKNGA
DVNAYDYIGWTPLHLAADGHLEIVEVLLKNGADVNASDYIGDTPLHLAAHNGHLEIVEVLLKHGADVNAQDKEGK
TAFDISIDNGNEDLAEILQKLN
>Key_EpCAM Original Key targeted to EpCAM by DARPin SEQ ID NO: 27450
(MGSHHHHHHGSGSENLYFQGSGGS)DEARKAIARVKRESKRIVEDAERLIREAAAASEKISREAERLIR(GGGS
GSGSGSGKPGQASGS)DLGKKLLEAARAGQDDEVRILVANGADVNAYEGTTPLHLAAAHGRLEIVEVLLKNGADV
NAQDVWGITPLHLAAYNGHLEIVEVLLKYGADVNAHDIRGWIPLHLAAINGHLEIVEVLLKNVADVNAQDRSGKT
PFDLAIDNGNEDIAEVLQKAAKLN
>Key_N3_EpCAM Tuned Key targeted to EpCAM by DARPin (3 aa deletion near
N-term of Key) SEQ ID NO: 27451
(MGSHHHHHHGSGSENLYFQGSGGS)DEAIARVKRESKRIVEDAERLIREAAAASEKISREAERLIR(GGGSGSG
SGSGKPGQASGS)DLGKKLLEAARAGQDDEVRILVANGADVNAYEGTTPLHLAAAHGRLEIVEVLLKNGADVNAQ
DVWGITPLHLAAYNGHLEIVEVLLKYGADVNAHDIRGWIPLHLAAINGHLEIVEVLLKNVADVNAQDRSGKTPFD
LAIDNGNEDIAEVLQKAAKLN
>Key_N7_EpCAM Tuned Key targeted to EpCAM by DARPin (7 aa deletion near
N-term of Key) SEQ ID NO: 27452
(MGSHHHHHHGSGSENLYFQGSGGS)DEVKRESKRIVEDAERLIREAAAASEKISREAERLIR(GGGSGSGSGSG
KPGQASGS)DLGKKLLEAARAGQDDEVRILVANGADVNAYEGTTPLHLAAAHGRLEIVEVLLKNGADVNAQDVWG
ITPLHLAAYNGHLEIVEVLLKYGADVNAHDIRGWIPLHLAAINGHLEIVEVLLKNVADVNAQDRSGKTPFDLAID
NGNEDIAEVLQKAAKLN
>Key_T7_EpCAM Tuned Key targeted to EpCAM by DARPin (7 aa deletion near
C-term of Key) SEQ ID NO: 27453
(MGSHHHHHHGSGSENLYFQGSGGS)DEARKAIARVKRESKRIVEDAERLIREAAAASEKISR(GGGSGSGSGSG
KPGQASGS)DLGKKLLEAARAGQDDEVRILVANGADVNAYEGTTPLHLAAAHGRLEIVEVLLKNGADVNAQDVWG
ITPLHLAAYNGHLEIVEVLLKYGADVNAHDIRGWIPLHLAAINGHLEIVEVLLKNVADVNAQDRSGKTPFDLAID
NGNEDIAEVLQKAAKLN
>Key_N3_Her2 Tuned Key targeted to Her2 by DARPin (3 aa deletion near
N-term of Key) SEQ ID NO: 27454
(MGSHHHHHHGSGSENLYFQGSGGS)DEAIARVKRESKRIVEDAERLIREAAAASEKISREAERLIR(GGGSGSG
SGSGKPGQASGS)DLGKKLLEAARAGQDDEVRILMANGADVNAKDEYGLIPLYLATAHGHLEIVEVLLKNGADVN
AVDAIGFTPLHLAAFIGHLEIAEVLLKHGADVNAQDKEGKTAFDISIGNGNEDLAEILQKLN
>Key_N3_EGFR Tuned Key targeted to EGFR by DARPin (3 aa deletion near
N-term of Key) SEQ ID NO: 27455
(MGSHHHHHHGSGSENLYFQGSGGS)DEAIARVKRESKRIVEDAERLIREAAAASEKISREAERLIR(GGGSGSG
SGSGKPGQASGS)DLGKKLLEAARAGQDDEVRILMANGADVNADDIWGWIPLHLAAYQGHLEIVEVLLKNGADVN
AYDYIGWTPLHLAADGHLEIVEVLLKNGADVNASDYIGDTPLHLAAHNGHLEIVEVLLKHGADVNAQDKEGKTAF
DISIDNGNEDLAEILQKLN
>Key anti-EGFR-scFv Original Key targeted to EGFR by an anti-EGFR scFv
SEQ ID NO: 27456
(METDILLLWVLLLWVPGSTGDYKDEHHHHHHGGSENLYFQGSGS)DEARKAIARVKRESKRIVEDAERLIREAA
AASEKISREAERLIR(GGGSGSGSGSGKPGQASGS)QVQLKQSGPGLVQPSQSLSITCTVSGFSLTNYGVHWVRQ
SPGKGLEWLGVIWSGGNIDYNTPFTSRLSINKDNSKSQVFFKMNSLQSNDTAIYYCARALTYYDYEFAYWGQGIL
VIVSAGGGGSGGGGSGGGGSDILLTQSPVILSVSPGERVSFSCRASQSIGINIHWYQQRTNGSPRLLIKYASESI
SGIPSRFSGSGSGTDFILSINSVESEDIADYYCQQNNNWPTIFGAGTKLELKRT
>Key_T3_EpCAM Tuned Key targeted to EpCAM by DARPin (3 aa deletion near
C-term of Key) SEQ ID NO: 27457
(MGSHHHHHHGSGSENLYFQGSGGS)DEARKAIARVKRESKRIVEDAERLIREAAAASEKISREAER(GGGSGSG
SGSGKPGQASGS)DLGKKLLEAARAGQDDEVRILVANGADVNAYEGTTPLHLAAAHGRLEIVEVLLKNGADVNAQ
DVWGITPLHLAAYNGHLEIVEVLLKYGADVNAHDIRGWIPLHLAAINGHLEIVEVLLKNVADVNAQDRSGKTPFD
LAIDNGNEDIAEVLQKAAKLN
>Key_EpCAM_I35S_I43A Tuned Key targeted to EpCAM by DARPin (I35S, I43A)
SEQ ID NO: 27458
(MGSHHHHHHGSGSENLYFQGSGGS)DEARKAIARVKRESKRIVEDAERLIREAAAASEKSSREAERLAR(GGGS
GSGSGSGKPGQASGS)DLGKKLLEAARAGQDDEVRILVANGADVNAYFGTTPLHLAAAHGRLEIVEVLLKNGADV
NAQDVWGITPLHLAAYNGHLEIVEVLLKYGADVNAHDIRGWIPLHLAAINGHLEIVEVLLKNVADVNAQDRSGKT
PFDLAIDNGNEDIAEVLQKAAKLN
>Key_EGFR_EpCAM Key targets both EGFR and EpCAM SEQ ID NO: 27459
(MGSHHHHHHGSGSENLYFQGSGGS)DEARKAIARVKRESKRIVEDAERLIREAAAASEKISREAERLIR(GGGS
GSGSGSGKPGQASGS)DLGKKLLEAARAGQDDEVRILMANGADVNADDIWGWIPLHLAAYQGHLEIVEVLLKNGA
DVNAYDYIGWIPLHLAADGHLEIVEVLLKNGADVNASDYIGDTPLHLAAHNGHLEIVEVLLKHGADVNAQDKFGK
TAFDISIDNGNEDLAEILQKLN(GGGSGGGS)DLGKKLLEAARAGQDDEVRILVANGADVNAYFGTTPLHLAAAH
GRLEIVEVLLKNGADVNAQDVWGITPLHLAAYNGHLEIVEVLLKYGADVNAHDIRGWIPLHLAAINGHLEIVEVL
LKNVADVNAQDRSGKTPFDLAIDNGNEDIAEVLQKAAKLN

TABLE 13
Effector proteins

>Bcl2-cys (SEQ ID NO: 27460)
(MGSHHHHHHGSGSENLYFQGSGGS)AHAGRTGYDNREIVMKYIHYKLSQRGYEWDAGDVGAAPPGAAPAPGIFS
SQPGHTPHPAASRDPVARTSPLQTPAAPGAAAGPALSPVPPVVHLTLRQAGDDFSRRYRRDFAEMSSQLHLTPFT
ARGRFATVVEELFRDGVNWGRIVAFFEFGGVMCVESVNREMSPLVDNIALWMTEYLNRHLHTWIQDNGGWDAFVE
LYGPSMRGC
>GFP1-10 (SEQ ID NO: 27461)
(M)SKGEELFTGVVPILVELDGDVNGHKESVRGEGEGDATIGKLTLKFICTTGKLPVPWPTLVTTLTYGVQCFSR
YPDHMKRHDFFKSAMPEGYVQERTISFKDDGKYKTRAVVKFEGDTLVNRIELKGTDFKEDGNILGHKLEYNENSH
NVYITADKQKNGIKANFTVRHNVEDGSVQLADHYQQNTPIGDGPVLLPDNHYLSTQTVLSKDPNEK
>Bcl2_opt1 (SEQ ID NO: 27462)
AHAGRTGYDNREIVMKYIHYKLSQRGYEWDAGDDAEEGRTEAPEGTESEVVHLTLRQAGDDFSRRYRRDFAEMSS
QLHLTPFTARGRFATVVEELFRDGVNWGRIVAFFEFGGVMCVESVNREMSPLVDNIALWMTEYLNRHLHTWIQDN
GGWDAFVELYGPSMR
>Bcl2_opt2 (SEQ ID NO: 27463)
AHAGRTGYDNREIVMKYIHYKLSQRGYEWDAGDDAEEGSGSGSGTESEVVHLTLRQAGDDFSRRYRRDFAEMSSQ
LHLTPFTARGRFATVVEELFRDGVNWGRIVAFFEFGGVMCVESVNREMSPLVDNIALWMTEYLNRHLHTWIQDNG
GWDAFVELYGPSMR
>Bcl2_opt3 (SEQ ID NO: 27464)
AHAGRTGYDNREIVMKYIHYKLSQRGYEWDAGDDAEEGRTEAPEGTESEVVHQTLRQAGDDFERRYRRDFSDMSS
QLHLTPDTARGRFATVVEELFRDGVNWGRIVAFFEFGGVMCVESVNREMSPLVDRIADWMTEYLNRHLHPWIQDN
GGWDAFVELYGPSMR
>Bcl2_opt4 (SEQ ID NO: 27465)
AHAGRTGYDNREIVMKYIHYKLSQRGYEWDAGDDAEEGSGSGSGTESEVVHQTLRQAGDDFERRYRRDFSDMSSQ
LHLTPDTARQRFATVVEELFRDGVNWGRIVAFFEFGGVMCVESVNREMSPLVDRIADWMTEYLNRHLHPWIQDNG
GWDAFVELYGPSMR
>Bcl2_opt5 (SEQ ID NO: 27466)
AHAGRTGYDNREIVMKYIHYKLSQRGYEWDAGDDAEEGRTEAPEGTESEVVHQTLRQAGDDFERRYRRDFSDMSS
QLHLTPDTARQRFATVVEELFRDGVNWGRIVAFFEFGGVMAVEMVNRGGSPLVDRIADWMTEYLNRHLHPWIQDN
GGWDAFVELYGPSMR
>Bcl2_opt6 (SEQ ID NO: 27467)
AHAGRTGYDNREIVMKYIHYKLSQRGYEWDAGDDAEEGSGSGSGTESEVVHQTLRQAGDDFERRYRRDFSDMSSQ
LHLTPDTARQRFATVVEELFRDGVNWGRIVAFFEFGGVMAVEMVNRGGSPLVDRIADWMTEYLNRHLHPWIQDNG
GWDAFVELYGPSMR
>Bcl2_opt7 (SEQ ID NO: 27468)
AHAGRTGYDNREIVMKYIHYKLSQRGYEWDAGDDAEENRTEAPEGTESEVVHRALRDAGDDFERRYRRDFAEMSS
QLHLTPDTARQRFETVVEELFRDGVNWGRIVAFFEFGGVMCVESVNREMSPLVDNIAEWMTEYLNRHLHTWIQDN
GGWDAFVELYGPSMR
>Bcl2_opt8 (SEQ ID NO: 27469)
AHAGRTGYDNREIVRKYIHYKLSQRGYEWDAGDDAEEGSGSGSGTESEVVHRALRDAGDDFERRYRRDFAEMSSQ
LHLTPDTARQRFETVVEELFRDGVNWGRIVAFFEFGGVMCVECVNREMSPLVDNIAEWMTEYLNRHLHTWIQDNG
GWDAFVELYGPSMR

Nucleic Acids

The present disclosure provides one or more nucleic acids that encode a first cage polypeptide and/or one or more key polypeptides. In some aspects, the nucleic acid encoding a first cage polypeptide and the nucleic acid encoding a first key polypeptide are on the same vector. In some aspects, the nucleic acid encoding a first cage polypeptide and the nucleic acid encoding a first key polypeptide are on different vectors. In another aspect the disclosure provides nucleic acids encoding the fusion protein (e.g., chimeric antigen receptor) of any embodiment or combination of embodiments disclosed herein. The nucleic acids encoding a CAR can be on the same vector as the nucleic acid encoding the first cage polypeptide and/or one or more of the key polypeptides.

The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In another aspect, the disclosure provides expression vectors comprising the nucleic acid of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, enhancers, introns, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF, EF1alpha, MND, MSCV) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

Cells In a further aspect, the disclosure provides host cells that comprise the nucleic acids, expression vectors (i.e.: episomal or chromosomally integrated), or polypeptides disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection. In one embodiment, the viral vector comprises an adenoviral vector, a vaccinia viral vector, an AAV vector, a retroviral vector, a lentiviral vector, an alphaviral vector, or any combination thereof.

In one embodiment, the cells comprise T cells.

Chimeric Antigen Receptor T Cells

The present disclosure also provides a method of increasing tumor cell selectivity in a subject in need of a chimeric antigen receptor T cell therapy. In some aspects, the disclosure provides administering a CAR T cells. In some aspects, the CAR can be expressed as a fusion protein.

In another aspect, the disclosure provides CAR fusion proteins, comprising:

(a) an extracellular binding domain;

(b) a transmembrane domain;

(c) an intracellular signaling component; and

(d) optionally, a selection marker.

The fusion proteins can be used, for example, as a chimeric receptor antigen for use in generating cells, such as CAR-T cells, for use in the compositions of the disclosure described above. The fusion protein comprises an extracellular component comprising an binding domain specific for an antigen, such as the bioactive peptides as contemplated herein; an optional extracellular spacer domain to optimize binding; a transmembrane domain; and an intracellular signaling component comprising an intracellular activation domain (e.g., an immunoreceptor tyrosine-based activation motif (ITAM)-containing T cell activating motif), an intracellular costimulatory domain, or both. In certain embodiments, an intracellular signaling component of a CAR has an ITAM-containing T cell activating domain (e.g., CD3ζ) and an intracellular costimulatory domain (e.g., CD28, 41BB). In certain embodiments, a CAR is synthesized as a single polypeptide chain or is encoded by a nucleic acid molecule as a single chain polypeptide. In some aspects, the CARs useful for the present disclosure are capable of specifically binding to one or more bioactive peptides described elsewhere herein. In some aspects, the CARs of the present disclosure does not target a tumor antigen, but instead a bioactive peptide.

In any of the embodiments described herein, the chimeric antigen receptor may further comprise a self-cleaving polypeptide, wherein a polynucleotide encoding the self-cleaving polypeptide is located between the polynucleotide encoding the fusion protein and the polynucleotide encoding the transduction marker. In certain embodiments, a self-cleaving polypeptide comprises a 2A peptide from porcine teschovirus-1 (P2A), Thosea asigna virus (T2A), equine rhinitis A virus (E2A), foot-and-mouth disease virus (F2A), or variant thereof. Further exemplary nucleic acid and amino acid sequences of 2A peptides are set forth in, for example, Kim et al. (PLOS One 6:e18556 (2011), which 2A nucleic acid and amino acid sequences are incorporated herein by reference in their entirety).

In one embodiment, the extracellular component includes a binding domain specific to one or more bioactive molecule. In a further embodiment, the binding domain comprises a peptide, wherein the peptide may optionally be selected from the group consisting of Fab′, F(ab′)₂, Fab, Fv, rIgG, recombinant single chain Fv fragments (scFv), V_{H} single domains, bivalent or bispecific molecules, diabodies, triabodies, and tetrabodies; Bcl or a variant thereof; and computationally designed proteins. In another embodiment, the one or more bioactive molecule comprises one or more bioactive peptide. In exemplary embodiments, the one or more bioactive peptides comprise one or more bioactive peptide selected from the group consisting of SEQ ID NOS:60, 62-64, 66, 27052, 27053, and 27059-27093. In a further embodiment, the binding domain comprises a stabilized variant of human Bcl2.

In another embodiment, the fusion protein (e.g., chimeric antigen receptor) further comprises a selection marker. In non-limiting embodiments, the selection marker is a truncated EGFR (EGFRt), truncated low-affinity nerve growth factor (tNGFR), a truncated CD19 (tCD19), a truncated CD34 (tCD34), or any combination thereof. In another embodiment, the fusion protein further comprises a self-cleaving peptide. In non-limiting embodiments, the self-cleaving peptide is a 2A peptide from porcine teschovirus-1 (P2A), Thosea asigna virus (T2A), equine rhinitis A virus (E2A), foot-and-mouth disease virus (F2A), or variant thereof.

In one embodiment, the fusion protein (e.g., chimeric antigen receptor) comprises a stabilized variant of human Bcl2, a flexible extracellular spacer domain, CD28/CD3ζ signaling domains, and a truncated EGFR (EGFRt) selection marker linked by a T2A ribosomal skipping sequence. In a further embodiment, the fusion protein comprises an amino acid sequence at least 75%, 80%, 85%, 90%, 91%, 92%, 93%, 94%, 95%, 96%, 97%, 98%, 99%, or 100% to the amino acid sequence of SEQ ID NO: 27,489.

>Bcl2 CAR Co-LOCKR CAR T cell recruitment
SEQ ID NO: 27489
METDTLLLWVLLLWVPGSTGDYKDEYPYDVPDYAGSAHAGRTGYDNREI
VMKYIHYKLSQRGYEWDAGDDAEENRTEAPEGTESEVVHRALRDAGDDF
ERRYRRDFAEMSSQLHLTPDTARQRFETVVEELFRDGVNWGRIVAFFEF
GGVMCVESVNREMSPLVDNIAEWMTEYLNRHLHTWIQDNGGWDAFVELY
GPSMRGGGGSGGGGSESKYGPPCPPCPAPPVAGPSVFLFPPKPKDTLMI
SRTPEVTCVVVDVSQEDPEVQFNWYVDGVEVHNAKTKPREEQFQSTYRV
VSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGKMF
WVLVVVGGVLACYSLLVTVAFTIFWVRSKRSRGGHSDYMNMTPRRPGPT
RKHYQPYAPPRDFAAYRSRVKFSRSADAPAYQQGQNQLYNELNLGRREE
YDVLDKRRGRDPEMGGKPRRKNPQEGLYNELQKDKMAEAYSEIGMKGER
RRGKGHDGLYQGLSTATKDTYDALHMQALPPRLEGGGEGRGSLLTCGDV
EENPGPRMLLLVTSLLLCELPHPAFLLIPRKVCNGIGIGEFKDSLSINA
TNIKHFKNCTSISGDLHILPVAFRGDSFTHTPPLDPQELDILKTVKEIT
GFLLIQAWPENRTDLHAFENLEIIRGRTKQHGQFSLAVVSLNITSLGLR
SLKEISDGDVIISGNKNLCYANTINWKKLFGTSGQKTKIISNRGENSCK
ATGQVCHALCSPEGCWGPEPRDCVSCRNVSRGRECVDKCNLLEGEPREF
VENSECIQCHPECLPQAMNITCTGRGPDNCIQCAHYIDGPHCVKTCPAG
VMGENNTLVWKYADAGHVCHLCHPNCTYGCTGPGLEGCPTNGPKIPSIA
TGMVGALLLLLVVALGIGLFM

In a further aspect, the disclosure provides methods of targeting an effector molecule to a cell comprising contacting a biological sample containing cells with the compositions, fusion proteins, nucleic acids, vectors, and/or the cells of any embodiment or combination of embodiments herein. In one embodiment, the methods further comprise contacting the cell with an effector molecule.

### Binding Domains/Cell Moieties

In another embodiment of the compositions of any embodiment or combination of embodiments of the disclosure, the first, second, third, fourth, fifth, sixth, and/or seventh binding domains are selected from the non-limiting group comprising an antigen-binding polypeptide directed against a cell surface moiety to be bound, including but not limited to Fab′, F(ab′)₂, Fab, Fv, rIgG, recombinant single chain Fv fragments (scFv), V_{H} single domains, bivalent or bispecific molecules, diabodies, triabodies, and tetrabodies; DARPins; nanobody; affibody; monobody; adnectin; alphabody; Albumin-binding domain; Adhiron; Affilin; Affimer; Affitin/Nanofitin; Anticalin; Armadillo repeat proteins; Atrimer/Tetranectin; Avimer/Maxibody; Centyrin; Fynomer; Kunitz domain; Obody/OB-fold; Pronectin; Repebody; and computationally designed proteins. In another embodiment, the first, second, third, fourth, fifth, sixth, and/or seventh binding domains bind to a cell surface protein on a cell selected from the non-limiting group comprising tumor cells, cancer cells, immune cells, leukocytes, lymphocytes, T cells, regulatory T cells, effector T cells, CD4+ effector T cells, CD8+ effector T cells, memory T cells, autoreactive T cells, exhausted T cells, natural killer T cells (NKT cells), B cells, dendritic cells, macrophages, NK cells, cardiac cells, lung cells, muscle cells, epithelial cells, pancreatic cells, skin cells, CNS cells, neurons, myocytes, skeletal muscle cells, smooth muscle cells, liver cells, kidney cells, bacterial cells, and yeast cells. In a further embodiment, the first, second, third, fourth, fifth, sixth, and/or seventh binding domains bind to a cell surface protein selected from the non-limiting group comprising Her2, EGFR, EpCAM, B7-H3, ROR1, GD2, GPC2, αvβ6, Her3, L1CAM, BCMA, GPCR5d, EGFRvIII, CD20, CD22, CD3, CD4, CD5, CD8, CD19, CD27, CD28, CD30, CD33, CD48, IL3RA, platelet tissue factor, CLEC12A, CD82, TNFRSF1B, ADGRE2, ITGB5, CD96, CCR1, PTPRJ, CD70, LILRB2, LTB4R, TLR2, LILRA2, ITGAX, CR1, EMC10, EMB, DAGLB, P2RY13, LILRB3, LILRB4, SLC30A1, LILRA6, SLC6A6, SEMA4A, TAG72, FRα, PMSA, Mesothelin, LIV-1, CEA, MUC1, PD1, BLIMP1, CTLA4, LAG3, TIM3, TIGIT, CD39, Nectin-4, a cancer marker, a healthy tissue marker, and a cardiac marker. In a further embodiment, the first, second, third, fourth, fifth, sixth, and/or seventh binding domains comprise a polypeptide having at least 40%, 45%, 50%, 55%, 60%, 65%, 70%, 75%, 80%, 85%, 90%, 91%, 92%, 93%, 94%, 95%, 96%, 97%, 98%, 99%, or 100% identity to the amino acid sequence selected from the group consisting of SEQ ID NOS: 27,399-27,403.

### III. Method of Disclosure

Some aspects of the present disclosure are directed to methods of increasing selectivity of a cell in vitro, ex vivo, or in vivo for a CAR T cell therapy. Other aspects of the present disclosure are directed to methods of increasing selectivity of cells that are interacting with each other in vitro, ex vivo, or in vivo for a CAR T cell therapy. Other aspects of the present disclosure are directed to methods of targeting heterogeneous cells (more than two different cell types) in vitro, ex vivo, or in vivo for a CAR T cell therapy. Other aspects of the present disclosure are directed to methods of reducing off-target activity in vitro, ex vivo, or in vivo for a CAR T cell therapy.

In some aspects, the present disclosure is directed to a method of increasing selectivity of a cell comprising expressing a first cage polypeptide disclosed herein and a first key polypeptide disclosed herein in vitro, in vivo, or ex vivo for a CAR T cell therapy. In some aspects, the present disclosure is directed to a method of increasing selectivity of a cell comprising adding a first cage polypeptide disclosed herein and a first key polypeptide disclosed herein in vitro, in vivo, or ex vivo for a CAR T cell therapy. The first cage polypeptide and one or more key polypeptides can be added to the cells in vitro, in vivo, or ex vivo together (concurrently) or separately. Some aspects of the present disclosure are directed to a method of increasing selectivity of a cell in vitro, ex vivo, or in vivo for a CAR T cell therapy comprising (a) contacting cells with (e.g. expressing or adding) a first cage polypeptide fused to a first binding domain, and (b) contacting ((e.g. expressing or adding) the cell with a first key polypeptide fused to a second binding domain. In some aspects, the first cage polypeptide comprises (i) a structural region and (ii) a latch region further comprising one or more bioactive peptides.

Some aspects of the present disclosure are directed to a method of increasing selectivity of cells that are interacting with each other in vitro, ex vivo, or in vivo for a CAR T cell therapy comprising: (a) contacting two or more cells with a first cage polypeptide fused to a first binding domain, wherein the first cage polypeptide comprises (i) a structural region and (ii) a latch region further comprising one or more bioactive peptides, wherein the structural region interacts with the latch region to prevent activity of the one or more bioactive peptides in the absence of colocalization with a key polypeptide and wherein the first binding domain is capable of binding to a first cell moiety present on a synapse between the two or more cells; and (b) contacting the two or more cells with a first key polypeptide fused to a second binding domain, wherein upon colocalization with the first cage polypeptide, the first key polypeptide is capable of binding to the cage structural region to activate the one or more bioactive peptides, wherein the second binding domain is capable of binding to a second cell moiety present on the synapse between the two or more cells.

In some aspects, the method further comprises contacting a second key polypeptide fused to a third binding domain with a synapse of two or more cells that also express a first cell moiety, wherein upon colocalization with the first cage polypeptide, the second key polypeptide is capable of binding to the cage structural region to activate the one or more bioactive peptides, wherein the third binding domain is capable of binding to a third cell moiety present on the synapse of the two or more cells.

In some aspects, the method further comprises contacting the two or more cells with one or more decoy cage polypeptide fused to one or more decoy binding domain with the two or more cells, wherein each decoy cage polypeptide comprises a decoy structural region, which upon colocalization with the first key polypeptide and the first cage polypeptide, is capable of preferentially binding to the first key polypeptide and wherein each decoy binding domain is capable of binding to a decoy cell moiety in the synapse of the two or more cells.

Some aspects of the disclosure are directed to a method of targeting heterogeneous cells (i.e., more than two different cell types) in vitro, ex vivo, or in vivo for a CAR T cell therapy, wherein a first cell moiety and a second cell moeity are present on the first cell and a first cell moiety and a third cell moiety are present on the second cell, comprising: (a) contacting two or more cells with a first cage polypeptide fused to a first binding domain, wherein the first cage polypeptide comprises (i) a structural region and (ii) a latch region further comprising one or more bioactive peptides, and wherein the structural region interacts with the latch region to prevent activity of the one or more bioactive peptides in the absence of colocalization with a key polypeptide and wherein the first binding domain is capable of binding to a first cell moiety present on or within the two or more cells; (b) contacting the two or more cells with a first key polypeptide fused to a second binding domain, wherein upon colocalization, the first key polypeptide is capable of binding to the cage structural region to activate the one or more bioactive peptides and wherein the second binding domain is capable of binding to a second cell moiety present on a cell that also comprises the first cell moiety, and (c) contacting the two or more cells with a second key polypeptide fused to a third binding domain, wherein upon colocalization, the second key polypeptide is capable of binding to the cage structural region to activate the one or more bioactive peptides and wherein the third binding domain is capable of binding to a third cell moiety in a cell that comprises the first cell moiety.

In some aspects, the method further comprises contacting the two or more cells for a CAR T cell therapy with one or more decoy cage polypeptide fused to one or more decoy binding domain, wherein each decoy cage polypeptide comprises a decoy structural region, which upon colocalization with the first key polypeptide, the second key polypeptide, and/or the first cage polypeptide, is capable of preferentially binding to the first key polypeptide or the second key polypeptide, and wherein each decoy binding domain is capable of binding to a decoy cell moiety in a cell that comprises the first cell moiety and the second cell moiety.

Some aspects of the present disclosure are directed to a method of reducing off-target activity in vitro, ex vivo, or in vivo for a CAR T cell therapy comprising (a) contacting two or more cells with a first cage polypeptide fused to a first binding domain, wherein the first cage polypeptide comprises (i) a structural region and (ii) a latch region further comprising one or more bioactive peptides, and wherein the structural region interacts with the latch region to prevent activity of the one or more bioactive peptides in the absence of colocalization with a key polypeptide and wherein the first binding domain is capable of binding to a first cell moiety present on a cell; (b) contacting the two or more cells with a first key polypeptide fused to a second binding domain, wherein upon colocalization, the first key polypeptide is capable of binding to the cage structural region to activate the one or more bioactive peptides and wherein the second binding domain is capable of binding to a second cell moiety present on a cell that also comprises the first cell moiety, and (c) contacting the two or more cells with a decoy cage polypeptide fused to a third binding domain, wherein the decoy cage polypeptide comprises a decoy structural region, which upon colocalization with the key polypeptide and the first cage polypeptide, is capable of preferentially binding to the first key polypeptide and wherein the third binding domain is capable of binding to a third cell moiety in a cell that comprises the first cell moiety and the second cell moiety. In some aspects, the third cell moiety is only present on a healthy cell.

As used herein, “contacting” refers to any means of bring a first element into contact with a second element. In some aspects, contacting includes directly adding a first element, e.g., a polypeptide, to second element, e.g., a cell, such as, for example, by adding a protein into a cell culture. In some aspects, contacting includes expressing the first element, e.g., a protein, by a nucleotide encoding the protein in the target cell or in a cell that is in the same culture as the target cell. In some aspects, the contacting of (a) the cell with a first cage polypeptide fused to a first binding domain, and (b) the contacting of the cell with a first key polypeptide fused to a second binding domain are performed concurrently. In some aspects, the contacting (a) is performed prior to the contacting (b). In some aspects, the contacting (b) is performed prior to the contacting (a). In some aspects, the contacting includes introducing a polynucleotide encoding a polypeptide (e.g., the first cage polypeptide, the first key polypeptide, the second key polypeptide, and the decoy cage polypeptide).

The method disclosed herein increases the selectivity of a cell for a target cell. In some aspects, the colocalization of the first cage polypeptide and the key polypeptide increases the selectivity of a cell that highly comprises the first cell moiety and the second cell moiety. In some aspects, the colocalization of the first cage polypeptide and the key polypeptide increases the selectivity of a cell that highly expresses the first and second cell moiety. In some aspects, the colocalization of the first cage polypeptide and the key polypeptide increases the selectivity of a cell that highly expresses the first and second cell moieties and a cell that highly expresses the first and third cell moieties.

In another embodiment, the disclosure provides methods for cell targeting, comprising

(a) contacting a biological sample containing cells with
(i) a cage polypeptide comprising (i) a structural region, (ii) a latch region further comprising one or more bioactive peptides, and (iii) a first binding domain that targets a cell of interest, wherein the structural region interacts with the latch region to prevent activity of the one or more bioactive peptides; and
(ii) a key polypeptide comprising a second binding domain that targets the cell of interest, wherein the first binding domain and the second binding domain bind to (i) different moieties on the surface of the same cell, (ii) the same moiety on the surface of the same cell, (iii) different moieties at the synapse between two cells that are in contact, or (iv) the same moiety at the synapse between two cells that are in contact;

wherein the contacting occurs for a time and under conditions to promote binding of the cage polypeptide and the key polypeptide to the cell of interest, and to promote binding of the key polypeptide to the cage structural region to displace the latch region and activate the one or more bioactive peptides only when the cage polypeptide and the key polypeptide are co-localized to the cell of interest;

(b) contacting the biological sample with one or more effector molecule(s) under conditions to promote binding of the one or more effector molecules selected from the fusion proteins, nucleic acids, vectors, and/or cells of any embodiment of the disclosure under conditions to promote binding of the one or more effector molecules to the one or more activated bioactive peptides to produce an effector molecule-bioactive peptide complex; and

(c) optionally detecting the effector molecule-bioactive peptide complex, wherein the effector molecule-bioactive peptide complex provides a measure of the cell of interest in the biological sample.

These methods can be used, for example, to specifically target cells of interest such as CAR T cells. As described in the examples that follow, the methods, fusion proteins, and compositions have been used for ultra-specific CAR T cell targeting, and directing CAR T cell cytotoxicity against certain cells within a complex milieu. In one embodiment, the biological sample is present within or obtained from a subject having a disease to be treated, and wherein the method serves to treat the disease. Such disease may include, for example, cancer, and the biological sample comprises tumor cells. In one such embodiment, step (a) of the method comprises intravenous infusion into the subject. In another embodiment, step (b) is carried out after step (a).

Other aspects of the disclosure are directed to methods of preparing a subject in need of a therapy comprising administering a composition disclosed herein. Some aspects of the disclosure are directed to methods of preparing a subject in need of a CAR T cell therapy comprising administering a cell disclosed herein.

Some aspects are directed to a method of treating a disease or condition in a subject in need thereof comprising administering an effector molecule to the subject, wherein the subject is further administered a composition disclosed herein together with administration of the effector molecule. In some aspects, the administering of the effector molecule administration of the effector kills the cell that comprises the first binding moiety and the second binding moiety, results in receptor signaling (e.g., cytokine) in the cell that comprises the first binding moiety and the second binding moiety; results in production of signaling molecules (e.g., cytokine, chemokine) nearby the cell that comprises the first binding moiety and the second binding moiety; or results in differentiation of the cell that comprises the first binding moiety and the second binding moiety. Any effector molecule disclosed herein can be used in the method. In some aspects, the effector molecule binds to the one or more bioactive peptides. In some aspects, the effector molecule comprises an antibody or antigen binding fragment thereof, T cell receptor, DARPin, bispecific or bivalent molecule, nanobody, affibody, monobody, adnectin, alphabody, albumin binding domain, adhiron, affilin, affimer, affitin/nanofitin; anticalin; armadillo repeat protein; atrimer/tetranectin; avimer/maxibody; centyrin; fynomer; Kunitz domain; obody/OB-fold; pronectin; repebody; a computationally designed protein; a protease, a ubiquitin ligase, a kinase, a phosphatase, and/or an effector that induces proteolysis; or any combination thereof. In certain aspects, the effector molecule comprises an antibody or antigen binding fragment thereof. In some aspects, the antigen binding portion thereof comprises a Fab′, F(ab′)₂, Fab, Fv, rIgG, recombinant single chain Fv fragment (scFv), and/or V_{H} single domain.

In some aspects, the effector molecule is a therapeutic cell. in some aspects, the therapeutic cell comprises a T cell, a stem cell, an NK cell, a B cell, or any combination thereof. In some aspects, the therapeutic cell comprises an immune cell. In some aspects, therapeutic cell comprises a T cell. In some aspects, therapeutic cell comprises a stem cell. In some aspects, the stem cell is an induced pluripotent stem cell. In some aspects, therapeutic cell comprises an NK cell.

### EXAMPLES

### Summary

Natural biological systems integrate multiple protein binding inputs through post-translational signaling cascades that are hardcoded to specialized functions; a synthetic system capable of integrating multiple binding inputs through conformational switching could be a general solution for predictively controlling diverse biological functions. We describe the computational design of proximity-activated de novo protein switches that perform ‘AND’, ‘OR’, and ‘NOT’ Boolean logic operations and combinations thereof in response to precise combinations of protein-binding events. The switches activate via a conformational change only when all logic conditions are met, and a high-resolution x-ray crystal structure confirms the design model. We demonstrate the utility of this system for ultraspecific targeting of mammalian cells that are distinguished in a complex cell population only by their precise combination of surface markers. We implement 2- and 3-input logic gates to redirect T cell specificity against tumor cells expressing precise combinations of surface antigens while avoiding off-target recognition of cells expressing single antigens or, in the case of ‘NOT’ logic, a unique third antigen. Our work shows that de novo designed proteins can perform computations on the surface of cells, integrating multiple distinct binding interactions into a single biological output.

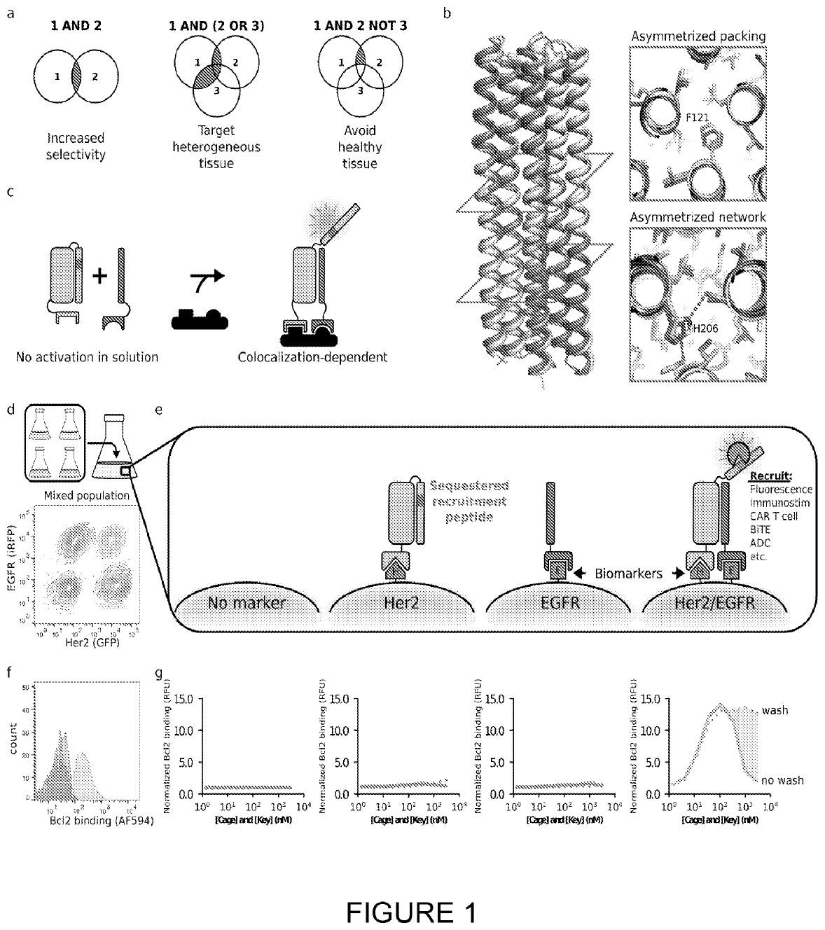

We set out to design a generalizable protein system from scratch that is capable of performing complex logic in response to combinatorial binding events. We aimed for a modular system capable of computing combinations of Boolean logic operations (‘AND’, ‘OR’, and ‘NOT’) when the components are brought into close proximity and actuating a single binding interaction as output (FIG. 1a). Such a system would be broadly useful for modulating a wide range of cellular transactions in the nucleus, cytoplasm, and cell surface. Herein, we develop such a system and apply it to cellular targeting applications: we sought to distinguish cell subpopulations using Boolean logic to integrate multiple protein binding inputs into a single output biological function, taking advantage of the property that antigen binding at the cell surface increases the local concentration of the bound protein. For this system to be generally useful, the actuation must be modular and independent of target antigen identity.

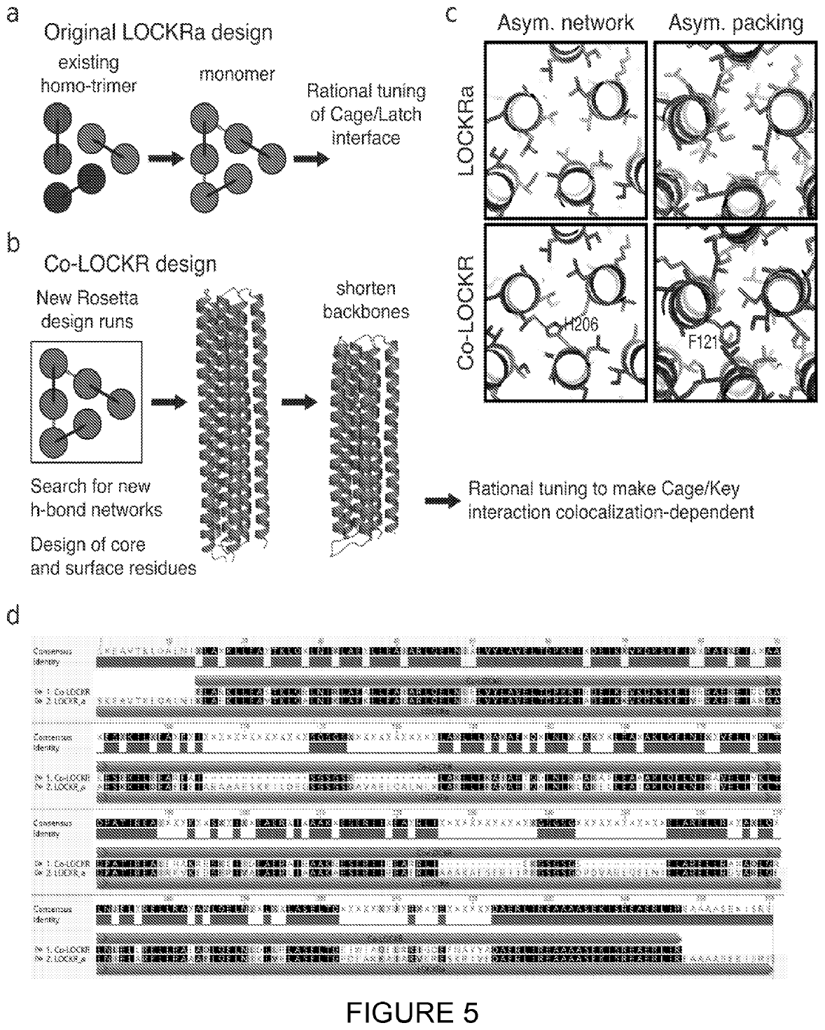

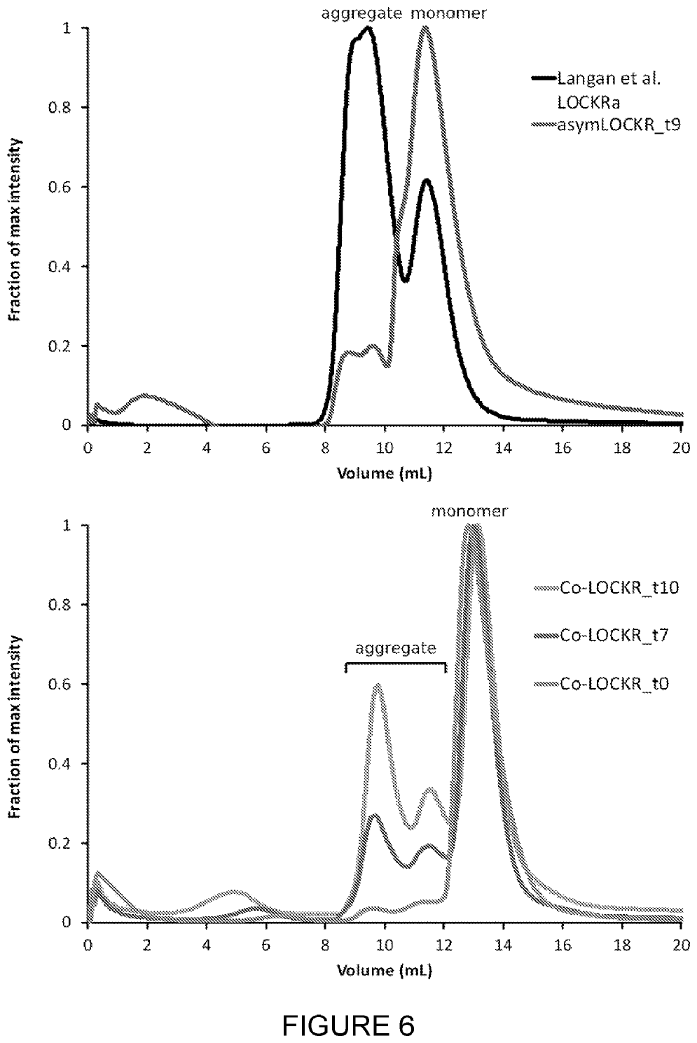

We set out to design de novo protein switches for which the actuation domain is activated by the proximity of additional designed components. We designed protein switches that activate in solution: Latching Orthogonal Cage-Key pRotein (LOCKR) switches are composed of a structural “Cage” protein that uses a “Latch” domain to sequester a functional peptide in an inactive conformation until binding of a separate “Key” protein induces a conformational change that permits binding to an “Effector” protein. Cage, Key, and Effector bind in a three-way equilibrium, and the sensitivity of the switch can be tuned by adjusting the relative Cage-Latch and Cage-Key affinities. We designed new LOCKR proteins to be inert in solution and strongly activated only when the Cage and Key are colocalized. We designed new LOCKR switches with shorter helices, improved hydrophobic packing, and an additional hydrogen bond network to promote interaction specificity among the helices (FIG. 5a-c and the Computational Protein Design portion of the Methods section provide a detailed description of the design process). The new design was nearly 100% monomeric and showed substantially reduced aggregation compared to other exemplary LOCKR switches (FIG. 6a). The improved solution behavior of the new design enabled us to solve a 2.1 Å x-ray crystal structure, which closely matched the design model (FIG. 1b, Table 16) with 1.1 Å root mean squared deviation (RMSD) across all backbone atoms and 0.5 Å RMSD across all sidechain heavy atoms in the newly designed hydrogen bond network (FIG. 1b).

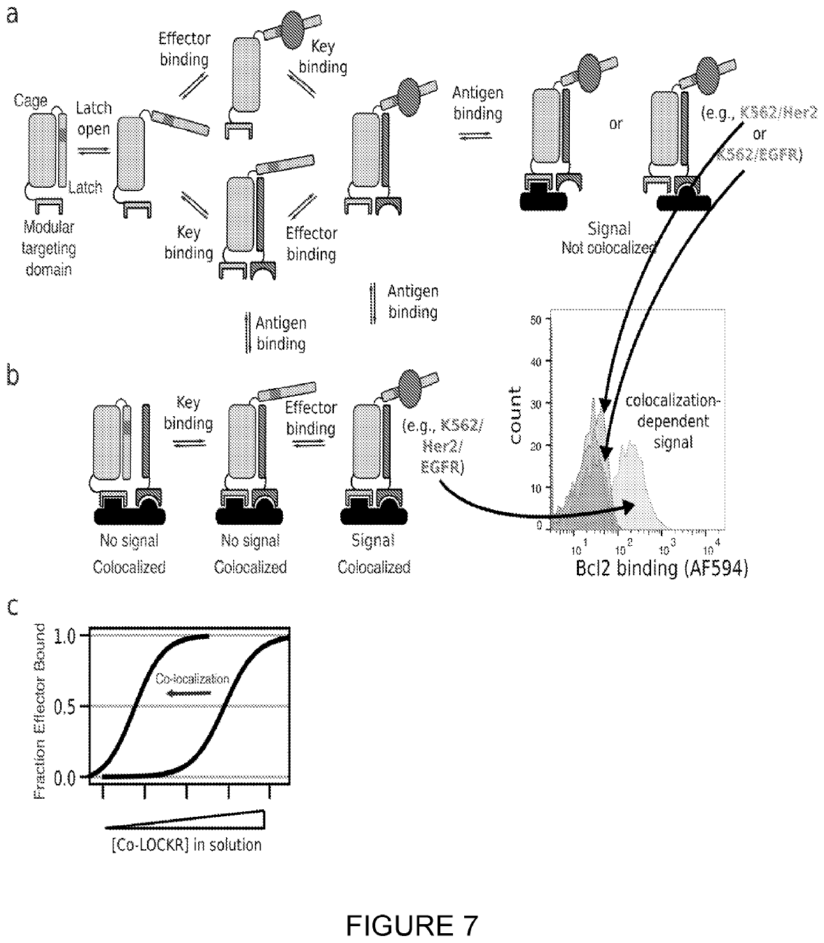

We used the new design as the starting point to develop colocalization-dependent LOCKR (Co-LOCKR) switches (FIG. 1c). To install an output function into Co-LOCKR, we chose the Bim-Bcl2 pair as a well-studied model system for peptide-protein binding (12). Bim was encoded into the Latch as a sequestered peptide; Bcl2 was used as the Effector. We then added targeting domains that recruit the Co-LOCKR Cage and Key to cells expressing target antigens. While the targeting domains should bind to any cell expressing their target antigens, only cells with both antigens should recruit both Cage and Key proteins, achieving colocalization-dependent activation (FIG. 1d-e). Co-LOCKR actuates via a thermodynamic mechanism based on reversible protein-protein interactions; therefore, complex formation can occur in solution (FIG. 7a) or on a surface (FIG. 7b), where Cage-Key colocalization increases local concentration and shifts the binding equilibrium in favor of complex formation (FIG. 7c). We demonstrate below the use of Co-LOCKR switches to regulate the recruitment of Effector proteins comprising a fluorophore or a chimeric antigen receptor (CAR).

To evaluate the ability of Co-LOCKR to target cells co-expressing a precise combination of surface antigens, we developed a mixed population flow cytometry assay by combining four K562 cell lines expressing Her2-eGFP, EGFR-iRFP, both, or neither (FIG. 1d). We used Designed Ankyrin Repeat Protein (DARPin) domains (13, 14) to target the Cage and Key to Her2 and EGFR, respectively. If the system functions as designed, only cells co-expressing both Her2 and EGFR should activate Co-LOCKR and bind Bcl2: the Cage contains the sequestered Bim peptide and the Key is required for its exposure. We refer to this Co-LOCKR configuration as CL_C_{H}K_{E}; in this nomenclature “CL” refers to Co-LOCKR, C_{H} indicates that the Cage is targeted to Her2, and K_{E} indicates that the Key is targeted to EGFR (Table 17). When the mixed population of cells was co-incubated with an equimolar dilution series of Cage and Key (3 μM to 1.4 nM) and washed before adding AlexaFluor™594-labeled Bcl2 (Bcl2-AF594), the expected sigmoidal binding curve was observed for the Her2/EGFR cells but not for cells expressing either protein alone (FIG. 1f). When the cells were co-incubated with Cage, Key, and Bcl2-AF594 together without washing, binding was likewise observed only for the Her2/EGFR cells, but Bcl2-AF594 signal peaked at 111 nM CL_C_{H}K_{E} and decreased at higher concentrations; free Cage and Key likely compete for binding to the limited number of surface Her2 and EGFR proteins with Cage-Key-Bcl2 formed in solution.

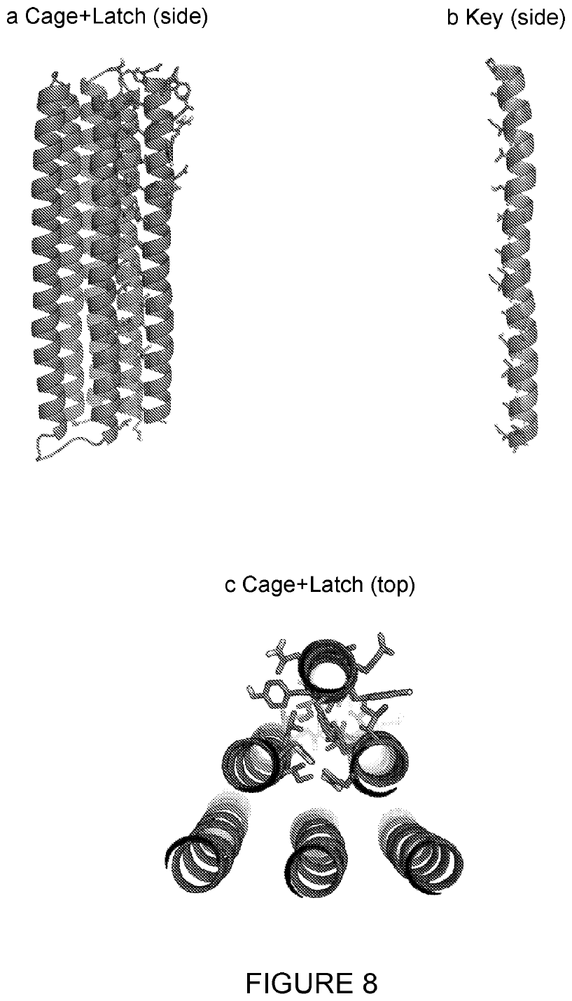

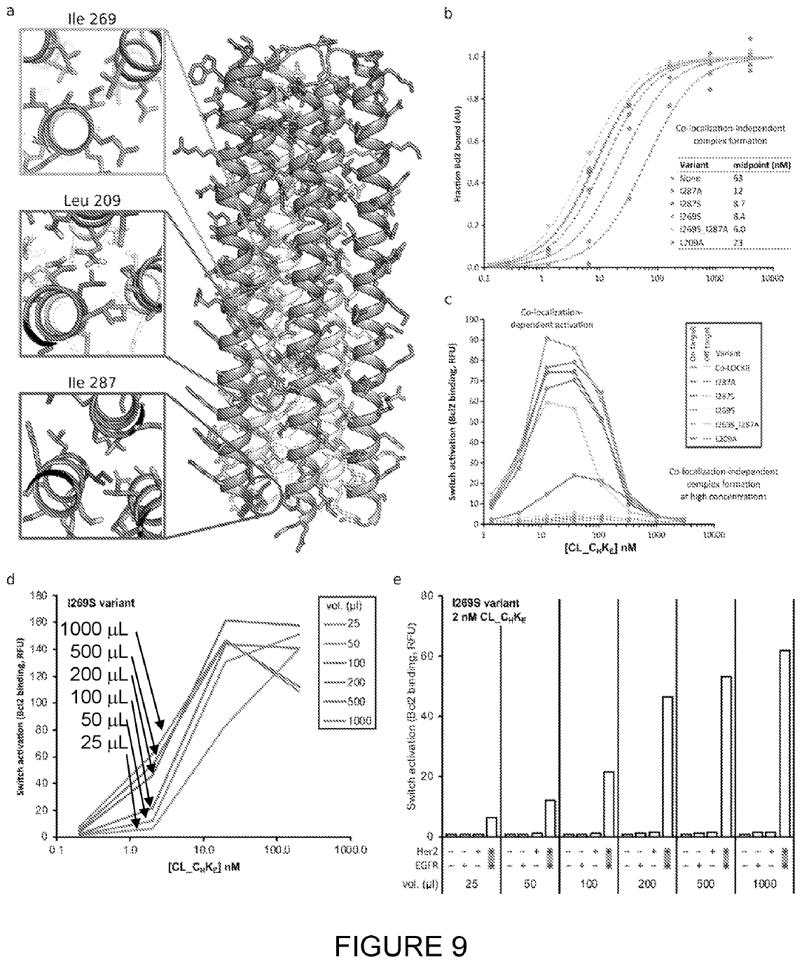

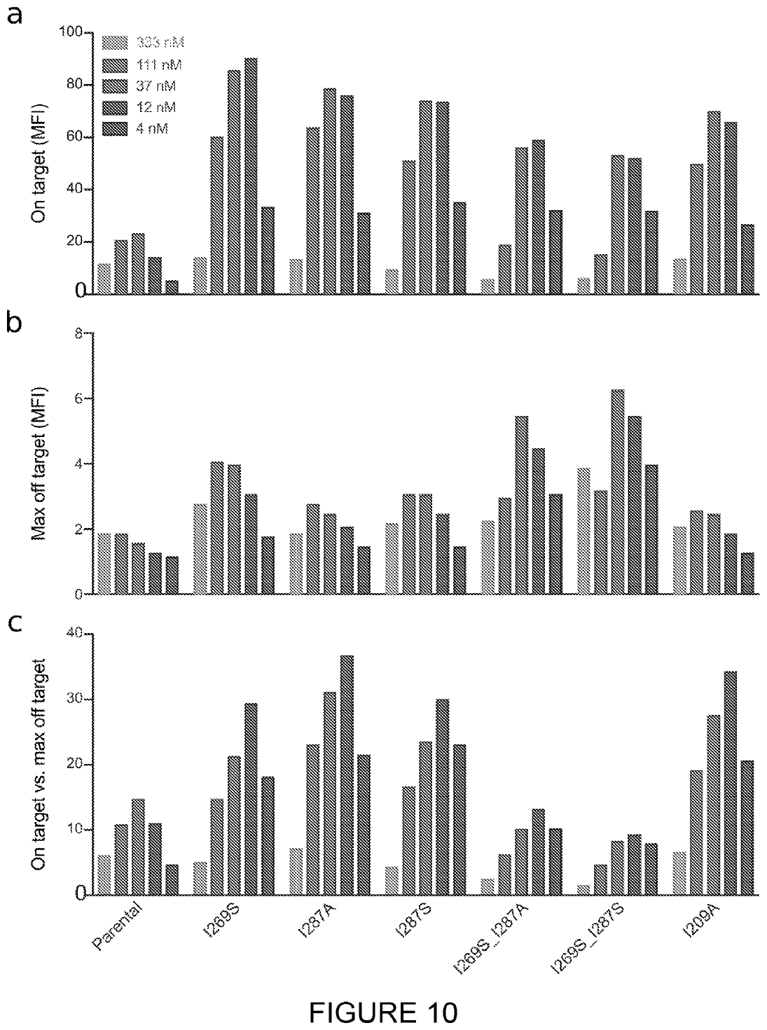

We next sought to tune the dynamic range of Co-LOCKR activation to increase colocalization-dependent activation sensitivity and responsiveness. Our initial design was intended to maximize Cage-Latch affinity so as to ensure colocalization-dependence, leading us to ask whether weakening the Cage-Latch affinity could enhance signal intensity without compromising the ability to compute logic. The sensitivity of previous LOCKR switches was tuned by shortening the Latch to produce a ‘toehold’, but this also promoted aggregation (FIG. 6b). We therefore focused on rationally designed mutations to tune the relative interaction affinities of the Co-LOCKR system to be colocalization-dependent (FIG. 8a-c). We mutated large, hydrophobic residues in the Latch region of the polypeptide of SEQ ID NO: 27359 (I287A, I287S, I269S) or Cage (L209A) to weaken Cage-Latch affinity (FIG. 2a). Biolayer interferometry indicated that increasingly disruptive mutations improved responsiveness (FIG. 9b), and flow cytometry showed that tuning the Cage-Latch interface enhanced colocalization-dependent activation: the tuned variants of CL_C_{H}K_{E} exhibited greater Bcl2-AF594 fluorescence on the same K562/Her2/EGFR cells (FIG. 2b, FIG. 9c). Colocalization-dependent activation occurred even at low nanomolar concentrations of CL_C_{H}K_{E}, likely limited by the number of LOCKR proteins available in small incubation volumes (FIG. 9d-e). Very little Effector binding was observed for cells expressing Her2 or EGFR alone, suggesting that Co-LOCKR avoids targeting nearby cells in trans. Of the switches tested, I269S exhibited the greatest activation (FIG. 10a), the parental Co-LOCKR design exhibited the lowest off-target activation (FIG. 10b), and I287A exhibited the highest fold specificity (FIG. 10c).

Co-localization dependent activation was also observed at the sub-cellular level by confocal microscopy. CL_C_{H}K_{E} recruited Bcl2-AF680 to the plasma membrane of HEK293T/Her2/EGFR cells but not HEK293T/Her2 or HEK293T/EGFR (FIG. 2c). There was a close correspondence between regions of the plasma membrane exhibiting colocalized Her2-eGFP and EGFR-iRFP signal with Co-LOCKR activation (FIG. 2c, column 6, quantified in FIG. 2d).

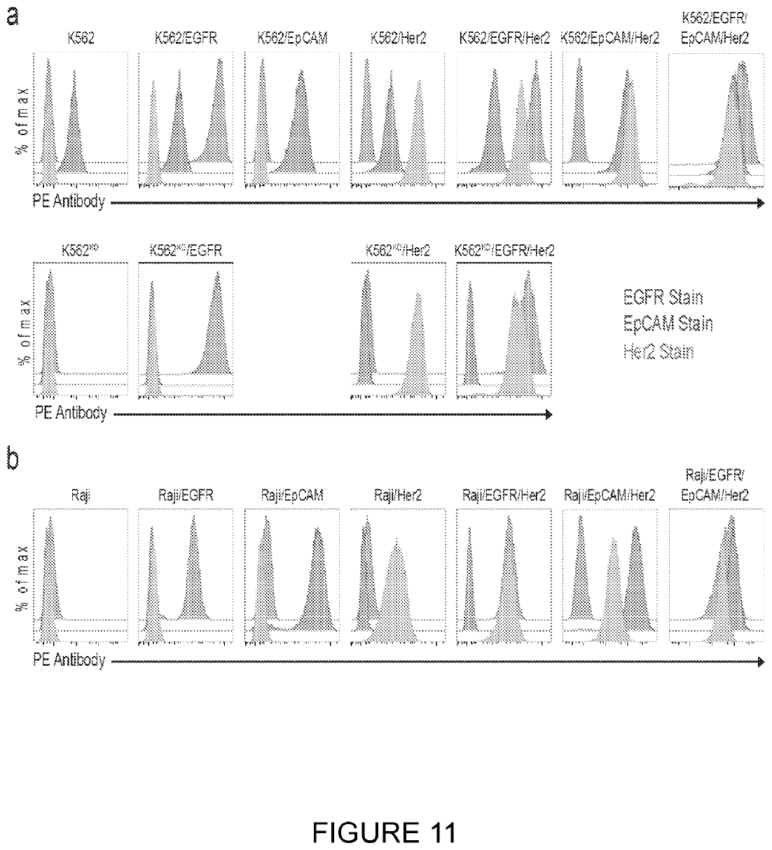

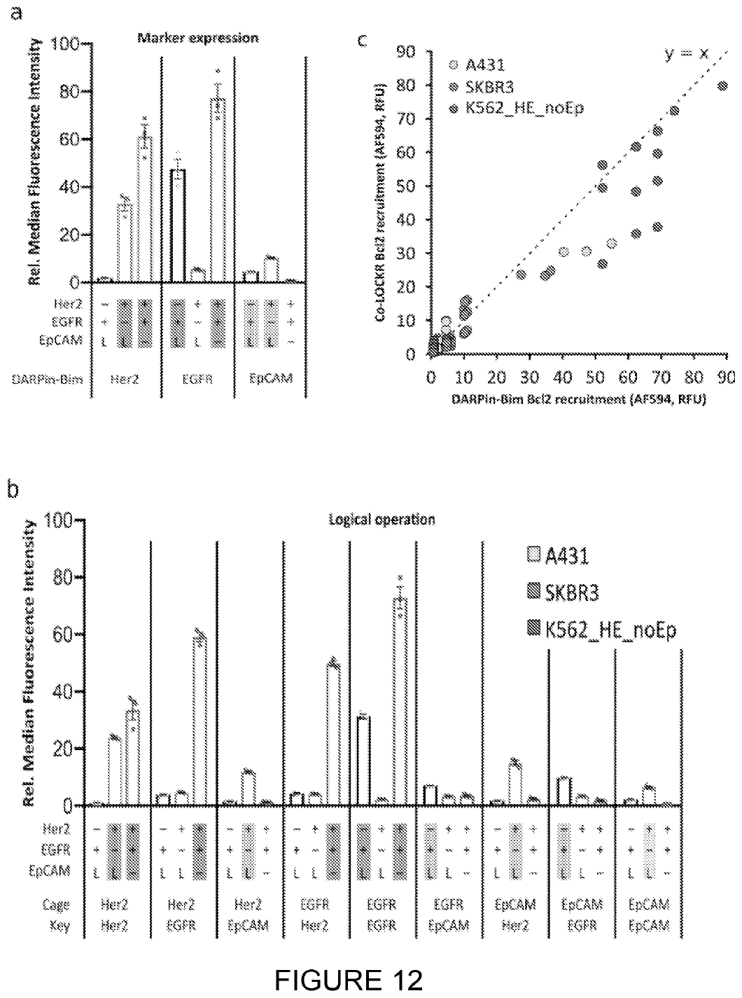

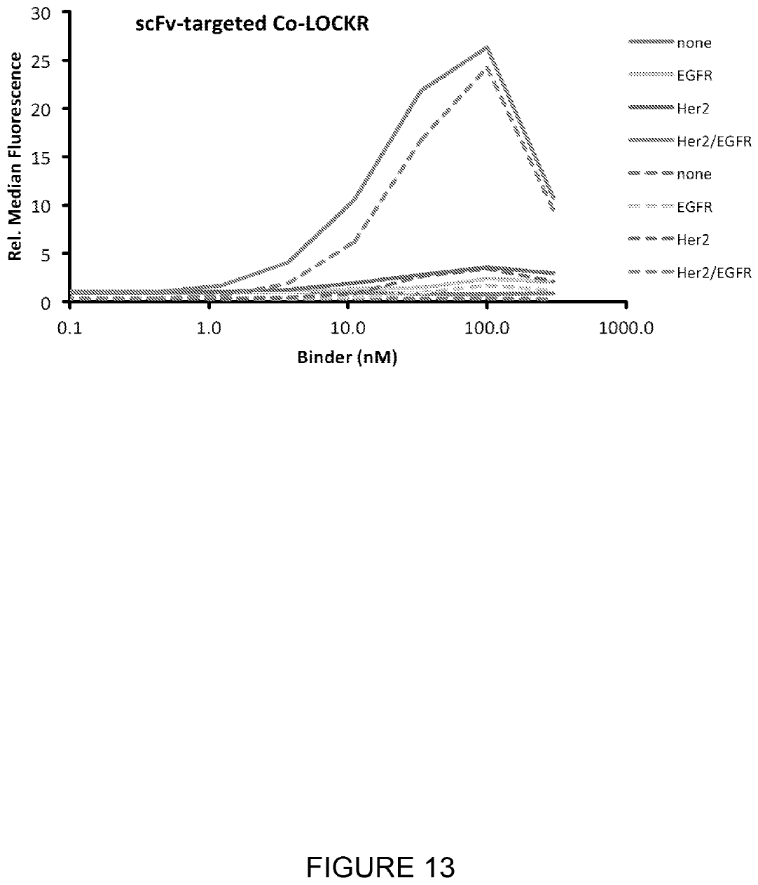

To assess the flexibility of Co-LOCKR, we attempted to specifically target alternative pairwise combinations of three cancer-associated antigens (Her2, EGFR, and EpCAM). Each of these antigens is expressed at differing levels by engineered K562 cell lines or human cancer cell lines (FIG. 11a, FIG. 12a). Using the I269S variant to maximize detection of low levels of antigen, we found that (1) Co-LOCKR could distinguish the correct pair of antigens in every case, and (2) the magnitude of Bcl2 binding corresponded with the expression level of the lower-expressed of the two target antigens (FIG. 3a, FIG. 12b-c), consistent with a stoichiometric binding mechanism for colocalization-dependent activation. Taken together, these results demonstrate the modularity of Co-LOCKR to target several antigens produced at a wide range of differing expression levels. While we chose DARPins as targeting domains so as to enable facile expression of Co-LOCKR variants, any binding domain can be substituted, including single chain variable fragments (FIG. 13).

A truly general technology for targeting any cell type in situ requires more complex logic comprising combinations of ‘AND’, ‘OR’, and ‘NOT’ operations. In principle, the colocalization-dependent activation mechanism of Co-LOCKR should be particularly well suited to accomplish this. ‘OR’ logic can potentially be achieved by adding a second Key fused to a binding domain targeting an alternative surface marker (FIG. 3b). ‘NOT’ logic can potentially be achieved by adding a Decoy protein fused to a binding domain targeting a surface marker to be avoided; the Decoy acts as a sponge to sequester the Key, thereby preventing Cage activation (FIG. 3d).

Using Her2, EGFR, and EpCAM as model antigens (Ag), we first explored [Ag₁ AND either Ag₂ OR Ag₃] logic on the surface of cells (FIG. 3b). To assess the composability of Co-LOCKR targeting, we tested all three combinations: [Her2 AND either EGFR OR EpCAM], [EGFR AND either Her2 OR EpCAM], and [EpCAM AND either Her2 OR EGFR]. In all cases, the correct cell sub-population was targeted at levels consistent with the limiting target antigen (FIG. 3c). For example, CL_C_{E}K_{H}K_{Ep} targeted cells expressing EGFR/EpCAM^{lo} 10-fold over background, Her2/EGFR/EpCAM^{lo} 59-fold over background, and Her2/EGFR/EpCAM^{hi} 56-fold above background, but exhibited minimal off-target activation on cells missing at least one antigen (middle panel of FIG. 3c).

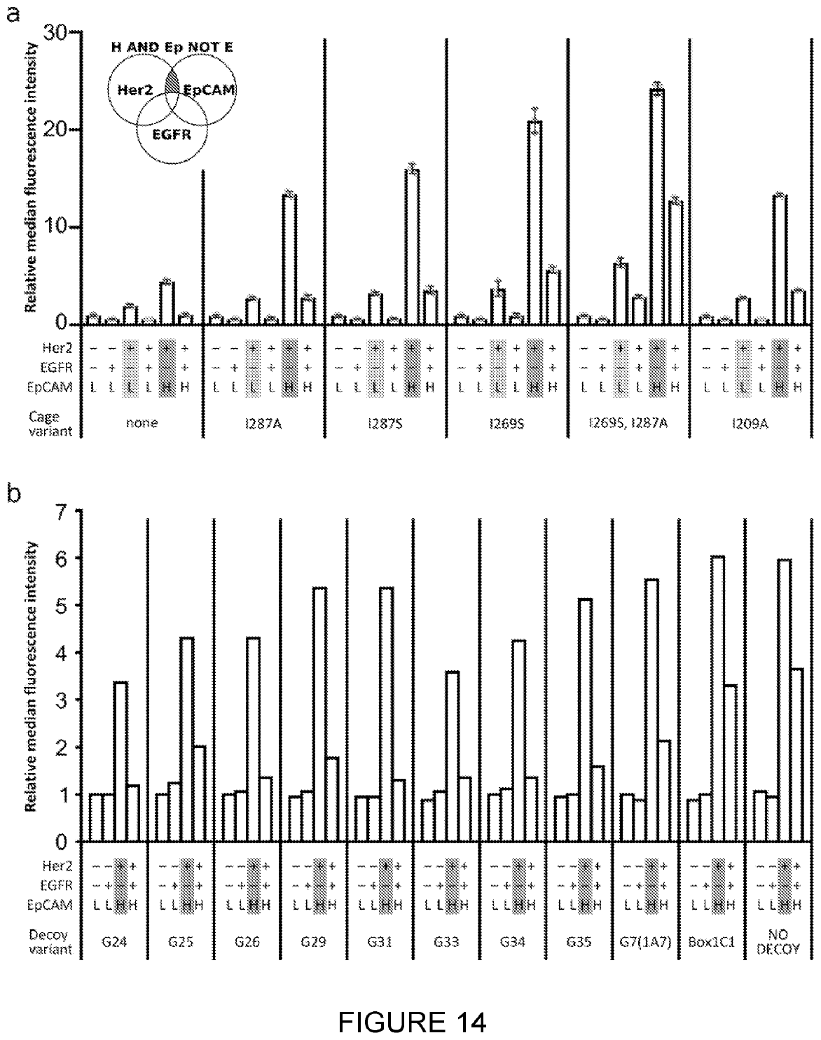

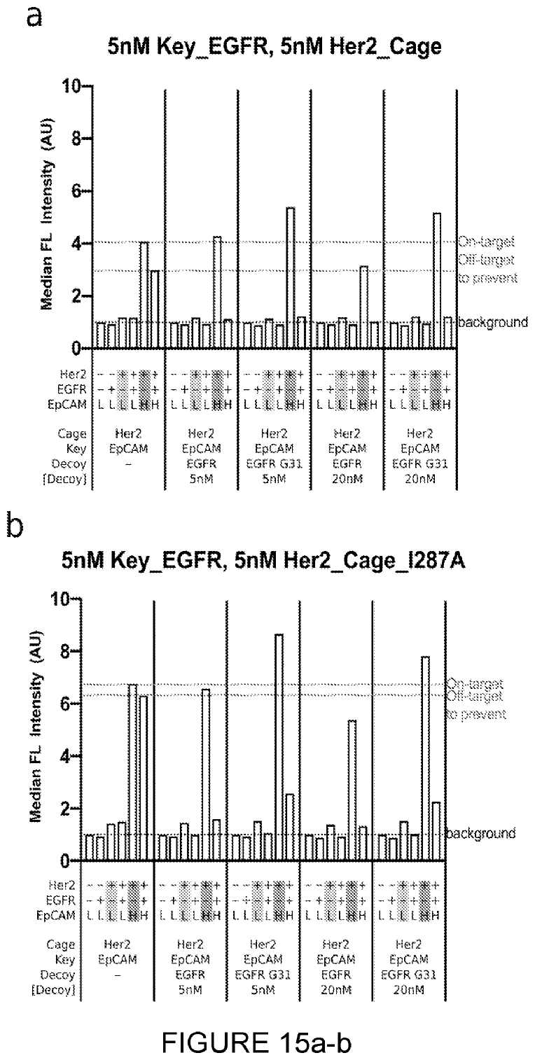

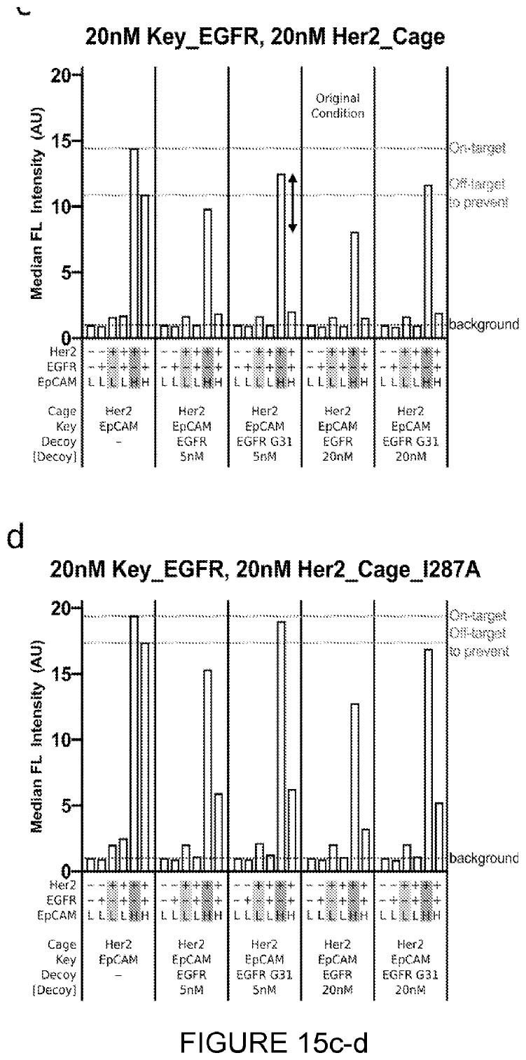

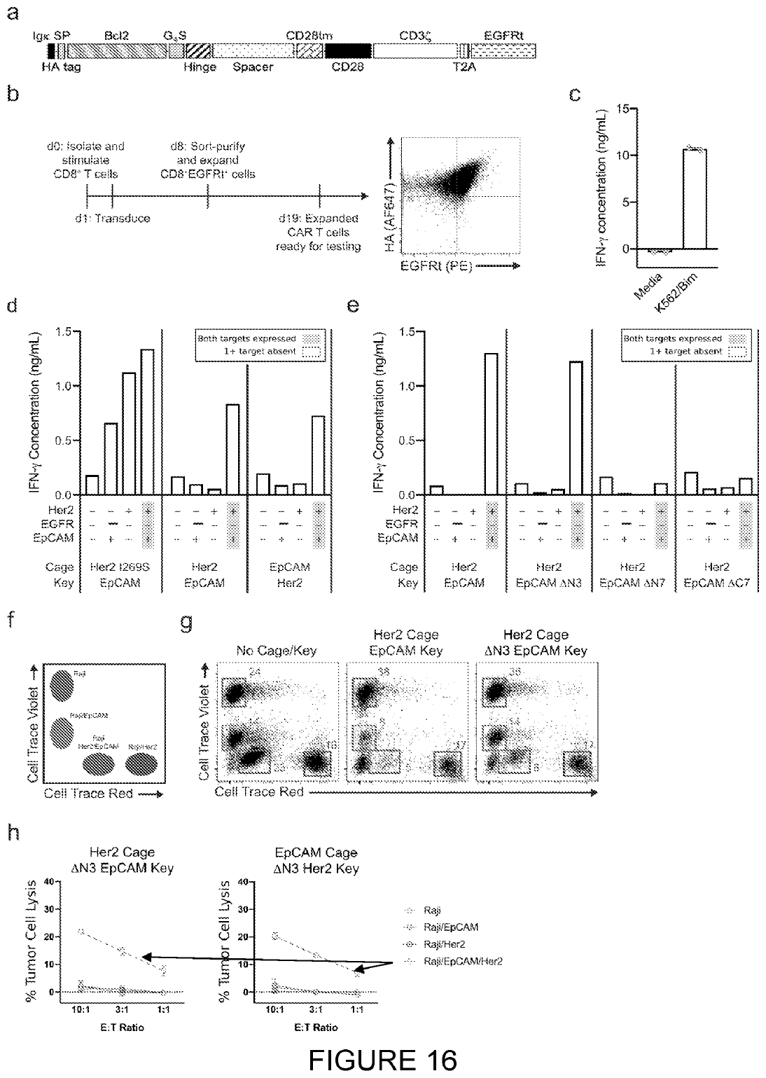

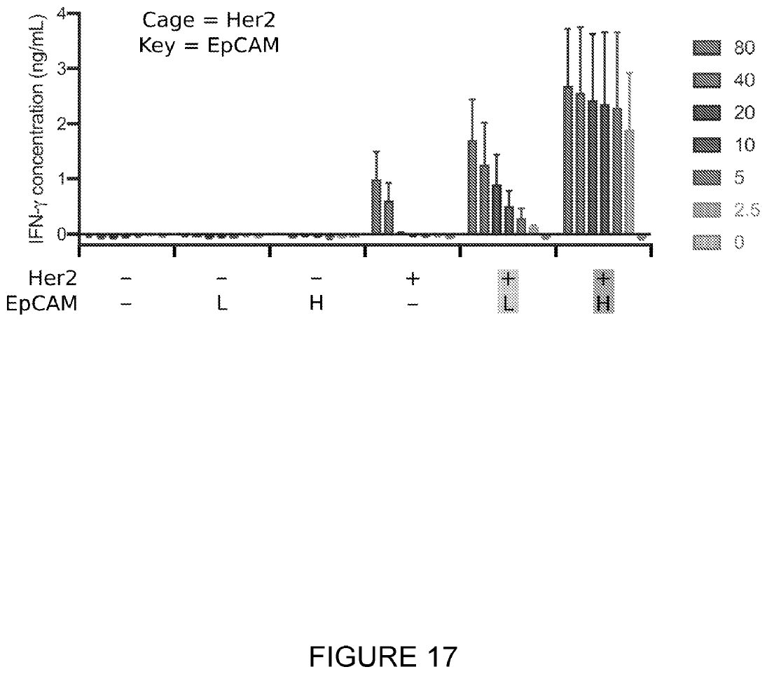

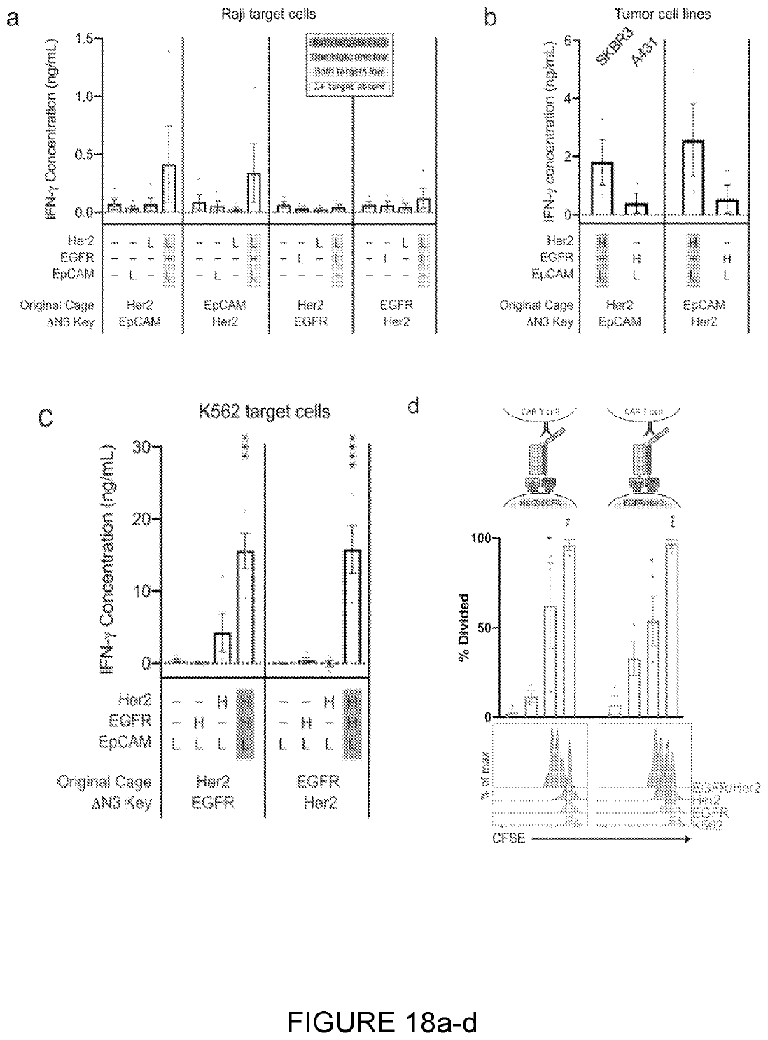

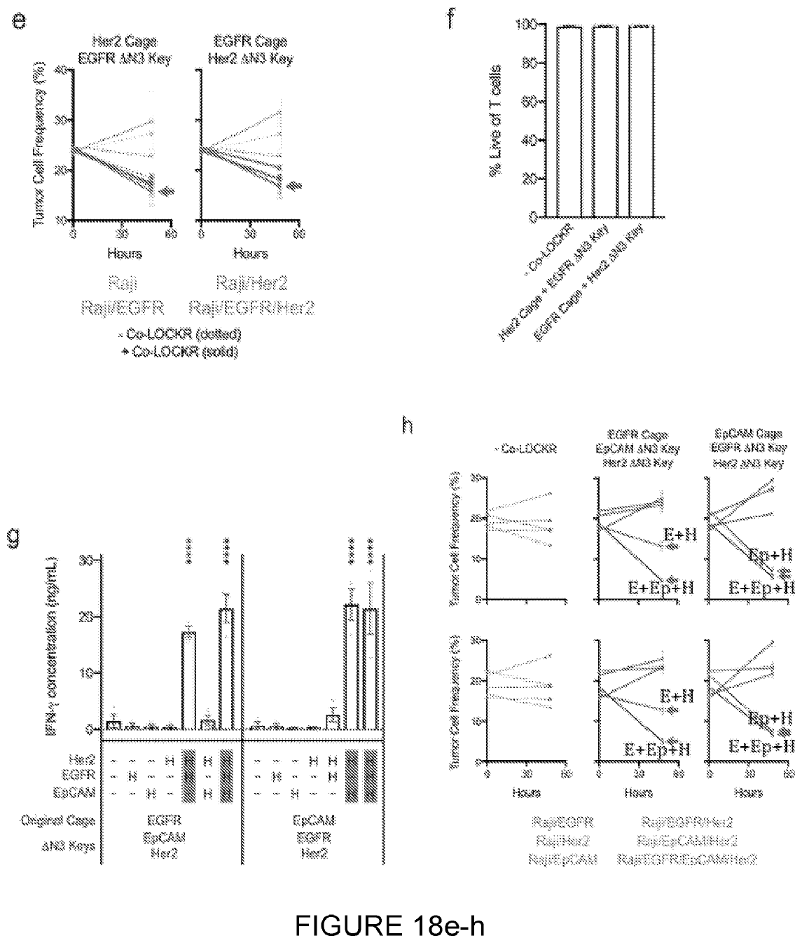

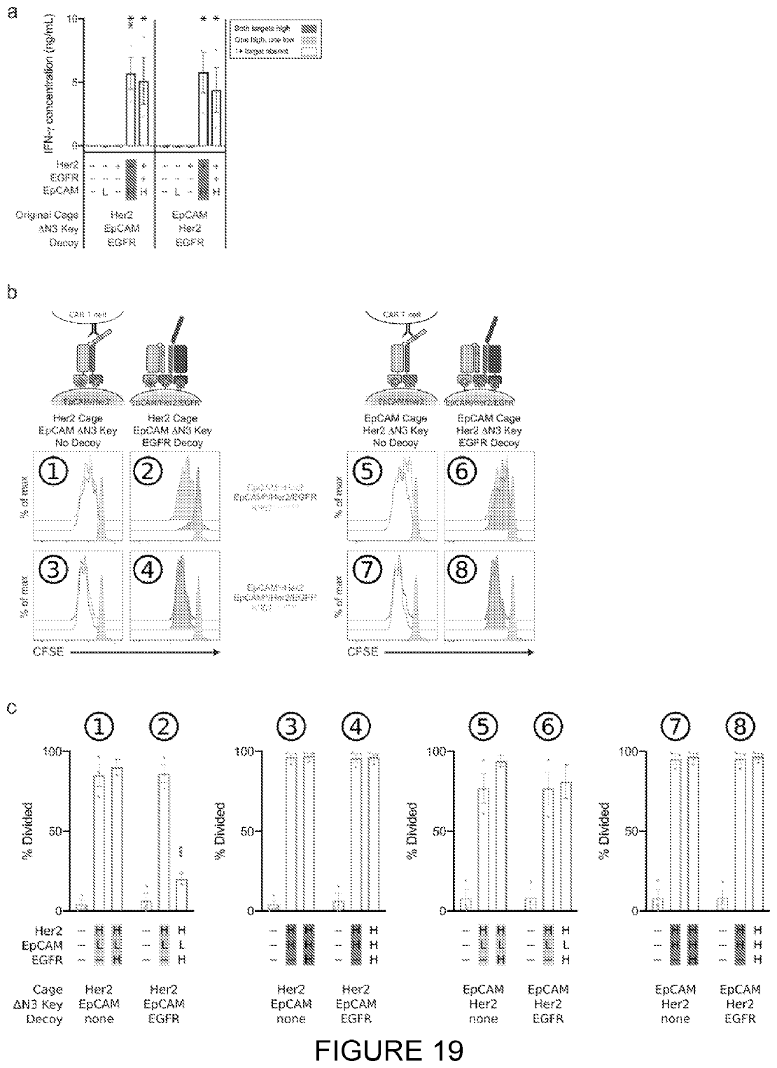

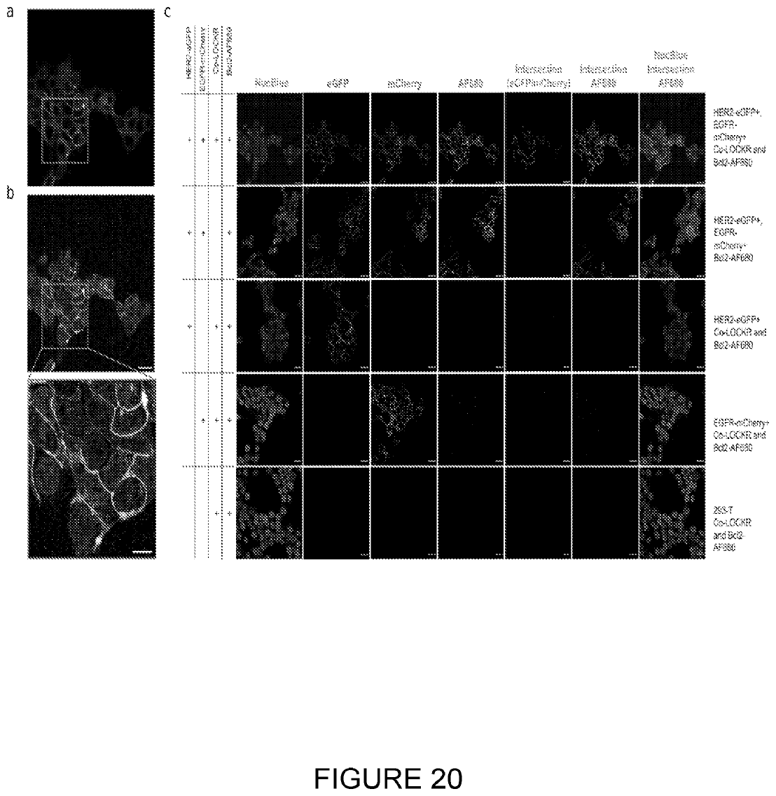

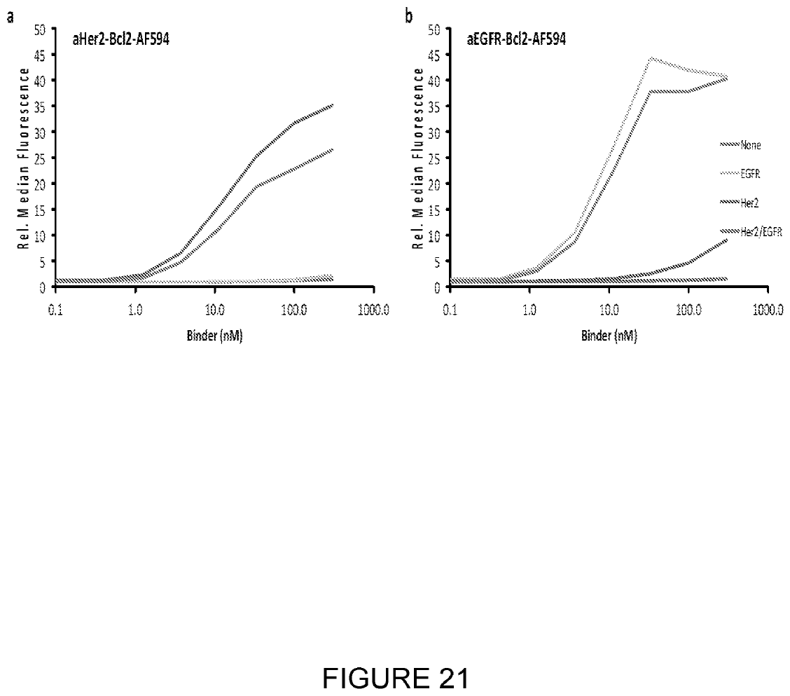

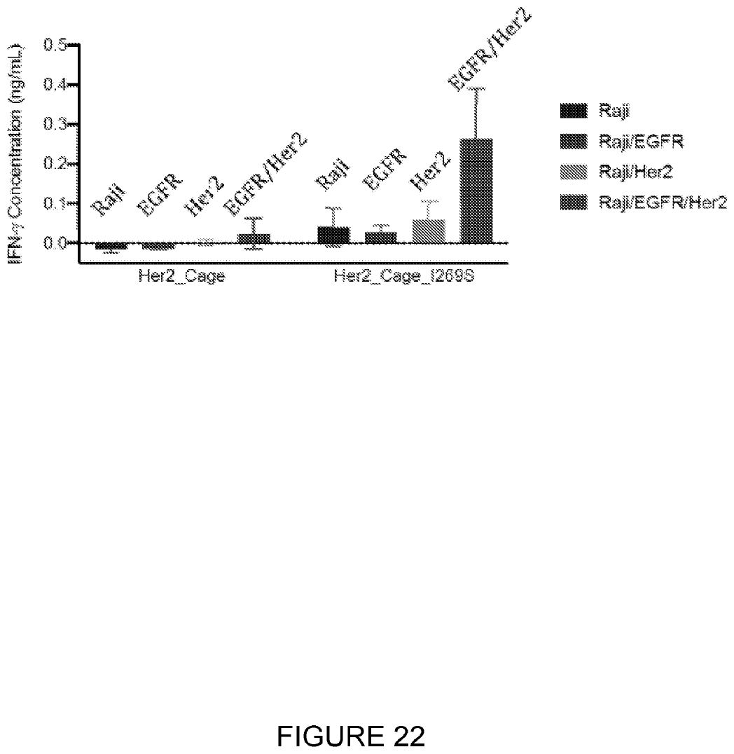

We next explored [Ag₁ AND Ag₂ NOT Ag₃] logic using CL_C_{H}K_{Ep}D_{E} (D for Decoy) and the same set of model antigens (FIG. 3d). Consistent with the expected stoichiometric mechanism of activation, Ag₃ needed to be expressed at higher levels than Ag₂ so that an excess of the Decoy could sequester all molecules of the Key: targeting the Decoy to highly expressed EGFR completely abrogated activation by a Key targeted to low levels but not high levels of EpCAM. The Cage-Latch affinity (FIG. 3d, FIG. 14a) and Decoy-Key affinity (FIG. 14b, FIG. 15a-d) can be readily tuned to either minimize leakiness or maximize activation.

The ability to perform complex logic operations using Co-LOCKR affords a level of control and flexibility not reported by previous targeting technologies. Furthermore, the ability to tune responsiveness with rationally designed point mutations enables the rapid optimization of Co-LOCKR for a wide range of applications.

### Co-LOCKR Mediates Ultraspecific T Cell Targeting

CD19-targeted adoptively transferred chimeric antigen receptor-modified (CAR) T cells have achieved unprecedented clinical success for relapsed or refractory B cell malignancies (15, 16). However, most cancers lack a surface antigen like CD19 that is expressed only on the tumor and a dispensable normal cell lineage (B cells). Thus, cell-based immunotherapies require a flexible strategy to target precise combinations of surface markers that are not found together in vital, healthy cells. Boolean ‘AND’ logic would afford increased tumor selectivity, ‘OR’ logic would enable flexible targeting of heterogeneous tumors or cancers prone to antigen loss, and ‘NOT’ logic would help avoid off-target tissues that share similar expression profiles with the target cancer cells (FIG. 1a).

We explored whether Co-LOCKR could perform logic to restrict T cell targeting to cells expressing multiple specified antigens. We designed a Bcl2 CAR that targets Bim peptides displayed on the surface of a target cell; the CAR contains a stabilized variant of human Bcl2, a flexible extracellular spacer domain (17), CD28/CD3ζ signaling domains, and a truncated EGFR (EGFRt) selection marker (18) linked by a T2A ribosomal skipping sequence (FIG. 16a). The Bcl2 CAR functions as designed: purified CD8⁺ EGFRt⁺ Bcl2 CAR T cells efficiently recognized K562 cells stably expressing a surface-exposed Bim-GFP fusion protein (FIG. 16b-c).

With Bcl2 CAR T cells in hand, we first investigated whether the presence of Co-LOCKR proteins would permit T cell activation against target cells expressing the relevant target antigens. We functionally tested Co-LOCKR-mediated T cell targeting of Raji and K562 cells expressing Her2, EGFR, and EpCAM. Because the Raji cells expressed lower levels of transduced antigens than did the K562 cell lines (FIG. 117a-b), the Raji cells more stringently test Co-LOCKR sensitivity, whereas the K562 cells better assess specificity. To maximize the likelihood of T cell activation, we initially chose to evaluate CL_C_{H}K_{Ep} using the I269S Cage against a panel of Raji cells expressing Her2, EpCAM, both, or none. Although Raji/EpCAM/Her2 cells correctly induced IFN-γ release, we also observed leaky IFN-γ release with Raji/EpCAM and Raji/Her2 cells (FIG. 16d). In contrast, both CL_C_{H}K_{Ep} and CL_C_{Ep}K_{H} using the parental unmutated Cage promoted IFN-γ release only when co-cultured with Raji/EpCAM/Her2 cells, demonstrating that Co-LOCKR can be tuned for CAR T cell retargeting. By titrating the concentration of CL_C_{H}K_{Ep} to assess the impact on cytokine production, we found that CAR T effector function could be specifically targeted using between 2.5 nM to 20 nM of Co-LOCKR without causing unintended activation by off-target cells (FIG. 17).

Next, we assessed the ability of Co-LOCKR to direct CAR T cell cytotoxicity against specific subsets of cells within a complex milieu. Raji, Raji/EpCAM, Raji/Her2, and Raji/EpCAM/Her2 were differentially labeled with fluorescent Cell Trace dyes and mixed together with CAR T cells and CL_C_{H}K_{Ep} (FIG. 16f). We simultaneously measured killing of the four tumor cell lines in the mixed population using flow cytometry. After 48 hours, Raji/EpCAM/Her2 cells were preferentially killed, but a fraction of Raji/EpCAM cells were also targeted (FIG. 16g), suggesting that even the parental Cage and Key were too leaky for CAR T cell recruitment. We sought to overcome this basal activation by tuning the length of the Key (FIG. 16e). The combination of parental Cage and ΔN3 Key (three N-terminal amino acids deleted) selectively targeted Raji/EpCAM/Her2 and mitigated unintended killing of Raji/EpCAM and Raji/Her2 cells (FIG. 16f-g). We confirmed these results using a 4-hour Chromium release assay which showed that CL_C_{H}K_{Ep} specifically targeted Raji/EpCAM/Her2 cells and initiated rapid cell killing (FIG. 16h). Thus, Co-LOCKR can be used to restrict IFN-γ release and cell killing to only those tumor cells that express a specific pair of antigens. T cell cytotoxicity is highly sensitive, and we are aware of no other technology capable of rapidly targeting double-positive cells while sparing single-antigen cells in a mixed population.

After establishing that Co-LOCKR could selectively target Raji/EpCAM/Her2 cells, we turned to our K562 cell lines (FIG. 11a) as well as solid tumor lines (FIG. 12a) to more systematically evaluate Co-LOCKR ‘AND’ logic for additional tumor antigen pairs ([Her2 AND EpCAM], [Her2 AND EGFR]). Using the parental Cage and ΔN3 Key, we observed a positive association between antigen density and the magnitude of on-target CAR T cell IFN-γ production: Raji cells with low antigen density yielded modest IFN-γ (FIG. 18a), K562/Her2/EpCAM^{lo} and SKBR3 breast cancer cells yielded intermediate IFN-γ (FIG. 4a, FIG. 18b), and both K562/Her2/EpCAM^{hi} and K562/Her2/EGFR cells yielded high IFN-γ release for their respective Co-LOCKRs (FIG. 4a, FIG. 18c). For example, CL_C_{Ep}K_{H} induced IFN-γ release in response to Raji/Her2/EpCAM 3.9-fold above background, SKBR3 4.8-fold above background, K562/Her2/EpCAM^{lo} 16-fold above background, and K562/Her2/EpCAM^{hi} 51-fold above background, with minimal off-target cytokine release. Off-target IFN-γ production did not increase appreciably when the target cells expressed high levels of a single antigen.

Consistent with our earlier cytokine secretion results, CAR T cells proliferated only upon co-culture with target cells co-expressing the correct pair of antigens (FIG. 4b, FIG. 18d). To evaluate cell-specific killing, we returned to the Raji cell lines because the K562 cells expressed eGFP and iRFP, which complicated the use of fluorescent Cell Trace dyes. Our flow cytometry-based killing assay revealed ‘AND’ gate selective cytotoxicity with both CL_C_{H}K_{Ep} and CL_C_{Ep}K_{H} against Raji/EpCAM/Her2 without depleting single antigen-positive cells (FIG. 4c). A similar result was observed for both CL_C_{H}K_{E} and CL_C_{E}K_{H} against Raji/Her2/EGFR (FIG. 18e), although killing was less effective, likely due to the lower expression levels of EGFR compared to EpCAM in Raji/Her2/EGFR and Raji/EpCAM/Her2, respectively. We also did not observe fratricide of the EGFRt⁺ CAR T cells used in the experiment, which could have been targeted by the anti-EGFR DARPin binder of CL_C_{H}K_{E} or CL_C_{E}K_{H} (FIG. 18f).

Encouraged by robust ‘AND’ logic, we evaluated more complex operations involving combinations of ‘AND’ and either ‘OR’ or ‘NOT’ logic. CAR T cells co-cultured with ‘AND/OR’ Co-LOCKRs (CL_C_{H}K_{E}K_{Ep}, CL_C_{E}K_{H}K_{Ep}, and CL_C_{Ep}K_{H}K_{E}) each carried out [Ag₁ AND either Ag₂ OR Ag₃] logic with respect to IFN-γ production (FIG. 4d, FIG. 18g) and proliferation (FIG. 4e) against K562 cell lines, as well as selective killing in a mixed population of Raji cell lines (FIG. 4f, FIG. 18h). CART cells co-cultured with an ‘AND/NOT’ Co-LOCKR (CL_C_{H}K_{Ep}D_{E}) carried out [Her2 AND EpCAM NOT EGFR] logic, eliminating IFN-□ production and proliferation in the presence of K562/EGFR/Her2/EpCAM^{lo} cells (FIG. 4g-h). However, the NOT logic exhibited leaky cytokine production and proliferation when EpCAM was overexpressed (FIG. 19a-b) or when the Cage and Key specificities were reversed (CL_C_{Ep}K_{H}D_{E}, FIG. 4g-h). For Raji cells that expressed approximately two-fold more EpCAM than EGFR and 1.6-fold less Her2 than EGFR (Table S3), CL_C_{Ep}K_{H}D_{E} avoided cytotoxicity against Raji/EpCAM/Her2/EGFR cells while CL_C_{H}K_{Ep}D_{E} did not (FIG. 4i). These results support our earlier findings that Ag₃ in the ‘NOT’ operation must be expressed at higher levels than Ag₂: EGFR was expressed at sufficiently higher levels than EpCAM for K562/EGFR/Her2/EpCAM^{lo} cells and Her2 for Raji/EpCAM/Her2/EGFR cells (FIG. 11a-b). While these data indicate that careful tuning will likely be necessary for eventual therapeutic use, the ability of CL_C_{Ep}K_{H}D_{E} to perform [EpCAM AND Her2 NOT EGFR] logic redirecting CAR T cell cytolysis against Her2/EpCAM cells but not Her2/EpCAM/EGFR cells (right hand panel of FIG. 4i) demonstrates the power of Co-LOCKR for ultraspecific cell targeting.

By contrast, Co-LOCKR computes logic on a single cell expressing precise combinations of antigens in cis, specifically directing cytotoxicity against target cells without harming neighboring off-target cells that only provide a subset of the target antigens (FIG. 4c, f, i). ‘OR’ and ‘NOT’ logic combined with ‘AND’ logic have not been described for CAR T cells. The ability to implement complex logic (e.g., [Ag₁ AND either Ag₂ OR Ag₃] (FIG. 3c) and [Ag₁ AND Ag₂ NOT Ag₃] (FIG. 3d, FIG. 4g-i)) is unique to Co-LOCKR and cannot be achieved with existing technologies.

Our CAR T cell experiments demonstrate the potential for Co-LOCKR to mediate unprecedented targeting specificity.

Generally, the power of the Co-LOCKR system results from the integration of multiple coherent or competing inputs that determine the magnitude of a single response. The output signal—exposure of the functional peptide on the Latch—is increased by Key binding and countered by Decoy competition. In principle, there are no limits on the numbers of each molecule, allowing for arbitrarily complex logic operations. Although our present work has focused on describing the system and demonstrating its ability to improve T cell-based cancer immunotherapies in vitro, the Co-LOCKR system is powerful for engineering biology in any setting that requires proximity-based activation or specific targeting through calculations on the surface of cells.

### REFERENCES

1. N. M. Daringer, R. M. Dudek, K. A. Schwarz, J. N. Leonard, Modular Extracellular Sensor Architecture for Engineering Mammalian Cell-based Devices. ACS Synth. Biol. 3, 892-902 (2014).
2. L. Morsut et al., Engineering Customized Cell Sensing and Response Behaviors Using Synthetic Notch Receptors. Cell. 164, 780-791 (2016).
3. N. H. Kipniss et al., Engineering cell sensing and responses using a GPCR-coupled CRISPR-Cas system. Nat. Commun. 8, 2212 (2017).
4. Z. Eshhar, T. Waks, G. Gross, D. G. Schindler, Specific activation and targeting of cytotoxic lymphocytes through chimeric single chains consisting of antibody-binding domains and the gamma or zeta subunits of the immunoglobulin and T-cell receptors. Proc. Natl. Acad. Sci. U.S.A. 90, 720-4 (1993).
5. S. Wilkie et al., Selective expansion of chimeric antigen receptor-targeted T-cells with potent effector function using interleukin-4. J. Biol. Chem. 285, 25538-44 (2010).
6. M. E. Prosser, C. E. Brown, A. F. Shami, S. J. Forman, M. C. Jensen, Tumor PD-L1 co-stimulates primary human CD8+ cytotoxic T cells modified to express a PD1:CD28 chimeric receptor. Mol. Immunol. 51, 263-272 (2012).
7. C. Sellmann et al., Balancing Selectivity and Efficacy of Bispecific Epidermal Growth Factor Receptor (EGFR)×c-MET Antibodies and Antibody-Drug Conjugates. J. Biol. Chem. 291, 25106-25119 (2016).
8. Y. Mazor et al., Enhanced tumor-targeting selectivity by modulating bispecific antibody binding affinity and format valence. Sci. Rep. 7, 40098 (2017).
9. R. A. Langan et al., De novo design of bioactive protein switches. Nature. 572, 205-210 (2019).
10. S. E. Boyken et al., De novo design of protein homo-oligomers with modular hydrogen-bond network-mediated specificity. Science. 352, 680-7 (2016).
11. A. Leaver-Fay et al., ROSETTA3: an object-oriented software suite for the simulation and design of macromolecules. Methods Enzymol. 487, 545-74 (2011).
12. L. Delgado-Soler, M. Pinto, K. Tanaka-Gil, J. Rubio-Martinez, Molecular Determinants of Bim(BH3) Peptide Binding to Pro-Survival Proteins. J. Chem. Inf. Model. 52, 2107-2118 (2012).
13. C. Zahnd et al., A Designed Ankyrin Repeat Protein Evolved to Picomolar Affinity to Her2. J. Mol. Biol. 369, 1015-1028 (2007).
14. D. Steiner, P. Forrer, A. PlUckthun, Efficient Selection of DARPins with Sub-nanomolar Affinities using SRP Phage Display. J. Mol. Biol. 382, 1211-1227 (2008).
15. M. Sadelain, I. Rivière, S. Riddell, Therapeutic T cell engineering. Nature. 545, 423-431 (2017).
16. A. I. Salter, M. J. Pont, S. R. Riddell, Chimeric antigen receptor-modified T cells: CD19 and the road beyond. Blood. 131, 2621-2629 (2018).
17. M. Hudecek et al., The Nonsignaling Extracellular Spacer Domain of Chimeric Antigen Receptors Is Decisive for In Vivo Antitumor Activity. Cancer Immunol. Res. 3, 125-135 (2015).
18. X. Wang et al., A transgene-encoded cell surface polypeptide for selection, in vivo tracking, and ablation of engineered cells. Blood. 118, 1255 (2011).
19. C. C. Kloss, M. Condomines, M. Cartellieri, M. Bachmann, M. Sadelain, Combinatorial antigen recognition with balanced signaling promotes selective tumor eradication by engineered T cells. Nat. Biotechnol. 31, 71-75 (2013).
20. K. T. Roybal et al., Precision Tumor Recognition by T Cells With Combinatorial Antigen-Sensing Circuits. Cell. 164, 770-779 (2016).
21. S. Srivastava et al., Logic-Gated ROR1 Chimeric Antigen Receptor Expression Rescues T Cell-Mediated Toxicity to Normal Tissues and Enables Selective Tumor Targeting. Cancer Cell. 35, 489-503.e8 (2019).
22. J. H. Cho, J. J. Collins, W. W. Wong, Universal Chimeric Antigen Receptors for Multiplexed and Logical Control of T Cell Responses. Cell. 173, 1426-1438.ell (2018).
23. E. Zah, M.-Y. Lin, A. Silva-Benedict, M. C. Jensen, Y. Y. Chen, T Cells Expressing CD19/CD20 Bispecific Chimeric Antigen Receptors Prevent Antigen Escape by Malignant B Cells. Cancer Immunol. Res. 4, 498-508 (2016).
24. V. D. Fedorov, M. Themeli, M. Sadelain, Sci. Transl. Med., in press, doi:10.1126/scitranslmed.3006597.
25. S. Tammana et al., 4-1BB and CD28 Signaling Plays a Synergistic Role in Redirecting Umbilical Cord Blood T Cells Against B-Cell Malignancies. Hum. Gene Ther. 21, 75-86 (2010).
26. Y. Kagoya et al., A novel chimeric antigen receptor containing a JAK-STAT signaling domain mediates superior antitumor effects. Nat. Med. 24, 352-359 (2018).
27. C. Sun et al., THEMIS-SHP1 Recruitment by 4-1BB Tunes LCK-Mediated Priming of Chimeric Antigen Receptor-Redirected T Cells. Cancer Cell. 37, 216-225.e6 (2020).
28. B. Kuhlman, D. Baker, Native protein sequences are close to optimal for their structures. Proc. Natl. Acad. Sci. U.S.A. 97, 10383-8 (2000).
29. S. J. Fleishman et al., RosettaScripts: A Scripting Language Interface to the Rosetta Macromolecular Modeling Suite. PLoS One. 6, e20161 (2011).
30. J. W. Chico et al., α/β-Peptide Foldamers Targeting Intracellular Protein-Protein Interactions with Activity in Living Cells. J. Am. Chem. Soc. 137, 11365-11375 (2015).
31. A. Goldenzweig et al., Automated Structure- and Sequence-Based Design of Proteins for High Bacterial Expression and Stability. Mol. Cell. 63, 337-346 (2016).
32. D. G. Gibson, H. O. Smith, C. A. Hutchison, J. C. Venter, C. Merryman, Chemical synthesis of the mouse mitochondrial genome. Nat. Methods. 7, 901-3 (2010).
33. N. Stefan et al., DARPins Recognizing the Tumor-Associated Antigen EpCAM Selected by Phage and Ribosome Display and Engineered for Multivalency. J. Mol. Biol. 413, 826-843 (2011).
34. W. Kabsch, IUCr, XDS. Acta Crystallogr. Sect. D Biol. Crystallogr. 66, 125-132 (2010).
35. A. J. McCoy et al., Phaser crystallographic software. J. Appl. Crystallogr. 40, 658-674 (2007).
36. P. D. Adams et al., PHENIX: a comprehensive Python-based system for macromolecular structure solution. Acta Crystallogr. Sect. D Biol. Crystallogr. 66, 213-221 (2010).
37. T. C. Terwilliger et al., Iterative model building, structure refinement and density modification with the PHENIX AutoBuild wizard. Acta Crystallogr. Sect. D Biol. Crystallogr. 64, 61-69 (2008).
38. P. Emsley, K. Cowtan, IUCr, Coot: model-building tools for molecular graphics. Acta Crystallogr. Sect. D Biol. Crystallogr. 60, 2126-2132 (2004).
39. I. W. Davis et al., MolProbity: all-atom contacts and structure validation for proteins and nucleic acids. Nucleic Acids Res. 35, W375-W383 (2007).
40. A. D. Bandaranayake et al., Daedalus: a robust, turnkey platform for rapid production of decigram quantities of active recombinant proteins in human cell lines using novel lentiviral vectors. Nucleic Acids Res. 39, e143-e143 (2011).

### Methods

### Computational Protein Design

### Design of New LOCKR Switches

As a starting point, the backbone of LOCKRa (SEQ ID NO:6) was used as input coordinates to Rosetta protein design software. Latch residues, residues on the Cage making contacts to the Latch (defined by the InterfaceByVector ResidueSelector in Rosetta™), and existing hydrogen bond networks were held fixed to coordinates of their input rotamers while the remaining residue positions were redesigned as follows: first, additional hydrogen bond networks were designed using HBNet™; second, RosettaDesign™ calculations were performed to optimize hydrophobic packing while the new hydrogen bond networks were maintained using AtomPair restraints on the heavy atoms of each sidechain hydrogen bond. This design procedure produced a new asymmetric Cage scaffold dubbed asymLOCKR. We then created a shorter version of this design by truncating the helical bundle by 12 residues based on visual inspection, reconnecting the helices with SGSGS linkers, and mutating several surface-exposed Arg and Lys residues to Glu to reduce the pI (FIG. 1b). Finally, we encoded the Bim sequence into the Latch to convert these scaffolds into LOCKRs. The shorter version (SEQ ID NO: 27359) was used as the parental Co-LOCKR. The RosettaScripts™ XML, file used to perform these design calculations is provided below.

### Design to Tune Relative Cage-Latch-Key Affinities to Achieve Colocalization Dependent Activation

We rationally mutated large, hydrophobic residues in the Latch of SEQ ID NO: 27359 (I287A, I287S, I269S) or Cage (L209A) to Alanine or Serine to weaken the Cage-Latch interface and increase Co-LOCKR sensitivity. We deleted several amino acids at the N- or C-terminus of the Key so as to weaken the Cage-Key interface and decrease Co-LOCKR sensitivity/leakiness.

### Design to Optimize Bcl2

Native Bcl2 was redesigned to improve its solution behavior and stability. As a starting point, the C-terminal 32 residues of the transmembrane domain were deleted, and the long loop between residues 35-91 of Bcl2 was replaced with residues 35-50 of the homolog Bcl-xL, as described previously (30). Additional mutations were made using Rosetta™ and PROSS™ (31) to improve hydrophobic packing and stability. Additional surface mutations were made rationally to improve solubility and remove glycosylation sites.

### Experimental Methods

### Cloning and Assembly of Synthetic Genes and a Bim-Specific Chimeric Antigen Receptor

Synthetic genes were purchased as gBlocks™ from Integrated DNA Technologies (IDT). All primers for mutagenesis were ordered from IDT. Synthetic genes were amplified using Kapa HiFi Polymerase according to the manufacturer's protocols with primers incorporating the desired mutations. The resulting amplicons were isothermally assembled (32) into a BamHI/XhoI digest of pET21b and transformed into chemically competent E. coli XL1-Blue cells (Agilent) or E. coli Lemo21™ (DE3) cells (New England Biolabs). Co-LOCKR Cage and Key components were targeted to cells using DARPins specific for Her2 (13), EGFR (14), and EpCAM (33).

The chimeric antigen receptor contained a murine IgK signal peptide, mini-FLAG and hemagglutinin (HA) tags, optimized Bcl2 binder, modified IgG4 long spacer with 4/2NQ mutations (17), CD28 transmembrane domain, and CD28/CD3ζ signaling domains (Fig S12a). The CAR transgene was codon-optimized and isothermally assembled into a HIV7 lentiviral vector upstream of a T2A sequence and truncated epidermal growth factor receptor (EGFRt) and transformed into chemically competent E. coli Turbo cells (New England Biolabs). Monoclonal colonies were verified by Sanger sequencing.

### Bacterial Protein Expression and Purification

E. coli Lemo21™ (DE3) cells harboring a pET21 plasmid encoding the gene of interest were grown overnight (10-16 hours) in 3 ml Luria-Bertani (LB) medium supplemented with 50 μg ml⁻¹ carbenicillin with shaking at 225 rpm at 37° C. Starter culture were added to 500 ml Studier TBM-5052 autoinduction media supplemented with carbenicillin, grown at 37° C. for 4-7 hours, and then grown at 18° C. for an additional 18-24 hours. Cells were harvested by centrifugation at 5000 g and 4° C. for 15 minutes and resuspended in 20 ml lysis buffer (25 mM Tris pH 8.0 at room temperature, 300 mM NaCl, 20 mM Imidazole, 1 mg ml⁻¹ lysozyme (Sigma L6876, from chicken egg), 0.1 mg ml⁻¹ DNase I (Sigma, DN25, from bovine pancreas). Cells were lysed by microfluidization in the presence of 1 mM phenylmethanesulfonyl fluoride (PMSF). Lysates were clarified by centrifugation at 24,000 g at 4° C. for 30 minutes and passed through 2 ml of nickel-nitrilotriacetic acid agarose (Ni-NTA, Qiagen, 30250) pre-equilibrated in lysis buffer. Immobilized protein was washed twice with 15 column volumes (CV) of wash buffer (25 mM Tris pH 8.0 at room temperature, 300 mM NaCl, 40 mM Imidazole), washed once with 5 CV of high-salt wash buffer (25 mM Tris pH 8.0 at room temperature, 1 M NaCl, 40 mM Imidazole), washed once more with 15 CV of wash buffer, and then eluted with 10 ml of elution buffer (25 mM Tris pH 8.0 at room temperature, 300 mM NaCl, 250 mM Imidazole). The eluted proteins were then concentrated (Amicon® Ultra-15 Centrifugal Filter Units, 10 kDa NMWL) and further purified by FPLC gel filtration using a Superdex™ 75 Increase 10/300 GL (GE) size exclusion column in Tris Buffered Saline (TBS; 25 mM Tris pH 8.0 at room temperature, 150 mM NaCl). Fractions containing non-aggregated protein were pooled, concentrated, and supplemented with glycerol to a final concentration of 10% v/v before being quantitated by absorbance at 280 nm (Nanodrop™), aliquoted, and snap frozen in liquid nitrogen. Protein aliquots were stable at −80° C.

### X-Ray Crystallography

For crystallography screening, the hexahistidine tag was removed via TEV cleavage followed by Ni-NTA affinity chromatography prior to SEC/FPLC. Purified protein samples were concentrated to approximately 12 mg ml⁻¹ and screened using JCSG+ and JCSG Core I-IV screens (Qiagen) on a 5-position deck Mosquito crystallization robot (ttplabtech) with an active humidity chamber. Crystals were obtained after 2 to 14 days by sitting drop vapor diffusion with drop ratios of 1:1, 2:1 and 1:2 protein solution to reservoir solution. The condition that resulted in the crystals that were used for structure determination was 0.2M di-Sodium tartrate, 20% (w/v) PEG 3350 and no cryoprotectant added.

### X-Ray Data Collection and Structure Determination

Protein crystals were looped and flash-frozen in liquid nitrogen. Datasets were collected at the Advanced Light Source at Lawrence Berkeley National Laboratory with beamlines 8.2.1 and 8.2.2. Data sets were indexed and scaled using XDS (34) and phase information was obtained by molecular replacement (MR) using PHASER™ (35) from the Phenix™ software package (36); design models were used for the initial MR searches. Following MR, models were improved using Phenix.autobuild (37); efforts were made to reduce model bias by setting rebuild-in-place to false, and using simulated annealing and prime-and-switch phasing. Iterative rounds of manual building in COOT™ (38) and refinement in Phenix™ were used to produce the final models. Translational non-crystallographic symmetry was present in the data as report by Phenix.Xtriage, which complicated structure refinement and may explain the higher than expected R-values reported. RMSDs of bond lengths, angles and dihedrals from ideal geometries were calculated using Phenix™ (36). The overall quality of the final models was assessed using MOLPROBITY™ (39). Table 16 summarizes diffraction data and refinement statistics.

### Bcl2 Labeling

For BLI experiments, wild-type non-optimized Bcl2 with C-terminal Avi and 6×His-tags was enzymatically biotinylated using BirA according to manufacturer protocols (Avidity), purified by Ni-NTA, eluted into TBS, concentrated, snap frozen in liquid nitrogen, and stored at −80° C. For flow cytometry experiments, Bcl2 with a C-terminal cysteine was purified by Ni-NTA and gel filtration as described above with the addition of 0.5 mM TCEP to the buffers. All fractions containing monomeric Bcl2 were combined, concentrated to 100 in TBS supplemented with 2% glycerol and 1 mM TCEP, and labeled overnight at 4° C. with a 5-fold molar excess of Alexa Fluor™ 594 C₅ Maleimide (Invitrogen A10256) or Alexa Fluor™ 680 C₂ Maleimide (Invitrogen A20344). The labeling reaction was then dialyzed overnight into TBS supplemented with 10% glycerol and purified by gel filtration as described above. Fractions containing monomeric protein were pooled, concentrated, and supplemented with glycerol to a final concentration of 10% v/v before being quantitated by absorbance at 280 nm, aliquoted, and snap frozen in liquid nitrogen. Protein aliquots were stable at −80° C. After thawing, protein aliquots were stored at 4° C. for up to one week.

### Bio-Layer Interferometry (BLI)

BLI measurements were made on an Octet® RED96 System (ForteBio) with streptavidin (SA) coated biosensors and analyzed using ForteBio Data Analysis Software version 9.0.0.10. Assay buffer was HBS-EP+ Buffer (10 mM HEPES, 150 mM NaCl, 3 mM EDTA, 0.05% v/v Surfactant P20, 0.5% non-fat dry milk, pH 7.4 at room temperature). Biotinylated Bcl2 protein was loaded onto the SA tips using a programmed threshold of 0.5 nm. Baseline was obtained by dipping the loaded biosensors into HBS-EP+ buffer; association kinetics were observed by dipping loaded biosensors into wells containing a range of LOCKR Cage and Key concentrations. Dissociation kinetics were observed by dipping tips into the HBS-EP+ Buffer wells that were used to obtain baseline. For FIG. 2 and FIG. 9b-e, Cage and Key were diluted simultaneously to maintain a 1:1 stoichiometric ratio.

### Mammalian Protein Expression and Purification

The scFv-targeted Co-LOCKR proteins (anti-Her2_Cage_I269S and Key_cetuximab) were produced using the Daedalus system as previously described (40). Proteins were purified on a HisTrap™ FF Crude protein purification column (GE cat #17528601) followed by size exclusion chromatography (GE Superdex 200 10/300 GL) and eluted in Dulbecco's phosphate-buffered saline supplemented with 5% glycerol.

Acquisition of T Cells from Healthy Donors

Healthy individuals >18 years-old were enrolled in Institutional Review Board-approved studies for peripheral blood collection. Informed consent was obtained from all enrollees. Researchers were provided donor age, nondescript donor ID number, and were blinded to all other personally-identifiable information about study participants. Peripheral blood mononuclear cells (PBMC) were isolated by density gradient using Lymphocyte Separation Media (Corning). CD8⁺ T cells were isolated using the EasySep™ Human CD8⁺ T Cell Isolation Kit (StemCell Technologies) in accordance with manufacturer's instructions.

### Cell Culture

K562 (CCL-243), Raji (CCL-86), A431 (CRL-1555), and HEK293T (CRL-3216) cells were obtained from American Type Culture Collection (ATCC). 293T LentiX cells were purchased from Clontech. SKBR3 cells were a gift from David Hockenbery (Fred Hutchinson Cancer Research Center). K562 and Raji cells were cultured in RPMI-1640 (Gibco) supplemented with 5% fetal bovine serum (FBS), 1 mM L-glutamine, 25 mM HEPES, and 100 U ml⁻¹ penicillin/streptomycin. A431, SKBR3, HEK293T, and LentiX cells were cultured in DMEM high glucose (Gibco) supplemented with 10% FBS, 1 mM L-glutamine, 25 mM HEPES, 100 U ml⁻¹ penicillin/streptomycin, and 1 mM pyruvate. Primary human T cells were cultured in CTL medium consisting of RPMI-1640 supplemented with 10% human serum, 2 mM L-glutamine, 25 mM HEPES, 100 U ml⁻¹ penicillin/streptomycin and 50 μM β-mercaptoethanol. All cells were cultured at 37° C. and 5% CO₂, and tested bi-monthly for the absence of mycoplasma using MycoAlert™ Mycoplasma Detection Kit (Lonza).

### Generation of K562 and HEK293T Cell Lines

HEK293T or LentiX cells were transiently transfected with psPAX2 (Addgene Plasmid #12260), pMD2.G (Addgene Plasmid #12259) packaging plasmids as well as a lentiviral vector encoding either Her2-eGFP, EGFR-iRFP (for K562 cells), or EGFR-mCherry™™ (for HEK293T cells) using linear 25-kDa polyethyleneimine (PEI; Polysciences). Two days later, viral supernatant was concentrated by centrifugation at 8000 g for 18 hours and added to K562 cells or HEK293T with 4 μg ml⁻¹ Polybrene (Sigma). Flow cytometry indicated that the Her2-eGFP and EGFR-iRFP cell lines were transduced to 98%, and the Her2-eGFP/EGFR-iRFP cell line was transduced to 88%.

Because K562 cells endogenously expressed low levels of EpCAM, EpCAM knockout (KO) cell lines were generated by nucleofection with the Alt-R® CRISPR-Cas9 system (IDT). Pre-designed crRNAs specific for the human EpCAM gene (Hs.Cas9.EPCAM.1.AA and Hs.Cas9.EPCAM.1.AB, IDT) were reconstituted in Nuclease-Free Duplex Buffer, mixed with tracrRNA at equimolar concentrations, annealed by heating to 95° C. for 5 minutes, followed by slow cooling to room temperature. crRNA-tracrRNA duplexes were combined and complexed with S.p. Cas9 Nuclease V3 and Cas9 Electroporation Enhancer for 15 minutes at room temperature. RNP complexes were added to K562 cell lines and nucleofection was performed using a 4D Nucleofector mCherry™ (Lonza) using SF Cell Line Buffer and FF-120 program according to manufacturer's instructions. Four days later, cells that stained negative for EpCAM were FACS-sorted to greater than 99% purity.

EpCAM high K562 cell lines were generated by transducing Her2-eGFP, Her2-eGFP/EGFR-iRFP, and parental K562 cells with an EpCAM-expressing lentivirus that had been prepared by transiently transfecting LentiX cells with psPAX2, pMD2.G and a lentiviral vector encoding human EpCAM (UniProt: P16422, aa1-314) using CalPhos™ Mammalian Transfection Kit (Clontech). Two days after transfection, viral supernatant was filtered using a 0.45 μm PES syringe filter (Millipore) and added to the cell lines with 4 μg ml⁻¹ Polybrene. Five days later, transduced cells that stained high for EpCAM, EGFR, or Her2 were FACS-sorted to greater than 95% purity. Bim-eGFP-expressing K562 cells were generated in an identical manner using a lentivirus encoding a membrane-tethered Bim-eGFP fusion protein (mIgK signal peptide, GS linker, Bim peptide, SGSG linker, eGFP, PDGFR transmembrane domain), and FACS-sorted for eGFP expression five days after transduction.

### Generation of Raji Cell Lines

LentiX™ cells were transiently transfected with psPAX2, pMD2.G as well as a lentiviral vector encoding either human EGFR (UniProt: P00533, aa1-1210), EpCAM (UniProt: P16422, aa1-314), or Her2 (UniProt: P04626, aa1-1255) using CalPhos™ Mammalian Transfection Kit. Two days later, viral supernatant was filtered using a 0.45 μm PES syringe filter and added to Raji cells. Five days later, transduced cells that stained positive for EGFR, EpCAM, or Her2 were FACS-sorted to greater than 95% purity.

### Flow Cytometry and Cell Phenotyping

Cells were stained with a 1:100 dilution of fluorophore-conjugated monoclonal antibodies specific for human CD5 (UCHT2), CD8 (SK1), EGFR (AY13), EpCAM (9C4), HA1.1 (16B12), or Her2 (24D2) purchased from ThermoFisher or Biolegend. Cells were also stained with isotype control fluorophore-conjugated antibodies when appropriate. For sorting EGFRt⁺ CAR T cells, Cetuximab (anti-EGFR, Bristol-Myers Squibb) was biotinylated using the EZ-Link™ Sulfo-NHS-Biotin Kit (ThermoFisher) followed by cleanup with the Zeba™ Spin Desalting Column (ThermoFisher) and used to stain T cells in conjunction with streptavidin-allophycocyanin (ThermoFisher). For Bcl2-AF594 binding measurements, K562 cell lines were combined into mixed populations with equal numbers of each cell type. Because EpCAM was not tagged with a fluorescent protein, two distinct populations were evaluated for each logic operation in FIG. 3: a “Low EpCAM” population contained K562/EpCAM^{lo}, K562/Her2-eGFP/EpCAM^{lo}, K562/EGFR-iRFP/EpCAM^{lo}, and K562/EGFR-iRFP/EpCAM^{lo} and the “High EpCAM” population contained K562/EpCAM^{lo}, K562/Her2-eGFP/EpCAM^{hi}, K562/EGFR-iRFP/EpCAM^{lo}, and K562/EGFR-iRFP/EpCAM^{hi}. The cell mixtures were washed with flow buffer (20 mM Tris pH 8.0, 150 mM NaCl, 1 mM MgCl₂, 1 mM CaCl₂ and 1% BSA) and aliquoted into V-bottom plates with 200,000 cells/well. Samples were incubated for one hour at room temperature with Bcl2-AF594 at 50 nM and Cage, Key and/or Decoy at a final concentration of 20 nM unless stated otherwise. Samples were washed once in 150 μl flow buffer, and then resuspended in 150 μl flow buffer 15-30 minutes before analysis.

Data were acquired on a LSRII or FACSCelesta™ (BD Biosciences). K562, Raji, and human T cells were FACS-purified using a FACSAria II™ (BD Biosciences). The absolute number of EGFR, EpCAM, and Her2 molecules on the surface of K562 and Raji cells was determined using Quantibrite™ beads (BD Biosciences) according to manufacturer's protocols. All flow cytometry data were analyzed using FlowJo™ (Treestar).

### Confocal Microscopy

HEK293T cells were grown in ibidi μ-slide 8 well coverslips for 1 day at 37° C. and 5% CO2 (ibidi 80826). Cell staining and incubation were performed in DMEM, high glucose, HEPES, no phenol red (Gibco 21063029). Cell nuclei were stained with Invitrogen Molecular Probes NucBlue™ Live ReadyProbes™ Reagent according to manufacturer's instructions (Invitrogen R37605). Cells were incubated in culture medium containing 1% BSA, 20 nM Her2_Cage-I269S, 20 nM Key_EGFR, and 50 nM Bcl2-AF680 for 1-2 hours at 37° C. and 5% CO₂. Images were acquired on a Leica SP8X confocal microscope and analyzed in Fiji.

### Confocal Microscopy Heat Map Analysis

Red, green, and blue (RGB) pseudocolors were assigned to the mCherry™, eGFP, and AF680 channels, respectively, in Fiji. Using a custom python script (see supplement), the ImageIO Python library was used to read the RGB PNG files, the SciPy Python library was used to generate a bidimensional binned statistic from the pseudocolored pixel intensities, and the Matplotlib™ library was used to visualize the results as a heat map.

### Human T Cell Culture and Transduction

To prepare CAR T cells, LentiX™ cells were transiently transfected with the CAR vector, psPAX2 and pMD2.G. One day later (day 1), primary T cells were activated using Dynabeads™ Human T-Activator CD3/CD28 (ThermoFisher) at a 3:1 bead to T cell ratio and cultured in CTL supplemented with 50 U ml⁻¹ IL-2 (Prometheus). The next day (day 2), lentiviral supernatant was harvested from LentiX™ cells, filtered using a 0.45 μm PES filter, and added to activated T cells. Polybrene was added to reach a final concentration of 4.4 mg ml⁻¹, and cells were spinoculated at 800 g for 90 minutes at 32° C. Viral supernatant was replaced 8 hours later with fresh CTL medium supplemented with 50 IU ml⁻¹ IL-2. Half-media changes were then performed every 48 hours using CTL supplemented with 50 IU ml⁻¹ IL-2. Dynabeads were removed on day 6, CD8⁺EGFRt⁺ transduced T cells were FACS-sorted on day 9, and purified CD8⁺EGFRt⁺ cells were rapidly expanded using 30 ng ml⁻¹ purified OKT3, γ-irradiated LCL, and γ-irradiated allogeneic PBMC at a LCL to T cell ratio of 100:1 and a PBMC to T cell ratio of 600:1. 50 IU ml⁻¹ IL-2 was added on day 1, OKT3 was washed out on day 4, cultures were fed with fresh CTL medium supplemented with 50 IU ml⁻¹ IL-2 every 2-3 days and resting T cells were assayed 11-12 days after stimulation. Non-transduced T cells (CD8⁺EGFRt⁻ T cells that were not transduced with lentivirus) were cultured identically and used as negative controls for CAR T assays.

### T Cell Functional Assays

T cell cytokine secretion was measured by co-culturing 50,000 non-transduced or CAR T cells with 25,000 γ-irradiated (10,000 rad) K562 or Raji cell lines to reach a T cell to tumor cell ratio of 2:1. Cytokine concentrations in cellular supernatant after 24 hours were quantified by human IFN-γ ELISA (ThermoFisher). T cell proliferation was assessed by staining CAR T cells with a 0.2 μM solution of carboxyfluorescein succinimidyl ester (CFSE) (ThermoFisher) or 1 μM solution of Cell Trace Violet (ThermoFisher) prior to co-culture with K562 or Raji cell lines at a 2:1 T cell to tumor cell ratio. After 72 hours, cells were harvested, stained with fluorescently labeled anti-human CD8 antibody, washed once, and analyzed by flow cytometry. The frequency of divided cells was calculated by drawing a “% Undivided” gate on the undivided peak in negative control samples and then setting a “% Divided” that bordered the first “% Undivided” gate. Mixed population tumor cell killing was assessed by labeling various Raji cell lines with either 1 μM or 2 nM solutions of Cell Trace Far Red (ThermoFisher) or 1 μM or 5 nM solutions of Cell Trace Violet for 10 minutes at 37° C. Labeling was quenched using FBS and equal numbers of cells were combined to form mixed populations. 150,000 T cells and 150,000 Raji cells were distributed into a 48-well plate to reach a T cell to total tumor cell ratio of 1:1. Killing was quantified after 48 hours by harvesting all cells and performing surface staining with PE-Cy7 anti-human CD5 (to identify T cells) and Live/Dead Fixable Green Stain (ThermoFisher). For Chromium release assays, tumor cells were labeled with ⁵¹Cr (PerkinElmer) for 16 hours at 37° C., washed with RPMI, and plated with 1,000 ⁵¹Cr-labeled target cells per well. T cells were added in triplicate at various effector to target (E:T) ratios and incubated at 37° C., 5% CO₂ for four hours. Supernatants were then harvested for γ-counting, and specific lysis was calculated by comparing counts to standardized wells in which target cells were lysed with a NP40-based soap solution. Each Cage, Key, and/or Decoy protein was used at 20 nM unless otherwise specified.

### Statistical Analysis

Statistical analyses were performed using Prism™ (GraphPad). An ordinary one-way ANOVA test followed by Dunnett's post-hoc test was used to compare Co-LOCKR-induced targeting (FIG. 3a, c, e) and CAR T cell cytokine production (FIG. 4). For ‘AND’ targeting, the control group was set as the double-negative cell line; for ‘OR’ and ‘NOT’ targeting the control group was set as the triple-negative cell line. Only p-values meeting a statistically significant cutoff of alpha=0.05 are indicated on graphs. * denotes p<0.05, ** denotes p<0.01, *** denotes p<0.001, **** denotes p<0.0001.

TABLE 16
X-ray crystallography data collection and refinement statistics.

| Wavelength | 0.9999 | |
|---|---|---|
| Resolution range | 42.38-2.10 (2.18-2.10) | |
| Space group | P 1 21 1 | |
| Unit cell | 61.841 52.914 75.591 | |
| | 90 102.638 90 | |
| Total reflections | 123167 | (10501) |
| Unique reflections | 27650 | (2448) |
| Multiplicity | 4.5 | (4.3) |
| Completeness (%) | 85.21 | (69.05 |
| Mean I/sigma(I) | 4.93 | (0.92) |
| Wilson B-factor | 19.07 | |
| R-merge | 0.1749 | (1.117) |
| R-meas | 0.1993 | (1.276) |
| R-pim | 0.09414 | (0.6093) |
| CC1/2 | 0.996 | (0.671) |
| CC* | 0.999 | (0.896) |
| Reflections used in refinement | 23829 | (1892) |
| Reflections used for R-free | 1736 | (137) |
| R-work | 0.2574 | (0.3125) |
| R-free | 0.2881 | (0.2983) |
| CC (work) | 0.938 | (0.792) |
| CC (free) | 0.922 | (0.842) |

| | |
|---|---|
| Number of non-hydrogen atoms | 4467 |
| macromolecules | 4277 |
| solvent | 190 |
| Protein residues | 564 |
| RMS (bonds) | 0.005 |
| RMS (angles) | 0.89 |
| Ramachandran favored (%) | 99.10 |
| Ramachandran allowed (%) | 0.90 |
| Ramachandran outliers (%) | 0.0 |
| Rotamer outliers (%) | 1.29 |
| Clashscore | 10.5 |
| Average B-factor | 32.95 |
| macromolecules | 32.96 |
| solvent | 32.58 |

Statistics for the highest-resolution shell are shown in parentheses.

TABLE 17
Co-LOCKR logic.

| Co-LOCKR | Logic operation |
|---|---|
| CL_C_{H}K_{H} | H |
| CL_C_{E}K_{E} | E |
| CL_C_{Ep}K_{Ep} | Ep |
| CL_C_{H}K_{E} | H AND E |
| CL_C_{E}K_{H} | H AND E |
| CL_C_{H}K_{Ep} | H AND Ep |
| CL_C_{Ep}K_{H} | H AND Ep |
| CL_C_{E}K_{Ep} | E AND Ep |
| CL_C_{Ep}K_{E} | E AND Ep |
| CL_C_{H}K_{E}K_{Ep} | H AND either E OR Ep |
| CL_C_{E}K_{H}K_{Ep} | E AND either H OR Ep |
| CL_C_{Ep}K_{H}K_{E} | Ep AND either H OR E |
| CL_C_{H}K_{Ep}D_{E} | H AND Ep NOT E |
| CL_C_{Ep}K_{H}D_{E} | H AND Ep NOT E |

### #Custom Python Script for Confocal Microscopy Heat Map Analysis:

```
#!/usr/bin/env python3
# -*- coding: utf-8 -*-
″″″
Created on Thu Jun 6 13:47:38 2019
@author: audreyolshefsky
Heat map for co-LOCKR confocal RGB image pixel intensities.
Red is on the x-axis, green is on the y-axis, and blue is the heat.
″″″
import imageio
from scipy import stats
import matplotlib.pyplot as plt
def heatmap(image):
  im = imageio.imread(image)
  # copy of image data is quicker to access
  imcopy = im
  #print(imcopy.shape) #RGB shape is (1024, 1024, 3)
  pixel_list =[ ]
  counter = 0
  for y in range(imcopy.shape[0]):
    for x in range(imcopy.shape[1]):
      counter += 1
      #print(counter)
      color =tuple(imcopy[y][x])
      #r, g, b =color
      pixel_list.append(color)
r =[x[0]for x in pixel_list]
g =[x[1]for x in pixel_list]
b =[x[2]for x in pixel_list]
binned_data =stats.binned_statistic_2d(r, g, b)
im =plt.imshow(binned_data[0].T, cmap=′plasma′, origin=′lower′,
extent=[0, 255, 255])
cb = plt.colorbar( )
cb.set_label(′AF680 mean pixel intensity (Bcl2)′)
plt.xlabel(′mCherryTM pixel intensity (EGFR)′)
plt.ylabel(′eGFP pixel intensity (HER2)′)
plt.savefig(image [:-4]+′_heatmap.png′)
```

### #RosettaScripts XML Used to Design Co-LOCKR

```
# Rosetta XML script to redesign LOCKR to be more asymmetric
# Original scaffold was built from symmetric homotrimer (Boyken 2016)
# Scott Boyken and Marc Lajoie
<ROSETTASCRIPTS>
  <SCOREFXNS>
    <ScoreFunction name=″hard″ weights=″beta/>
  </SCOREFXNS>
  <RESIDUE_SELECTORS>
    <Index name=″latch″ resnums=″302-350″/>
    <Index name=″cage″ resnums=″1-301″/>
      #preserve original hydrogen bond networks
    <Index name=″HBNet″
resnums=″9,12,24,27,30,45,48,75,93,111,130,133,148,151,166,169,196,214,232,251,254,269,272,287,290,335,
339″/>
    <InterfaceByVector name=″switch_interface″grp1_selector=″latch″grp2_selector=″cage″/>
    <And name=″main_scaffold″>
      <Not selector=″switch_interface″/>
      <Not selector=″HBNe″t/>
      <Secondary Structure ss=″H″/>
    </And>
    <Not name=″no_design″selector=″main_scaffold/>
  </RESIDUE_SELECTORS>
  <TASKOPERATIONS>
      <OperateOnResidueSubset name=″repack_new″selector=″no_design″>
        <PreventRepackingRLT/>
      </OperateOnResidueSubset>
  </TASKOPERATIONS>
  <MOVERS>
    <SwitchChainOrder name=″rechain″chain_order=″12″/>
      #search for new hydrogen bond networks while preserving the original networks
    <HBNetStapleInterface scorefxn=″hard″ name=″hbnet″ design_residues=″HYNQST″
task_operations=″repack_new″ hb_threshold=″-0.5″ minimize=″true″ show_task=″true″ verbose=″true″
all_helical_interfaces=″true″ min_connectivity=″0.6″ min_helices_contacted_by_network=″2″
min_networks_per_pose=″1″ max_networks_per_pose=″3″ min_network_size=″3″ min_core_res=″2″
max_unsat=″3″ max_replicates_before_branch=″3″ use_aa_dependent_weights=″true″
write_network_pdbs=″true″write_cst_files=″false/>
    <MultiplePoseMover name=″switch_peptide_design_MPM″ max_input_poses=″100″>
      <ROSETTASCRIPTS>
        <SCOREFXNS>
        <ScoreFunction name=″hard″ weights=″beta″/>
        <ScoreFunction name=″hard_cart″ weights=″beta_cart″/>
        <ScoreFunction name=″soft_cst″ weights=″/home/sboyken/weights/beta_soft_rep_cst.wts″/>
        <ScoreFunction name=″hard_cst″weights=″beta_cst″/>
        <ScoreFunction name=″up_ele″weights=″beta″>
          <Reweight scoretype=″fa_elec″weight=″1.4″/>
          <Reweight scoretype=″hbond_sc″weight=″2.0″/>
        </ScoreFunction>
      </SCOREFXNS>
      <RESIDUE_SELECTORS>
        <Layer name=″hbnet_core″ select_core=″true″ core_cutoff=″3.6″/>
        <And name=″terminal_loop″>
          <Secondary Structure ss=″L″ include_terminal_loops=″true″ use_dssp=″true″/>
          <Index resnums=″1-15″/>
          <Chain chains=″A″/>
        </And>
        <Secondary Structure name=″loops″ use_dssp=″true″ ss=″L″/>
        <Not name=″not_redesign″ selector=″loops″/>
        <Layer name=″pick_core_and_boundary″ select_core=″true″ select_boundary=″true″
core_cutoff=″5.2″/>
        <Layer name=″pick_core_and_surface″ select_core=″true″ select_surface=″true″
core_cutoff=″5.2″/>
        <Layer name=″pick_surface_and_boundary″ select_surface=″true″ select_boundary=″true″
core_cutoff=″5.2″/>
        <Chain name=″chain_a″ chains=″A″/>
        <Layer name=″core″ select_core=″true″ core_cutoff=″5.2″/>
        <ResidueName name=″ala_and_mer residue_name3=″ALA,MET″I>
        <Not name=″not_ala_or_met″ selector=″ala_and_met″/>
        <!-- <ResiduePDBInfoHasLabel name=″hbnet_residues″ property=″HBNet″ /> -->
        <Index name=″latch″ resnums=″302-350″/>
        <Index name=″cage″ resnums=″1-301″/>
        <Index name=″HBNet″
resnums=″9,12,24,27,30,45,48,75,93,111,130,133,148,151,166,169,196,214,232,251,254,269,272,287,290,335,
339″/>
        <InterfaceByVector name=″switch_interface″ grp1_selector=″latch″ grp2_selector=″cage″/>
        # select all residues to not touch during design
        <And name=″main_scaffold″>
          <Not selector=″switch_interface″>
            # to repack HBNet res with AtomPair csts during design, comment this line out; use
″hbnet_task″
            # leave it in to make HBNet rotamers fixed
          <Not selector=″HBNet″/>
          <Secondary Structure ss=″H″/>
        </And>
        <Not name=″no_design″ selector=″main_scaffold″/>
      </RESIDUE_SELECTORS>
      <TASKOPERATIONS>
        <ConsensusLoopDesign name=″disallow_non_abego_aas″/>
        <LayerDesign name=″layer_all″ layer=″core_boundary_surface_Nterm_Cterm″
make_pymol_script=″0″ use_sidechain_neighbors=″True″ core=″4.2″>
          <core>
            Helix append=″M″ />
            <Helix exclude=″WY″ />
          </core>
          <boundary>
            <Helix exclude=″WMY″ />
          </boundary>
          <surface>
            <Helix append=″A″/>
          </surface>
        </LayerDesign>
        <OperateOnResidueSubset name=″loop_design″ selector=″not_redesign″>
          <PreventRepackingRLT/>
        </OperateOnResidueSubset>
        <OperateOnResidueSubset name=″repack_new″s elector=″no_design″>
          <PreventRepackingRLT/>
        </OperateOnResidueSubset>
        <OperateOnResidueSubset name=″design_core″ selector=″pick_surface_and_boundary″>
          <PreventRepackingRLT/>
        </OperateOnResidueSubset>
        <OperateOnResidueSubset name=″design_boundary″ selector=″pick_core_and_surface″>
          <PreventRepackingRLT/>
        </OperateOnResidueSubset>
        <OperateOnResidueSubset name=″design_surface″ selector=″pick_core_and_boundary″>
          <PreventRepackingRLT/>
        </OperateOnResidueSubset>
        <OperateOnResidueSubset name=″repack_not_ala_or_met″ selector=″not_ala_or_met″>
          <RestrictToRepackingRLT/>
        </OperateOnResidueSubset>
        <OperateOnResidueSubset name=″redesign_ala_met″ selector=″ala_and_met″>
          <RestrictAbsentCanonicalAASRLT aas=″AMIL VF″ />
        </OperateOnResidueSubset>
        <InitializeFromCommandline name=″init″/>
        <ConstrainHBondNetwork name=″hbnet_task″ />
        <IncludeCurrent name=″current″/>
        <LimitAromaChi2 name=″arochi″/>
        <ExtraRotamersGeneric name=″ex1_ex2″ ex1=″1″ ex2=″1″/>
        <ExtraRotamersGeneric name=″ex1″ ex1=″1″/>
        <RestrictAbsentCanonicalAAS name=″ala_only″ resnum=″0″ keep_aas=″A″/>
        <RestrictToRepacking name=″repack_only″/>
        BundleReporter name=bundle_filter scorefxn=hard/>
      </TASKOPERATIONS>
      <MOVERS>
        <PackRotamersMover name=″softpack_core″ scorefxn=″soft_cst″
task_operations=″layer_all,design_core,current,arochi,disallow_non_abego_aas,repack_new,hbnet_task″/>
        <PackRotamersMover name=″softpack_boundary″ scorefxn=″soft_cst″
task_operations=″layer_all,design_boundary,currentarochi,disallow_non_abego_aas,repack_new,hbnet_task″/>
        <PackRotamersMover name=″softpack_surface″ scorefxn=″soft_cst″
task_operations=″layer_all,design_surface,currentarochi,disallow_non_abego_aas,repack_new,hbnet_task″/>
        <PackRotamersMover name=″hardpack_core″ scorefxn=″hard_cst″
task_operations=″layer_all,design_core,current,arochi,exl_ex2,disallow_non_abego_aas,repack_new,hbnet_
task″/>
        <PackRotamersMover name=″hardpack_boundary″ scorefxn=″hard_cst″
task_operations=″layer_all,design_boundary,currentarochi,exl_ex2,disallow_non_abego_aas,repack_new,
hbnet_task″/>
        <PackRotamersMover name=″hardpack_surface″ scorefxn=″up_ele″
task_operations=″layer_all,design_surface,current,arochi,exl,disallow_non_abego_aas,repack_new,hbnet_task″
/>
        <PackRotamersMover name=″design_loops″ scorefxn=″hard_cst″
task_operations=″currentarochi,ex1_ex2,loop_design,layer_all,disallow_non_abego_aas,hbnet_task″/>
        <DumpPdb name=″dump1″ fname=″dump1.pdb″ scorefxn=″hard″/>
        ConnectChainsMover name=closer chain_connections=″[A+B],[B+A]″/>
        InterfaceAnalyzerMover name=interface_analyzer scorefxn=hard packstat=1 pack_separated=0 />
        <MinMover name=″hardmin_sconly″ scorefxn=″hard_cst″ chi=1″bb=″0″ bondangle=″0″
bondlength=″0″/>
      </MOVERS>
      <PROTOCOLS>
        <Add mover=″softpack_core″/>
        <Add mover=″softpack_boundary″/>
        <Add mover=″softpack_surface″/>
        <Add mover=″hardmin_sconly″/>
        <Add mover=″hardpack_core″/>
        <Add mover=″hardpack_boundary″/>
        <Add mover=″hardpack_surface″/>
      </PROTOCOLS>
    </ROSETTASCRIPTS>
  </MultiplePoseMover>
  <MultiplePoseMover name=″MPM_filters″>
    <ROSETTASCRIPTS>
      <SCOREFXNS>
        <ScoreFunction name=″hard_cst″weights=″beta_cst″/>
      </SCOREFXNS>
      <RESIDUE_SELECTORS>
        <Chain name=″peptide″chains=″B″/>
        <Chain name=″SB76_trunc″chains=″A″/>
        <InterfaceByVector name=″scaffold_interface″>
          <Chain chains=″A″/>
          <Not selector=″SB76_trunc″/>
        </InterfaceByVector>
      </RESIDUE_SELECTORS>
      <TASKOPERATIONS>
        <LayerDesign name=″layer_all″ layer=″core_boundary_surface_Nterm_Cterm″
make_pymol_script=″0″ use_sidechain_neighbors=″True″ core=″5.2″>
          <core>
            Helix append=″M″ />
              <Helix exclude=″WY″ />
          </core>
          <boundary>
            <Helix exclude=″DWMY″ />
          </boundary>
          <surface>
            <Helix append=″A″/>
          </surface>
        </LayerDesign>
      </TASKOPERATIONS>
      <FIL lERS>
        <PreProline name=″prepro″ use_statistical_potential=″0″ />
        <ScoreType name=″scorefilter″ scorefxn=″hard_cst″ score_type=″total_score″ threshold=″0.0″
confidence=″0″ />
        <EnzScore name=″cst_score″ score_type=″cstE″ scorefxn=″hard_cst″ whole_pose=″1″
energy_cutoff=″5″ confidence=″0″!>
        <BuriedUnsatHbonds name=″buns3″ scorefxn=″beta″ cutoff=″10″ print_out_info_to_pdb=″true″
use_hbnet_behavior=″true″ confidence=″0″/>
        <ResidueCount name=″ala_count″ max_residue_count=″15″ residue_types=″ALA″
residue_selector=″scaffold_interface″ confidence=″0″/>
      </FILTERS>
      <PROTOCOLS>
        <Add filter=″scorefilter″/>
        <Add filter=″cst_score″/>
        <Add filter=″ala_coune/>
        <Add filter=″buns3″/>
      </PROTOCOLS>
    </ROSETTASCRIPTS>
  </MultiplePoseMover>
</MOVERS>
<PROTOCOLS>
    <Add mover=″hbnet″/>
    <Add mover=″switch_peptide_design_MPM″/>
    <Add mover=″MPM_filters″/>
  </PROTOCOLS>
</ROSETTASCRIPTS>
```

### All Amino Acid Sequences

### Modular Sequences:

1. Co-LOCKR is comprised of one or more Cage polypeptides, one or more Key polypeptides, and optionally one or more Decoy polypeptides, wherein
   a. The Cage polypeptide is comprised of one or more modular targeting moieties, one or more modular Co-LOCKR Cage domains, and optionally one or more modular Co-LOCKR linkers
   b. The Key polypeptide is comprised of one or more modular targeting moieties, one or more modular Co-LOCKR Key domains, and optionally one or more modular Co-LOCKR linkers
   c. The Decoy polypeptide is comprised of one or more modular targeting moieties, one or more modular Co-LOCKR Decoy domains, and optionally one or more modular Co-LOCKR linkers
      Modular targeting moieties: See Table 10

TABLE 14
Modular Co-LOCKR Linkers

>Linker1 YKDEHHHHHHGGSENLYFQGSG (SEQ ID NO: 27479)
>Linker2 GGGSGSGSGSGKPGQASGS(SEQ ID NO: 27480)
>Linker3 GSGSGKPGQASG (SEQ ID NO: 27481)
>Linker4 GGS (SEQ ID NO: 27482)
>Linker5 SGSGSGKPGQASGS (SEQ ID NO: 27483)
>Linker6 YPYDVPDYA (SEQ ID NO: 27484)
>Linker7 SGSG (SEQ ID NO: 27485)
>Linker8 NWSHPQFEK (SEQ ID NO: 27486)
>Linker9
SGSGSGSGSGSGSGSGSGSGSGSGSGSGSGSGSGSGSGSGSGSGSGSGSG
SGSGSGSGKPGQASGS (SEQ ID NO: 27487)
>Linker10 GGGGS (SEQ ID NO: 27488)

Modular Co-LOCKR Cage domains: See Table 1.
Co-LOCKR Cage and Decoy proteins: See Table 11.
Modular Co-LOCKR Key domains: See Table 4.
Co-LOCKR Key proteins: See Table 12.
Effector proteins: See Table 13.

TABLE 15
DARPin positive controls with N-terminal fusions
to Bim
Residues in parentheses are optional

>Bim_Her2 Her2 DARPin tagged with N-terminal Bim
peptide (SEQ ID NO: 27476)
(MGSHHHHHHGSGSENLYFQGS)TDEIWIAQELRRIGDEFNAYYASGSGD
LGKKLLEAARAGQDDEVRILMANGADVNAKDEYGLTPLYLATAHGHLEIV
EVLLKNGADVNAVDAIGFTPLHLAAFIGHLEIAEVLLKHGADVNAQDKFG
KTAFDISIGNGNEDLAEILQKLN
>Bim_EGFR EGFR DARPin tagged with N-terminal Bim
peptide (SEQ ID NO: 27477)
(MGSHHHHHHGSGSENLYFQGS)TDEIWIAQELRRIGDEFNAYYASGSGD
LGKKLLEAARAGQDDEVRILMANGADVNADDTWGWTPLHLAAYQGHLEIV
EVLLKNGADVNAYDYIGWTPLHLAADGHLEIVEVLLKNGADVNASDYIGD
TPLHLAAHNGHLEIVEVLLKHGADVNAQDKFGKTAFDISIDNGNEDLAEI
LQKLN
>Bim_EpCAM EpCAM DARPin tagged with N-terminal
Bim peptide (SEQ ID NO: 27478)
(MGSHHHHHHGSGSENLYFQGS)TDEIWIAQELRRIGDEFNAYYASGSGD
LGKKLLEAARAGQDDEVRILVANGADVNAYFGTTPLHLAAAHGRLEIVEV
LLKNGADVNAQDVWGITPLHLAAYNGHLEIVEVLLKYGADVNAHDTRGWT
PLHLAAINGHLEIVEVLLKNVADVNAQDRSGKTPFDLAIDNGNEDIAEVL
QKAAKLN

TABLE 16
Target proteins

>Her2-eGFP; hsHER2_ecd_tm-eGFP; Lentiviral transduction for making Her2+ K562 cells
(enhancedGFP+) (SEQ ID NO: 27470)
MELAALCRWGLLLALLPPGAASTQVCTGTDMKLRLPASPETHLDMLRHLYQGCQVVQGNLELTYLPTNASLSFLQDIQEVQGY
VLIAHNQVRQVPLQRLRIVRGTQLFEDNYALAVLDNGDPLNNTTPVTGASPGGLRELQLRSLTEILKGGVLIQRNPQLCYQDT
ILWKDIFHKNNQLALTLIDTNRSRACHPCSPMCKGSRCWGESSEDCQSLTRTVCAGGCARCKGPLPTDCCHEQCAAGCTGPKH
SDCLACLHFNHSGICELHCPALVTYNTDTFESMPNPEGRYTFGASCVTACPYNYLSTDVGSCTLVCPLHNQEVTAEDGTQRCE
KCSKPCARVCYGLGMEHLREVRAVTSANIQEFAGCKKIFGSLAFLPESFDGDPASNTAPLQPEQLQVFETLEEITGYLYISAW
PDSLPDLSVFQNLQVIRGRILHNGAYSLTLQGLGISWLGLRSLRELGSGLALIHHNTHLCFVHTVPWDQLFRNPHQALLHTAN
RPEDECVGEGLACHQLCARGHCWGPGPTQCVNCSQFLRGQECVEECRVLQGLPREYVNARHCLPCHPECQPQNGSVTCFGPEA
DQCVACAHYKDPPFCVARCPSGVKPDLSYMPIWKFPDEEGACQPCPINCTHSCVDLDDKGCPAEQRASPLTSIISAVVGILLV
VVLGVVFGILIKRRQQKGGTMVSKGEELFTGVVPILVELDGDVNGHKFSVSGEGEGDATYGKLTLKFICTTGKLPVPWPTLVT
TLTYGVQCFSRYPDHMKQHDFFKSAMPEGYVQERTIFFKDDGNYKTRAEVKFEGDTLVNRIELKGIDFKEDGNILGHKLEYNY
NSHNVYIMADKQKNGIKVNFKIRHNIEDGSVQLADHYQQNTPIGDGPVLLPDNHYLSTQSALSKDPNEKRDHMVLLEFVTAAG
ITLGMDELYK
>EGFR-iRFP; hsEGFR_ecd_tm-iRFP; Lentiviral transduction for making EGFR+ K562 cells
(infraredRFP+) (SEQ ID NO: 27471)
MRPSGTAGAALLALLAALCPASRALEEKKVCQGTSNKLTQLGTFEDHFLSLQRMFNNCEVVLGNLEITYVQRNYDLSFLKTIQ
EVAGYVLIALNTVERIPLENLQIIRGNMYYENSYALAVLSNYDANKTGLKELPMRNLQEILHGAVRFSNNPALCNVESIQWRD
IVSSDFLSNMSMDFQNHLGSCQKCDPSCPNGSCWGAGEENCQKLTKIICAQQCSGRCRGKSPSDCCHNQCAAGCTGPRESDCL
VCRKFRDEATCKDTCPPLMLYNPTTYQMDVNPEGKYSFGATCVKKCPRNYVVTDHGSCVRACGADSYEMEEDGVRKCKKCEGP
CRKVCNGIGIGEFKDSLSINATNIKHFKNCTSISGDLHILPVAFRGDSFTHTPPLDPQELDILKTVKEITGFLLIQAWPENRT
DLHAFENLEIIRGRTKQHGQFSLAVVSLNITSLGLRSLKEISDGDVIISGNKNLCYANTINWKKLFGTSGQKTKIISNRGENS
CKATGQVCHALCSPEGCWGPEPRDCVSCRNVSRGRECVDKCNLLEGEPREFVENSECIQCHPECLPQAMNITCTGRGPDNCIQ
CAHYIDGPHCVKTCPAGVMGENNTLVWKYADAGHVCHLCHPNCTYGCTGPGLEGCPTNGPKIPSIATGMVGALLLLLVVALGI
GLFMRRRHIVRKRGGTMAEGSVARQPDLLTCDDEPIHIPGAIQPHGLLLALAADMTIVAGSDNLPELTGLAIGALIGRSAADV
FDSETHNRLTIALAEPGAAVGAPITVGFTMRKDAGFIGSWHRHDQLIFLELEPPQRDVAEPQAFFRRTNSAIRRLQAAETLES
ACAAAAQEVRKITGFDRVMIYRFASDFSGEVIAEDRCAEVESKLGLHYPASTVPAQARRLYTINPVRIIPDINYRPVPVTPDL
NPVTGRPIDLSFAILRSVSPVHLEFMRNIGMHGTMSISILRGERLWGLIVCHHRTPYYVDLDGRQACELVAQVLAWQIGVMEE
>Bim-eGFPligand Lentiviral transduction for making K562 positive control target
cells with membrane-tethered Bim-eGFP (SEQ ID NO: 27472)
METDTLLLWVLLLWVPGSTGDGSEIWIAQELRRIGDEFNAYYASGSGMVSKGEELFTGVVPILVELDGDVNGHKFSVSGEGEG
DATYGKLTLKFICTTGKLPVPWPTLVTTLTYGVQCFSRYPDHMKQHDFFKSAMPEGYVQERTIFFKDDGNYKTRAEVKFEGDT
LVNRIELKGIDFKEDGNILGHKLEYNYNSHNVYIMADKQKNGIKVNFKIRHNIEDGSVQLADHYQQNTPIGDGPVLLPDNHYL
STQSALSKDPNEKRDHMVLLEFVTAAGITLGMDELYKNAVGQDTQEVIVVPHSLPFKVVVISAILALVVLTIISLIILIMLWQ
KKPR
>hsHer2 Lentiviral transduction for making Her2+ Raji cells (no fluorescent
protein) (SEQ ID NO: 27473)
MELAALCRWGLLLALLPPGAASTQVCTGTDMKLRLPASPETHLDMLRHLYQGCQVVQGNLELTYLPTNASLSFLQDIQEVQGY
VLIAHNQVRQVPLQRLRIVRGTQLFEDNYALAVLDNGDPLNNTTPVTGASPGGLRELQLRSLTEILKGGVLIQRNPQLCYQDT
ILWKDIFHKNNQLALTLIDTNRSRACHPCSPMCKGSRCWGESSEDCQSLTRTVCAGGCARCKGPLPTDCCHEQCAAGCTGPKH
SDCLACLHFNHSGICELHCPALVTYNTDTFESMPNPEGRYTFGASCVTACPYNYLSTDVGSCTLVCPLHNQEVTAEDGTQRCE
KCSKPCARVCYGLGMEHLREVRAVTSANIQEFAGCKKIFGSLAFLPESFDGDPASNTAPLQPEQLQVFETLEEITGYLYISAW
PDSLPDLSVFQNLQVIRGRILHNGAYSLTLQGLGISWLGLRSLRELGSGLALIHHNTHLCFVHTVPWDQLFRNPHQALLHTAN
RPEDECVGEGLACHQLCARGHCWGPGPTQCVNCSQFLRGQECVEECRVLQGLPREYVNARHCLPCHPECQPQNGSVTCFGPEA
DQCVACAHYKDPPFCVARCPSGVKPDLSYMPIWKFPDEEGACQPCPINCTHSCVDLDDKGCPAEQRASPLTSIISAVVGILLV
VVLGVVFGILIKRRQQKIRKYTMRRLLQETELVEPLTPSGAMPNQAQMRILKETELRKVKVLGSGAFGTVYKGIWIPDGENVK
IPVAIKVLRENTSPKANKEILDEAYVMAGVGSPYVSRLLGICLTSTVQLVTQLMPYGCLLDHVRENRGRLGSQDLLNWCMQIA
KGMSYLEDVRLVHRDLAARNVLVKSPNHVKITDFGLARLLDIDETEYHADGGKVPIKWMALESILRRRFTHQSDVWSYGVTVW
ELMTFGAKPYDGIPAREIPDLLEKGERLPQPPICTIDVYMIMVKCWMIDSECRPRFRELVSEFSRMARDPQRFVVIQNEDLGP
ASPLDSTFYRSLLEDDDMGDLVDAEEYLVPQQGFFCPDPAPGAGGMVHHRHRSSSTRSGGGDLTLGLEPSEEEAPRSPLAPSE
GAGSDVFDGDLGMGAAKGLQSLPTHDPSPLQRYSEDPTVPLPSETDGYVAPLTCSPQPEYVNQPDVRPQPPSPREGPLPAARP
AGATLERPKTLSPGKNGVVKDVFAFGGAVENPEYLTPQGGAAPQPHPPPAFSPAFDNLYYWDQDPPERGAPPSTFKGTPTAEN
PEYLGLDVPV
>hsEGFR Lentiviral transduction for making EGFR+ Raji cells (no fluorescent
protein) (SEQ ID NO: 27474)
MRPSGTAGAALLALLAALCPASRALEEKKVCQGTSNKLTQLGTFEDHFLSLQRMFNNCEVVLGNLEITYVQRNYDLSFLKTIQ
EVAGYVLIALNTVERIPLENLQIIRGNMYYENSYALAVLSNYDANKTGLKELPMRNLQEILHGAVRFSNNPALCNVESIQWRD
IVSSDFLSNMSMDFQNHLGSCQKCDPSCPNGSCWGAGEENCQKLTKIICAQQCSGRCRGKSPSDCCHNQCAAGCTGPRESDCL
VCRKFRDEATCKDTCPPLMLYNPTTYQMDVNPEGKYSFGATCVKKCPRNYVVTDHGSCVRACGADSYEMEEDGVRKCKKCEGP
CRKVCNGIGIGEFKDSLSINATNIKHFKNCTSISGDLHILPVAFRGDSFTHTPPLDPQELDILKTVKEITGFLLIQAWPENRT
DLHAFENLEIIRGRTKQHGQFSLAVVSLNITSLGLRSLKEISDGDVIISGNKNLCYANTINWKKLFGTSGQKTKIISNRGENS
CKATGQVCHALCSPEGCWGPEPRDCVSCRNVSRGRECVDKCNLLEGEPREFVENSECIQCHPECLPQAMNITCTGRGPDNCIQ
CAHYIDGPHCVKTCPAGVMGENNTLVWKYADAGHVCHLCHPNCTYGCTGPGLEGCPTNGPKIPSIATGMVGALLLLLVVALGI
GLFMRRRHIVRKRTLRRLLQERELVEPLTPSGEAPNQALLRILKETEFKKIKVLGSGAFGTVYKGLWIPEGEKVKIPVAIKEL
REATSPKANKEILDEAYVMASVDNPHVCRLLGICLTSTVQLITQLMPFGCLLDYVREHKDNIGSQYLLNWCVQTAKGMNYLED
RRLVHRDLAARNVLVKTPQHVKITDFGLAKLLGAEEKEYHAEGGKVPIKWMALESILHRIYTHQSDVWSYGVTVWELMTFGSK
PYDGIPASEISSILEKGERLPQPPICTIDVYMIMVKCWMIDADSRPKFRELIIEFSKMARDPQRYLVIQGDERMHLPSPTDSN
FYRALMDEEDMDDVVDADEYLIPQQGFFSSPSTSRTPLLSSLSATSNNSTVACIDRNGLQSCPIKEDSFLQRYSSDPTGALTE
DSIDDTFLPVPEYINQSVPKRPAGSVQNPVYHNQPLNPAPSRDPHYQDPHSTAVGNPEYLNTVQPTCVNSTFDSPAHWAQKGS
HQISLDNPDYQQDFFPKEAKPNGIFKGSTAENAEYLRVAPQSSEFIGA
>hsEpCAM Lentiviral transduction for making EpCAM+ K562 and Raji cells (no
fluorescent protein) (SEQ ID NO: 27475)
MAPPQVLAFGLLLAAATATFAAAQEECVCENYKLAVNCFVNNNRQCQCTSVGAQNTVICSKLAAKCLVMKAEMNGSKLGRRAK
PEGALQNNDGLYDPDCDESGLFKAKQCNGTSTCWCVNTAGVRRTDKDTEITCSERVRTYWIIIELKHKAREKPYDSKSLRTAL
QKEITTRYQLDPKFITSILYENNVITIDLVQNSSQKTQNDVDIADVAYYFEKDVKGESLFHSKKMDLTVNGEQLDLDPGQTLI
YYVDEKAPEFSMQGLKAGVIAVIVVVVIAVVAGIVVLVISRKKRMAKYEKAEIKEMGEMHRELNA

>Bcl2 CAR Co-LOCKR CAR T cell recruitment
SEQ ID NO: 27489
METDTLLLWVLLLWVPGSTGDYKDEYPYDVPDYAGSAHAGRTGYDNRE
IVMKYIHYKLSQRGYEWDAGDDAEENRTEAPEGTESEVVHRALRDAGD
DFERRYRRDFAEMSSQLHLTPDTARQRFETVVEELFRDGVNWGRIVAF
FEFGGVMCVESVNREMSPLVDNIAEWMTEYLNRHLHTWIQDNGGWDAF
VELYGPSMRGGGGSGGGGSESKYGPPCPPCPAPPVAGPSVFLFPPKPK
DTLMISRTPEVTCVVVDVSQEDPEVQFNWYVDGVEVHNAKTKPREEQF
QSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKS
LSLSLGKMFWVLVVVGGVLACYSLLVTVAFTIFWVRSKRSRGGHSDYM
NMTPRRPGPTRKHYQPYAPPRDFAAYRSRVKFSRSADAPAYQQGQNQL
YNELNLGRREEYDVLDKRRGRDPEMGGKPRRKNPQEGLYNELQKDKMA
EAYSEIGMKGERRRGKGHDGLYQGLSTATKDTYDALHMQALPPRLEGG
GEGRGSLLTCGDVEENPGPRMLLLVTSLLLCELPHPAFLLIPRKVCNG
IGIGEFKDSLSINATNIKHFKNCTSISGDLHILPVAFRGDSFTHTPPL
DPQELDILKTVKEITGFLLIQAWPENRTDLHAFENLEIIRGRTKQHGQ
FSLAVVSLNITSLGLRSLKEISDGDVIISGNKNLCYANTINWKKLFGT
SGQKTKIISNRGENSCKATGQVCHALCSPEGCWGPEPRDCVSCRNVSR
GRECVDKCNLLEGEPREFVENSECIQCHPECLPQAMNITCTGRGPDNC
IQCAHYIDGPHCVKTCPAGVMGENNTLVWKYADAGHVCHLCHPNCTYG
CTGPGLEGCPTNGPKIPSIATGMVGALLLLLVVALGIGLFM

We claim:

1. A method of increasing selectivity of a cell for a chimeric antigen receptor (CAR) T cell therapy comprising
(a) contacting cells with a first cage polypeptide fused to a first binding domain, wherein the first cage polypeptide comprises (i) a structural region and (ii) a latch region further comprising one or more bioactive peptides, wherein the structural region interacts with the latch region to prevent activity of the one or more bioactive peptides in the absence of colocalization with a key polypeptide and wherein the first binding domain is capable of binding to a first cell moiety present on or within a cell; and
(b) contacting the cell with a first key polypeptide fused to a second binding domain, wherein upon colocalization with the first cage polypeptide, the first key polypeptide is capable of binding to the cage structural region to activate the one or more bioactive peptides, wherein the second binding domain is capable of binding to a second cell moiety present on or within the cell,
wherein the first cell moiety and the second cell moiety are different or the same.

2. The method of claim 1, wherein the first cell moiety and the second cell moiety are different.

3. The method of claim 1, wherein the first cell moiety and the second cell moiety are the same.

4. The method of claim 3, wherein the colocalization of the first cage polypeptide and the key polypeptide first key polypeptide increases selectivity of an effector toward a cell comprising the first cell moiety and the second cell moiety.

5. The method of any one of claims 1 to 4, wherein the contacting (a) and contacting (b) are performed concurrently or sequentially.

6. The method of any one of claims 1 to 5, wherein the first cell moiety and the second cell moiety are in close proximity to each other; optionally wherein:
(a) the first cell moiety and the second cell moiety are colocalized as a result of directly or indirectly forming a complex; and/or
(b) the first cell moiety and the second cell moiety are colocalized as a result of being expressed in sufficient numbers in the same subcellular compartment.

7. The method of any one of claims 1 to 5, wherein the first cell moiety and/or the second cell moiety are present at least about 500 copies per cell, at least about 1000 copies per cell, at least about 1500 copies per cell, at least about 2000 copies per cell, at least about 2500 copies per cell, at least about 3000 copies per cell, at least about 3500 copies per cell, at least about 4000 copies per cell, at least about 4500 copies per cell, at least about 5000 copies per cell, at least about 5500 copies per cell, at least about 6000 copies per cell, at least about 6500 copies per cell, or at least about 7000 copies per cell.

8. The method of any one of claims 1 to 7, further comprising allowing the first cage polypeptide and the first key polypeptide to colocalize, thereby forming a complex and activating the one or more bioactive peptides.

9. The method of any one of claims 1 to 8, wherein the first cell moiety and the second cell moiety are present on the surface of the cell.

10. The method of any one of claims 1 to 8, wherein the first cell moiety and the second cell moiety are present within the cytoplasm of the cell.

11. The method of any one of claims 1 to 8, wherein the first cell moiety and the second cell moiety are present within the nucleus of the cell.

12. The method of any one of claims 1 to 11, further comprising contacting the cells with a second key polypeptide fused to a third binding domain, wherein upon colocalization with the first cage polypeptide, the second key polypeptide is capable of binding to the cage structural region to activate the one or more bioactive peptides, wherein the third binding domain is capable of binding to a third cell moiety present on or within the cell that also comprises the first cell moiety and/or the second cell moiety, wherein the third cell moiety is different from the first cell moiety or the second cell moiety; and optionally, further comprising a third key polypeptide, a fourth key polypeptide, a fifth key polypeptide, a sixth key polypeptide, or a seventh key polypeptide, wherein one or more of the third, fourth, fifth, sixth, or seventh key polypeptides are fused to a binding domain, wherein the binding domain is capable of binding to a cell moiety present on or within the cell that comprises the first cell moiety.

13. The method of any one of claims 1-11, wherein
(i) the first key polypeptide comprises a third binding domain, wherein the second binding domain and/or the third binding domain bind to (i) different moieties than the first binding domain on the surface of the same cell, or (ii) different moieties than the first binding domain at the synapse between two cells that are in contact, wherein upon colocalization with the first cage polypeptide, the first key polypeptide is capable of binding to the cage structural region to activate the one or more bioactive peptides, wherein the third binding domain is capable of binding to a third cell moiety present on or within the cell that also comprises the first cell moiety, wherein the third cell moiety is different from the first cell moiety or the second cell moiety; and/or
(ii) further comprising contacting the cells with at least a second cage polypeptide comprising (A) a second structural region, (B) a second latch region further comprising one or more bioactive peptides, and (C) a sixth binding domain, wherein the second structural region interacts with the second latch region to prevent activity of the one or more bioactive peptides, wherein the first key and/or the second key polypeptide are capable of binding to the second structural region to activate the one or more bioactive peptides, and wherein the sixth binding domain and/or the first binding domain bind to (I) different moieties than the second binding domain, third binding domain and/or fourth binding domain on the surface of the same cell, or (II) different moieties than the second binding domain, third binding domain and/or fourth binding domain at the synapse between two cells that are in contact; wherein upon colocalization with the first cage or the second cage polypeptide, the first key polypeptide is capable of binding to the first cage or the second cage structural region to activate the one or more bioactive peptides.

14. The method of any one of claims 1 to 11, further comprising contacting a second key polypeptide fused to a third binding domain with the cells comprising a second cell that also comprises a first cell moiety, wherein upon colocalization with the first cage polypeptide, the second key polypeptide is capable of binding to the cage structural region to activate the one or more bioactive peptides, wherein the third binding domain is capable of binding to a third cell moiety present on or within the second cell.

15. The method of any one of claim 1 to 11 or 14, further comprising contacting the cells with a third key polypeptide fused to a fourth binding domain, wherein upon colocalization with the first cage polypeptide, the third key polypeptide is capable of binding to the cage structural region to activate the one or more bioactive peptides, wherein the third binding domain is capable of binding to a third cell moiety present on or within the cell that also comprises the first cell moiety, wherein the third cell moiety is different from the first cell moiety or the second cell moiety.

16. The method of claim 15, further comprising contacting the cells with a fourth key polypeptide, a fifth key polypeptide, a sixth key polypeptide, or a seventh key polypeptide, wherein one or more of the fourth, fifth, sixth, or seventh key polypeptides are fused to a binding domain, wherein the binding domain is capable of binding to a cell moiety present on or within the cell.

17. The method of any one of claims 1 to 16, further comprising contacting the cells with one or more decoy cage polypeptide fused to one or more binding domain (“decoy binding domain”), wherein each decoy cage polypeptide comprises a decoy structural region, which upon colocalization with the first key polypeptide and the first cage polypeptide, is capable of preferentially binding to the first key polypeptide and wherein each decoy binding domain is capable of binding to a cell moiety (“decoy cell moiety”) in the cell that comprises the first cell moiety and/or the second cell moiety.

18. The method of claim 17, wherein each decoy cell moiety is present only on a healthy cell.

19. The method of claim 17 or 18, wherein upon colocalization with the first key polypeptide, the decoy cage polypeptide binds to the first key polypeptide and wherein the one or more bioactive peptides in the first cage polypeptide are not activated.

20. A method of increasing selectivity of cells that are interacting with each other for a chimeric antigen receptor T cell therapy comprising:
(a) contacting two or more cells with a first cage polypeptide fused to a first binding domain, wherein the first cage polypeptide comprises (i) a structural region and (ii) a latch region further comprising one or more bioactive peptides, wherein the structural region interacts with the latch region to prevent activity of the one or more bioactive peptides in the absence of colocalization with a key polypeptide and wherein the first binding domain is capable of binding to a first cell moiety present on a synapse between the two or more cells; and
(b) contacting the two or more cells with a first key polypeptide fused to a second binding domain, wherein upon colocalization with the first cage polypeptide, the first key polypeptide is capable of binding to the cage structural region to activate the one or more bioactive peptides, wherein the second binding domain is capable of binding to a second cell moiety present on the synapse between the two or more cells,
wherein the first cell surface moiety and the second cell surface moiety are the same or different.

21. The method of claim 20, wherein the first cell moiety and the second cell moiety are in close proximity to each other.

22. The method of claim 20 or 21, further comprising allowing the first cage polypeptide and the first key polypeptide to colocalize, thereby forming a complex and activating the one or more bioactive peptides.

23. The method of any one of claims 20 to 22, wherein the first cell moiety and the second cell moiety are different or the same.

24. The method of any one of claims 20 to 23, wherein the contacting (a) and contacting (b) are performed concurrently or sequentially.

25. The method of any one of claims 20 to 24, further comprising contacting a second key polypeptide fused to a third binding domain with a synapse of two or more cells that also express a first cell moiety, wherein upon colocalization with the first cage polypeptide, the second key polypeptide is capable of binding to the cage structural region to activate the one or more bioactive peptides, wherein the third binding domain is capable of binding to a third cell moiety present on the synapse of the two or more cells.

26. The method of any one of claims 20 to 25, further comprising contacting the two or more cells with one or more decoy cage polypeptide fused to one or more decoy binding domain with the two or more cells, wherein each decoy cage polypeptide comprises a decoy structural region, which upon colocalization with the first key polypeptide and the first cage polypeptide, is capable of preferentially binding to the first key polypeptide and wherein each decoy binding domain is capable of binding to a decoy cell moiety in the synapse of the two or more cells.

27. A method of targeting heterogeneous cells (more than two different cell types) for a chimeric antigen receptor T cell therapy, wherein a first cell moiety and a second cell moeity are present on the first cell and a first cell moiety and a third cell moiety are present on the second cell, comprising:
(a) contacting two or more cells with a first cage polypeptide fused to a first binding domain, wherein the first cage polypeptide comprises (i) a structural region and (ii) a latch region further comprising one or more bioactive peptides, and wherein the structural region interacts with the latch region to prevent activity of the one or more bioactive peptides in the absence of colocalization with a key polypeptide and wherein the first binding domain is capable of binding to a first cell moiety present on or within the two or more cells;
(b) contacting the two or more cells with a first key polypeptide fused to a second binding domain, wherein upon colocalization, the first key polypeptide is capable of binding to the cage structural region to activate the one or more bioactive peptides and wherein the second binding domain is capable of binding to a second cell moiety present on a cell that also comprises the first cell moiety, and
(c) contacting the two or more cells with a second key polypeptide fused to a third binding domain, wherein upon colocalization, the second key polypeptide is capable of binding to the cage structural region to activate the one or more bioactive peptides and wherein the third binding domain is capable of binding to a third cell moiety in a cell that comprises the first cell moiety,
wherein the first cell moiety, the second cell moiety, and the third cell moiety are different and the cell that comprises the second cell moiety and the cell that comprises the third cell moiety are different.

28. The method of claim 27, wherein the first key polypeptide and the second key polypeptide are identical.

29. The method of claim 27, wherein the first key polypeptide and the second key polypeptide are not identical.

30. The method of any one of claims 27 to 29, further comprising contacting the two or more cells with one or more decoy cage polypeptide fused to one or more decoy binding domain, wherein each decoy cage polypeptide comprises a decoy structural region, which upon colocalization with the first key polypeptide, the second key polypeptide, and/or the first cage polypeptide, is capable of preferentially binding to the first key polypeptide or the second key polypeptide and wherein each decoy binding domain is capable of binding to a decoy cell moiety in a cell that comprises the first cell moiety and the second cell moiety.

31. A method of reducing off-target activity for a chimeric antigen receptor T cell therapy comprising
(a) contacting two or more cells with a first cage polypeptide fused to a first binding domain, wherein the first cage polypeptide comprises (i) a structural region and (ii) a latch region further comprising one or more bioactive peptides, and wherein the structural region interacts with the latch region to prevent activity of the one or more bioactive peptides in the absence of colocalization with a key polypeptide and wherein the first binding domain is capable of binding to a first cell moiety present on a cell;
(b) contacting the two or more cells with a first key polypeptide fused to a second binding domain, wherein upon colocalization, the first key polypeptide is capable of binding to the cage structural region to activate the one or more bioactive peptides and wherein the second binding domain is capable of binding to a second cell moiety present on a cell that also comprises the first cell moiety, and
(c) contacting the two or more cells with a decoy cage polypeptide fused to a third binding domain, wherein the decoy cage polypeptide comprises a decoy structural region, which upon colocalization with the key polypeptide and the first cage polypeptide, is capable of preferentially binding to the first key polypeptide and wherein the third binding domain is capable of binding to a third cell moiety in a cell that comprises the first cell moiety and the second cell moiety.

32. The method of claim 31, wherein the third cell moiety is only present on a healthy cell.

33. The method of any one of claims 1 to 32, wherein the first cage polypeptide comprises no more than 7 alpha helices, 6 alpha helices, 5 alpha helices, no more than 4 alpha helices, no more than 3 alpha helices, or no more than 2 alpha helices, wherein the structural region comprises at least one alpha helices and the latch region comprises at least one alpha helices.

34. The method of any one of claims 1 to 33, wherein the structural region of the first cage polypeptide comprises one alpha helix, two alpha helices, three alpha helices, four alpha helices, five alpha helices, or six alpha helices, and the latch region of the first key polypeptide comprises no more than one alpha helix.

35. The method of claims 17 to 19, and 26 to 34, wherein each decoy cage polypeptide comprises at least one alpha helix, at least two alpha helices, at least three alpha helices, at least four alpha helices, at least five alpha helices, at least six alpha helices, or at least seven alpha helices.

36. The method of any one of claims 17 to 19 and 26 to 35, wherein the binding affinity of the decoy cage polypeptide to a key polypeptide (e.g., K_{D}) is stronger (e.g., lower) than the binding affinity of the first cage polypeptide to a key polypeptide (e.g., K_{D}) by at least about 1.1 fold, at least about 1.5 fold, at least about 2 fold, at least about 3 fold, at least about 4 fold, at least about 5 fold, at least about 6 fold, at least about 7 fold, at least about 8 fold, at least about 9 fold, at least about 10 fold, at least about 20 fold, at least about 30 fold, at least about 40 fold, at least about 50 fold, at least about 60 fold, at least about 70 fold, at least about 80 fold, at least about 90 fold, at least about 100 fold, at least about 150 fold, at least about 200 fold, at least about 300 fold, at least about 400 fold, at least about 500 fold, at least about 600 fold, at least about 700 fold, at least about 800 fold, at least about 900 fold, or at least about 1000 fold.

37. The method of any one of claims 1 to 36, wherein the binding of the first cage polypeptide and the first key polypeptide in a solution is less efficient than the binding of the first cage polypeptide and the first key polypeptide when colocalized on or within the cell.

38. The method of any one of claims 1 to 37, wherein the colocalization of the first cage polypeptide and the first key polypeptide increases the local concentration of the first cage polypeptide and the first key polypeptide and shifts the binding equilibrium in favor of complex formation between the first cage polypeptide and the first key polypeptide.

39. The method of any one of claims 1 to 38, wherein the contacting includes introducing a polynucleotide encoding a polypeptide (e.g., the first cage polypeptide, the first key polypeptide, the second key polypeptide, and the decoy cage polypeptide).

40. The method of any one of claims 1 to 39, wherein the first cage polypeptide, the first key polypeptide, the second key polypeptide, and/or the decoy polypeptide are further modified to change (i) hydrophobicity, (ii) a hydrogen bond network, (iii) a binding affinity to each, and/or (iv) any combination thereof.

41. The method of any one of claims 1 to 40, wherein an interface between the latch region and the structural region of the first cage polypeptide includes a hydrophobic amino acid to polar amino acid residue ratio of between 1:1 and 10:1, e.g., 1:1, 2:1, 3:1, 4:1, 5:1, 6:1, 7:1, 8:1, 9:1, or 10:1.

42. The method of any one of claims 1 to 41, wherein the latch region is mutated to reduce the hydrophobicity.

43. The method of claim 42, wherein 1, 2, 3, or more large hydrophobic residues in the latch region, e.g., isoleucine, valine, or leucine, are mutated to serine, threonine, or a smaller hydrophobic amino acid residue, e.g., valine (if the starting amino acid is isoleucine or leucine) or alanine.

44. The method of any one of claims 1 to 43, wherein the first cage polypeptide comprises buried amino acid residues at the interface between the latch region and the structural region of the first cage polypeptide, wherein the buried amino acid residues at the interface have side chains comprising nitrogen or oxygen atoms involved in hydrogen bonding.

45. The method of any one of claims 1 to 44, wherein the cells that the first cell moiety and/or the second cell moiety are present on or within tumor cells.

46. The method of any one of claims 1 to 45, wherein one or more of the first, second, third, fourth, fifth, sixth, seventh, and/or decoy binding domains comprise an antibody or antigen binding portion thereof, Fab′, F(ab′)₂, Fab, Fv, rIgG, recombinant single chain Fv fragments (scFv), V_{H} single domains, bivalent or bispecific molecules, diabodies, triabodies, and tetrabodies, DARPins, nanobody, affibody, monobody, adnectin, alphabody, Albumin-binding domain, Adhiron, Affilin, Affimer, Affitin/Nanofitin, Anticalin, Armadillo repeat proteins, Atrimer/Tetranectin, Avimer/Maxibody, Centyrin, Fynomer, Kunitz domain, Obody/OB-fold, Pronectin, Repebody, computationally designed proteins, or any combination thereof.

47. The method of any one of claims 1 to 46, wherein one or more of the first, second, third, fourth, fifth, sixth, seventh, and/or decoy binding domains bind to a cell surface protein comprising Her2, EGFR, EpCAM, B7-H3, ROR1, GD2, GPC2, αvβ6, Her3, L1CAM, BCMA, GPCR5d, EGFRvIII, CD20, CD22, CD3, CD4, CD5, CD8, CD19, CD27, CD28, CD30, CD33, CD48, IL3RA, platelet tissue factor, CLEC12A, CD82, TNFRSF1B, ADGRE2, ITGB5, CD96, CCR1, PTPRJ, CD70, LILRB2, LTB4R, TLR2, LILRA2, ITGAX, CR1, EMC10, EMB, DAGLB, P2RY13, LILRB3, LILRB4, SLC30A1, LILRA6, SLC6A6, SEMA4A, TAG72, FRα, PMSA, Mesothelin, LIV-1, CEA, MUC1, PD1, BLIMP1, CTLA4, LAG3, TIM3, TIGIT, CD39, Nectin-4, a cancer marker, a healthy tissue marker, a cardiac marker, or any combination thereof.

48. The method of any one of claims 1 to 47, wherein one or more of the cage polypeptides and the key polypeptides further comprises a linker connecting the cage or key polypeptide and the one or more binding domains.

49. The method of any one of claims 1 to 49, further comprising administering a chimeric antigen receptor T cell to the cells.

50. The method of any one of claims 1 to 49, wherein the cells are present in vivo.

51. The method of any one of claims 1 to 49, wherein the cells are present in vitro or ex vivo.

52. The method of any one of claims 49 to 51, wherein the CAR T cell binds to the one or more bioactive peptides.

53. The method of claim 52, wherein the CAR T cell comprises an antibody or antigen binding fragment thereof, T cell receptor, DARPin, bispecific or bivalent molecule, nanobody, affibody, monobody, adnectin, alphabody, albumin binding domain, adhiron, affilin, affimer, affitin/nanofitin; anticalin; armadillo repeat protein; atrimer/tetranectin; avimer/maxibody; centyrin; fynomer; Kunitz domain; obody/OB-fold; pronectin; repebody; or computationally designed protein.

54. The method of claim 53, wherein the antigen binding portion thereof comprises a Fab′, F(ab′)₂, Fab, Fv, rIgG, recombinant single chain Fv fragment (scFv), and/or V_{H} single domain.

55. The method of any one of claims 49 to 54, wherein the administering kills the cell that comprises the first binding moiety and the second binding moiety.

56. A protein complex formed by any one of the methods 1 to 55.

57. A polynucleotide encoding the protein complex of claim 56.

58. A protein complex comprising (i) a first cage polypeptide fused to a first binding domain and (ii) a first key polypeptide fused to a second binding domain, wherein the first cage polypeptide comprises (i) a structural region and (ii) a latch region further comprising one or more bioactive peptides, wherein the first key polypeptide binds to the cage structural region, wherein the one or more bioactive peptides are activated, and wherein the first binding domain binds to a first cell moiety present on or within a cell or on a synapse of two interacting cells and the second binding domain binds to a second cell moiety present on or within the cell or on a synapse of the two interacting cells, wherein the first cell moiety and the second cell moiety are different or the same.

59. A protein complex comprising (i) a first key polypeptide fused to a first binding domain and (ii) a decoy cage polypeptide fused to a second binding domain, wherein the first key polypeptide binds to the decoy cage polypeptide, and wherein the first binding domain binds to a first cell moiety present on or within a cell or on a synapse of two interacting cells and the second binding domain binds to a second cell moiety present on or within the cell or on a synapse of the two interacting cells, wherein the first cell moiety and the second cell moiety are different or the same.

60. A composition comprising
(a) a first cage polypeptide fused to a first binding domain or a polynucleotide encoding the same, wherein the first cage polypeptide comprises (i) a structural region and (ii) a latch region further comprising one or more bioactive peptides, wherein the structural region interacts with the latch region to prevent activity of the one or more bioactive peptides in the absence of colocalization with a key polypeptide and wherein the first binding domain is capable of binding to a first cell moiety present on or within a cell; and
(b) a first key polypeptide fused to a second binding domain or a polynucleotide encoding the same, wherein upon colocalization with the first cage polypeptide, the first key polypeptide is capable of binding to the cage structural region to activate the one or more bioactive peptides, wherein the second binding domain is capable of binding to a second cell moiety present on or within the cell,
wherein the first cell moiety and the second cell moiety are different or the same and wherein the cell is a target for a chimeric antigen receptor (CAR) T cell therapy.

61. The composition of claim 60, wherein the first cell moiety and the second cell moiety are different.

62. The composition of claim 60, wherein the first cell moiety and the second cell moiety are the same.

63. The composition of claim 62, wherein the colocalization of the first cage polypeptide and the first key polypeptide increases selectivity of an effector toward a cell comprising the first cell moiety and the second cell moiety.

64. The composition of any one of claims 60 to 63, wherein the first cage polynucleotide and the first key polynucleotide are encoded on the same or different nucleic acid sequence.

65. The composition of any one of claims 60 to 64, wherein the first cell moiety and the second cell moiety are in close proximity to each other; optionally wherein:
(a) the first cell moiety and the second cell moiety are colocalized as a result of directly or indirectly forming a complex; or
(b) the first cell moiety and the second cell moiety are colocalized as a result of being present in sufficient numbers in the same subcellular compartment.

66. The composition of any one of claims 60 to 65, wherein the first cell moiety and/or the second cell moiety are present at least about 500 copies per cell, at least about 1000 copies per cell, at least about 1500 copies per cell, at least about 2000 copies per cell, at least about 2500 copies per cell, at least about 3000 copies per cell, at least about 3500 copies per cell, at least about 4000 copies per cell, at least about 4500 copies per cell, at least about 5000 copies per cell, at least about 5500 copies per cell, at least about 6000 copies per cell, at least about 6500 copies per cell, or at least about 7000 copies per cell.

67. The composition of any one of claims 60 to 66, wherein the first cage polypeptide and the first key polypeptide are colocalized, thereby forming a complex and activating the one or more bioactive peptides.

68. The composition of any one of claims 60 to 67, wherein the first cell moiety and the second cell moiety are present on the surface of the cell.

69. The composition of any one of claims 60 to 67, wherein the first cell moiety and the second cell moiety are present within the cytoplasm of the cell.

70. The composition of any one of claims 60 to 67, wherein the first cell moiety and the second cell moiety are present within the nucleus of the cell.

71. The composition of any one of claims 60 to 70, further comprising a second key polypeptide fused to a third binding domain or a polynucleotide encoding the same, wherein upon colocalization with the first cage polypeptide, the second key polypeptide is capable of binding to the cage structural region to activate the one or more bioactive peptides, wherein the third binding domain is capable of binding to a third cell moiety present on or within the cell that also comprises the first cell moiety and/or the second cell moiety, wherein the third cell moiety is different from the first cell moiety or the second cell moiety.

72. The composition of claim 71, further comprising a third key polypeptide, a fourth key polypeptide, a fifth key polypeptide, a sixth key polypeptide, or a seventh key polypeptide, or a polynucleotide encoding the same, wherein one or more of the third, fourth, fifth, sixth, or seventh key polypeptides are fused to a binding domain, and wherein the binding domain is capable of binding to a cell moiety present on or within the cell that comprises the first cell moiety, the second cell moiety, and/or the third cell moiety.

73. The composition of any one of claims 60 to 70, further comprising a second key polypeptide fused to a third binding domain or a polynucleotide encoding the same, wherein upon colocalization with the first cage polypeptide, the second key polypeptide is capable of binding to the cage structural region to activate the one or more bioactive peptides, and wherein the third binding domain is capable of binding to a third cell moiety expressed on or within a second cell that also expresses a first cell moiety.

74. The composition of any one of claim 60 to 70 or 73, further comprising a third key polypeptide fused to a fourth binding domain or a polynucleotide encoding the same, wherein upon colocalization with the first cage polypeptide, the third key polypeptide is capable of binding to the cage structural region to activate the one or more bioactive peptides, wherein the third binding domain is capable of binding to a third cell moiety expressed on or within the cell that also expresses the first cell moiety, and wherein the third cell moiety is different from the first cell moiety or the second cell moiety.

75. The composition of claim 74, further comprising a fourth key polypeptide, a fifth key polypeptide, a sixth key polypeptide, or a seventh key polypeptide, or a polynucleotide encoding the same, wherein one or more of the fourth, fifth, sixth, or seventh key polypeptides are fused to a binding domain, wherein the binding domain is capable of binding to a cell moiety present on or within the cell.

76. The composition of any one of claims 60 to 75, further comprising one or more decoy cage polypeptide fused to one or more binding domain (“decoy binding domain”) or a polynucleotide encoding the same, wherein each decoy cage polypeptide comprises a decoy structural region, which upon colocalization with the first key polypeptide and the first cage polypeptide, is capable of preferentially binding to the first key polypeptide and wherein each decoy binding domain is capable of binding to a cell moiety (“decoy cell moiety”) in the cell that comprises the first cell moiety and/or the second cell moiety.

77. The composition of claim 76, wherein each decoy cell moiety is present only on a healthy cell.

78. The composition of claim 76 or 77, wherein upon colocalization with the first key polypeptide, the decoy cage polypeptide binds to the first key polypeptide and wherein the one or more bioactive peptides in the first cage polypeptide are not activated.

79. The composition of any one of claims 60 to 78, wherein the first cage polypeptide comprises no more than 5 alpha helices, no more than 4 alpha helices, no more than 3 alpha helices, or no more than 2 alpha helices, wherein the structural region comprises at least one alpha helices and the latch region comprises at least one alpha helices.

80. The composition of any one of claims 60 to 79, wherein the structural region of the first cage polypeptide comprises one alpha helix, two alpha helices, or three alpha helices, and the latch region of the first key polypeptide comprises no more than one alpha helix.

81. The composition of claims 76 to 80, wherein the decoy cage polypeptide comprises at least one alpha helix, at least two alpha helices, at least three alpha helices, at least four alpha helices, or at least five alpha helices.

82. The composition of any one of claims 76 to 81, wherein the binding affinity of the decoy cage polypeptide to a key polypeptide (e.g., K_{D}) is stronger (e.g., lower) than the binding affinity of the first cage polypeptide to a key polypeptide (e.g., K_{D}) by at least about 1.1 fold, at least about 1.5 fold, at least about 2 fold, at least about 3 fold, at least about 4 fold, at least about 5 fold, at least about 6 fold, at least about 7 fold, at least about 8 fold, at least about 9 fold, at least about 10 fold, at least about 20 fold, at least about 30 fold, at least about 40 fold, at least about 50 fold, at least about 60 fold, at least about 70 fold, at least about 80 fold, at least about 90 fold, at least about 100 fold, at least about 150 fold, at least about 200 fold, at least about 300 fold, at least about 400 fold, at least about 500 fold, at least about 600 fold, at least about 700 fold, at least about 800 fold, at least about 900 fold, or at least about 1000 fold.

83. The composition of any one of claims 60 to 82, wherein the binding of the first cage polypeptide and the first key polypeptide in a solution is less efficient than the binding of the first cage polypeptide and the first key polypeptide when colocalized on or within the cell.

84. The composition of any one of claims 60 to 83, wherein the colocalization of the first cage polypeptide and the first key polypeptide increases the local concentration of the first cage polypeptide and the first key polypeptide and shifts the binding equilibrium in favor of complex formation between the first cage polypeptide and the first key polypeptide.

85. The composition of any one of claims 60 to 84, wherein the first cage polypeptide, the first key polypeptide, the second key polypeptide, and/or the decoy polypeptide are further modified to change (i) hydrophobicity, (ii) a hydrogen bond network, (iii) a binding affinity to each, and/or (iv) any combination thereof.

86. The composition of any one of claims 60 to 85, wherein an interface between the latch region and the structural region of the first cage polypeptide includes a hydrophobic amino acid to polar amino acid residue ratio of between 1:1 and 10:1, e.g., 1:1, 2:1, 3:1, 4:1, 5:1, 6:1, 7:1, 8:1, 9:1, or 10:1.

87. The composition of any one of claims 60 to 86, wherein the latch region is mutated to reduce the hydrophobicity.

88. The composition of claim 87, wherein 1, 2, 3, or more large hydrophobic residues in the latch region, e.g., isoleucine, valine, or leucine, are mutated to serine, threonine, or a smaller hydrophobic amino acid residue, e.g., valine (if the starting amino acid is isoleucine or leucine) or alanine.

89. The composition of any one of claims 60 to 88, wherein the first cage polypeptide comprises buried amino acid residues at the interface between the latch region and the structural region of the first cage polypeptide, wherein the buried amino acid residues at the interface have side chains comprising nitrogen or oxygen atoms involved in hydrogen bonding.

90. The composition of any one of claims 60 to 89, wherein the cells that the first cell moiety and/or the second cell moiety are present on or within tumor cells, cancer cells, immune cells, leukocytes, lymphocytes, T cells, regulatory T cells, effector T cells, CD4+ effector T cells, CD8+ effector T cells, memory T cells, autoreactive T cells, exhausted T cells, natural killer T cells (NKT cells), B cells, dendritic cells, macrophages, NK cells, cardiac cells, lung cells, muscle cells, epithelial cells, pancreatic cells, skin cells, CNS cells, neurons, myocytes, skeletal muscle cells, smooth muscle cells, liver cells, kidney cells, bacterial cells, yeast cells, or any combination thereof.

91. The composition of any one of claims 60 to 90, wherein one or more of the first, second, third, fourth, fifth, sixth, seventh, and/or decoy binding domains comprise an antibody or antigen binding portion thereof, Fab′, F(ab′)₂, Fab, Fv, rIgG, recombinant single chain Fv fragments (scFv), V_{H} single domains, bivalent or bispecific molecules, diabodies, triabodies, and tetrabodies, DARPins, nanobody, affibody, monobody, adnectin, alphabody, Albumin-binding domain, Adhiron, Affilin, Affimer, Affitin/Nanofitin, Anticalin, Armadillo repeat proteins, Atrimer/Tetranectin, Avimer/Maxibody, Centyrin, Fynomer, Kunitz domain, Obody/OB-fold, Pronectin, Repebody, computationally designed proteins, or any combination thereof.

92. The composition of any one of claims 60 to 91, wherein one or more of the first, second, third, fourth, fifth, sixth, seventh, and/or decoy binding domains bind to a cell surface protein comprising Her2, EGFR, EpCAM, B7-H3, ROR1, GD2, GPC2, αvβ6, Her3, L1CAM, BCMA, GPCR5d, EGFRvIII, CD20, CD22, CD3, CD4, CD5, CD8, CD19, CD27, CD28, CD30, CD33, CD48, IL3RA, platelet tissue factor, CLEC12A, CD82, TNFRSF1B, ADGRE2, ITGB5, CD96, CCR1, PTPRJ, CD70, LILRB2, LTB4R, TLR2, LILRA2, ITGAX, CR1, EMC10, EMB, DAGLB, P2RY13, LILRB3, LILRB4, SLC30A1, LILRA6, SLC6A6, SEMA4A, TAG72, FRα, PMSA, Mesothelin, LIV-1, CEA, MUC1, PD1, BLIMP1, CTLA4, LAG3, TIM3, TIGIT, CD39, Nectin-4, a cancer marker, a healthy tissue marker, a cardiac marker, or any combination thereof.

93. The composition of any one of claims 60 to 92, wherein one or more of the cage polypeptides and the key polypeptides further comprises a linker connecting the cage or key polypeptide and the one or more binding domains.

94. The composition of any one of claims 60 to 93, further comprising a chimeric antigen receptor T cell.

95. A cell comprising the composition of any one of claims 60 to 93.

96. The cell of claim 95, which is a tumor cell.

97. A method of preparing a subject in need thereof comprising administering the composition of any one of claims 60 to 93 to the subject.

98. The method of claim 97, wherein one or more cells of the subject exhibit activated one or more bioactive peptide.

99. A method of treating a disease or condition in a subject in need thereof comprising administering a chimeric antigen receptor T cell that binds to one or more bioactive peptides to the subject, wherein the subject is further administered the composition of any one of claims 60 and 93.

100. The method of claim 100, wherein the chimeric antigen receptor T cell comprises an antibody or antigen binding fragment thereof, T cell receptor, DARPin, bispecific or bivalent molecule, nanobody, affibody, monobody, adnectin, alphabody, albumin binding domain, adhiron, affilin, affimer, affitin/nanofitin; anticalin; armadillo repeat protein; atrimer/tetranectin; avimer/maxibody; centyrin; fynomer; Kunitz domain; obody/OB-fold; pronectin; repebody; or computationally designed protein.

101. The method of claim 100, wherein the antigen binding portion thereof comprises a Fab′, F(ab′)₂, Fab, Fv, rIgG, recombinant single chain Fv fragment (scFv), and/or V_{H} single domain.

102. The method of any one of claims 99 to 101, wherein the administering kills the cell that comprises the first binding moiety and the second binding moiety.

103. A composition comprising
(a) a first cage polypeptide comprising (i) a structural region, (ii) a latch region further comprising one or more bioactive peptides, and (iii) a first binding domain wherein the structural region interacts with the latch region to prevent activity of the one or more bioactive peptides;
(b) a first key polypeptide capable of binding to the cage structural region to activate the one or more bioactive peptides, wherein the key polypeptide comprises a second binding domain,
wherein the first binding domain and the second binding domain bind to (i) different moieties on the surface of the same cell, (ii) the same moiety on the surface of the same cell, (iii) different moieties at the synapse between two cells that are in contact, or (iv) the same moiety at the synapse between two cells that are in contact; and
(c) cells comprising one or more chimeric antigen receptor(s) that bind to the one or more bioactive peptides when the one or more bioactive peptides are activated.

104. The composition of claim 103, wherein the first key polypeptide comprises a third binding domain, wherein the second binding domain and/or the third binding domain bind to (i) different moieties than the first binding domain on the surface of the same cell, or (ii) different moieties than the first binding domain at the synapse between two cells that are in contact.

105. The composition of claim 104, wherein the second binding domain and the third binding domain bind to different moieties on the surface of different cells.

106. The composition of any one of claims 103-105, further comprising:
(d) at least a second key polypeptide capable of binding to the first cage structural region, wherein the key polypeptide comprises a fourth binding domain,
wherein the second binding domain and/or the fourth binding domain bind to (i) different moieties than the first binding domain on the surface of the same cell, or (ii) different moieties than the first binding domain at the synapse between two cells that are in contact.

107. The composition of claim 106, wherein the second binding domain and the fourth binding domain bind to (i) different moieties on the surface of the same cell, or (ii) different moieties at the synapse between two cells that are in contact; or wherein the second binding domain and the fourth binding domain bind to different moieties on the surface of different cells.

108. The composition of any one of claims 103-107, wherein the first cage polypeptide further comprises a fifth binding domain, wherein the fifth binding domain and/or the first binding domain bind to (i) different moieties than the second binding domain, third binding domain and/or fourth binding domain on the surface of the same cell, or (ii) different moieties than the second binding domain, third binding domain and/or fourth binding domain at the synapse between two cells that are in contact.

109. The composition of claim 108, wherein the fifth binding domain and the first binding domain bind to (i) different moieties on the surface of the same cell, or (ii) different moieties at the synapse between two cells that are in contact.

110. The composition of any one of claims 103-109, further comprising:
(e) at least a second cage polypeptide comprising (i) a second structural region, (ii) a second latch region further comprising one or more bioactive peptides, and (iii) a sixth binding domain, wherein the second structural region interacts with the second latch region to prevent activity of the one or more bioactive peptides,
wherein the first key and/or the second key polypeptide are capable of binding to the second structural region to activate the one or more bioactive peptides, and
wherein the sixth binding domain and/or the first binding domain bind to (i) different moieties than the second binding domain, third binding domain and/or fourth binding domain on the surface of the same cell, or (ii) different moieties than the second binding domain, third binding domain and/or fourth binding domain at the synapse between two cells that are in contact.

111. The composition of claim 110, wherein the sixth binding domain and the first binding domain bind to (i) different moieties on the surface of different cells, or (ii) different moieties at the synapse between two cells that are in contact.

112. The composition of any one of claims 103-111, further comprising:
(f) a decoy cage polypeptide comprising (i) a decoy structural region, (ii) a decoy latch region optionally further comprising one or more bioactive peptides, and (iii) a seventh binding domain, wherein the decoy structural region interacts with the first key polypeptide and/or the second key polypeptide to prevent them from binding to the first and/or the second cage polypeptides, and wherein the seventh binding domain binds to a moiety on the surface of the same cell as the second binding domain, third binding domain, and/or fourth binding domain.

113. The composition of claim 112, wherein the seventh binding domain and the first binding domain and/or second binding domain bind to (i) different moieties on the surface of the same cell, or (ii) different moieties at the synapse between two cells that are in contact.

114. The composition of claim 112 or 113, wherein the seventh binding domain binds to a moiety that is present on the cell at an equal or higher level than the moieties to which the second binding domain, the third binding domain, and/or the fourth binding domain bind to.

115. The composition of any one of claims 1-12, wherein the first binding domain, the second binding domain, the third binding domain (when present), the fourth binding domain (when present), the fifth binding domain (when present), the sixth binding domain (when present), and/or the seventh binding domain (when present) comprise polypeptides capable of binding moieties present on the cell surface, including proteins, saccharides, and lipids; or comprise cell surface protein binding polypeptides.

116. A composition comprising
(a) one or more expression vectors encoding and/or cells expressing:
(i) a first cage polypeptide comprising (i) a structural region, (ii) a latch region further comprising one or more bioactive peptides, and (iii) a first binding domain wherein the structural region interacts with the latch region to prevent activity of the one or more bioactive peptides; and
(ii) a first key polypeptide capable of binding to the cage structural region to activate the one or more bioactive peptides, wherein the key polypeptide comprises a second binding domain,
wherein the first binding domain and the second binding domain bind to (i) different moieties on the surface of the same cell, (ii) the same moiety on the surface of the same cell, (iii) different moieties at the synapse between two cells that are in contact, or (iv) the same moiety at the synapse between two cells that are in contact; and
(b) (i) cells comprising one or more chimeric antigen receptor(s) that bind to the one or more bioactive peptides when the one or more bioactive peptides are activated; and/or (ii) one or more fusion protein, nucleic acid, vector, and/or the cell of any one of claims 166-187

117. The composition of claim 116, wherein the first key polypeptide comprises a third binding domain, wherein the second binding domain and/or the third binding domain bind to (i) different moieties than the first binding domain on the surface of the same cell, or (ii) different moieties than the first binding domain at the synapse between two cells that are in contact.

118. The composition of claim 117, wherein the second binding domain and the third binding domain bind to different moieties on the surface of different target cells.

119. The composition of any one of claims 116-118, further comprising:
(c) an expression vector encoding and/or a cell expressing at least a second key polypeptide capable of binding to the first cage structural region, wherein the key polypeptide comprises a fourth binding domain,
wherein the second binding domain and/or the fourth binding domain bind to (i) different moieties than the first binding domain on the surface of the same cell, or (ii) different moieties than the first binding domain at the synapse between two cells that are in contact.

120. The composition of claim 119, wherein the second binding domain and the fourth binding domain bind to (i) different moieties on the surface of the same cell, or (ii) different moieties at the synapse between two cells that are in contact; or wherein the second binding domain and the fourth binding domain bind to different moieties on the surface of different cells.

121. The composition of any one of claims 116-120, wherein the first cage polypeptide further comprises a fifth binding domain, wherein the fifth binding domain and/or the first binding domain bind to (i) different moieties than the second binding domain, third binding domain, and/or fourth binding domain on the surface of the same cell, or (ii) different moieties than the second binding domain, third binding domain, and/or fourth binding domain at the synapse between two cells that are in contact.

122. The composition of claim 121, wherein the fifth binding domain and the first binding domain bind to (i) different moieties on the surface of the same cell, or (ii) different moieties at the synapse between two cells that are in contact.

123. The composition of any one of claims 116-122, further comprising:
(d) an expression vector encoding and/or a cell expressing at least a second cage polypeptide comprising (i) a second structural region, (ii) a second latch region further comprising one or more bioactive peptides, and (iii) a sixth binding domain, wherein the second structural region interacts with the second latch region to prevent activity of the one or more bioactive peptides,
wherein the first key and/or the second key polypeptide are capable of binding to the second structural region to activate the one or more bioactive peptides, and
wherein the sixth binding domain and/or the first binding domain bind to (i) different moieties than the second binding domain, third binding domain, and/or fourth binding domain on the surface of the same cell, or (ii) different moieties than the second binding domain, third binding domain, and/or fourth binding domain at the synapse between two cells that are in contact.

124. The composition of claim 123, wherein the sixth binding domain and the first binding domain bind to (i) different moieties on the surface of different cells, or (ii) different moieties at the synapse between two cells that are in contact.

125. The composition of any one of claims 116-124, further comprising:
(e) an expression vector encoding and/or a cell expressing a decoy cage polypeptide comprising (i) a decoy structural region, (ii) a decoy latch region optionally further comprising one or more bioactive peptides, and (iii) a seventh binding domain, wherein the decoy structural region interacts with the first key polypeptide and/or the second key polypeptide to prevent them from binding to the first and/or the second cage polypeptides, and wherein the seventh binding domain binds to a moiety on the surface of the same cell as the second binding domain, third binding domain, and/or fourth binding domain.

126. The composition of claim 125, wherein the seventh binding domain and the first binding domain and/or second binding domain bind to (i) different moieties on the surface of the same cell, or (ii) different moieties at the synapse between two cells that are in contact.

127. The composition of claim 125 or 126, wherein the seventh binding domain binds to a moiety that is present on the cell at an equal or higher level than the moieties to which the second binding domain, the third binding domain, and/or the fourth binding domain bind to.

128. The composition of any one of claims 116-127, wherein the first binding domain, the second binding domain, the third binding domain (when present), the fourth binding domain (when present), the fifth binding domain (when present), the sixth binding domain (when present), and/or the seventh binding domain (when present) comprise polypeptides capable of binding moieties present on the cell surface, including proteins, saccharides, and lipids; or comprise cell surface protein binding polypeptides.

129. The composition of any one of claims 103-128, further comprising one or more effector molecules.

130. The composition of claim 129, wherein the effector molecule(s) are selected from the non-limiting group comprising Bcl2, GFP1-10, small molecules, antibodies, antibody drug conjugates, immunogenic peptides, proteases, T cell receptors, cytotoxic agents, fluorophores, fluorescent proteins, cell adhesion molecules, endocytic receptors, phagocytic receptors, magnetic beads, and gel filtration resin, and polypeptides comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27460-27469.

131. The composition of any one of claims 102-130, wherein the first cage polypeptide, the second cage polypeptide, and/or the decoy cage polypeptide comprise:
(a) an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence of a cage polypeptide disclosed herein, or selected from the group consisting SEQ IDS NOS: 27359-27392, SEQ ID NOS: 1-49, 51-52, 54-59, 61, 65, 67-14317, 27094-27117, 27120-27125, 27278 to 27321 not including optional amino acid residues, or cage polypeptides listed in Table 7, Table 8, or Table 9, wherein the N-terminal and/or C-terminal 60 amino acids of the polypeptides are optional; and
(b) one or more first, fifth, sixth, or seventh binding domains.

132. The composition of any one of claims 103-131, wherein the first cage polypeptide, the second cage polypeptide, and/or the decoy cage polypeptide comprise:
(a) an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence of selected from the group consisting SEQ IDS NOS: 27359-27392, not including optional amino acid residues; and
(b) one or more first, fifth, sixth, or seventh binding domains.

133. The composition of any one of claims 103-131, wherein the first cage polypeptide, the second cage polypeptide, and/or the decoy cage polypeptide comprise:
(a) an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting SEQ IDS NOS: 27359-27392, including optional amino acid residues; and
(b) one or more first, fifth, sixth, or seventh binding domains.

134. The composition of any one of claims 103-133, wherein the first key polypeptide and/or the second key polypeptide comprise:
(a) a polypeptide comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NOS:14318-26601, 26602-27015, 27016-27050, 27322 to 27358, and key polypeptides listed in Table 7, Table 8, and/or Table 9, and SEQ ID NOS: 27393-27398; and
(b) one or more second, third, or fourth binding domains.

135. The composition of any one of claims 103-133, wherein the first key polypeptide and/or the second key polypeptide comprise:
(a) a polypeptide comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27393-27398, not including optional residues; and
(b) one or more second, third, or fourth binding domains.

136. The composition of any one of claims 102-133, wherein the first key polypeptide and/or the second key polypeptide comprise:
(a) a polypeptide comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27393-27398, including optional residues; and
(b) one or more second, third, or fourth binding domains.

137. The composition of any one of claims 103-133, wherein the first key polypeptide and/or the second key polypeptide comprise:
(a) a polypeptide comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27394-27395; and
(b) one or more second, third, or fourth binding domains.

138. The composition of any one of claims 103-137, wherein the one or more bioactive peptides comprise one or more bioactive peptide selected from the group consisting of SEQ ID NOS:60, 62-64, 66, 27052, 27053, and 27059-27093.

139. The composition of any one of claims 103-138, wherein the first, second, third, fourth, fifth, sixth, and/or seventh binding domains are selected from the non-limiting group comprising an antigen-binding polypeptide directed against a cell surface moiety to be bound, including but not limited to Fab′, F(ab′)₂, Fab, Fv, rIgG, recombinant single chain Fv fragments (scFv), V_{H} single domains, bivalent or bispecific molecules, diabodies, triabodies, and tetrabodies; DARPins; nanobody; affibody; monobody; adnectin; alphabody; Albumin-binding domain; Adhiron; Affilin; Affimer; Affitin/Nanofitin; Anticalin; Armadillo repeat proteins; Atrimer/Tetranectin; Avimer/Maxibody; Centyrin; Fynomer; Kunitz domain; Obody/OB-fold; Pronectin; Repebody; and computationally designed proteins.

140. The composition of any one of claims 103-139, wherein the first, second, third, fourth, fifth, sixth, and/or seventh binding domains bind to a cell surface protein on a cell selected from the non-limiting group comprising tumor cells, cancer cells, immune cells, leukocytes, lymphocytes, T cells, regulatory T cells, effector T cells, CD4+ effector T cells, CD8+ effector T cells, memory T cells, autoreactive T cells, exhausted T cells, natural killer T cells (NKT cells), B cells, dendritic cells, macrophages, NK cells, cardiac cells, lung cells, muscle cells, epithelial cells, pancreatic cells, skin cells, CNS cells, neurons, myocytes, skeletal muscle cells, smooth muscle cells, liver cells, kidney cells, bacterial cells, and yeast cells.

141. The composition of any one of claims 103-140, wherein the first, second, third, fourth, fifth, sixth, and/or seventh binding domains bind to a cell surface protein selected from the non-limiting group comprising Her2, EGFR, EpCAM, B7-H3, ROR1, GD2, GPC2, αvβ6, Her3, L1CAM, BCMA, GPCR5d, EGFRvIII, CD20, CD22, CD3, CD4, CD5, CD8, CD19, CD27, CD28, CD30, CD33, CD48, IL3RA, platelet tissue factor, CLEC12A, CD82, TNFRSF1B, ADGRE2, ITGB5, CD96, CCR1, PTPRJ, CD70, LILRB2, LTB4R, TLR2, LILRA2, ITGAX, CR1, EMC10, EMB, DAGLB, P2RY13, LILRB3, LILRB4, SLC30A1, LILRA6, SLC6A6, SEMA4A, TAG72, FRα, PMSA, Mesothelin, LIV-1, CEA, MUC1, PD1, BLIMP1, CTLA4, LAG3, TIM3, TIGIT, CD39, Nectin-4, a cancer marker, a healthy tissue marker, and a cardiac marker.

142. The composition of any one of claims 103-141, wherein the first, second, third, fourth, fifth, sixth, and/or seventh binding domains comprise a an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27399-27403.

143. The composition of any one of claims 103-142, wherein (i) the first cage polypeptide, the second cage polypeptide, and/or the decoy cage polypeptide; and (ii) the first and/or second key polypeptide, comprise at least one cage polypeptide and at least one key polypeptide comprising an amino acid sequence having at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence of a cage polypeptide and a key polypeptide, respectively, in the same row of Table 7, 8, or 9 (i.e.: each cage polypeptide in row 2 column 1 of the table can be used with each key polypeptide in row 2 column 1 of the table, and so on), with the proviso that each cage polypeptide and each key polypeptide comprises a binding domain.

144. The composition of any one of claims 103-142, wherein the first cage polypeptide, the second cage polypeptide, and/or the decoy cage polypeptide comprise:
(a) an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the non-limiting group consisting of SEQ ID NOS: 27359-27392, and
(b) a binding domain comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27399-27403.

145. The composition of claim 144, wherein the first cage polypeptide, the second cage polypeptide, and/or the decoy cage polypeptide comprise:
(a) an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the non-limiting group consisting of SEQ ID NOS: 27359-27392, including optional amino acid residues; and
(b) a binding domain comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27399-27403.

146. The composition of any one of claims 103-145, wherein the first key polypeptide and/or the second key polypeptide comprise:
(a) an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27393-27398; and
(b) a binding domain comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical the amino acid sequence selected from the group consisting of SEQ ID NOS: 27399-27403.

147. The composition of claim 146, wherein the first key polypeptide and/or the second key polypeptide comprise:
(a) an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27393-27398, including optional amino acid residues; and
(b) a binding domain comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical the amino acid sequence selected from the group consisting of SEQ ID NOS: 27399-27403.

148. The composition of claim 147, wherein the first key polypeptide and/or the second key polypeptide comprise:
(a) an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27394-27395; and
(b) a binding domain comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical the amino acid sequence selected from the group consisting of SEQ ID NOS: 27399-27403.

149. The composition of any one of claims 103-148, wherein the first cage polypeptide, the second cage polypeptide, and/or the decoy cage polypeptide comprise an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27404-27446.

150. The composition of any one of claims 103-149, wherein the first key polypeptide and/or the second key polypeptide comprise an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27448-27459.

151. A method of targeting an effector molecule to a cell comprising contacting a biological sample containing cells with the compositions of any one of claims 1-48 and 86-89.

152. The method of claim 151, further comprising contacting the cell with an effector molecule.

153. A method for cell targeting, comprising
(a) contacting a biological sample containing cells with
(i) a cage polypeptide comprising (i) a structural region, (ii) a latch region further comprising one or more bioactive peptides, and (iii) a first binding domain that targets a cell of interest, wherein the structural region interacts with the latch region to prevent activity of the one or more bioactive peptides; and
(ii) a key polypeptide comprising a second binding domain that targets the cell of interest, wherein the first binding domain and the second binding domain bind to (i) different moieties on the surface of the same cell, (ii) the same moiety on the surface of the same cell, (iii) different moieties at the synapse between two cells that are in contact, or (iv) the same moiety at the synapse between two cells that are in contact;
wherein the contacting occurs for a time and under conditions to promote binding of the cage polypeptide and the key polypeptide to the cell of interest, and to promote binding of the key polypeptide to the cage structural region to displace the latch region and activate the one or more bioactive peptides only when the cage polypeptide and the key polypeptide are co-localized to the cell of interest;
(b) contacting the biological sample with one or more effector molecule(s) under conditions to promote binding of the one or more effector molecules selected from the fusion proteins, nucleic acids, vectors, and/or cells of any one of claims 64-85 under conditions to promote binding of the one or more effector molecules to the one or more activated bioactive peptides to produce an effector molecule-bioactive peptide complex; and
(c) optionally detecting the effector molecule-bioactive peptide complex, wherein the effector molecule-bioactive peptide complex provides a measure of the cell of interest in the biological sample.

154. The method of claim 153, wherein the biological sample is present within or obtained from a subject having a disease to be treated, and wherein the method serves to treat the disease.

155. The method of claim 154, wherein step (a) comprises intravenous infusion into the subject.

156. The method of any one of claims 153-155, wherein step (b) is carried out after step (a).

157. The method of any one of claims 153-156, wherein the detecting step is carried out.

158. The method of any one of claims 153-157, wherein the method comprises the use of the compositions of any one of claims 1-48 and 188-191.

159. The method of any one of claims 151-158, wherein the method comprises the use of AND, OR, and/or NOT logic, using any embodiment or combination of embodiments disclosed herein.

160. The method of any one of claims 151-159, wherein the method comprises use of AND logic.

161. The method of claim 160, wherein the method comprises use of the composition of any one of claim 102-105 or 116-118, or claims depending therefrom.

162. The method of any one of claims 151-160, wherein the method comprises use of OR logic.

163. The method of claim 162, wherein the method comprises use of the composition of any one of claim 106-111 or 119-124, or claims depending therefrom.

164. The method of any one of claims 151-163, wherein the method comprises use of NOT logic.

165. The method of claim 164, wherein the method comprises use of the composition of any one of claims 112-114 and 125-137, or claims depending therefrom.

166. A fusion protein comprising:
(a) an extracellular binding domain;
(b) a transmembrane domain;
(c) an intracellular signaling component; and
(d) optionally, a selection marker.

167. The fusion protein of claim 166, wherein the extracellular component includes a binding domain specific to one or more bioactive molecule.

168. The fusion protein of claim 167, wherein the binding domain comprises a peptide, wherein the peptide may optionally be selected from the group consisting of Fab′, F(ab′)₂, Fab, Fv, rIgG, recombinant single chain Fv fragments (scFv), V_{H} single domains, bivalent or bispecific molecules, diabodies, triabodies, and tetrabodies; Bcl or a variant thereof; and computationally designed proteins

169. The fusion protein of claim 167 or 168, wherein the one or more bioactive molecule comprises one or more bioactive peptide.

170. The fusion protein of claim 169, wherein the one or more bioactive peptides comprise one or more bioactive peptide selected from the group consisting of SEQ ID NOS:60, 62-64, 66, 27052, 27053, and 27059-27093.

171. The fusion protein of any one of claims 166-170, wherein the binding domain comprises a stabilized variant of human Bcl2.

172. The fusion protein of any one of claims 166-171, wherein the extracellular component, includes a flexible spacer or hinge region.

173. The fusion protein of any one of claims 166-172, wherein the intracellular signaling component comprises a costimulatory signaling domain.

174. The fusion protein of claim 173, wherein the costimulatory signaling domain is selected from the group consisting of CD27; CD28; 4-1BB; ICOS; OX40; CD30; LFA-1; CD2; CD7; LIGHT; NKG2C; B7-H3; GITR; BAFF-R; CD5; HVEM; CD160; LFA-1; SLAMF7; NKp80; ICAM-1; CD94; DAP12; a ligand that specifically binds with CD83; or any combination thereof.

175. The fusion protein of any one of claims 166-174, wherein the intracellular signaling component comprises an ITAM-signaling domain.

176. The fusion protein of claim 175, wherein the ITAM-signaling domain is CD3.

177. The fusion protein of any one of claims 166-176, further comprising a selection marker.

178. The fusion protein of claim 177, wherein the selection marker is a truncated EGFR (EGFRt), truncated low-affinity nerve growth factor (tNGFR), a truncated CD19 (tCD19), a truncated CD34 (tCD34), or any combination thereof.

179. The fusion protein of any one of claims 166-178, further comprising a self-cleaving peptide.

180. The fusion protein of claim 179, wherein the self-cleaving peptide is a 2A peptide from porcine teschovirus-1 (P2A), Thosea asigna virus (T2A), equine rhinitis A virus (E2A), foot-and-mouth disease virus (F2A), or variant thereof.

181. The fusion protein of any one of claims 166-179, comprising a stabilized variant of human Bcl2, a flexible extracellular spacer domain, CD28/CD3ζ signaling domains, and a truncated EGFR (EGFRt) selection marker linked by a T2A ribosomal skipping sequence.

182. The fusion protein of any one of claims 166-181, comprising an amino acid sequence at least 75%, 80%, 85%, 90%, 91%, 92%, 93%, 94%, 95%, 96%, 97%, 98%, 99%, or 100% the amino acid sequence of SEQ ID NO: 27489.

183. The fusion protein of any one of claims 166-181, comprising an amino acid sequence at least 85%, 90%, 91%, 92%, 93%, 94%, 95%, 96%, 97%, 98%, 99%, or 100% the amino acid sequence of SEQ ID NO: 27489.

184. A nucleic acid encoding the fusion protein of any one of claims 166-183.

185. A vector, including but not limited to an expression vector, comprising the nucleic acid of claim 184 operatively linked to a promoter.

186. The vector of claim 185, wherein the vector is a viral vector, including but not limited to an adenoviral vector, a vaccinia viral vector, an AAV vector, a retroviral vector, a lentiviral vector, an alphaviral vector, or any combination thereof.

187. A cell comprising the fusion protein of any one of claims 166-183, the nucleic acid of claim 184, and/or the vector of any one of claims 185-186, optionally wherein the nucleic acid and/or the expression vector are integrated into a cell chromosome, or optionally wherein the nucleic acid and/or the expression vector are episomal.

188. The composition of any one of claims 102-150, wherein an interface between a latch region and a structural region of the first cage polypeptide, the second cage polypeptide, and/or the decoy polypeptide includes a hydrophobic amino acid to polar amino acid residue ratio of between 1:1 and 10:1.

189. The composition of any one of claims 103-150 and 188, wherein 1, 2, 3, or more large hydrophobic residues in the latch region of the first cage polypeptide, the second cage polypeptide, and/or the decoy polypeptide, including but not limited to isoleucine, valine, or leucine, are mutated to serine, threonine, or a smaller hydrophobic amino acid residue including but not limited to valine (if the starting amino acid is isoleucine or leucine) or alanine.

190. The composition of any one of claims 103-150 and 188-189, wherein 1, 2, 3, or more large hydrophobic residues in the structural region of the first cage polypeptide, the second cage polypeptide, and/or the decoy polypeptide, including but not limited to isoleucine, valine, or leucine, are mutated to serine threonine, or a smaller hydrophobic amino acid residue including but not limited to valine (if the starting amino acid is isoleucine or leucine) or alanine.

191. The composition of any one of claims 103-150 and 188-190, comprising buried amino acid residues having side chains comprising nitrogen or oxygen atoms involved in hydrogen bonding at the interface between the latch domain and the structural domain of the first cage polypeptide, the second cage polypeptide, and/or the decoy polypeptide.

192. Use of the fusion proteins, nucleic acids, expression vectors, cells, and/or compositions of any one of claims 103-191 for any suitable purpose, including but not limited to those disclosed herein.
